SEQUENCE LISTING (166 sequences)

SEQ ID NO: 1 - DNA, 1000 bp, Arabidopsis thaliana
gagaattttagaaaaagaagttccgtgaatatcgcaaacattaaggcaagaacatttgcaaaaaaaaaagagaaacaaattcaacatccagcagaactgcagaagtaatgggggtgtttagggcagaagacaagaacattagcaaaaaaaaggtaaagattgactggttccatatgcattcatgtcatgcaaaatattgaatcgaaccatcacaagcataaacattaaggatttctgttagacaaattaacgttgtaagtactaaaactagctagatttgtgattgtcacatatcaacttgttctacataaagtttgtaagagctaaaactttaagcgtgagctttttgtgaaacaaatgaaagttagatattaaccgttctcttattctcgagatgattttcaatcgatggttatgctacaaagttggctcgttcaatataatgagctcttgtgtgttcaacatgaatacatgatatttttgtgctcgttaaatactcggtttttacgaaatgaatttttttaagagttcgaacctaaaacccgaacttgattgaccaatgtacattcatagtagaatatttgctttgtacctgaatctgcgtttatgaaactacgtctaaagattgaaccaaacaaataaaccgagttaaaccaaaatctagatcctaccaaatcaaaacgaatgaacattcaatttagaaaccaaaaaaataaaccggacattcctcattagattctctatattattcttttgtctgtaattgcgtttgtacaaaaccacgtttgcgtttagattgtttaaagatatttattgaaaaaagacccgcgtgattaaaatgtgtaaagaaagttcctccgttattaccgtccaagtggatctctaagagaccgtacaactaactccactcacggcggcgcattcccaatctgctgacacgtttctgcttatcactctctctttttcttctcttcttaccacacctggttgactcaaccac

SEQ ID NO: 2 - DNA, 1000 bp, Arabidopsis thaliana
tatggtagaaaaatatgagagagaatgattatttacataatgaaaagtgattgaagttaccttttaaatgtaactaaattacatgagttacaattctaatttgtttgaatgacaatgagtgaagaagaatataattaatacacaattagtatgatagttacaatttagtattcaatggttgattaataaatgtgaataaatttattgtttttgtgggttaattggagataaatattattctatttttatttttgaaaaattggttgttaaaaatgagaccttatttttatatatacatgattaaactattttgaacttgaatccatttgttttggatttactcacttttccaatgttacttatagtttcgccagtggaatataaggttagaaaatatgagagtaaaaatcaggaattaggacatgtaaaatccagaaatatctgaggacggatctcacgattgattataagattcgcattacattttctcttcatttttttcagataagaaaattggattctatcaatttgtcgtcctattgttcttttcttgtttgatatacaaatacgagtcactgattttttttagttgattggtagagtcttaaaggaaacatcaaattcagacagtccttttacaaccatttcatccatgtcggctcattatttcgcaccacagaagaataacccaaaattattagaaaacttggaccactataagcacttgaactgtggtcgtgcactgactaatgtgagcccgttttggtaattctcagctgtgtgcgtaattgatgtcggcgtcaccttcagaaacttcgaaattatgtgtgatacaccgtgtttcataaattcatacacgtcattttagatacaaaattagatattttcatttgataaaaccgaaagaaataataaactttttctttcttgttctcattttaagtctctctttatatcactttctatatatattgaatcatgcaaggaatacacataaaat

SEQ ID NO: 3 - DNA, 1000 bp, Arabidopsis thaliana
tgggtgaccaactctttcctacttttctaacagtttttggtttgttttgttcagattcggacaaaatgatgagtgttgtgttgtgaacagagaactggtccgtttgtaatatttggggtcggtggtctttgtgacggtctaggctttggttgttgaactacttcctttgattaaggtttatggagttttgctatatctacacaataatacataattgttttgtattattgacttgaccaataacaggtgtcgaaaattctcagttagtacatcatcttttacaagtcttttgacgttctagaccaaattatgttgatttttgaatcaatggtgagttattttagtcatctggttgccgtgttttgaaactaagaaaagttgaattatataccgtaaagaagaagataatactaaaaatccgaatacgacctacaatgaaactaagaaaatcagtagccaagaatgattttgagtcaatggtactaaatggcaaaaaacattacaaactaaataaatgacttttggtggacgtctaataaagtaatagcaatgaataaaagtaaagtttttgcaatagagtctttcatttaattgtaaatggatgtcttgttaaccaaacaatattttgattgttttcattgctatgtgctttattgagaggatgagagcacatgcatgtcttgttttgtaagctgtacctttttttgtcataactcaacatgatagtaatggaatggaatctctttgatctcttgttaagttaccaaaacagaacaaccgtgagagagtggttcatctaatacaataacaagacatgtatctaccacaatcctcacatacacatgtacgcatcccattggccaattcacttccaccaacgaccccaattaacaaaaaccaacatgagttagttattagtagttgacctatactcacttcttttatcttccctccactctctccaatctataaaaccaaatccaacaattgcttccttctc

SEQ ID NO: 4 - DNA, 1000 bp, Arabidopsis thaliana
ttgaattgaatgaagggtgtggtcggaagagaagacgtgtagaagagacgagacaagtaaatttaagcattggccccatttacagccacaagtccgctacaacaaattatttccaagaaactctgagataacgtcgtgatgaaacggctcatgctgctgttgtgattcgtgaattagaggtttatcttttgggtttttgaatgttacttaattggacggtcgatttttcaaactgggtgtgaaatgtgaatgggtcattcataatgggcttttgttttaatgtgaagccattcacacactctttgtccttcttttctattattcataactgtcactctttgttcttcgaaatagtaaagagcaaatcgattctttgttgatctgggccgtaaaatttccatggttgtgggaagtattctcgcagctgatctgggccgtcaatgctacagtttcatgtcagagagaggtcaagaatcaacacgtggccaaccatgattttaaaccaaagcaaacacacgattagaccccacattgtttgttcaccaacccccgtggaccctcctttagccgacgtgtccacgtcaatagtggtttttcttcctttcaaagtacacaaattccattctttctcattttactttttggattacgttgttgttataaactggtaaaatgaattatgaatgcaaataaatttcatttaagttttgttggcttctaatatttttttcacctaaaattctaataaactacacagccatgagccatcgtatgaaaagaagaagaaaaaaaatgtctttttctagaaggatctttcaacgactaaaaaagattttaagcttttgactaattttgtcaataatatacacaaatttacactcaattatagccatcaaatgtgtgctatgcagaaacaccaattatttcatcacacatacgcatacgttacgtttccaactttctctatatatatatatagtaatacacacacataaacagc

SEQ ID NO: 5 - DNA, 1000 bp, Arabidopsis thaliana
tttgtcgaattggtggggtcttaaggctaaaaaagggttcaccacatgtgtatgaatcgaaatctactcatccgctgtcgggaccaaactacatatcaactaccacgtttttctcatatgaatattcaaagctaccgtaaatttttataaaattacgtcattagcataagaatgtatgtacatattaatgactattataaaccctaaggcacggataccttatccttccaccaccgaatttcacggattccatatcctttcaccggactagacgaatatgaagctaagatattaatagattggtgaccaaatttgaatcttttcggataaaacttgtttggcatactttcccttagagtcaattattcttcaaacaatgtttaacttaaatctcaagatcttatgtagttaatggtagatatcgtacagtttttttcgtgtttgtcttttgatcgtgtgtaaagtgttggaaaatctaactttgtatataagttctacacctccaaattaacgttcgagcttatcggtattactgctagtgctttctcaaattcctttttagatcatatagtagcccactaggaaaccttacaccaaaacgcccaaataatatgaaaagcccatcatattgtccatccaaagtaggacaattgctgaaaaagcccaacgaatcaaacctgcattgttaccttgcacttggaaaatgaattaaacggcgacgcacctatggatggagactaagttcactcatcaaaaatctaatttcaactctaattatcgtatttaaggtcattggagattcaactcttgtacctgtagtttcttcttctgaaaatcagcaacaaactacaaatcatccaatgatagaatttcagctttaattatcaaacaaattaaagataaccccattgataatatcgccacgtctcatcggatgaaatagtactatctgacacgacacgaatctcttatgagagaaacagaagagcacggaatctcc

SEQ ID NO: 6 - DNA, 1000 bp, Arabidopsis thaliana
attcatttctccgagaagaatatgtacttttttactttcgacaaaagaaccatatttttagcttgggaatcttggattaaatattaggacaatatttttaacttgagaatcttggtttaaatatgagaaccatatttcagaaaaaaaaagtttatatatgtgtaattttgtcggaaccggatttactaacattcgtaggtagtcggatttggtcccgtaaaataaaatggtaaataaaacaaaattaatagtaaagaaaatgaaaaataatttacaaacacaaattaaattgaatacaaactagctatcataatatttttcaagttggttttcatgaacattttagagcactaagctatataacaaaataataataattaaacatcttttaatcattaagtatttaaagatccatacaaatgttggttacttggtacaaccaacaaggcaaaggcggtactgaataagaatataagatacatgaaaaattgatctactatactttacaaaacgcgttagatattatataattgctactcgttcaattcatggggatgtagctcagatggtagagcgctcgcttagcatgcgagaggcacggggatcgataccccgcatctccacttttattgttttctttttaaggttctttttcttttaatttattttcaatcttctagtcttcaattctgtttttgtttctgtcggaatctcttaaaagtcaatttaaatactttaaattccttgcacaacactcaattttgatactttgaattccttgcacaacacaacacgtcgctatcaatcacagacaccacaaacttggacacttatctttagttctgtcttaaactgaatctccctcttatcaattctgtttttgtttctgtcggaatatcttaaaagtcaattttaatactttgaattccttgcacaacacaacacgtcgctctcaatcacagacaccacaaacttcgacacttatctttagttccgtctt

SEQ ID NO: 7 - DNA, 1000 bp, Arabidopsis thaliana
ggtttttataataaaaagttgaaatttgtgcccttttcaagtctttcttcatcatcttcattctcattctcatttccatcctctgcttgctctcttctcacagccccatcctctttaattaccatctccaatcccctgaaccaggatttataccattgcgattccgcaatccaacactactatctctctagtttcctttcttcattaggaaagagcaaaagcattcttttatggtaaagtttcgatttttcaaattcatacactgtcggattcatacccttttcgctccgggtctggaatttgggtttttgaggtttctacggagtcaattcgagtctgcggaaactcaatttgatagactcgtttcaaaatgttgcttaagggttttcttttgttcatggattgaagttttgcttaaaccagttacgaggtttcttggttcttccatgtatccgtgcgttatctttgatgacccttcactcaaagtcttcgttttttcatgtcaagttgcttcctagacttagaaaattctcgtttttaatggatttttgttcatagaacaaacatttaacgtttcatgctttttctgtgagtcacatgtttttttttcttgagctgattaagtctctttttttgtttgcagccatcaattgaattatatgtcttttagccaataattctgataaacgtcaagaggtaggtgagaaaatcaaaactcttgccttgttatcggttttcgataaccacatctatcaaacgatgtcatttgcttgtttgtttctgcttggacattagtttccaaattattagagagttttagtcacgtagaattatgaagtttgtagtatggcagatggctcgtgagcttgtttaactttatcttatgaatttagtggacgagaaagaagttgggttgtttttggttggacatttattaattttcgtctctttgtgccatgttccatgaagtatcatttcatcatctctagcttaatctctg

SEQ ID NO: 8 - DNA, 1000 bp, Arabidopsis thaliana
ataaaatttttagatatttatatttataaatatgactgcagaagttttaagtttatgatagtttttaaaaagcaattatgatagttagtaacaatttagaaacacgtataatttaatttctttgtttatttttcaaaaatatacaaaaaatttatgtcagcgattttattttattttgtatggtatatgactaatttaaaagtggtactaattagtattataaaaaattattaaaaagtatttttaaccttacgactctatttttttacaagttacaaatgtttcataaaattttattgttaaaacactatgatctaagatattgaaacccacggtcaaagtattgctaacatggtcattacattgaaaaagaaaattaattgtctttactcatgtttattctatacaaataaaaatattaaccaaccatcgcactaacaaaatagaaatcttattctaatcacttaattgttgacaattaaatcattgaaaaatacacttaaatgtcaaatattcgttttgcatacttttcaatttaaatacatttaaagttcgacaagttgcgtttactatcatagaaaactaaatctcctaccaaagcgaaatgaaactactaaagcgacaggcaggttacataacctaacaaatctccacgtgtcaattaccaagagaaaaaaagagaagataagcggaacacgtggtagcacaaaaaagataatgtgatttaaattaaaaaacaaaaacaaagacacgtgacgacctgacgctgcaacatcccaccttacaacgtaataaccactgaacataagacacgtgtacgatcttgtctttgttttctcgatgaaaaccacgtgggtgctcaaagtccttgggtcagagtcttccatgattccacgtgtcgttaatgcaccaaacaagggtactttcggtattttggcttccgcaaattagacaaaacagctttttgtttgattgatttttctcttctctttttccatct

SEQ ID NO: 9 - DNA, 1000 bp, Arabidopsis thaliana
cgtatatagcagatatagttaaatctgttttgtatgttgataaactgcttgatataacgaattgttatatggaagattcaaaattgatgatcctatgataaagatcaacatggatacaagacacgttttttcttgcgatagtacttatatctgacgtcagtaatagtcgtggtggctatgtcagaagaggacacatctgtgactcatgttatgtggtcgagaatgaagtctcgtgaaattgtatttacactttgttgtcatgtagggtttgactttacttagtcggcaacgtatatatccgatttattttattttcttcaaactggaagcttttagaccaaaatttaaactaattttctgaaccacagaaatccaatctttttttgtcactgaaagcggttataatcataaatgtccaaaatgtgatttgtttttgaaatagttaaaaggaagtgaatttctaattaaccttaatccacgtaaaaactttatatacctcaaacaaaatcccagcacaacgaggttaaaatcaaatatagccagcttcaattttaatttattctaaaatgtcgaagggtccggaaagcagtcaaattgtcgcacattcatatttacgttaattagagaagtcgcttcttactcacgtttctcgttccaaacacaatcttttcatgtttctcttttaatttgaccgtcattttttatgatgtgaaatattaattgcgcgaatacttcaaacgtacatctgtgatctgtccgtcattttccttcacgttacgataagtttcaatctaaaaactaacatcgtcgccttcgttgaatcaatgcatggattcgtcgcagcatttattctttattagacaactggcccttcaagcgaaatgagggatacgtgtatatatatgcatccacataaacaataagaaaaatataacccttatttttggatgttatataaaattgctttacctatatgtgagctagcaacatttactcatac

SEQ ID NO: 10 - DNA, 1000 bp, Arabidopsis thaliana
gggaatctagcagaatttttcttcctaataactattttcgagctttctgtttttgttctttctttttaaaaaacttattaagttcttatgaataatgacttgtgaagtttgagttcgtctccttcacaagcaagttgtattggtgttttctactttatgaatatgggttttatatacctaaagacttgttatgttattattcttaaatgttgctgctatgatgattactattatcgatttttactattatatttgttttttttaacgattcgttgaaaaataaacgagattgacactaatgtagcaaaatgtgcaatattatcttcaaaacattacaaaattcagttgtcttaatctttttatttccgatgacaaattatggttacgtaaatagtactatattaaaaaaaaaagaaaaaaaaagacgaaaataaaacaaaaagggagaatttgagtattaaaccaaaaaaaaagggagaagtggacttttgatcaaaagaagagaacaacttgcatgagtaaattagttttagagctgactatttaaaattttaagaactaaaaagaaagtggtagtataatttaacaaaaggatgatgactcatatgaataatagtaggcaaaaaaaacttcaaaaccacaatttcttgttttcacttagaaccttattctgccataattacgtttagttaaattaagaaaaaattgcttaaaacgacagtacaagaagaaggaaacattttctttcactcaaaatcctattatcgcgcaacagaggagtctaagtgaaataataccgtgtcgtttgcggatcagaaatcgagagaaccacgtatgtgtcttttatgtcctggtcaaacggttatccaacacgtggatcccaaaactagccaccgactgcagcaagtttctcgaaccgatgcacgtgtcctcgacacgtggagactcccaagcacgtctctgggttataaatagcaacccaatgctccaatcattttcc

SEQ ID NO: 11 - DNA, 1000 bp, Arabidopsis thaliana
gtcaacattgatttttcaatttattaagattttttttactaatgtaacatagatgaaagtgaggttttctcgttacaataaaaaaaaaagaagctacatagccagtaagccagagaactaaacgtttcttgtacatgcatgttgcatgagaactcttctagtgagagaactcttggcccaaacaatcaattttttgaaatattaaattaagcttacaaatttgcaagagtgatgattgctatattgcataagaggtatttaacttggatttttttaaaaaaaaaaacaaattgggacgaaaatgtagtctttctttttttgtgtgtgtaaagattaatatgttagaagaatagcttatatattgctaaaaaaaataaaaatgaatagcttatatataaaatagcaaaatcaagaaaacgacattacttttaagctgaatagatatgaattttttgaaatgccaaaagttattatataatagatgaacaaagttttttttaaaatatagttataattttaaaacgtacgggataaattaaaagatcactttttcacgttttctttttttaacaaaacttttgttagataatctgaaatattactcattatttcccatattctattaagtatttgtgaataaattgtatacagctaaacacacataaattacaaatttattgatttaatatactttactactttggttaaaaagtaaacatattcaaattcctttatttcttctactttggagctgcattaaacaacttccttgaataattacgtaattaaaaacaaattaccaaatatttagtcaaaaaataatttagtgaatatatcaaaaataaaaaataaaggaaagtcggacgatactaacaaaattattaaaggaagatgaatttctctttccttgtgcgcaaaatagcaacaagtctccgcgtttcctctctttctctctttcccttatttaaacacacttcgtcttctacctctaaacaatctc

SEQ ID NO: 12 - DNA, 1000 bp, Arabidopsis thaliana
caacatgttcagaataataattcacagctgggtaaattttaatttacaattattgagtttttttttttttttgtttatgtcaaactcaaaactagtgccacgtggaggaagctaagcctataaacgattatgaccgacagttgtcactctagaagtagcggtccttgtgacggcataagctggtcataaccgcttagtggtgatgatttgacgaaattgcccattaagcgactgtgagatgtcggtagactacgatggccacgttccacagagagtgacactaacaaccaaaacctattttattactattttttcttttgtcattagtattttattactaattgtttgaatatttcctcgtggtctattattattaaacactctagtcctcttattgttcattaaaattgtctgacgcttgtctcttcctttcgttttctactagaaaattcagctattttcttaatgttttatatctcgtaaactcttaagttgtaacagtgtttcaatataatttgaacacatattgaagtttagtccaaaattcttcttctccagaatatatccaaatcgagtcacaatggaaaacaaattctcctttgtaggtaaatagaaacaaacaataattgtggcattaaagtgttttaaaacgagatcccaataatttacatcagtaagatttcaaaagtgtgtaattagaataaacaataaacctccacaatttggaagatattttgtttccatatttgtcgcgtataatttattatattgtcaattctcgagaatttttaacgttttgttttgaccaggtaacgactgttccggtaaatatgtaggtccaaaaacttcccgcggacttaactcgttttgaaatccgctgtttggttttctttttttattaccggctctaaccggtcctacccaaaaccttgtcatgtcctctcattcctccaccgtttataagtaggcccttaactccaattcccaccgtttcttcgct

SEQ ID NO: 13 - DNA, 1000 bp, Arabidopsis thaliana
tcacgtgttacggctgagagcttcgaagcccacgtcattgctgacatatcttcattcgtacggatcatgtaatctgttaaatttcccaaaacgtttgctacctaaacccgtgaattatttggaagtcaaacttcctctttaatttcggaaatatttacaaattcaggtcaatctaatgtatttcatacaacgagtgtaaatgtattattttgacaaatttgcgtatcttctagttgggttgacaatttaatatacttttaggtcaattttgcatagtagagatatggataagaaagtctatctatatacacacgcacatctacgtattataacaactgtacatcacattcacactatatacagaacattcatctaaaacaaccgtatttattgtatccacacgttttactatgggtgtgtggtccttttaattatcagttactataaattataaacaaaattccactagctaatcaatatgaaattacatttgagtttagaatagtcaataggaaacgatttaaccaaaataaaataactaattcaatttaaatcactgtttttttttgtatccatcaacaatcatatgaaccatttgactacattctctggtcaatttgaattggtatttactactactaatagatactcttataccaatttagataaataaacttaatcctgagtacggactaaaggacaatttgtacagtgagttaaaaatagaaaatttacctaacaatactttttaaatcagtcaaacattttttgactttaataagaaataatatttaatattggccttgtaaaacaagactactctcacaacagtcaacacacacagctaagcatacgcgtcagcttccggtttaaacaaaaaaaaacacaaaccgtaatttggtttccgtttcttcctcttgtaacgaccggttaaataaaatgctgacgtcaccatcttcttctatatatatcctacctggaaccaactctgtatatacgc

SEQ ID NO: 14 - DNA, 1000 bp, Arabidopsis thaliana
tttatacatttcacatattataaatattataaaacgtttttccgttaaatattttaaatgtttaactaactataatatttatttttaataaagtataatattaataaattcctaagatttctcctttttaaaagtaattagaattgatggaaatatattagataaagaaatgctaataattaagaatattaattagttataataattaaatatcaatgacatccactgtaaataagtcccaacttaaggatttatttactaaaatagctccaaaaatgtatatatagatgttatcaaaattcgaattaaaaagttgtaaaaagactcggaggcggtcaaaatcaatttttttattttcttgcataaatttgtgaactccagagaattaagaatgaccagtgatagagctccacgtggatagattgggttccaaagtgggacggcgttaaatataaaaaggccggaagttgtcgaagtagtcttttttcccattatttctgttttcggttccttattttgttcctttgtaataatgagtgacatcaaaaatatttggggacgaattaacaggtaatgtaaatagaaagaaatagaaaattttctttgattttacaatttggattcggattgcataagcaatgacatcaacagtaatacatagctgagaggctgagatgcagtgtctctctctcagggaagatgacaaaaagaatctgatatacacgtacgtatgttgcgagttacatattgatatatcacatgtcataagtcacatgctaacgataaactaataaactaaaacaattatgtcaacatatatcggtgaattgcattatcatcgacctcggtcactgttccgcacatcttggtcgccacggaacatagacaatttttggattgtagtccaattaatgagtcccccaccaaagccgtagtaaaatcgaagtcgttctctaatccaatcatatatatatccataacacacaccaacaacacccac

SEQ ID NO: 15 - DNA, 1000 bp, Arabidopsis thaliana
tgccaaaacccattcttggatcctgttgtcaccaactgcaaccattacttctgcgacaagtgtgctctaaaggtaatcttttccttacaacaagtttcgtaatttctgaaaacacactcaaaacagattttccttgttattttttttcataatgcagcatcacactgagaacgatacttgttttgtgtgtaacgagccaactctagggcttttcgacacagccgtggagatcaaggaaagaatagaggaagaacgtgagaaagccagaggtttgttaaaaacgcaaaactctctgttttgacaccaaaaacctactcttacaaaactcgctcggtgatgtctctttgtagccatggtaaaggaagtgacagcaatgttagaaaaggcatcgacaatggcggatgacgccaagggcgtagcgcaaaaggttgtaaaaatggtggaggagattgaaacaatggtggaaaaagtggcggctatggccacaaaggcgggagaaacggcgacaatggcggcagatatggtgaaagaagctgaggagacgatggaaacagctaaagctaatatgtccaaagcctttgtggtaatgaagtcggtgaattggaacgtgtaaatcgggtcaaaacagagttttattgtgatctcatgctgacatcagctacaatctttatctaataagatagattctcacaagattcttttatctatctactttttagagaagatgagatcatacttcggagatagataggtgtcgtaaaaattgggaagcttacttggcaggagaagagagatatacacacgtgcttaaagtcaacagctaatccaaaaaggtagacgacaaaccagttaaactaagacacgtaatctatcttaaagatttgtcggttcatcgtaaatccgttggtggattaatatatgtcggtgtcttcgttgattttcttagcctaaccaaccaaacataaataaaaatcgaagctttactttgtttgcttctatt

SEQ ID NO: 16 - DNA, 1000 bp, Arabidopsis thaliana
ataatataattctgaaaataactaataatttactcttttcaagtaattattcaaaataaatttagaaaactaattcaatgtatttcctcattgtcactcttttatttccctctacaaaattatatatatgaagtagttttttttaaaatagagccccataactaacattaggggtccatatatttgcattgtaatttataaacacgtgtagaacattataattatttaaaataaaatttagaacaataacttaatgtatttcctcctcgttagttttcctttttttccctctacaaaattacatatatggaatagttgttctttacttcttttaagtagagccccttaactaacacattaggggtccatatatttgcattataatttataaacacgtgtagaacctggcactgaagtctgatgaatatatatccatttgttttcacaactaccctagctttttgtatcatcggttactttttttttatcacaagaaatttctcgattttgattttgatttttcaatgttgtgaagttgagaagttcaacatgttttggacagatgtttttgaacagtaaattagtattttaaacatgtgaatctatttgcaaacgtaatgacgtgtcgtctgaccatttcttgaatgttagttaattttttagctctagttttgattaatctaccaattaggtcattcatttcagtttaagtcaaatcaacaattaggctcgtcctaacgttttcatttagccaaaacaaaaaaaaagatttcaacagtataaataattcgtccttatcaaaaaattaataaataaaaaagatatacatcattacttacgtaattgtttgtattttgatagttttcgataatttctatgtgacggtttttaaaataaaaataaatgttgaataacaaaaaatagtagtacaatagaatactgaattggtttgtgtagggtcaaaccaacaaatatggaaagaaaagtattatttagttac

SEQ ID NO: 17 - DNA, 1000 bp, Arabidopsis thaliana
gccctagcctaaatcaaaccagagataactgtggaaatgagactagaggattcaataaaaacacaaacttttttggatgtgcaaaaaaatcaaaattggtacctcttgttcctttttaagatcttgttctggcttaatccagagaaagtaagcaatagttccggcgacgacccaagaagcggcactctgaccacctctaagacctcccatggaattaccgatgaaagatctcacgtttccaattgatcttctccagctgctagccattgattgatgcccaactatacgaactggtctaaggttcttctggatctcttctctgacaattgcttgactttgatgatgattcgatttggaactgaaattgcaagaaaagtaaaagcaggcgatttgattttgattcgcatttgcttggtgaacactgccaaaggctaaaccggtcgccgagacggttaatctgatgtttgggattgacacatcatgcggctgttgtttctccacgtaacatttggagacccgctcaaatatattgtgggccggagaacattgttttaccatatagggcccatagactttgaatatgtgtaggtaaataaaatacaatctgtggaacaaaaatggcaaaaatatttgaagtcagcaggattggttaacaatttgagcacagaaatacatttggtaacatctgagcatatcattcatatcatatcgctgtcgaatttgaaggaaaaaaaaaagactaaggaagtggatatattggtgaatgctgatgatgagaaaacttattattatatacgaaaaatttactaaagacagcaatattccaaaataatgatagggaagattctgagatggtgtgtggtcagtgtggaggcactacagatggtcaaacagtagcatacccacctggctatttggacaaggacaaggaagaagaaaaatcgagaaccatcttctcagccgattttagtaaccatcttcttgtttgctccgaacaatc

SEQ ID NO: 18 - DNA, 1000 bp, Arabidopsis thaliana
aataataataactaattagtaattagtgggtattttaaataccattatgccatttagctaaggtaaaaaatcaattgttaattataacacccaccaccaacatagattcgtctatggttcattcatcgtgtttaagagttgaaagaaataaattcgcccatcgtgaataatagatatttattcatctcaaacttataactcaaatatatttcaactatatatcgatcaaatagtatagcttttggttaatgacgaatttttattcttttgatccataattcttcttcttcttttttttctgtccggatacatgaaattgatgatacacatttaaaacaacaattcgaatcagggaacaaattacagccacaaccagtgatgatagaccatatatatgatttagtttatttaataaataatgtgtaagcaaatttggctttgctgtaaaaaaagaacacgaattttggcaaaagtttctgttaggaatctggttctattctcctctgcaacctccagtctctcatgaatctggttcggattctctttttccttgtttctatataatttagatacatggttttataattctatcatatgtctattttggatatagtattttaaaaatatatatatttttcataaatggttatggtctattctatgttaatgataatcattagtctttttgtcaactatgtttttttttccaacaaatttagtatgtaaacttttttttactaccgttttattaaatcgacggttgatcagatcaactccggtataacacaacataagtttcgttatcaaaacaaaaacaaaaacagatttttttttgtcaactaccagtgaagattagtcttacgtgtcaagaaaccggataaaaatatataacgtatttgggcaatcagctaagatattaactaacgcggagttcattattaaaatggagtaatgatgttttcagttttctatataaatcacgtcgagaccgtagagtctt

SEQ ID NO: 19 - DNA, 1000 bp, Arabidopsis thaliana
taatttttttttctttattcaaattatttcaaaatacaattatatataatttattttttgtttgaacaaaattttacatttattccgaaaaaaaaaactatgcaaaagaaaattttaatccaacttctcatcatcctcaaaataatgataacaatatatccgcttcattagtaactgtaaccttcttcgatcaaacaactcatctgcaaagactagactatttcatttatctaacattaccgttaacaaacaaaaaacaaaaatttacttgggagtcgaagaagtcaaatatctagaagaatacttcttcttacaaaattataaggaaaatttcccctactgcttcgagaaatacctttttccccattcacgttatgttatgcaacgtgtgaggataacgagcggacaacacagcccttatccttattttattggtcaaccaaccccaaaacagattttaagacggagtgtatttcttttgtcaactgtttacatgggttaaataaaacagtttcactaaaacttattaaattatcatgtcatattgtcatgtatataagctatagcatatatacggatggaatatcgattaaaccattttctttttcaccattggaccataatcagataatatacatagattgggaaccccaaatattttcacattttgacatatttagtagcgacattaaactacttcgtgaaaatacataggatattatgtcaaattgtcaacgaaactttttatatttaataagaaaacgaaattaaataataattaacagaaaatactcacaagtgactttcaatgattttgtctctaacgttttcaatgtttattttttggtctctgactcttcaaggaaactggtaaaaccggtgtcccggtagtcatcactcacatctcgtccaccacgtgtcactcttacacgttcataagttttcccacctttctacgtccgattctgtatttatataccctaaacccatcgtcttctat

SEQ ID NO: 20 - DNA, 1000 bp, Arabidopsis thaliana
gaataatactcttatagagattttagtagattttcttctgcacccatcttcttttttgccactagaaaaagttacataaaaactgtaagttgataaataaataaaactgtaaattccaacaattagtttggattttccacctgaaatctttatagactatgtttattcctcacaataaatagaagatatgaagaataacatcacgcatagtcgatatgcacgtgagataatatgttgagatttagttaccaaaaaagaatgattttgtctgagaaatgaaattcctctagttgtgaatttttcaaacttggtcggtcttgtgagttgagtatctttttgaggggaggtgggaataataataagcaaaaatatcttaaaaaaaagcgaaagctgagtttgaccaatcaccgacccggaagattgaaactataattagtactacgtttggttgtcatcaaaatggtcattcttatggttatgggatattatcctttgccgcggctagccgaccattacaccaacatcatttttttttgtctccattcaatttgcataaacctcttaaattatagatgttttttacagtctaatcttatcttttaactttgtaagtggtgtcgccttacgaaaattaaagcttggaaaataactaaataaagttgaaggatatgaactaatgaatgccttcaccgtccaaaaaaacagagaatgccacgtattcgtacggacgttaatgttttcatctctgcgcgttgtgtttttatgtgtcaatatctctctatttttcaaatgctcattttcttaactttcttattggccaatgaattggagtaaaagcaaaggaataaccccaaaaagatagttttgaatagtcgtcaatagatagatgggacatacaaattacaaaagcggttagcaaaatctttcgttgaaaaaaataaatatctaattcttgccctcatatataaacccatgtagagtcgtctctctcttaccaaat

SEQ ID NO: 21 - DNA, 1000 bp, Arabidopsis thaliana
aatggatgaaaggatagatcatacttacgtttgattcttgattttgattttgattttcgttagtttgggtagatgccataattgagagaatagggtttcaatatattgattaggattttctgataagaataagatatatagggtttgggtggaagacataattattttagttaattaaaaaaaaaaattaacctaaccgctaccgcccgcaaccgcaaacgcttgcgggaaggaacttttaaaatatggcgatttcgagcggtccaaagcggtatctaacggtttttatgattggtgtcaaacgctaacaactgctaccacccgcaaacgcagcgtttgcgggtgacagagggagaaccaatcaagctctaagactaatcggaccaatttagcaacttgtgtgcttcgatgtgttggactgggctagattgggcccaaatagcctgttttaacattcccaaagatccaaacaattccttcttctttcttccgaacaattcaatgtagatccgagcgaatctcctaaattactaaaatgcacaattgtgctcagcttaccaaaaaggcccaaaagaccaaaacatattctgattttttctctttagatacaataaaagtaatttatatagattagaggcccaataaggttcacgtaggcccaaacatattaaaagtaactcagaagagatccatcaaaattttgattcacacatctgtttaggaattaaaaatattatttgatcggttattcattccttttcatgaaatcatgcaaaaaatcaaaaatcatttttttctctagaaactacgtggcgagaaagcagagcaccagttgtcttcttgctctgattatctcgttgaaaccgctttcaaagcagagcaaaagagacgacaccggagcctccactgctttacttttcctttaaactgtgactgctttcatttatataataaaatacatacactctcagagtcacatgtactctcctctaacataaac

SEQ ID NO: 22 - DNA, 1000 bp, Arabidopsis thaliana
aggacacctcattctcattacataatgtgacgagattgagctgtcgttttgttgtaaatatatggtcgggggtcaattattcatatgcagttttaggtcatttataaagaccattaaaaacgtctttaatatttcaacaatcttataattcaattattaagaagcatctagatatggattcacattagagttcgtattctttgacagcaaattcacattagacttttgatatatatattgtacatttgtaatatagtatacgaaaatattacttgaaaactgatgttatgtgttgccatgatgccattggtccatgacttttccacacgaaggccaaagcatactcttatattatatgagttgagtcattcttttttccttttgacggcaagttgagtaattcaattctcttgtcacttaccggtgatataagagttaatcttataaatagttttctgaaacttaatatactataacaatgtaaaagtcgtcgctttgttatttgaagtgaaaattaagcaatgttatgatatttttactaattaactcaatatgaaaaacaaaaatcctcttaactaaaacagaaacataaaagacgacttagtttttgctttagatctagactcataactcaaaaaacaatttcattataaacttttgtagatcttacaattttaaaataaaatgtacattaatgttgaaaagcaaaatcttaaattagtgtatactactactttttttttatcaccgtgatagatcattagatccttaacctcaatccctagagcctgcttattgcctttaagcattgtgcaatcactaccaaacacactcaaaactaaataaatataatttataacttatcaaataaaataaatactatcaagtgtgtatgaaatcgatgacaatttatttattaccttattaccatttgtgtcttgaagtacacgttaagctatttttatgtgttataaaggcttcttcgtcaaccaattacgaa

SEQ ID NO: 23 - DNA, 1000 bp, Arabidopsis thaliana
acatgcatgattgttaacgttttctttttgtacaaggaaaaaaaaaaacgtgtacctctagacaagagttagagagaaatgagaaactggaccaggagatgtgaatggtttcattcctactttggttaatcattgcaaatgccaataattatatagagaacggcgtcaattttctgcaaagaaatatctttactggtacttttttgtgctacctataaatgtgttggtttaatctagccatcacacgtatatttctagctatgttagatctttaatcattctcacctagactcctttacaaaaaccaatatagatgaaaccagtcccacccctctcatttaagtcatagtatatatttgtgtaacgagaatacagacaaaacaattaaattcattgtaatcatacttgaaaatatagtcaatgaaattaattatcctacatttttgaaaacagtgtctataattattatgtacattgaatagatacgtgggatattttggattccagttcatatatatgtagattaaaaataaagagagggcgataaagcctaaagaaaatgtacaaatcgtaatgtaatctagtgatgatttcctctatataggttgatgttgggtagacttttggtcaccatgatatttatctatcgtctataaagtacaaaactgtggtactaaatgtgatttatgaaagctaattaaaaagaaggcgtgaagaaaatgaaatcgtaatagacgacgcgcgtacgagatgagagtagtggaggaaagaagtaagattgagtgaatgataaaatgcaaacgctacctactaatatctccacttgtcatgcaaccagactgagttcgttttccttttcgagtcttatttttttgttttttattctactcaacacgttacacgcttctaataaactctaaacattaaaatcaaaatattttgactacaatggttattttggagctatatataaaccacctgagcctcctcagtttcctcc

SEQ ID NO: 24 - DNA, 1000 bp, Arabidopsis thaliana
catacatatattacgatgcaaacaccgtattttagagctacccataaaattgcttacacaaacaatagctgtctgttttcacaaatttataactccaaaccccacaattacaataaccaagagacacttaaccagatttcggtttacactagaattatatccccattggtcatctggctcggtccggtagagttccccaggcacaccggttacatacatgtatttcggcggaggtggagatgaagatgacttgctgcagtatgtctttcttggtggtggtggccgcaacggagttgtaattccggtatgtcctccgaggtaagaatcggaattggtttgattagtgagatagtgtaacacgaggaacattgagaaaaagactagaatcagaggtttaagtttcatcatgatcatgattattatgattaatgggaaatgtcttttgatttcaagatttgcaaaaaataacacatataaagcatacatatataaacgaaatgcatgtgcatgtaaagtatatgcagcgtcatatctctacatcacctatatgcatttggtggagaccagatcatacacattaaaatgcaaaattgtaaggttcttcagttttgaataagtcttagattgacgaggtcaattttttctggttaccattagtcctatataaggttataggatttttgtaatggactggcctggttgcttttgaatccggcctgttttaaggcccatctctttttcagtgaagaagctaaaaaaagagttttaatttcgtttgaaaattttcgggagcataaagaactatcgaagcaacacgtatccaccttaaaccacatgtatactctagaacacaacacgtgttaaagtgaaagatgagaataatacggaccgaccctacgtacttgataaaaacaattaagaagaagaatttaaaaagagagtgaaatgattcgcttgagtctctatataaacatcacaatccaactctccatcatcttcc

SEQ ID NO: 25 - DNA, 1000 bp, Arabidopsis thaliana
aggtagtgataaatttattaagataagtttgtttgtaatttattataaatcgtcatttgctccttgcacaagtggtaatgtgtctatggccttggcaaggtcaaaggattccaaattcgtctttgcaatattgttctttttttgtttaatgtccaaaacaaacatagttgcattactctaacatatttagcagcacaaatatacaattttgtactgattaaaacacaaaatatttttctgattttcataaactaactcaaacttggaaaatgatcttttaattcttaactcaaacaattttgacatgactgatcgtttttttggatacgtaaatattatgatagaagtaataagaaatgtgagaaatttttagttttaagaagatgaaagacctagtaaatagatgttcaaactttgaatacatcaaacagatgagttaatgctagtgagcaaaattccatagtcaagtcttccaaaaaaaagtcaaaacaatcatgttataaaagtttgtaaccaatatttacatttaacacatttataaaatgtgacatcaaaaacgcggctataaaataacgttacgatattaaaaatcaaaggactaataaaactcagaatttaatttggggaaagaaaaataaaccctgcaagatttgaagtgttcctcctaatcaaccgtaatttagaaatggtccttaatggacggtggtcgtcgtttcacactctccgtccaccattttttttttttgtttctcctcctctaaaaagcaatggtctttcaagctccatcaatggctactactacttagcgtctacgaataataacccctttaccataagtccatagatttatctctcttcttctcaccactcatgtccctttctttgtcttcttcctctcaccgccttcaaagttttcatctttatccaaataaaaaaatcgtttcttttttagtttcttcttaacatatttaaaggcgtaccctttctcttacttatcctc

SEQ ID NO: 26 - DNA, 1000 bp, Arabidopsis thaliana
cactaacgccgtttactttcccgcttggacgataacacccttgatatatggattagtctctcttttgcttttggatatcttaatatattttaatatgtccatttttatattgacgaccatttaattaggtctcagtttgtattattgatttttaggtgccaagcattactttcgttacgtgtaatcttttgattattcaatcaaattatcatatggttcatggcattttatcaattgtgacggtcgacatgtgggttgactttgatgtatgacttttagcttgtgcctcatttttattccaactcgacaactagcagactttatattttaatttatttctttctagatattagacaattcttagcttgtctctctgcatgaatcacgcgttaatatacttataatagtaagttttaaaatgtttcgagtataatatcatattagatttcaattataattgtaagattatttagtctcaatgaagagcatttaaaaatgtgaaagagtttaggctttgactgttgactggttctaacgcgttttgcttcttatttatacaaaaagattttattttattttaattagaaatattaagtactttttagacgtgtttaggttgattaatttcttgttaacatatccgataaaaaaaaacagcaccaaaatcacgacgagaattatgcctacgttacgttgataataaagattagagataaaatgtaacctctcttctctttacatcattacatgcatcttgtgttttatttgactttattcttaccaacaaacaaccaatggataaattaggctcgtgaaggagggcaaactcgtaatttcaaagaaacggaaagcgtcagaacgtggagaacagtgaaccgagacacattcccgatttttctaaagaaacaaaataaaacacgtgttttttataccataagtaaagtagtagtatttattaaactattaatatatagctttgttatattggaggtaggaagaa

SEQ ID NO: 27 - DNA, 1000 bp, Arabidopsis thaliana
aaataaagttggacaaagaaaagaaaaaagtgtttggcatttaataaaacgtctcattcattgcgaagagattagatagtagagaggtcaaattcaatgtcgcagccgacaatagataagaagagataaggtcttcgttatatttttttgtttgttttttaactgtcacgtgaaactgatatccacaaagagcgaatgagagacgtagaagagtcaaagattaaaaacccaacgaactttgactcatcctttgaaggtgttcgttaattatccattttttggtctgactcgtggcatgtggcaactaccgaccttagataagcctggtcctactctcgtattcgatcaccacgtgtcgatcggcttatccgacacctcgagtgggagttgtatgttagtaactagataacggtcataggtacgattatgacattgacatgaaatcatattccaactatcaacgttagtgtccttgtttttatcccctgtaattcagtcaattaagccatcgtaccaggtgagtctttgatattgttgttgtctacgaaaaaccattagatgatctctaattgatatttgattcaacctatggtaaaattatcccaaaactcaaatattacttcaattgatatcatcccaaatattacctagagaggatcaagctttttaatcgtcaattttggttatacaaaacgataaaaaaaaaattgtaagccaaaaataaaaagtaaaacgaaattgtgaatttttaataattcttttgcataatacacaaaagaaaaaaaactcatactccacatgtcaagtgatgacacaataaatgtctaaatttttacaatcaaaaacaaaaaaatgtataaaaaattcgtgtaaccttttttttttgttgtctaaaaaaatgacatgattttggtaaatagccaacaaatttgtagtagagtagtaaagttaggtttcatcatccatctctataaattctcaagaccgacctatacatttt

SEQ ID NO: 28 - DNA, 1000 bp, Arabidopsis thaliana
tcaatcattaatgtatttatgatttgatcgaataacaataatatagcttattttgttctattctatcgtatttgattcttctttcgttttttttttgtttgacttaagaaaccgattgtttatagtagtaaacatttgtttttaatgttgctcgattccagtgcacatgtccaggctagacacttgtcgttataaaggttgctttggttcaatattgatccactagagatgttacaactattgttgacatctgagattgtgtgataagaaaatatgaaactggatttagtgaaagttacaatatataatcatacatcatagataggaaataaggaaatgtcagatatacttgaagaatacatcaaatagacaaggtcctttttcttattgtcgactattatagagccgtacagaaccttttcacgtctttagtaattagtacattctccatttcggctctctcttattttttttccatctcttttacttctccaaataataacaataaaagcttcgattttgtgtgtgtttgtatttacatcttgacatcgatattcttttcatcaattttttaccaaaaatgtaataaaaacaaaaaaaaaccaacgctgaacacagacatggtttctccatccgtttatattcatcgtttgtatgtttacttaacaacttatttcaaaatagtacatatcatggttgtgtttttaaaaaaagtatacagaacagaaaagcacatggtagacaaaataatgaagccaaaattaatacaaagaagaagttcaacttgtatttattaacacattttctttccttgtcaaagacatgcaaattggttttgttttcttattcccattttttttttataataaaaagaagaagagtaaaacaaaaaaactatcatttcttcttatcgcaaaactcttatctaagcaagaaaccgacaaaacctatatctacatatattctcatcaacatctcttgagacatattcattttggtt

SEQ ID NO: 29 - DNA, 1000 bp, Arabidopsis thaliana
cttttgtctttttccagcctttgtttacgtggaatctcggtttctaggacccactacactccttaaataagaggtgcataattttatatttaacaaatacaattagcattagattagtccaaatatccggagtgattttttttggtttttagaatatttatttttttattgtaaattataaaaaatgttatatataatttttttatagataatttttttaattttattagatttatgatatacaatgtattctttttttatgttataaatatttttaataaaatttgtttttaataaaattttggtgaacgattataattatattttgttgtaaattttttatacatttatttttattaatctgtaaatttgtaataaattataatatttgaaattcgaattttaaattttttggtataaaattattaataaacttttaatcaattaatagatcaaaaaaatttatgtccaaattaattaattaaaatttttttaacctacactattgtttaatgttatcacaaattataaatttttaaattaattattttgtttggtcggcaaattaagatattattatcaaaaacatttttttttttttcgcaacacattagttactaaatgaactattaagttcacttatcttatccaatttgtgtctaccctacataaaccttgtacttatccctaaatcactttagataaattgttgaaaatttaatttataaattttgtatttaccaaattaggaaaacaaatatctgaaaatatttatttttaatatcttaacaactcgaagaactgagaaacgcgacaaaaccaatcgtcctcttccgatagccacaaaacaaaaatcagacaagaagaaagaagaacgtttcttctaacagatagagattacaatcaaattgactcttaatttctcaattccgtatctctcatttcatcttcttcttcttctcctttacttaaggatctctggtttctctttctctcctct

SEQ ID NO: 30 - DNA, 1000 bp, Arabidopsis thaliana
ttagcaataatatataacccaaaaacttatctctgaacaaattaattaggaagtaacgcacaaaaagaaatgcacaaaaataaatgtcagaagtgggatttgaacccacgccctctttcgaagaccagaacttgagtctggcgccttagaccactcggccatcctgactttttgttaaacttatacgtatatagtgtaaataatcgtataattttcaatatccataccaatcttcagactcttttttttggtgacaaagtgtatgtattattttcagaagctatatgtttttcctataaaacatccaagactggcccatacatatttttaagaaaccctatagtgtgtatatgacttcaaaatttcgaatggttagtttttctttcgaggaccaaaacaaaaagcccattcaatcactagaaaaatatcactagtcaatcaatagaccaaaagattgaaagtaggatatatttgtttaataatgcctacgattctgcgaagacaggagaagccatacctttcaatctaagccgtcaacttgttcccttacgtgggatcctattatacaatccaacggttctaaatgagccacgccttccagatctaacacagtcatgctttctacagtctgcaccccttttttttttagtgttttatctacattttttcctttgtgtttaattttgtgccaacatctataacttacccctataaaaatattcaattatcacagaatacccacaatcgaaaacaaaatttaccggaataatttaattaaagctggactataatgacaattccgaaactatcaaggaataaattaaagaaactaaaaaactaaagggcattagagtaaagaagcggcaacatcagaattaaaaaactgccgaaaaaccaacctagtagccgtttatatgacaacacgtacgcaaagtctcggtaatgactcatcagttttcatgtgcaaacatattacccccatgaaataaaaaagcagaga

SEQ ID NO: 31 - DNA, 1000 bp, Arabidopsis thaliana
aagtgagtataggtcaactactaataactaactcatgttggtttttgttaattggggtcgttggtggaagtgaattggccaatgggatgcgtacatgtgtatgtgaggattgtggtagatacatgtcttgttattgtattagatgaaccactctctcacggttgttctgttttggtaacttaacaagagatcaaagagattccattccattactatcatgttgagttatgacaaaaaaaggtacagcttacaaaacaagacatgcatgtgctctcatcctctcaataaagcacatagcaatgaaaacaatcaaaatattgtttggttaacaagacatccatttacaattaaatgaaagactctattgcaaaaactttacttttattcattgctattactttattagacgtccaccaaaagtcatttatttagtttgtaatgttttttgccatttagtaccattgactcaaaatcattcttggctactgattttcttagtttcattgtaggtcgtattcggatttttagtattatcttcttctttacggtatataattcaacttttcttagtttcaaaacacggcaaccagatgactaaaataactcaccattgattcaaaaatcaacataatttggtctagaacgtcaaaagacttgtaaaagatgatgtactaactgagaattttcgacacctgttattggtcaagtcaataatacaaaacaattatgtattattgtgtagatatagcaaaactccataaaccttaatcaaaggaagtagttcaacaaccaaagcctagaccgtcacaaagaccaccgaccccaaatattacaaacggaccagttctctgttcacaacacaacactcatcattttgtccgaatctgaacaaaacaaaccaaaaactgttagaaaagtaggaaagagttggtcacccaaattaagccacctttgcttcctcaattccttatttataccatcaaaagactccacaatatcc

SEQ ID NO: 32 - DNA, 1000 bp, Arabidopsis thaliana
aaaacatcaaatctaagtacaatgatagacgacaacacagttcctgcaaccatggaactcaaattgaatatgcaatgactcttggttaaagacagagcctttgggatattacatacataaacatataacttaagcactaattcatgaatttacatgttatatatgtatgatgacgaagttccaattccaagaaagatcaaaaactacaaagttacctcttttacttctggcggagtcgttgacggagattcactccggctcgttcttgctgcgacggtcgaaaaccacagaaaattgtcgcaggtgggtctggttagtgtaaaaaccgataaaggagggattttgacattttgttttggaggaggaagacgaggagatttcttgaaccgaatgtcccggtttttctaaaaccacttctcagactcccggttcatattaaccagcgcgtgcgtgtcgtctggttttcatcatgaccaacggtcaagatcaaaaggatcgccttttttagtagatggtgttcgatgaaatgctcgttagagacgttaaagttgtagtgacaacaaattagtgtttatcattttcagaatcggtataatggaaatgtcagtttcttcattcatttgaaattgtgacaagatccaactccatcatttcaccacgcaataactaaactgttagggacaaaacgcaacatgaatgaatcaatgtacacaagtgtgagaacctgatattcctatcattgaattgtatggaacatgccaaacttagagagtaaagtgtacgtgttctattttgtaatgcaaatatgatgcaattcaaatcaaacacgagtttatctcctttggcacgtttctcaatatagattgctgcaccagaaaaagacaattttttctgtttgaagcctcttttgacatgaagcgaaatgcggcaagagaaccaagaagacaatagcatttttgcttttctttggttaccactctctaacaagataaagaaatgt

SEQ ID NO: 33 - DNA, 1000 bp, Arabidopsis thaliana
ccatgttagctaaattgatccatttggaattttggattctatgtatttagttcattatcaatgattcgttccaagagaaaaaaaaaaacacttaatagattattagttttttttgttttgaacaccaaaatttttagaaattactcaacagaaataatttatcttttgtttttttagtcgacccaaacttgtgcgttttagtctttctcttaatctttggaaaaaaatatctgcagtgtttttatatctactcaaaagcccaactttcaaagttctaaaattcaaagcccaaagcctaacagttaatccagttaaaactaagttcggaatccatgtaaggcttaatggtccggtcccgtcccggtctcaaaacggtagtcgtgactcgtgatatcttgcagaatccgtaattccgtatacttgacgaggtgctgtaagtattagatgttatcgtgatgtttctgctcttcttcgatcgccacatcaaaattataccatatttttgaatctaaagattgttacgaatgcataagcatatatgtaagaaaacaataatcacctatatttatcaaaattaaagatctatcgattgatctttctgacttgcaaagagagactccacataaatgtaaacacggttaaaatacttgaatacctcaatcattgtcgtgccacgccgacatagtcaattagtcataacttgaccctccaaaaaagcctaacgtaagagagtttcacaaacttatttccaattatcttaagccacgtgtttagtgacctttgtgctggccctgaaacgtgtctatcttatcgacccctctttaaactcaaaactcaaaaataataattaaatagtaacaggccgacataataacccaaaaatatctataacgattaaaaatagtaacacactgaccctacgtggaaacaagaaagatattaaaaaaactcatatgattcgtctctataaaagatccaactctcacaaatcaaacaaatc

SEQ ID NO: 34 - DNA, 1000 bp, Arabidopsis thaliana
ttatccaatccttttcaggccattgacttattttctaattaacctaattatatatataagaaaaagctaaggcatcttatatttatatatacatatatcaattgattgctaataatgttttgattaactctgcatatggcatgttttccagcgatttaagttcacaaagaagattttcgtttttgtcatcactaaaaattatctagttctttctactttttcagcgtacaataatctttttttctatcaattggtacgttttttattttctcaattttaattttgttttctaggtacatttttaatcgaattgcaccaaaatatccaaacaaatcaataaaacaggcagaatgaggtattaccgtattagttaaacgagaagtggctactttcctcagcgtgattaaaatgcttatgggttcagtttcataattctcatatacggaactacgcatttgacctcatgctctctataaatattaagatctgatgttgagagcccattagaactaagtttacgaatgagcccatgatagtttaacttgggcctaattagtgtctaatctgcttctaacgttaccgagtcaaacgcggtctaaactaatttgtagagtagtgtatttgtccaaagcgtacccacacaattgctgagaaacttcaaaccctaagtgtgtgtctagtattttgcaatatacgacgcataaagaaatatcctaacattgacaacaacaaaaaaaaatcttagcggagatatagacgtgtgagacaagagctcacacgtgttgcacattcgagacttgttgataatgacgacaacaagaggttctagaatattcgtgttggtttcattattattagcaaataagaccaagtcaacaaactaacttaaacattgtggatcgtccacttgtccgactcttacgtcaaacccatcattgttccacatatctccacgcgtcaatctctctataaatacgaacaaatgaacacgtatcaattc

SEQ ID NO: 35 - DNA, 1000 bp, Arabidopsis thaliana
catctggtacggatcttctagtttctatcaataatcagagtacacaagaactcatcatctcgaatatgtattaacgtctatattgtatgtttcaattcagaaatcagtaaactctggtattttgaagatgaaccggtccctaagtctcacatggataaatttacatcctggtacgagaattaccgaagtgaaatgtcccaagcgatgatggaaactgataaggttaagaggaatcaattaaccaatgaagttatccagaggtacaagcaggtaagctctcgctcaatccaatgcaaagatctgaccaatagatttcatgaatcacattttttgttgattagtccatataatgcttcttaacacaactcatcggttgaacggtttcttctgtctgtatcaggatttctatggcgctgcagggtttgaagacagcaataaaagtctggaagaactctacccccaagccttggcactctacaacgtcgtttacgattatgccattcaggaaggtgttgcgaaatgtacatttgcctggaatgttgcaggaccggtcctgtgcaaattttaccttaagaaaacgaaggataaatcagtagtggcttcaacgtctgtgcttaaaaagcttttgggttgaacagacttacttgtcctgtttgttgtcttcatgtatcataagatgcgttagacaagtatctgacctaggtaaccgaacttataggcgaagctatgtggtgtacttcctttgtttaagttataacttaagatttgtcatctagtgtacaagtaattccgtgtgtatgtttgcttatggaataaataaagaaaactaatgcttatatttaatttcaattaattgtacgtggcactttcagagtccatttggtgtacaaagctgtcttcttcagtgtgacacctctctgcatcctcaagcttccatttgtcttttccagacctttcttctttccattaagtttcttcctttgtgatcctagaaaaatct

SEQ ID NO: 36 - DNA, 1000 bp, Arabidopsis thaliana
cacataatctctctctactacccacaggtatattagagaaactacatataacatgcacataagtctttattagttctcaagcctgtcatcttgttcaggtgtttgaaacgctggttagcaagatgacccggtcgcaagattcagttgtgagagcagcatgctcatcagcctttgggttactgctaagatcaagcaagtcaacactgtggagaggagctcgacttgacgggaccgactcaggtagaaaagccaatgatctcgaatctgttaagaaataacatgtgcattacggtctgtgtgaataccaaccagttactgccgaaaactttgggggggtttgcgatatatatccataggtacagtgtgcaatgagaaaccatccaggtttttgtgcttgttgtaaagtgtattatgttttcatcttgtaaaggatattgtcactcgtgtgtcatctctattaccaaactcaggaaagaaacattcacacgaaattgttgtaggctgtggtcctctttttattgattataatttttgaggctagtgttggatatacttgtcttgacagagtccaacgtttgatttatcttcagttagtgtttcatatgagagtttaaacggtctttgaagtttcaaacacacattagattttggtaacattggtaatttatttatgtggcctctatgtttattttgatttttgagacttgacgatagatgtagctatcactatcagtgagccctccaagttgttgttttgtgtatgtgaattatcttcgttttctttatgaagatatgtttctaaacttttcctgagaaggaaggtcactacgatggtcacatacggacaaactaagtagcaagcacaatctgtggacttaaaaagaggtgtttggcaagtaaaaaactgtttacgtcatctgttacgtcttctgttcactttatgttttactctccacgcatcttatcctttataagctcgcacaaatcttaaccaaaacca

SEQ ID NO: 37 - DNA, 1000 bp, Arabidopsis thaliana
atacccaatatatctaaactttaaaacttaatttggtccaacaaacgtcaatcgatacaatcatgtgttcttctagcttatggattctatttgacgacatcatgaagagtttgtacaaatcaattaatttggatattacataaactaaaaatatgttgtttttttttacggatgatcttttaataactgaagatgaaaattaattttagttagaaatgtcttgaataataattaatgtcacagaaaagtcagaacgtaccaaagatttccgcgtgtattaaacttaataaatagacacaagccaagtcttagcagtgtactactactaacacacatccacatcaatgtgtcatctttgttgtatgacacaagttagtatgacaatcagtgtttgagtagtgatggtttaaatggaatgtttgagaatccaatacaattggcggtctgctaatggccagaatatctgccaaagttattttttatctactgcttataaatcgtacaacaatctatttagttctctttagttttgaccttgaagtccatgaatcttaaggacctcttctccacatcattcaaattcacatatcggaaacacagtactgcttgaatgcccctcccatacaatatgtctatccactgatccacaataactcgatggtgcatgtgtgtgtgtgtgtgtgtgtgtgtgtgtgaacttcctggctgtaataaatttaataaatacttaaatagacaggcgaagtcctcgtccacttttctacacctagcatcaatcatcaatgtgccatcttattgtttcaatttttcttaccataactttgtccttgcatgtaacttttccaattaaatacaagtctgtcttttattaacccccgagaatttactaaatgatattgacataatctattggacggaatgagttgtcaaaaaacgagagctgtcttttcctgctccactgtcctcttatatatcccacaacaatctttgctacattttc

SEQ ID NO: 38 - DNA, 1000 bp, Arabidopsis thaliana
taccttgtagagttccaaaacattgtcacaaaatatttataaagaatttattttaactaattaggtcgttaattgtccaagggtttttcatagttgatatagttctgttcaaatatagccatccttaatcgattcatgggatcgtaaattactacttcgagtgttgtaaaaaaaaatgaaacttctacattacaaactcgaatttaatgcatctggagtgatactataaaagtagggatgctctcaggtcgcatttgagagacacagaaatgattttaatggaattaatatattttcagtttttcacaaaaaaaaattgtgtttataacaactgcagattcaatgctgattttatgagtctcacctatagaatttatatttctatattcatagaggcagtataggtgttgacccaacatcgaaagaacacttcgtaaaaaattctttggaacaaggctgaaaatttactcccaaatttagctatccgatgaagataaatcatttaccgtttattaaagaattatcgagattttagtccaaaccaaaagagattatgagcctaagattttgaatttgtattggtaaaagaaattgaacgaaaatttcagaaaaaaatattaataaattgaacgatagagttcacttactacatagtcaactagtgcctagctataatagtttcaaaagacaaaaaaaaacaaaatcggttaactacttccgtgacataattctcattttgatttttgaatccagtctaatttgaaaagtatattcaaaatctttaaatccattaatgataacttttataatacgttgacacacgcaattgtatatacaatattcttgaattttaaatgtaaattctagaatatattgcgatcaccacactaatcaaaatctttgggacaacttgaacccacatttgacttttcttggtcaaatattttggcatcatgcatgatcttctctataaaaaccaaaaggcctcaacgacattcat

SEQ ID NO: 39 - DNA, 1000 bp, Arabidopsis thaliana
ttcactgatttggaccaaatcgaacgccaggtcattctggtgagaaaatcttaaggctatttatgccttttctttcttctattcattcaggagattacacgtggcattacttgtgtggttcgcatgtcgttgtttctacattgcaaattgtcacaaattggtccataatgagtgtttctagtacctttccctttgtctgtgtttatttatgaataataattcatgaatagaataaactatttttccaaaaatattcatatatgtccgaaaactcacatccatattgtaaatttaatcgaatctaatatattttccaaataatatattagtttttgccaaaatttaaacagaaaatccttaatacagtttcgaaaattctttttctttttctgttgaaatcatagtcaaattcatttatttgtagatatatatatatagtcaaattttctttttatacaaattgataagaaaataaatgttaacctacaaaattaatgtattatgagctgtcaaattattattttgactttatgagttgtcatattatacatagcatatcattcttaagaaaaacaaaaacaaaaacaaaaacaaaaaaattatcatcaatgatccgcttcataaagtaaccaccaacacaattattttggacgcctcaaatccaaaccttattttcaataattgtgaaaaaaaagaaaaaaaaaagctatattgtggtaggtggttcgaaccactcaaaaatatttatgataataacacccaaaaacaaactaaactattatttatttattgtggtcatgagatagttacagacacagagaatacatctgtcagaaagctgactcattgtatcttatctttcccatttcctcttaccttttttcctacgtgttgccacaaaactctcatcttcacccactcaatctccgccacgtgtactctctaacccttcagttcggtctttttaaggcttccccaagccttcgcatccaaatctctc

SEQ ID NO: 40 - DNA, 1000 bp, Arabidopsis thaliana
tcatcagaattaaaaaaaaaaagtcagtctaaaaatataccctaataaactgacatacctatttcaacgatacaattctataatgagttattaaattgatttgatttcgtattctctaaaatatactcatagacatagcagaaactagttaatttgcttatatgttcatattcttgtacatcatgtgatctcacgtggaataaaaataatgtgaccatctctaattctctacttcacttagtatctaagtatccactgtgataccttgttttctgctcaaagttgtacatgcgtagattaataaatacatttcaccccttcaattgatcccaaaagcctctacaagacagccaaaacaaatcaaggattgcgcaaagcattcaagtttcgaaacaataatttttttaatggttgacaaaaagcaataatttgtaaagcgattcataggtcagcttctaaatgttctcgaaataactcgaaccaacacctaaaccatttgttctaacgataatagacgaacgtattagagactcgttaggaaaacactacttgtatatattcacttttacatacatatttgtttccttcatatatttatttatttacataacaaaccaatatgaaaaacgtacgtttcatgaaagagaacgttgtagaacactatcaccgtttgcttgtaagaaaaaggcttatctcacctataaactcgtgagtcatatttatactcttaacaataataaataaaataaaactctattttggtaaccacaaaaaatattctgaggctaaaagcgcgtctgctggaaagttcacgcgctctgctaaccccaccgtccattctgactcttttagatcttaaccgtgtctttatccacatcatacgtttatacacgtgtcacacgatcacctattctataaaaaacattattttaatcttatcctttttatgcttcccctataaattatcttctcttcctccttctccgatctctcctccgtc

SEQ ID NO: 41 - DNA, 1000 bp, Arabidopsis thaliana
gactaattgtcagtattctaacaacgttaatttcgtggtgcacgaaaacaaaatgacgatgtggttgcacaaggtccacgttcttgtatattgttttttaatgtgctaattgtgcgctaaaataaatttaattgtataaaattagtatagtttatatttttttaatatgtatatgttcattcaaaacatttggataatatatgtttactatgtgatagactgatggtggaacattttatccatcttatatgcatatgaataaaaattattcaaaatgtttgttgttgttcagattattaattattaaagaattagaaaattttggaattacaggtatatatgtatgtgttgtatattgtatatttaaattattatgtatatttatatttaaatttttctattttcataaatcgttagtatataattgtgtcacatctaatattattagaagacaagttggacttttcataataaaataaaacatatcttttatcaaattaaccttttcctttaggtgcccaaatccgtagcaccatcaaatacctaagatatactttttatttccaaaaatcctttcgtttgatctaatattaataatataaacgatcaaacaaaacgatttttagaaatcaaacgttcaagtttgatcgttggtcgtcgctaatttaagtttgatcgactgctcatgttccgtgacaatgttaaaactgaatacatgaaacatgttttttttttgtgcatttgattttaataattttttaaaaactgataacacccgaaaaaaacagcaagaaactaaacaaggaagaaaagaacggtcggtgaggaactctcgtgaaagtcttcaaaagacttttaacggaaaaacaaaaaacaaaaaacaaaaactcttgtgaacgtaagtgcatggaccgaattgcacccgatcacgtcggttccacaatcctctcctatattaaggccttcactgctccttatcatctcaaacttctcatttatt

SEQ ID NO: 42 - DNA, 1000 bp, Arabidopsis thaliana
aacaaaaatggatttgatcaagtgaaaactttggtccacatactccactgagatatttacttggcttacaacatatgtcacaagatatatattccaaagagatattttacttggctcacataattcccaagatatttcatatgttttggttttgttttcgatgaagataagatatttcaaatgcttctccacctaaacccaacaacttttgagaattccaaattctgatcaagatagaaattttttatattgtttttcgaattaattgtttttcatatcactacaaaacaaaaatattatattaattaaagtataaaaaatggtactaatttaaattactcaaaataaatgatacacatatacatggtgttatatcaattacaataattgacactattattgaattgtttgacattgatttaataaaatcggtacaaattcacattaatttgcatctttttcaaaatattattacaacaacatttaaatcactatgttcttaatcgtcttttcgtaataataattaaattttgaggtttttcgtttttttagataagaaaaaaataatgtaaaaaaaattgacgagatcatattttggttgatcacaagtgaggaaaatgaagttattatgtaaagaaggattctataatcgattgaccaacattcctccaaagaaatatattttattttataaaaaaatctaataaataattggaaataaaatcatcattcaccaaactttatactgttttacaaatctacgttttagtcaatttcttccccatacttgacctcatgaagcaatgaacacaaatctatgtttcgattttaccatggaagagggtcttagtgatatgaatttaaatgtcgggataaaatataagattataaaactataaaggtttattttgaaatacagcacacacaagaaaaaaggcccatcaacctcgcagtcctaattagataaatacacgtcaacttctctgggaacaaaaaaact

SEQ ID NO: 43 - DNA, 1000 bp, Arabidopsis thaliana
agttaaaacgggcttacgaaaccataaaaaacatgaacatggatgctgattctgtcttatggagttctgttctcggaagctgtaaacttcatggagattttgtgttaggcaaggaaattgcagagtatctcattggactaaacatcaaaaactcaggaatttatgttcttctttcgaacatatatgcatcggtgggtgattatgaaggtgttgcaaaagtgaggaacttgatgaaggagaaaggaatagtgaaggagcctggtataagcaccatagaaatcgaaaacaaagttcatgaattccgagctggagatagagaacactcgaaaagcaaagaaatttacacaatgctaagaaagatcagcgagcggatcaagtctcatggttatgttccaaatacaaacacagtcttgcaggatcttgaagagacagagaaagagcaatctctacaagttcacagcgagagactcgcgattgcatacggtctaatcagcaccaaaccgggaagtcccttgaaaatattcaagaacctacgagtatgttctgattgtcatacggtgacaaaactgatatcgaaaatcacaggacggaaaatcgttatgagagatcggaataggttccatcatttcacagatggttcttgctcttgtggcgatttctggtaaaaccaaacaccacacattgtaatttgtacgtgaaaagaagatcaaagtgaacaaatttagttggattatctaaatatcattctttcatgggaattatgttgaacacgtggagggcattatttgtgaagttgaaacgaggactatttttcaaactttcttgaggaacaagacttgacttttgtaaccataaagatagaatcaagtcagattttgctacttcgagaacattggtcaatgagtcaaaatgtcaaatagtcaatactaaaatcaaaattgaactttgtttattaattttgttctaatctatttaaccccacaaaagtttccaaaactgc

SEQ ID NO: 44 - DNA, 1000 bp, Arabidopsis thaliana
acgaatctcgtattcattactatactgctaatgaatgcatcaattttaatttttattggttatctatccttaaaaaaaagttataagaatattttgataatttgttccaagatgtagtactagtagtagtacttggtctcgtaaacgacgacgccgtttaggaaatcaaacggccaaaaatgcttccactttgcgaaattttattggtgggaaacgcaaatgtatcgataatgatttaaagaaaaagcggcgtttctaattggaccacgttgcccaatctctacggctcacgcacgatctgacgtcaaattggcataatactataataaccttatcgagaataaaataatagtcgcatccaaacaaaagaaaccaacccttttatatcacgacgagtgtcacgccatggacacttgtccatctctctcgctctgacccctttcaggtatcttttatttctcaaagaggaattattgattttccatttccaaagaaaaaaaataaattcgaaggtcaggaaaattaacaaaaaacttcctttttttttttgttagtttgtgtgactgagctgcttcattttttttctttctttttttttttggtttgatgaatcgatttttgttgtctattactgattggttttcttgttcagattcactgattcgaagagaatcatgatttttttttcccgctgaataataagcatatgattgggtgttttggagatttgtttactgattaaaaggagattcctttccattttcaccatttgctctgtttgacttcattgtgcttatatttcatttagatcttttgtttgggtttagctttggaactgataaaaatctgattttgtctcacggctttggatttggttcttaaattttggtactttaaaactggataaagatcagtgcttttttagattcttcgtttgttgatgaatttatggatgtatgtataattaaaccataatctctctgcttgtttgttttcttat

SEQ ID NO: 45 - DNA, 1000 bp, Arabidopsis thaliana
ttgaattttatgtaaagttattctgtttttaggacttttatcctcactgaattatgttgtgttaaagtagtagcttatatggtcacatatctcacacgtgtttccattttttttctatctaagttttcggtttttacattgtatacaaaaataaatggcatgccttgggccttggccactgattcacatgatcacgtagagtttgtgtgtgattcaaatcctagatgagaagatcatccaattgatctaagatgtgtctttcttatatcacacgttttattttaataatactcttcagttgggtttgattccatttatgatttatctatgaaaacagaatatgtcctcatgctgattatttcgtttgtggttgcattttaccatctgtagttttttttgtttactgacaaacaactaagattttatgaattcatttgatgataaatagttaatcaaatatattattatgagtgactcgatggcgaaagctggtattatcttatggctgctaaagtctacttttagaagaagaaaaaaaaagggtctgttgcattaccctgttgttgcgactctcctacaataactctcacgtggcccgtttcttggtttttgccctattttcacaaaagtaaacagagattttcactagcaaattgggcccaattggagacaacttcatgtggtttgaatcgtatatttcagatagattagacatctagtcttctttgatgtgcggtgaggatcttaagtcgctataacgttgtgaaaaaagaaaagaaaaagtcactataacatagatcgcaactaaacaaataaaggacactatacatgtacgtaataatattcatttattttcaacgacaatgttccctagggtggtaacccaagtatttttcatagatattatacttctagcgaaaagccatcaattgagaaaaaaggcctccacgtttgacccgttttgcctataaaaggattctttaagatatggaccttactc

SEQ ID NO: 46 - DNA, 1000 bp, Arabidopsis thaliana
caaagctcctaaatgatatagcttcaagcaaagattcacagagactatcacatgcgagtatttaatcatgcctactataaaagagaggacaattagctacataaatcaaacgctataatcaataaagcgatggataataccgaaaacgacggcataatcgctgttgaggacatcaccgcaagtgccaccaccaatagggcaaaacgcatcggagccagtgaccttgaggtaagtcaaatacgcagtgtctaacatcccaataccgccgattccggtgtaccagttgtatgtcgaactgttactagtagatacttcacttgtagacgatgaggaggaggaagacagcgacggtgccgaatcctcaccgttttccggctcagatgaagaacatttaatcggaatcgcaggaaatctccgatcagagacctaattcagaacatctgacttcaaatgaccgatcacacactactgtcactgaagaaaacacatgacaagaatttacctcgaatcgtctaggataagaagttaccgacggaggagaaacctcacggaagccgaaatgaaactgacaagacgacacagaaacaaacctcgccatcatcgtcgtcgaagaatcgctattccaccattgttaacttcatcatcttctctcctccgctccactatctcagccacttgtttcaatattgggcctaaacgaggcccaaatacgataatttggatccataattagtttatcacaattgggccttacggtcttcttatccgtccgagtcagcaagttatcctcttcaacacgactcacgaggcaccgagttggacttatcatgattcattatctacgcattgtttacattttggaatcgactttggtacataattgaatttatccatagaaatgatagagactcgaagatcggacataaaacttgctgacgtgacacccatacctttgtgtctgtatgtattatataaacgctatagaactaagagaagaagt

SEQ ID NO: 47 - DNA, 1000 bp, Arabidopsis thaliana
ataccctcgtgacctttatcgaaggtttccaccttcacactcaagatacttctcctaggatcttctctacacacattcctgtagggtcactccctgagtcagttaaggactcgtcctgtaaagttgtgtattgctgcaggaacccgaaggacgcgtttgtctcactttggcatttcatgaaaaatttgattgttaaggagatggttggatgcacaatggaggaaatggtgaggtttttttgccgagggtctagtatatacggacccttttgggatcatgtattacagtattggaaagaaagccgagaaaacccgaaaaaggttatgtttgtaatgtatgaagagatgagagagcagcctcaggagtgggtgatgcggatcgccgagttcttgggatattcttttacagaagaagagatagagaatggagtattggaagatatcataaagctgtgtagtcttgagaatctgagtaaattggaggttaatgagaagggtaagttactgaatggaatggagactaaggcattttttaggaaaggagagattggtggatggagagatactttaactcctttgttagcagaggaaattgataaaaccactaaggagaaactaattggttctgattttagattcttttgctaagaatctgctttaccaacttagttctcaatttcttgagcttttttttttcttctttttattggcttaatacaagtaaggttgttgacttgttgtgatggaccaaagtcaccaaactggtctgttgagaaataaaactattgtttttgagttttagacataagagtcgtgattctccttctttcttgttttgttatctttaaatattatattctacgtgctcttcaagtaaaggatgtgttgaccatttcaccacccaaactaatatcttagaagcaaaagcattgtgaaatttacttgtagaacaaccttctcattcatatataaaagcagacaagcattagcatcatagatac

SEQ ID NO: 48 - DNA, 1000 bp, Arabidopsis thaliana
cagttgatattcataacattttgaaagaacataacccaaattagtagtatatttctattttagttcgttgaagaaactggatttatttctcgaaatattactaaatcgaaaaaagaaatgaaaacgtgcacatgttgttatagtgtttaacactgataaatttcagttttaccttcttcctttttttaaggagtctagcatgacaagagatctttgttatcattcggaatatgaacgtattataagaaaatgattgtattcaaaaatagaataataaataaataaacgagaaatgagctgtgctttctagaagaacatcgatatctctttttcgtatttttcgtctttaaggctggaaacaaacgaaggcgtactcctacgcctcaaatctttcatgccttttggcttttcccaattaagattatacatactatttagcaaacaaaacaatggtagtagcaattaatccaacaaaataagaatttttagaaacgtaagtattcctccaaaaaagataaaataggtgattagtttaccttgccagacaactaaataaaattaatgtgaaacttaagataaaaggcagacgagattttttaggttctgtgaagacaaatccctttctatttcgttttgacatgcatagaagttaacacatactacggaaatgctaaaaaaacgagatagtgtcgccatgataactgcaatgctgctgaaaccctcaactttattaatgaactaatgaagccaaatcatttttattttatgattaaaaagttgccaagattcgtgctcactctgtcaaagacaacaactttatcatttttcaaacaatagttgattaaactaaactaaaaatgggtatacaaaatgagttacaacgataaagacaacaacgactttccctttgtttgattatttaaaacaagtccgtgataagaagaagatcccactaatggaaaataaaatgttataaaaactggcggcaatacacaattgt

SEQ ID NO: 49 - DNA, 1000 bp, Arabidopsis thaliana
tggatggggatggcgtactatgcatggctgttgacattttacccacagaatttgcaaaagaggtatgtatgaaggttacagttatagtacttaagattaaatctaaagttaaaaaccttgtattgagtgggagttcttgtgtcctgaaaaaggcatcccagcattttggagatattctttccggatttgtcggtagtttggcttcaatgactgaaatttcagatctaccagcacatctgaagagggcttgcataagctataggggagaattgacatctttgtatgagtatattccacgtatgaggaagtcaaatccagagtatgttctgcttcgagcgttacttcatctgaaatatttaggcctcttctctaaactatgttttcatctttacccactttaactgcagagaggcacaagataatattatcgccaacggggtttccagccagagaacattcaacatattggttagttttgatgaagaaagtatatataactagtttccgaatcatatgatttaagctaatgaattaagaaaatatatagttcaagacttatgattcatatctctatcaactttttgaccaaagattgatactttttcgacatctgtcacagcattttgtgatgattttgattgagacaaatcatttgtaggtatctctgagcggacacctatttgataagtttctgataaacgaagctcttgatatgatcgaagcggctggtggctcatttcatttggctaaatgtgaactggggcagagcgctgatgctgaatcgtactcagaacttgaagtaagtttctttctggataaaacctaatcattcacatggaacaactgtcaagagtttttaatgtcacgtttaggttcaatgtccttttcactaagtctcgtaagtttttaaaacaagtaaacaaactacaagccaaaaacattctggccccacattaacctattcccacttgttaaagaacccatcttgcattatcttggt

SEQ ID NO: 50 - DNA, 1000 bp, Arabidopsis thaliana
gtaatttttatttaagttttggtttaggtttggtttgattaaaaaccgtaaaaccgaacgttttttttgtttttgatttaaattaaaaataattgtatatatatatatataatgttcatttgataacatgatatctatcaaactatcgaaaaacaaaaccctaactgtaacctaaactaaaattctatataaattacatgccgtcatttaggatttgagtttacaaattagattttgattttatttatgcatcacacttataattttttttggtaaaaacatgaaaaaaccggaaccaaaccggaaccgatccgaaccaaaatacatatggtttttaaatggttttaattttttaaaaccaaaaactgtaaaactgttaaaaccgaaccgtaaccaaaccgaattttatatggtttttatatggttttacttttcttaaaatcgaaaaaccgtaaaacctaaaaccgaaacgaaaccaaaccgaaaaactgaacacccagcccttaaatataatgaaaatcgaataaatttgtttgaaagaatcgaacaaaattgacaataaaatctaattaggactattttcgtctaattttgacttagttgaaacagaatattagcaaaaatactaaaacaccacaacgcgtaataatacccacacacgatatcattaaatttgaccaataagaatctagctcttggcgaccacgcaagtatcttccatcttgctctccaagaaaaatctacaccggctttaaatttacataaacaccctcagtcaaagaaaagtcgtaaacatagtctctctcatgaccacaagggtaacacagtcatcctaaatataaaccacacaagaaaactgttatactttatacacgtgtcatagtctcattacatctacgtgaagagtttcgatcatcaaccgttcgttttcttactatataaaccttgctcgagacctgcgtgtgaagcgtataaagacgacaaagtaaaccaaaaaaaa

SEQ ID NO: 51 - DNA, 1000 bp, Arabidopsis thaliana
acgaaacaacttgcattaaacaatttgcttctactatacttatattgatctatcgtgggcggtgagtaaaatgagttgggacatggatttgtcatttaggtcatttttagtaaatgtgaaacttgtaaccaaaatatgcataaaaactttagcctttctaaaagctaaaaatggtttgaggaacaccaagacgtaagctcttttaagctatttgatgtttgatgtgataaaataactaaaaactatttgttatgttttttcctccccttaaagatataagagaagagaaggtaaagaagttgtgtaagttagtcacaatcaattgttggaaataaatttgaaaactctgttaaaagtcacccaaatgagtttcaaacaagagttaaaagattaaaaaaaaaaagtaactgatataaagaatggtagtttactttttttttaaagaatggtagttttttaaaaactatttaggttgatggtatttttgtaaagaaatccaaaaattgtagttgatacaagaaaattttgatttgtgttgatttatttctcttttggcatgtgaatgtaagtaatctttttccacatgatttctaattctggacttgcatgtcttgttacgccagtcaacattcgttggataagttacaatgtcatttacaacttgcagattaattcggaaatcaagaaggaaaaaatctgaattagaaatagtaggttggatttttcacaaaaaaaaaaaaagtacaatattttgagatatgaccattagattcttaaattttacgattctaacttgtaagatttcaaattaaaccatgtataattcaatttaggggtaatttaatactttagggtagtaacgataaaatagataattaaattgaagtttaaggaccatcctgtaaattcaagttcggtaccggaaacacgtcagcattgatctaatagatacgagtcctcctttctctataaataacgtaatctctctcccgtcttcatt

SEQ ID NO: 52 - DNA, 1000 bp, Arabidopsis thaliana
aatcctactttttcaaattgttttttttttctttgtattaatgctcccaagatgtttattgaactgaacacactagaatgaattatagcttttgtaatcagtcgagtagactagtttttttttttccgatttaattttattaaaatgtcaacataacaaacttccaaacagaagtcttttaagaattttggaggctcaaagatttttttttctcgatttttttttactctaatttggaccatttggtctgggaatacatttagaaattgattcatctctatctcccgtatatattttaatatatttagaaaaaatgctaaaataaagaaaagtagacgagcttataattttatatattaaataaaaacttaaataaaagcttataattttatatattaaataaaatcttcaccttaaaactattaaaataaataaatatatatatatatatatatatatatatatatatatatatagttttgttatcggtattaaagctctattttgtagtcgaaagaataaaatcatataaaacaattttgctttagctttatatcgattttattttgtgactaaatactagttttgcaaattactattcattatgttaatttaaaatattttcaaatagaaataaaataattattaatattttttaaaaagatataaaaaaaatcgattacaaacaatgatataacgagaaaaggatagcattaaaaattcattcctaacaattttgtactcaattattttccatttttagaaaaacaactgcttcccaaatatttgatcttagaatatttttccaagcttttaaaatctttttgacagatacgtggcacataaaactaggtggtgacatagatatcgacgtgtgtaacgtttcttgtgtcctaaatcaattaaaaaagcctcacatctctttgtacactacaaagaaaagatcattgattcactcacaactttcatctatttatagtctttaacccatttcagtatc

SEQ ID NO: 53 - DNA, 1000 bp, Arabidopsis thaliana
tttgaaaacgttaaaaatataactaaaagaattattgttataaaatataattactcccaaccagaacatcaaactaaaaacagaaactcatgagaaagatttgatctgattaatgacaatacctcaggcgatgtaaaagacatcattgtaccatcaggaccgggtctagtcataacactgtcgtcggcataccacatcatctgaactacctcgggatagactcctagattcaggaagtaagattggagaaagcaattggctaatgcttcggaagtatttaagttcgtgaagtgggattgaataaagccattggctaacaatacggaaggatcaacatctttgatcatggaatccatatctgcaccagtaattatgccctaggggagacgaagaaatatccaaagtaaaccctagctagatatcaaaatctcttaaaaacaaacttatctctctgttgagatcaaccgctgatctataggctaaaggaatcaccggaagagagagagagagaggaaagaggcggaagttgtgacttgtgagacggtttggattttggaaacctacgttccattgaacttctttatatattggtttttcattatgggatatagtaatatctttataaaaaaaaaaaaaaaaattatatatatatatatatctttaatacattattaaattaaataatatacggataggaattactataaatctttataaaataaaaataaaaggagaagtcccttttatctaatttggatggttaatttgtgaattttccatatttatctaatttcgatttgaaacatatccaacgttaagtcagattcttgtggtgaaattattgcatcataacataaagtcatgagagtaggagagaaagtaataagtggcaacatcaaaacgacgacgtttttaagctagtgcgtcagcaacagttaaaccatatgaagttaatggtctgtgttaatgaagacaaaaactatatcctcaggttagcgat

SEQ ID NO: 54 - DNA, 1000 bp, Arabidopsis thaliana
ttataccatcgtaatccacacgtacacattgtttttaacttttgacaaaataattgtttagctacctaacaaattgacatattgtgtggcaaatttataaataaataattacatatatctcactgagacgccatgcagattcgtatgtttatacaatgcctcacccatttcttgaattctctcaattgatcttcccatgatgttcatttattatttgctttatgtatatctttgtagaatggtagtattactattaaagtatcaaattaccatgacgacctatagaaaaatgtttttagaaaagcatagggatatgtgtcttatggacagtggataagagagtggtcctaaaaacagaccataaattatgggatcttcccttagttcctttattgttatcatattttaattactcttattagtatttttttaatggtcaagttgtttattggaattacgcatttaattatttttctaattcgtatacttgtgagaaacagaacagcacgctttgcttcttttactagacaaacccttaattagcgatatttgaggcagaaaaaaagacaaggttgcgtgtgttttactccacaaaagaactcctaaaaccaaaaacgttaaaacccacttcttttttttcacagtattctcttttcatgtcaagaacaattactgtttttcttggtatgcaatctgtaaatttattgtagttttcatttctacttttggggtttaggagtaaaagtggagtcactctactttgctttacctttttctgagtttttttttgttcatacaaatccaaatttccttttctttgtattcctattttttctcttctcgtccctttcagaatctttagagagagagaacaacaaattgagaaagaaaaaaaggtaaggctgtgtgagagagaacccaccaaagcgtgcacgagagagagaaaaaaaaagctttgattttgtataaaatcccaccactgctctcttaccataccttc

SEQ ID NO: 55 - DNA, 1000 bp, Arabidopsis thaliana
tgtgaaaacagccaaaagctacggagagcgacactcctcgacgacgtccatgtctctttagattgtgattgcattgttaattatcatatttatcaattgattagttaagtcttaagtatcatcacacaagtttatacattaatctatacagtgtttaacactacaaaactccccaaaatcacgtgtttcatatcatacgtacatttgtctaccgtctttattaacaatattaaaacagaatttatcaattttctagttgaatagactatatactattatagagactcttataaattatgaaactcctaatcaattaaaaatagttaatttgaaacatatcactcgaaactagggtattagttggattgacgatatttttcttattaatatcaacgagaagaatcaagtgtcgttagctcaattggtaaaaaatttaggcaaagcttagagatgtctatttgagtgacgcttggaacaaaaattacatgctatggtttcaggcctagagaaatgcttcgatccagaatctcttgatattcaaaaaaaaaaaaaaaatcaacaagaagaatgtataaatggtttctatactatctaagttatgaaaaatatttttggttacccaaattacaagatttgtaccgaagtgtcacactagattcacatgcatgcatagttataattaacttaaaacataaatgacttacaaaaatacttacaaacaaaaaccaaattaagttatttagtggccagctttgttactagagttttagccattatcataattcacaataaggtgtaggaaaaaacattaatatatagtatgtatacatccaaaaaaaactttttggaaaaaacaaattttgggattataaaggacacgtagcccaatcgatgaacagaggacggcaccgaatcaatttgacttcaccttttttccccatatgataagagtctcatctctttcctatatatatttcgtacctctctttaaggaccctac

SEQ ID NO: 56 - DNA, 1000 bp, Arabidopsis thaliana
aagactatggcgtgaagcgcatgagctgtatagaaaaatgcaaaaagaggatgggcttatgccaattaagtagacgtataaaagaggtaaatatgagacaccttagagatagcgagttaagggttcaccatcaccacagatgcgtcttaattttaagtttggctgttgtatgatacgaaattggacaaacattaagctaggaggcatgatggatttaatcatattttagatgggaatgagaggatattaaggtaccaaccctcaaggcatattgtagactggtttggtttggacatgtgaagagttttgaagctgaagtgtttggtcactctagcattagatgttgcaggtgtagttttgtacatacatgtaaatgatgtctctttcttacgcacatttgttgacatagagaagctaatatttgcttaagcatgttgtaagctgtaactttagaatttaacctccactgtaacatattacatatgcaatcaggcaaacgaagattcgatcaaacgggtcattgcaaatccagaagtgtgagaagattgaccattaggaaacttataaagtggttcagtcttttacaaattaaatttcctgtttcaaacaccaatcaaatccaccgaattaccaaagatttcatgtttgcctctatccataatggttggaaaattttcaattttttaagtaatagtttgatattgtggggaaaaaatactattctaaacggtagataggtcttacacgtgagcattccgtaactggacaaggatcaaccaaagagaaagcagggagatccatcacggaacattatctttgtaatcaaaatctgatcgtacacgtgtacactatgatgtccactaacagtccacatctgttgacctgacattctcagacacacgagaggatcttttttctttctaacctctaaaatattttaattttaaaataaaataataaaatccgtatatgtgtctatcgttagcttctacagctcaac

SEQ ID NO: 57 - DNA, 1000 bp, Arabidopsis thaliana
ctaatctgatttgatttttttttattttgtctacgattcttgagttacgaaatgttcatcatttgttacttcttgttgtcgggattgagtagttgcatttttttagttgtggagctgtgacaaagaggccagctagtgtgtggaatgtggatcatcatcctttggttgattccttgtagaatcctttttgtaaacatttaaactataataaagtgttatgattaagaaaactagttacttatttttttagtcatattgactctgagccataaactccataaaagtcctcctcgattcaattcggaatttaaacagtttcagttgaactctgaacaagaaaaaaaaaggaaaaagcaaaaggagtccgtgcaagataactctcattttattctctttgtttttgaactatttattttgcacaaattaggattgatattaaaattttaattaaatcaaataataactattaattttgcaccaattaggattaatactaaaaacattaaattaaatcaaataatagcatttttgtttagtaaatataaaatttaatctaaaataattaaaatacacaatacattaaaaaaaaaaaactcaaaaggcaaagcataaaagaagaaacagagtggaccatgtgtccgcgtggaaccaaagcttccaaataactcgaccgtttagtttattggaaacgaaaaaagaaaaatcatggcaacacaacggccataaacatccatcacgcgtctactaaacgaacacgcgtacggtccagattctcaaatgacagctgtcatagagccgttagcgccgtagtcaaagggtttaagcccaaccgcgtttctctttttcacctctttcttttgtagcataatatctcagccgttagatttaagtcggtccttactttttcatatcaacggtcacgattcactaaacacctcaatacagtgtaccccacttcttctctctatataaagaacaccatctcctaggtttcgttaac

SEQ ID NO: 58 - DNA, 1000 bp, Arabidopsis thaliana
aaatttcaactataccattaatacatgtattatattctagaaaccacggttcgattcaaattttgacctaacaaatattaatataatgtttggatgtttcgtaatattgtaaccattaatggagatgtcgtatatcatttgagtttttttgttaaccattcaaagtacactataattaatttagttactttaatataggtctattttccttgcgtcatagtctctatcgtggttttgtcggagatatgccacgttctaaaactttggacttctgcgtcgccaaagcgtttgcaacacgtcaaattaggaaacacaaaagcggcttagatagcaaacgagcgataaataaagggtcaagtaagccaaacaatatgtaataaacgaataaattaattaaaagaaatgtttgatgtcctattaattttataaattctttttttttgttttttttttaattttataaattccttatacacaaatgtggttttttcaggaaagttgttcaatgtgcttattacaatattaatcatttgatgatacatactcacagttacactaacagttcaatatcctgatggagcttatcactaaaagagaaaatctatactacagtattaggaactcgaaacaatatcgtcatcctaaaagagacaaaatagcctgactaaacatttatattttcgttaggacaattctcaaaaaaagatactatatattagactaaaaaaaggaatgaaacagtgacatacagctacactaaagacaaaaaaggagataaaataatatctccgtagacacataatagtataataccgtgttacgtcgatcaaaatgtgatataagaaatcaaattaatactaaaagcgatatgtttcgggacagattggtccggacccgccacctttccctttctatatacacacacgcaatacccaccaagaacacacacaaacacttaattagcaatataaaaagcacattcacaaactctttccaacac

SEQ ID NO: 59 - DNA, 1000 bp, Arabidopsis thaliana
ccgctatacacttgtgggaaaaggcttcttgtagtacagtttcgtcacggcgtcttattaattgcaaatctgtgacgttacggttttcgattcgggttactattttaatggaaatcttttgataatagctagagcctagagtatgtaaccctaaggttagatggtaataccgaatctacctaaatttttgttattttcggtttttcatgagatatatagactgcgtaatttaaaatctcaaaatttggtttcactttatttttatttttttttagtttcgaggtaggtatacaagcatggaccttttgacattatttaaaattttcataatttagaatcttcatgttaaacgtgtatggctacgatcttcggaactttattcggtttgtatgctcaaaggccaacgccatttattttctaattgattttttcccgttacggaaacaatctttttgcattattgaacttaattaccaaagtttatgaaaagaaacaataaagtctaaattggcctattttgatatactgatcacttacgcgaaaacatctacattgttatctactagactagtcattaactcgaagatagaatttcatttcttgaccaaaaaaaaactcaaaagatagttataatcacagattcattattgacttttgcttttaacactgtagccatttatgtactatttatacaaatacctaacttttcaaatttgtgatatttttcaccctacattttagcctataggttggatcattttgtcatattgcatcactgtttactagtaccaaacttaaatatcacacgagcagtaaatttgctatcaattttttcgttttcacaaacatatatctagtttccctaaccaataattaagttaaattgcattaggaatttaggatttataataaatagatatataacccactaacgacaaaagactatgtcttgctacgtctctgcctttattatcatgccacaagaaaataagaggggaac

SEQ ID NO: 60 - DNA, 1000 bp, Arabidopsis thaliana
actcctatgaaatcaagccttagagacactattgttagcctcaaggagaaatgtcttttatgattctgttcgtatatttgtaactattatgtgtatttttattttgttagtattactaattcaagtggtttaagttgttgagactctttaaaatctaagcattttataaacaataatatataattattgtttaggctaaatttgtcactaattaaggtttggatacatagtgtctaaactaagctaataatatcacttaacgtttacttgtaacgctaggtgatgatgtcgtcaagtcaattggtacaaggaataaacgagtggtcatatgacattatgaccatatgaattcaaactccagtaatccaatggtaattggattcaatgatcaagacttgaaccacgtaatccacccttatccttagaagctcataaatatcactaaagggacaggcaacacttaaccagtagttgtccaataatttagttttccaaaatgaaaaattattgttgtcatctattttaggtgttttagttcaatgtggattcctcgtcctaacaaatacttgacgaatatatctagactataaaattggttatgagttctacttttttttgtttgtgaaattatcaaaatttgttatatttatttatttattctcattaatttgagtactaatttttaaattatttatactaaaaacaattactaagatacaaaaatggataagagcatggtgtatagatatttaatgggatagaatatttcccataattgtatgtgtgtgagaggttttgttttcgtaaggaaagaaacaaaaaccatttgaccaaagaaaagcaaaagaaggcaaggaatcaaacaacaaatgttgcaaggcagaaataatggacgttatgttaatgtagtgtcgtcacacgtgacttaaaagagacgagtctgcgtgtcaaactaaaaatgtatgcaactataaaaatgggatttgattatctttttagt

SEQ ID NO: 61 - DNA, 1000 bp, Arabidopsis thaliana
aaagaacaaaatagtcccgcaggttaaaacctagtattttacaaaaatatagacaaataaaataaaaatgaactatcgtggatttagttagagaaatccatacaaaaataaattgcattgagaaactttagatgacgactaagtaatataagattttgcattgagaaagcttacatgtcaccctaatttctatcaaaagggtttctgatactaattggaaccatgtgagaaactatccatagaaatgatgatcttatctagttatcatgagctgatgactttgaatattccacagtcactaaacgcatgtttatttctcagctttatgaatctgtatgtagacacactttcgtattttcttcacttttgtgtatttgtattgcccattcctcttttaccaaccaataaaaaaagcagtcttttttttgtttggtcaagcattggcactctttgtcaatcaattacccaagggatatataataataaattggaataatgaaagtagttcatcataaggttcgtgattattaacttctacttttgtttatattcaatagattaagatgccgtgtatgataaggagagcaaaagtaatccgatagagatcacagcaatattcattacaaaatagttttcaaataaaaacacatgatcaaaaagtgactcacaaaaacacatgatacgtacgtgggatgctaatcctaaagcaacaaggccacaagggcaaaatatgtagctttcatgctttggcaaagcgaaaactctagttaagcttacctaacagaaaaaataacccccaaaaaagcgttacgttccaaacatttagacagggtacacgtgaatgactcccactttttttaaaaaaataatagtactatcaattcaaatgtgtaaacaacaaaaaaaaaaaatgtgtaagtaacgtctgtaattgatttgataagataaaaaaatcttgattaatttttctataaattgaagcctttcctctttacatttc

SEQ ID NO: 62 - DNA, 1000 bp, Arabidopsis thaliana
ttcatgcatgcattattttaagatattaaagagtaatagtctcgtcgaaattataacattaaaaagtggattattcgcctcgaagatatccgatgtcacaattttgtcttcttttagaacataatgttgaatccgagaatcgaggtatacctgaatatctcgccaatgtgataatgtgatataatcgagatacctgaatacccactgaaccagactcgtgaattgactttgtccttaagtttttcaaatttgatcagcaatacagatatgtcatttcctaggactataaaagatatcaaacaaatattaattaaagatatgtaaaacttatgggtgaatgatcggtgatgtatgtgtatatgttactggcattccataatacacgcgattcaactaggaaaaatatactataaaaagaggcgtatggaactacgttacacgtttcttgtacatttgagcttcgttacgatgatttgtatcaagagtgaaaaataactacgatttattatttacagtttcagaatatgatttgattcgatgtttaatatgatttcttgcacagctagaaacaaccataatttcaacatttcatattagcaattcctccgttaacagccgtttgacatttttaaagtaggctaaaatttgggtaaactgattttttaactaatattttttaattttatattaactacaactattattattattcgtttataaaaatatacgaaattcaatttaaatgctagtagtattttggtatttttaccaaaaaaaaaactgacacaatcgaaagaacgtagtaaaaaacaaagtatattttttttgtaatgagttgtctctgattcagcaataggagagcgacaaaaatatcacgattttttctagcaacttttccatctccacggaaaaagagtaataatatgacacgtggcaacatcgcaacggctcttaccaaatctcctcgcatatataaagcgtcttctccgccaccttcatt

SEQ ID NO: 63 - DNA, 1000 bp, Arabidopsis thaliana
atagtattgtcaatttttcaatctgaagtaaatatctttttgtaaggaaggggtcaacaaatgatcacaacagagttggcaaaaagttatcaaatcgcatgcacggaagttttacgtgtggtgaaggtaaacttgtattacacttatctataaaaattagtttaggctttgattctaaatcaaatctccgattagaaaaaattgcgtaagcaaatagctggaaaaaattgtatcccatcatacttaagtcacaatgttttgtttttgagatttgtgatgtaatcaatatatgttttacaatgcaagtataataatattaaagtcacattctaagaaaattatgatttgtgtcatacgtatacaaaaacacccgtcacacatcctgacttctgaacgttaaatctgtcgcacacaatcataaaaatttaaaaattcaccagagatgtactgaaaagaatataattaatcacatgatgatatatgcataggagatgaggattattcattttctgaaattccctatatgaaccattataattgtttagtaatcagttcagaaatgctaatcattatatgaaccattataattcccttcatttttatttaagatccacttaacaggatttgttaatatgcacccacatcactaaatacattggtacgcaaccgttgttccatttccattttcacatcgaccagaatgtttactatgcggtaaattgtgtagtatgcagatttttttgtatcatttaattttctaacacttgttaagtcgaaactaattttgtcacaagtaaaagaaataaaaaaggtggaaattattaatcagtagttagatgattagtttcgagttgaaatgaaactcgacttaacaagtgatagcgacgactctagaaacagccaaaatccgccctattgctacctgtcgacccacaaatcgtttactcaaaaatgaataaaaaatttacgataaagcaaacccaaagttatatcttattat

SEQ ID NO: 64 - DNA, 1000 bp, Arabidopsis thaliana
ttttgtgacgaaccaataaaagaggaactgctttttttcttcgcatgtccactatttactgtttggtatgacatcgaattggtatgatacattgttgtaccaaaaacttcaaaatttggtggcttgtaatcaacattgatccacttagccattccaagcagtgaattacaatccgtggaaggaaaaattatggtccggcaccaacaaattatcacctcatctcccgtcacaaacatatcatctagaagtcggtatgccacaccacataattaaggagcacattactcacatacccaaccaatgtgggacatatctaataagctcattcttggctggtacatgctctatttcatatttatcaaaaaaaaaaatattaggcattgtaaatagcgtttttgctgttgagcaaaatagttatatttgaaagtaacattggtatttataattatagtataacaattaggcattgaagtgtgagtttttgtttttgtttatttaacattggagtattaggttcttagaaatatatctatatactattagtagtttaactacagtttgtacttaattgaaaaaatgttaaaagttgttttaacctagctaattgctaaaaatgactaaatagacatacacaaagacttgtacattttcagcttaacgactaatacatttttcctttatatatatatctctatcgagtctagttattaatgttgaaagttgcaaataaaacagaaatgctaacatgtaaatatcgtagccaaaaatgctaacatgtgtataacggttataaccacaacttgatggccgacctcttttttcttttggtaaccatagaaatggttacacgtaactagtacgaaccaacgaaaactcttcttattcgatagttaaagataatagcaatgcgcaaaaatatctagcactcacacgtgtagttttggattctcattggtcgagagatctataaaacgatactattggaggttagatttttctc

SEQ ID NO: 65 - DNA, 1000 bp, Arabidopsis thaliana
aatatccaaaagaaggttacatgcataagcttagactggttttgctgattaggtgcagctgaaagtaatctcatgaaattgatttccgttttgccgattcccattttaagatatctgatcataggaatgccacattcagatggaagccagacaccagaacagaaacaaagttgaggtaatcaaagaagccatcttgagagcaaagtggttttgattccaagaactcatatttatcagcataacctctatgttcatgaaacccagaagagcacaatacataactatcaccactcaatctgttattaccagaactagcagtgttcaacttcactcaaatgtggacgaattacagtaaattggctaatcccactactgaattgaccttcaaggtaaaccccaatcatttgccactacgatcaaccaaaaatatagattcttcattcactcttatcaaaagctatacatgaaattcagagtgttctcattcctaaatatggaaagttgaattctatcaacacaatcagatcatgcgacaatgaactagaaaccacgaaccagaaattgttggtaatcgtttagtggacgagattgaatcaaaggttcaagtggtaatcgttttctcctgacgcaaaatcgaaagaaaaaagatcggtagcgtcgcatcctaatcgggtgacccggaaaccaatagttgattcgttttagtggcggtaaaacccggtttgatgaacaaatattaatgggcctggcccatacgaggatgatcgtggcaatgtcgatgataacaacaactcctctattcgggtttatgttgacccggaaaacgaaagcataggacacgtgacacatgtgatgtgagtgaagccaaaaataataatattgggaaaggatgaacacagcagctcagctttcgtcttctccgtcaatccaataaaaaaatcagcaaccgttgtttgtttttaagctttttttacaaaagacgtacacgtctctctctctc

SEQ ID NO: 66 - DNA, 1000 bp, Arabidopsis thaliana
gattgtgggtagaagtagagcgagtgctcttactttagcggtatcaacttggaagctaccctgcccattggattggatctaagcttcatatttgtggattaaaaaggccttagtgggactcatgtctcctccggtgggactagtgtcttttacaatgtcaagtttaaaaaattggataacaacaccgataaaaattcacatttgcaaattttattcagtcggaatatatatttgaaacaagttttgaaatccattggacgattaaaattcattgttgagaggataaatatggatttgttcatctgaaccatgtcgttgattagtgattgactaccatgaaaaatatgttatgaaaagtataacaacttttgataaatcacatttattaacaataaatcaagacaaaatatgtcaacaataatagtagtagaagatattaattcaaattcatccgtaacaacaaaaaatcataccacaattaagtgtacagaaaaaccttttggatatatttattgtcgcttttcaatgattttcgtgaaaaggatatatttgtgtaaaataagaaggatcttgacgggtgtaaaaacatgcacaattcttaatttagaccaatcagaagacaacacgaacacttctttattataagctattaaacaaaatcttgcctattttgcttagaataatatgaagagtgactcatcagggagtggaaaatatctcaggatttgcttttagctctaacatgtcaaactatctagatgccaacaacacaaagtgcaaattcttttaatatgaaaacaacaataatatttctaatagaaaattaaaaagggaaataaaatatttttttaaaatatacaaaagaagaaggaatccatcatcaaagttttataaaattgtaatataatacaaacttgtttgcttccttgtctctccctctgtctctctcatctctcctatcttctccatatatacttcatcttcacacccaaaactccac

SEQ ID NO: 67 - DNA, 1000 bp, Arabidopsis thaliana
agtctataactgataaataattagtcaatcagatcaaacaatagagaaataattagtctgcaatcagcgattattaaaaaataaaataaaatagaaacatatctcagcaccgatttttcaacagccggcccacgatccagaaccgtccagcttgaagccggcgccgcgtcgacttgttccaccgttcggatattgccacgtgtgtgagagacacctgccattacctcgtggaacatgttttgttagtataagttactaaccaagtcgctatcttcaattcttcattacctaagagttgaataaaaaaacaaccatatacgatgagacacgcaaataactgatatacgaaacaattatttattgatgtacattcttctaactataatactacaagattctgaatagttttagatgtaaaaagtacgagaagagttatgatcagagtaagttaagtgataagatcatgacacgtaggcagaggaaataaagtagaaactatcgacaacataataattgagtgagtacaaaaaccacgtaataattaggtgtacactgtagtctgtagatcgattgtacaaaatgaaaaaactatatgatttaaggaactagatatacatatgcatcgactcaagaacagttgctttatgtgaagactatcaattacattatcggctaaatttggcttcatgtgttgaatagatggattgtgtgctcaggtagttgtgtcagttaattataacagtaaaccttcactgcttcacatatataaaatttgggttaaaaaaactttaacattttatttttttaattgacaacaaacaaattctgtaacggttacatataatagtgcttcagttaaactccttttttaggcaactctaattcctttatacccttaatccattattattctatcgaacttatctttttaaaagcttccgcgttacgccatcacttctccaccatttaaatacctaaaccactttctttcaaatttctt

SEQ ID NO: 68 - DNA, 1000 bp, Arabidopsis thaliana
taaaaactaagttgtccaactactaacgcaattgacaaccacaaccaacaccaatattaagtatgattaccgaccaaatttgaattttataaacatatagaattttttttgccaactcataatcatatttttatatgtattttaatataaggtgaatgattatcttttgtttcatgtcatattgggcataacaaaactagtacaacaaagttgtgtgaaccactccttttatatagttttgttgttgtcttttcttccaaggaagattactagacttagacaagtggattatatatatatatatatatatatatatatatatatatatatatatattaactaatctttcaacacttgtggattgataattattcatacttttaataaaaattccatagttcaatgaatctgtaacaaaaccaagaagtgacgtgatatacttttggaactataatcatattgtgagctcaatggataagaattgattgtagtaaatgatgtcggcagttacaagtgtagttagatatatgcaataactcaaatcggacatgaactcattttaacttatctaaagagaccaccgacgaatcattttgggttcacaaaattgtacttcgatttctaagcctgaatgtgaacgcacgtttttgaatatttcaacacgtgtttcatatttcattacatgcattataacataaatattacatctttgagtctttaactagttgaccaacaaaaaaaaaaactttaactaagtctagctagttttgttactacatatataaaaacaaaaccgaaataaatatttaaaatttataatatatttgtgtggctaaatcaatcaacgtgtcatgaaggtctaattcaagttggtaaggaaatcttttgtttatgtccatttcccacgtgtcactatttgtatgacggctagagaaagacatgttgaattaactagtgactccggattatataagcaagcatctactaaaaagataggaac

SEQ ID NO: 69 - DNA, 1000 bp, Arabidopsis thaliana
gaatcgttcttgatctcaatttggtttgtgatgagaatgactttgatatttatagtgatgaaggaggctagggttgttggatctaatattttcaaaaatattcgaaaatatcttgcaggataagttgcaaatcaaagattggatcctcactaatgattcgaatttggtttagttcataatgattggctatatttagataagtggtaaatattgtttagttgattatggttagttttagatttaagagaataaatttataagatttttttctaatttatggaatgtattatcgtgttatggtagttagaagaatcgaataagctcgtctatgtcgattttatagtgattagtgaggagggtgaatatatgattgaagattcttaaaatcgtaattaaatatgcagactattttgatggaatatgcgtgtattagatactaaaaattgttgagatatacaactaatcgtgttatggttaagattgcattacttgatggtaacctattatgtagttttacatgtgatcaaaagcccattaaacataactcacccgtctatgtaaaacgcggatcctgtttaatttagttatttggtttggacgattttacccttgataaggaagaacaaaattcaaaattgaaatcaaaattgaggctaaattagacaaaaatttaatttgtacttcttttttaataataagaggatatgactatgatacctcgcctaattctggaaataattttttaaattaatcaagatttttttcttaataggtaagccaaagatagaacacgtctcgtactattacagaaatactcttcaatctgtcacaataaaataagaattagtatcatagatggtaacgtatctccatttatcttttaatattagacgttacttaattcttagtaagagaaatcgtaatttacttttgtactttaattgtggtcccttgataccacattttcctataagtaagtacacgcaacagcatcaacaac

SEQ ID NO: 70 - DNA, 1000 bp, Arabidopsis thaliana
catatttggaatccatattatgcaaggagacatcggttttactaatgcgaattgaggggcttgtataaaatcggtctgcttcagaaaacattccatttttcaacccttgaaagtccattcagtttaccatccaagagtttttccttgttgtgaatgataagaaaatccataacatttatctaattttatcgtattggaaaaataatttaatatttctgaatacatttattaaaaatatagttaaaaacgatgtatttttagcgttttataattatttagcattatttagatgttttcgttattttgttattacatcattttaattattattcatgtgacttgaaattaattactttttaaggtcgaatctttttttacgcctgcctttacacttcttattagaaggttatgtatcgaaacagccatccaaaatttaaataaattctaaaagaagttatatatattatgaatgtatggtgatactgacatgggtagggtacattctttttctattgagttagttgatacgttaagttggatgagatataacttatactatcatataagctttttttaatttgtgtgtttcaagtttggtcggttttgtttatgtcttttgtaggtggggtatacagaaaaataagagaaaaaaaaacaaagtcaaagctgactttgacgacaaatcgctaaccttcgagagatgatgataacaagcctaattaaaatacacgttacgtcgcaatcaattgcttcaatctcattctcattgcatgtcatccttagccgcggttagccgaccatactatcaacgatgtgcatactatcaacaatgtatttgtaataattaagtagaaaaataaagaatgttccaccgtccaaaagaaacacaagtacggtcctgagagggcttgagatattttgcttagtgatatatgtcaacatcttagttaagccccaccggttctgatataaaagtggggaaaatatttcataaccacac

SEQ ID NO: 71 - DNA, 1000 bp, Arabidopsis thaliana
atctcgagatagcctgattcagctcaaagaaagagtgtacgagaacattgccacagtgcctctagttgtagagagaatgcgagagagtaaagagagaatcgacaagtccatttctttcaatggaactactatgcacccggctttcacaaggcggaaagctagctaactgatcagtttctacttctccctattggctttaccactcaaagaaagctaatgcataggaggagatatggctggtatttctttctactatattagtatcaaccaacatctctcgcatttagaataagagtataccataacctctgttttgattgtgttctacattgaaacaggtctttctctgattttaatatggactcttatagaatcttgggctgattctacaccctgtatgtatatgtaagaatcacataatcagatggttgcacaattgtcaagaacacccaaatccaagcttgcgatttagcaatctcacccgtgtaagaaacttaaaaccataaaaccaattgatataagagcttttctagcaaaaacttaaaaatataattaacaccgtcgaaaatggtggaggctactagagcgagatataaactttatcaccgtttaatcgtgtgggattttgaaatgatatgaacctattgcaaaaagacaaaaaaaaaatataaacggaggaactaggcagtgggaccggagatccaccgaccggaaacgaagaatccatagtaagatagagcccaggggttcggtcgtatcggaccggtcggatttggattctaacgacaagtgagtggacccccagaaacgacagacgtaagcaatgacaacatcaaactcatcgccacgtcagcaacggagttctacttgcgaaacgtgtcccggcagcgtcatccacccacgtgtgttctagaagctcttctcctcctccgtatctcgacacgtgtccactccctctccttatataattgacttttctctcattctttccaagtttcaa

SEQ ID NO: 72 - DNA, 1000 bp, Arabidopsis thaliana
gcggttgttggatggctgaaggtaaaagctgctatgagatggggattcttcataaggaggaaagctgctgagaggagagcacagattgtggagcttcatgataataacgatggtaaatgaatagtcaggttgcttgctagtgtatagaagccatgtacagtttagggatgtataacaacatcacttaccgatatggatttgcttgatttagcttattagacaagaatataatcgatgaaccatgagaaatttgaagactaaccttcagcagcaagacaagaagctgattttctgtctctttaagtttcaactttcaacttgcagatatattagaagactgactaagtatgggcctataagcccattaatgtctctactgaacgcccaacttgtgaaaaacatgtctctcatctcaattttgttttcttttcacatcaacttaaagctacgagaaccaaacaagtgatgtttttggtcaaacaggacgaaacatgtatacataaaaagaggtaattatgttcgataaacataggtataaataaactataagaatttgttacaacattctttagatttgatattaactgctgtttacatttgaagaactcagttagttatttaatattttattttatagggtctagaaattacttgcgtgtggggactgatcaccgattctaaagaaggctcatcctttggataatagtatgaaacttgtcaataaagataagtcatcacaagtagggagatcttagctgtgttccatacgcccatctagaaaaagcgacgatggtcaagattaaataactgtatttgaaaaaccaaaaccgcgtcaccaactccaaagccattaccattagccatcactttccatcttccagctgttcgaatcaggacgcccctttttcttcaccaaacccatcggccgataacgaaccttcctctctgactgcctctgctcttactataaatacaaccaatacgacctcatccaaaaccc

SEQ ID NO: 73 - DNA, 1000 bp, Arabidopsis thaliana
gaccaccggtttctcaagtttccagaaatatgcaaggatctggtggtcccggcggcagattctcagggagaggagatccaggatcaggccctgtttcaatctttggtgcttctacttccaagatcagtgtagatgcttctttagccggtgccatcattggaaaaggtggaatccattccaaacagatatgccgtgaaacaggagcaaaattatcgattaaagatcatgaaagagacccaaacttgaagattatcgagctggaaggaacatttgaacagatcaatgtagcgagtgggatggtgagagagcttatagggaggcttggatcagtgaagaaacctcaagggattggtggtcctgaagggaaaccacatcctgggagcaactacaaaaccaagatctgtgataggtactctaaagggaactgtacatatggagatagatgccattttgctcatggtgaatctgagctgcgcaggtcaggaatcgcttagttatgtctttagactcttgagaacagattatgcattgttagctccattatcattgtgactttttgctctctctttttgttttatcaatttgttttatgcgactcgctttgaaaactttagcccattctgtattgagctctgaagattcgacgagttctgtaagttaccgatcacagttaaaagactttgatatgttaaaacccttatattacagctacatactatttttgtcttaactcttaagatatcatgcacaataatatacttgttttgtcttaacctatcgattacaaaaccgggttaaccgccgacatgaggcgaactctaaagcctaacacgcgtcaacatctatcttctcaacgactcaaaggctttccaacacgtgtaggaccaataactgaaacacaaagcctaccacctcttcttcctcttctttcatgacacgtctcactgacgtgtcgtcaagaacgtaattaaatattaaactatcgtgacgaacgcgaggc

SEQ ID NO: 74 - DNA, 1000 bp, Arabidopsis thaliana
taaggttttaggccaatgatagtgattatacctcttttgtttgtacttgattcgtcaccattattctgcttgcgtttattttagaatttagaggtggctttcggaattaaaaacaacaagacaacaacattttataattttgatttgtatactaataatagaaccaaactctgcttctctcagctattattatctaagtactgatatatttcaaattgtagcataagttctgtatatgtattagatagagttatcttttgatttattatcatatacggattaaatttgcagtttagagcaatgtatttgcaattttgtgaaaagtgaatttgctttatttttgtcttcattactaaagttgaattagcggtttgatgagtcattatcgctattaatttgtttccgtaataataatgataagcaatcatgtcacgaacatattactaatattgtggtgattcttttgccctcctcatccatattcattgattgtaaaagcacacgtaggtcacatacgtatcatcctaaaccccaaattaaaaaaaaaaactaagcaatttttatacatggcctttggcgatccaagcatgcaaaaatattccataaatggttaacacaaaatcatcgattctgaggggttaactagggtccactttattaccgtatggaaatagagacacgtcaaagctagtatttggacctacgtacgctcatgtattccgccacgtgtcgtggtttcactgacataaaacacatgtcggtcagataattctagctctcacgacgatgaagcaatgtaggctcacttccactcaccgccgcctatagcagtgacacacgtcctgtcaccggcgtgtcactcaataattatcagattttcattttaattaaataatgttatcctatatttgttaggtggatcccaacgggagggctttatggtaattaccatttactcctgagaagcttttatttttgtgtgtggagtagtaggagaagt

SEQ ID NO: 75 - DNA, 1000 bp, Arabidopsis thaliana
cagggtggtgaagcttctcctgtttggccaaccgatgcaacaatgcaatcaatctctactcaaaccactctcaaatgcttatcccggatgctagaggaaagcatactaaccgatgtaatcatccacacagccgatggaacactctctgctcacaaagctatcctctcagctagctcaactgttttcaaaagcatgttccaccacgacctcatggaaaaggaatcatccacaatccacatagacgacatgtcgagagaatcttgcatggctctactaagttacctctacggaaacataacccaagaagagttctggaaacacaggctcgccctcctcggcgctgcaaacaagtacgacataacggatttgaaagcagcctgcgaggagagcctaatggaagatataaactcgagtaatgtgctcgagaggctacaagaagcttggctttatcagctggagaaactgaagaaagggtgtttgatgtatttgtttgatttcggaaagatttatgatgtcagagaagaaatcagcagtttcttcaggcaagctgatcgagaactgatgctggagatgtttcaagaggttttgtctgtgtggaaaccggtctaatatacacttacacacatactttgaacatttcagcttgatggtgttttgtttatagtcttcttgttctcttctgtgtgtgtaactaacaacaactctggattttgttgtatcactctgtctaatgtatagtattgagtggatttgcctctagtttggtgtaccgagcctctcaaatgggccgagcctttcaaatgggctgtgactaggcctaatgactgtcgacatcgaatgtcggcacaaaatatgataattttgtcgatgttgaaatcgtacgttttttgttttaattagatgagtcagcatcaggaggatgaagtatcgaccgacacgtgtccgttgcgtcccaaagtaaagcttataacgtgaaggcatttgtatcgtctcttccgcc

SEQ ID NO: 76 - DNA, 1000 bp, Arabidopsis thaliana
ttctttattggtaactttttaatatattcatttggtcaaagctggcaggaaaaaagttttcaacttataattttcttatgaagaagatactgatacaatgcatattaaggtcaaagcaagagtctaacaatttattattcaaggaatataaataattgttaggtaggttcttattgaaatgaagcatttcacaccgtaataatgatatagctgtagaatatgaacaatgcgcgtttgcttgactgtttcgttgtaagtttgtaacaataatggccaccaagtaatctgtcactagaatattgcaacaaaataaaaaccataaatataactataatgatctactaatttattaatttaattaacatgaaaatatgacagcttttgccagcccttgctcgtataatcagtccagataacacattacaacgaaacatataaaataacttttttcaacagaaacgattataatatacatctaaaataattaacagttgaaaattttgatagacatactatatatgaatatgaacttaaataatgacccatttttcgtataatgttaattatttactcgtaaacgcgttatttccacgaaacattaggcaaaactcaagttaatttacgcctggcattgtaacgcggttaaccaaaaagcaaattacgcagagtcaaatcatatctaaaaaccaatataaacataacacgtgtcaatacttaactgatctcagaattaacatcgttaagagaaaacacgtggcagagatctgtgtatccgtttggtgctccttcatgtagatgattcttcaagaaaacttcaaaaactcaaacacgtcaagtttaagaaagaaaaaagacaacaattattttaaaccgccattgaaaagctaagccatgttgtatttttgtatgtggttcgcatgattagtgtcacaccaataattaattattaactatttcccaaccatcgcgtatatatagagctctcttctctcattgttct

SEQ ID NO: 77 - DNA, 1000 bp, Arabidopsis thaliana
tacttacaagcaattgtgaaagagactcttcgtttgcatcctgcagctcctttgatccctagaaaatcagaatccgatgttcagattatgggtttccttgttcctaaaaacacccaggtttttctcatacttatctttattacaccaacattcattacttgagatattacaccaataaaaaagtttggtttggttaaaatcttccacctctgtttctcgagattaggttgttgtgaacgtatgggcgataggacgagacgcgagcgtgtgggaaaatccaatgaagttcgagccagagaggttcttgttacgagaaaccgatgtaaaaggcagagatttcgagttgataccgtttggatcaggaagaaggatgtgtccgggaatctcgatggctcttaagacaatgcatatggtgcttgcctctcttctctattcctttgactggaagcttcaaaacggtgtcgtccccggaaacattgacatgagcgagactttcggtcttaccttacacaaggccaaatctctttgtgccgtacccgtcaagaaacctacaatatcgtcttcttattaataatcgtatcaatataaagttcgtgtacggatcaatattaataattgaagagaatgaaattataaaagatactgctttttatgtttcaagtaaaaatgttaatattagaaagtcaagaaagaggaaacacaatgcaatttgataaggtgttttatcgtctccgggattgaattgcgtaggtcaaaaaaagacatttgccatttgatatcgacatataatcactcattcacggatacgtatctcttgtgaatggtcgtcggtcaattacaactttactgctcggctttaactgacaagtggcgcctcctggtttcattcagaccctaccggtaggatgtaatttggaccataagtaagaaatatttgtgaaatatttactacacgaatatctttacgtatttaagttggcttttgaagctttgataattc

SEQ ID NO: 78 - DNA, 1000 bp, Arabidopsis thaliana
gataaaaacagatacttttagcgaatacttataacaacactgtgttttgtccaattcaggggaggttctacgatcaacccaatgcaacaattctatatgcttccgtttcataaaaattctgaatatcaatctaaacctatacaattacatccttactaatctagataaagtgtatacaataaaatgagagcaataaggttcattaatctttatacttagctcctccacaggacgatatactttgagactgacacaaaacaaaataaggaagaaagcttacgtttttgaaggaaaatcacaggagggacgcggaagcaagattggtctgagaggaaaattgaggaagaggggttaatcgagatgaaaatggagtttgaagtaatagagagaacgttgcgcagatcgaggaagaacagaggataggatcaacacaaggatcttgtgaaaatgaaaatggctgaggaaatgaaggactatttgttaaataagatataatattatttctgaaatatttccatctgactcctttaatttatacaagcctccttttttgtacatctattttcagaagatccaaataattgtttcttctatttgtgtatttttgatatttaaacgtaaatctttggattttgatcaataatagacaattagccaagttcagttttcattaattagatttataattttaattatttttagcattcgtctaaaattaaataatgcggcaaaagaggagaaattaatttttgttgttattaattcaaagcgttacaaataaaaaaggaaattccacgctggcaataaaataagcgaaaattccacgtgacatctacctgtcggatcaaaaagagtggaattgacatttgtttctctcacactctctctcgaattctctggtagcttctagttctatgcaaaaaaacgacgatagttctctatctttccagatgaatctccttccatatacaaaagcagtcatgcctcctcgctctctcgc

SEQ ID NO: 79 - DNA, 1000 bp, Arabidopsis thaliana
aattcacatgtgatccggtatattaaaatatacatgatttatggatacactcgaatacttctccgatattcatttcgcggaaagttgcatatgatccactatctcctatatatttgttatataacacatggcaaatctctctatatatattttttaaatacacatatatccaaaataggcttaactataacacaaagacttttgtttactgtatattagtaagaagtaaatgtattttttaatattatgataaagtttgtgaaatcaccatttgcaatagccatatagggtcgtgttttaattttacagtttgtattgttataattcgattccaaggttgagaatatgtgtgtactattagactatacaaataataattcgttgacgatattgaatatttactaattataggaagagaaaattatttactaactatagtacgatatatttcttctatatgtgtttttaacgtttttttttttttaaatttaagtcttaactttacttctcatttttaatcaaaaggaaaaaaataccaatcaatttttcctaacacagtttacttatcattttcatttgaaatgtgttcactttctgataaaatgctaatcctacaatcaaatacaccattgtcgtgataacacgtgtacggctctaaagcaatcagaacaatcattggacagtttttacaccgtcagataagtacctatccacttgctgactcagccggataaaccctaaaccggaagtttgccccaccgtcaaaattggaagaaaccggacaaaagagaatgtaaagactaagaagtaagaacccatcggacgtcgtaagaaggttaattaacacgtggaaacagctggtcagagttatccggtaacttatccggttacaagtaaaaaaataatttgttcccatacacgactccttcagaaccaaacgcgacatcacggcgccgtttagtgtctataaatagagcaatcggtcgtagaaaaccaagac

SEQ ID NO: 80 - DNA, 1000 bp, Arabidopsis thaliana
aagagattttgaaaggtcgtcgaggagttagtttattatcttacaattattatggatttcttctgactaaagttttagtgcaatagttttctgagttattatatatttttcgaaattcatccaaaggctaaaactttgtaaggtattctgagtctattacgctagaaatagacacgcttacgtttttaagtcattttaatctgatatatatatatatatatatatactacaattttataaccaattatctttgtttcagacaatttatataaattcatataaactatcagaaataaagagacaattggctgtaaaattgtattattttgcatctatgaaatttttatttctctttatcaaaattgaagacgagttgaaattctatttcttttttaaaaaaatttatatatgggagtgtcaaaaaataattttcctttgtaagagaatattcgtatttggacgagtcttgatttgtgttttccgttagcgttccacattagttgtaaagagcgagaaagatttaagtgataattaaaaagacaagatatgcattttgaatatactctagatattaatcttaagcatttttcatattggttatattttatggtcgaattttgacatccgaaaaatacatcccttattcttcaatttatattgaatacttaagtttttgatagtaaatatgtataaataaattttaaacaattgtcaaaaagacattataacttatacggttatagttgttaaaaaaaaaacttatacggttataccaatagataaactgacattttagatttgagttttttttttttttactttccatgtagatagcgtggaataatacctcatttatatgtatcgttagttgaaataattgacaaaaggaaactattcgtttgctgttctagataaaagcctttaattgagaaaaagaaaatgcctccagctttgacccgttttgcctataaaaggattctctaagatatggactttcctc

SEQ ID NO: 81 - DNA, 1000 bp, Arabidopsis thaliana
aataatccttcgttgccagcagtctaatgggactgtgttataggttaatctctctttcacggactattaatttttggctcaacctcaatgtaatttatttaaattatatttcaacgcatacacgtgaatttatacatcttttcataggatttgcaattccggattctcatccgtcctaggaggttgtatatgtatattattggtaactacagcattaaacaactttaacactaagtacgatgatattatatgtacatgtacgtatgctatagattgatataagaaaaaagtaaagagagagcggatgattgttgattgaactaattaacaatagtgtattaggtaggcttgtatccgacagtcaacttaaacttcatagttgaaaccaataaaaccagaagaaaaaaaatttatatactaatcataatcagcatgatgttagacgatttgatatctaactttcttcgttatgatagagaaataatattcgcaataaatcacatataaaaaaaattactttacgtcatagattatcaacttgcctccaaacacaacaacatgatcccctacgctacaatacataatatgaaaaataaattaatcgttgattaacttaaagtcactccgttaataacgttataagcaaagcttattagtttttttgaccgtcattatcgacgaccaaacaaagaaagagtcccaacaaattatgccacgtaagtccagaacgacgcctatgaaaccaataagacaaaaattcaaaaaccaaaacatccgtgaaaactcttacgtggcggtgacatgtgggtcccttatgtctccattaactaacataagcttattgtcgactgtaccttttgtcccccgtctcggctacaaccctgtgacaaacaaagatcaagtcaaaatatctaacctaaagtactctcgtccacgtggacattgctgacgtggaacttctcccttctataaatacaggcttctcttcgccgaattatac

SEQ ID NO: 82 - DNA, 1000 bp, Arabidopsis thaliana
caagagcttgactcaacagctcaacaaaggatgagatgggtgcagaggaactacatgatctataattattgtacggatgcgaagaggttccctcaaggtcttcctaaagagtgcttagctgcatagagagagtaaagagttgagagaggaacaagattttatttttctttgtggttataaaattctattcattttattgtagatcacgtgaattttattgatttgttttgtagtatactctatagttcgttaaagttataatattctctttgttacaatgtgcttttttgtttttttaagtctctcgtagttcgttccgttatattctccgagtcagtccaaaaatggccgcttctgttttaattttgttctagggttccacagtcaaactcaaaaccaaacccaatggagcaataaccttttttttagttttataatcgaaatcaaaccggaattttgcatgtaatttgattggtgtcgttactttaaatctttaatccacaaaacaaaatttactcgattttagtattaaccgaaccaattatagtttattgaaatttaattttaattctatcaaattgcatatgtattcttgagttattttttataaaaatactgaaaccaactaaaataatagagtttggcggaactaccgtaccaaatttgattgtatttggagtatcatttttgcaaacctaattagcctgaagactgagatatccttgtccactcttatgaagaaccaatttaacaaggtgaaaaccagaatctctaaaccaaacatggcatcaactgaaccggatcaggcagacttaaaccaaaacaaagaacaagcacacgtagcatgaggcaaaattaagcacatgcttgctttacttcaaaacaaaaaccagctgttcacagctaaaactacacaagagtcacaaacggcgaactatactacaaaaagactaagacttgcctcccttatataaaaccccccaacacataaggtcccaat

SEQ ID NO: 83 - DNA, 1000 bp, Arabidopsis thaliana
tctacgactattatacattcatactttgaatttggattacaaaaaaaaatcttcgagtatgagaaaactattaaaatagttttattgaaaaatcatataaaataaaaataaatttagtaatagtataattaaatatgtaaaataaaattaaataagaaatatatcattaacctataaattagaagttgaagagcttacattaattatttaatagacacatgtcaaatgctaaagtgatgatgtgtcaatcatatgaagagagttggccaactttcatatatatgattcttagtttcgtaccaattagtttctcaattcagtgcatgtgtagtcaaatgtccaaaagattgtttctaagcaattaaaatcttatcaaaacttcattgcccaaaatattacgaaagcttgtttctgtgtatgatatacccggtaaaattaaaatgatcattatacagaacaaatcagcatgattttcggataaagatgacacacatttgaaatcgtagccgtactacgcgaaatacatgcactcttcgttatgttaacactttaacagtgaacgtagccataatgttgaccacattcaacagtcaacacaaacattactttacacacaaatatatgattatatatacatatgtacatgtaagtgaatgtgagcaataatgacgggaatattcagagaagacgatggtgaatgttagcagtgatcacgggcacattcaaaactgactgtggacaaaaaaagctcctggccttaaatatgattgtgccaaaaatagtacaaaactaagaacccaaaatggaattcgagacctatataataatatatatgtatatagtctttccttggaaagaaatcttatgttattaagaaaaatactataagttatctctctatctagatatgatatatatgtccaaacatttccacgtagatgacgtatattaccgaggataatcctctatataaggaagagaagctcgagtaataaatctc

SEQ ID NO: 84 - DNA, 1000 bp, Arabidopsis thaliana
gtgtatattttgtcatgcaaactatatcgtggagaaaataatgttgcttatgacttttgatagttgggcttacatttggataatggatagggtagacaaagataggaggaaagcaataatagcgaaatgaagaacgaatatttggggaaataggacaaatgaatatacttctctttgaaatggagattcacctaaattattaatactaaagccatgcaatgcatccaaacaaatcagtggtcaagcacactcaattatatgtccacgaagacctttagaatcttcacaaccaaaagctattttctacgctacctgataattctgactcaattcttcttcataaaacgtataatgaagctttatgaatgattaattatagacacaaccggccctatctgcgatttctacaaacaatagaacacaaaactttaaaagttactacaaaataccgaattgactatatatatcatattatcagtataaacatgattagattgatcatgtttatcagtaatcatgaaagacaaagagtgtgactattgtaaaccaaattttagaataaaataaataatttatcatactatatacagtattttgttaagtatatgtcatccaatagtaacattatcatttaaactgaaaaatgtttcagctactttaaggaattatagctttattaaaagtatatacttttaggtcacgtgtttagaggtgaagaacaataataattactcaataagttcaccagtcacactccaacatcttattcaaattccttttaaaagctttttaaccgtggctgtttgatgaccatttgacaaaatttagtatattagaaaaaaacaataggatagggataatataggacattagactattagatggacaaaatgaagtattatttaattttccaatgtaccaaccaataagaaagaagtgacgcacagtaaacgacaaaaagctcaagcataaaaacccaaaccttctctgctttctaaac

SEQ ID NO: 85 - DNA, 1000 bp, Arabidopsis thaliana
gtaatcttaaaccattcattaattatgcgaataaaacgtaaaaaacgttatcaggtttcaactttatttaggctgcaatgggaactatggatggcataattgatacagtttctgcatcgcattcgatttcacctttgattggactactcaaatccaacggtaaacttgttctactcggtgcaacggagaagccatttgatatatctgcattttccttaatattgggtatgcatatataaccctacgtaatagaacgttatattagtctttcacatctagactatgtatcgagtatgatgaaaccatgatgcgacaggacgaaaatcgatagcgggaagtggtattggaggaatgcaagagacacaagagatgattgactttgcggcagagcatggcataaaagcagaaattgagatcatatccatggattatgtgaacaccgccatggatagacttgccaaaggggatgttagatatcgatttgtcattgacatttctaatacattggctgctactcgatcttaattaaagtcgatgttctatatgtattcaaaataatctggatttcaatcccacaaaacttaaggatatatatatatatatatatatagtctattttatataaatggagtatagtcaaataaatatgcattatcaacgatatatagtcttctattacatagatacgtgggagttcacccaacgtagatacgttcggttgaaacaagtcaatttcatcaatgcctcttccaaaaaaaaaacaaattgcattattgatgaacacatgcatcattatcaaataggttggttaaaatgaccaagatgactaaagccaatcacactactaccagatcgagtaaccattagggaccattaattcacgtggacgtagtgaatatggtccttgtgaattaatgagtacgtaattgtcctcattcatatatggatcggttccacaaacatttcctgtataaaattctacatctttcctctcattatt

SEQ ID NO: 86 - DNA, 1000 bp, Arabidopsis thaliana
cagcttaccacatgcggccacaattttaaccatgattaatcttcttttggaatttagcgagataaatcaagagtaggtttattttagaacatagtatataatacactaatgaaaagtctcgttaaaacgtgtttcacgtaaccgacctctaaagtatggcgtgcgagtaacgacaaatgaaatctcaaatgaaaatggagattattttaatcttaattataacacactaattaacacccgtcaaacatcctgatatgtgaacgtggaatctcgttacacaatcgaataaaattattgcttaatacgatccgtctttctctctctttcgtaatgattatctatcaatattatcttgactaataaaagtatcgcaaggccttggcagtcacatgttaaatgatgatatacgttgttgcatgttaaaacatagtacaacgatcatctaggctgctgctaaattctattttcaaaaatgccttttatatgcaagaaaagcaaaataatcgagtttttttttttgtttcattataaactgcttcatttttcttaggaacggccaaactgttaaaaagtaaaatatgtatggtgattaattgatgtaaccaaagccagttcgcccgtttgattgtcaacccacgttatcattcacttgatcacttccatgaaacatataaaagctgataatacttatattataaaggaaaaaaagtatgaaaatattatcagtagttagatgattagttcacatctaaatgaaatacgacttaaactgaaagagacatgagccaattcgtgtcgagccacaaattttggacgtatatttttagcccgtggttccacaatatttgcaggtctttttattcaatgagtttattttgtcttggttgaataatgaaatttccaaatataaaaaataatagaaatccgaggccctacacaagcacacatagtaactcccacattatatataagcggccaatatcagcaactcagagattcc

SEQ ID NO: 87 - DNA, 1000 bp, Arabidopsis thaliana
gagataggacatagtattcatcactttaaaacactcttattgataaatccaagagtcacttaactttttctggtaatcgctgttaaagactccaactcaaaaacaatcacattattccaagtcttaacattctgatacgattcattacaagaaactgagtttatctaagattttcttctcacttgtgcattaacatatatacacacaacataacacagatatgatacaatttcacttctttgaagaacgcttgggagtaacagcattaaagatagcttcaatctcttctttcagtcgttgaaagaaaccaggtcctttcacataatcaattggagtgctctcatcaatgtcgtcacttgttccatgagtctctttgtcatggtgacgactcttggacttttcatgatgacccattgcctctatttcttctttaactcgttcaaacacatttggcgatttcacagtcttctcctctgtggaaatacccaattaaaacaaagggtttagattcgtgtttcataagagacaaaacagagcaacgaatcaaaaatagacttatcatggcgtgtttctattgtttctttagagaaatttccgatcaaaaccatccattaatagacgtgtaattttgattgtgaaatttgaggaaacgaacataacttgatcaaaaagtcttcctttttaacaatatgcataaacctatgatgagatctgatcgttcattgtgtttacctgatcggttttgatccgccatcgatggtttttgtcttcttgatcttgcagctttctatttttggttcctgattcggtggcgttttgcgtcagatgcaaaagagtctttacagtataaatcaaaatcgatttcaacttaaatgggccttgttgggttataatgggcctttttgtttgtaagtgagtccaaggaaaaaaaacaccagaccagattgaaattgaatcacttgcaaaagcaaaattgattactctgttttggttttggatc

SEQ ID NO: 88 - DNA, 1000 bp, Arabidopsis thaliana
aaagtaaagctcaaagactgtcctcacttatagtttatgaatcagcaaaagaggaacttaccggcaaagcaactaaatcataaacttgatcagcacatttcgagataagaacatcagcaagcaatctggtccctgaggatccttcaacctctagcttctgctccacagaagcaacagtaacatcagcaccagctcgccgtaaaacatcaactaacacaacagcttctatttcctccgtcccatatccaattggaacaagcacctacatagtttctcagttatctccaatctcaatctctctacacaaacaagctggacaattttagttatagataacaagaaaaaaggagaaacctttttagtagttgcagaagacccgacgccaacatcggaatctagcgttgttaccatacccggagacatggaagatctaagctttaaagttcggtccctccttgttgtccctagtgaagaagaaactacagagactgaagaaaaactaggagcaacagtcatcgagacacaacccatcgaagagataagccttgactccatcaaagttggactcagcgaagctatcattgaaatcgaatatcctaaagaccccattgacgatagctaatgcagcagtctgataatcccgccgctagacggagagtgagttgcgtcttatctactagtatacacttcttgaagaaagctaaaaacgtcgtcgtatcgttgtcttattgcttgattggataaagcttcaattttttcctccaatttttgtcgaaaatgtaacattttttttgctttccactgtgagattgtcacacacgattctctaaagaaatgagacgaattttgaaaacgatttataaaaatagtatttttgtttccttgagcttatctcctcttttctctttggtttgagatatgaattacagacaatcaagtagtcagtcacaacagtgtgtataaatatgtcttttaagtgaaatcggtttcgagagacagt

SEQ ID NO: 89 - DNA, 1000 bp, Arabidopsis thaliana
ttgtgtttatctcaatgtttagatgttgaaaagtatatatgctgattaataaataacaatttcttagtcaacaactcaatgatagaatgttatataactctcttacatcatttacatttttaaattgaatgaattctcatactatgcaatctacctggttagggtgaagtcacaacaacaacaacattaactaataacaacaatttaagctgcatgtcctaatcagaatctcggttattggtcgctcataagaacagttagaggtctttcagtcacaattgagagacgacatacataattggatgggaatcaaaatcagtttaattgcgctatacgcaacttgaaacgtcattatacaccgataagctttaaaaataaaatgttgtgatggacacgcgcactatatatgtatgtgtttggacatgattgaagctaaacaagataatatttattaagaagaaaatacataattattactaaataatgtgtattattatacgattccaacttttatttgataatgatttttgagcaaatcaggttgataggtatgattgagaccatcgcccaaactacaccttaaggtcttactattagaaacaaacaattcatttgggaattgaaaccaaagtcatctatggaagtaacctcaaaatttatctaagaaacagtgatgtattagaatggtcaagtcaacttattcacaaaattacaaaactatcgtttataaatatataatctatttttcacttgtgttacacgattcctcttttgatatgcaggaagaagagaagatcttacaacggctagaaatagaaaacttaatgtgctattaattacaacttaccaactaattagaactttctatatgaaccggatcttctacagcaaatcacggtcaattcaaaatattcatctctctctctctctccacatggacaagacatgattaagaaagtatataaaagaaacagagcagagaaagacattttgttccggt

SEQ ID NO: 90 - DNA, 1000 bp, Arabidopsis thaliana
gtgagcagataaggatcgtgtattttcaagtattatattaattaataaattatcgcccatcttctattgttttgtattttcatttgaaaagaaagcagggctggcttatcaatatatacagtctctttcttttagtttactaaaatcatttcagataagacccacacaaaagtaatatcaaatggaattttgctttcaacatggataattttctttaataataaaattgtcaaccatgttaagcgtaatctctaaatccccaaaattaaaaacactaaaaatagtcggtcattgctccacgtttaacctgtccaattccaaaagaacttcgtgtttcattgacgatatagtttacgtacgtatgattctttctaaatgaggtatttttgtcccattagcaatttaaaatttaaaatggttgtgtaactttttgaaatgcttagtagttaaaaccgaactaagaagaaataatgatcggaacgtgtttatgtcagctaatgcattacccactgacattaactgatccagtttactgattttagacaatcatactctatttttactggagttgcagaaaaagggacttacaaaacgatgaattaaatttggacaagtatatatacataattagaaggttatagaatctagttttcgtaggatatttcttgaattatcctacgagtgtgacaactaaatgcacaacaaaacaacaatttaaatgctaaaaagcaaaaatagtaattactggtcacctattaaggaacaacaccaacacctatttagctgttatgtcctaataaaaataaaataaaataaaaacaataaaccaaaaaagtaaaaaataaaaatgtttgactaaaattctctttcttttccgattcgtcattcgatgaatcttcctttcttatacaatttggataatcttttctaatgtaaaaattcgagatttttttttataatgttaaatctataaataagagcaacctaaaacgaacaactt

SEQ ID NO: 91 - DNA, 23 bp, Artificial Sequence
  Description of Artificial Sequenceprimer
cgccagggttttcccagtcacga

SEQ ID NO: 92 - DNA, 24 bp, Artificial Sequence
  Description of Artificial Sequenceprimer
agcggataacaatttcacacagga

SEQ ID NO: 93 - DNA, 588 bp, Arabidopsis thaliana
  CDS (1)..(585)
atggtgaagcaagcgatgaaggaagaggagaagaagagaaacacggcgatgcagtcaaagtacaaaggagtgaggaagaggaaatggggaaaatgggtatcggagatcagacttccacacagcagagaacgaatttggttaggctcttacgacactcccgagaaggcggcgcgtgctttcgacgccgctcaattttgtctccgcggcggcgatgctaatttcaatttccctaataatccaccgtcgatctccgtagaaaagtcgttgacgcctccggagattcaggaagctgctgctagattcgctaacacattccaagacattgtcaagggagaagaagaatcgggtttagtacccggatccgagatccgaccagagtctccttctacatctgcatctgttgctacatcgacggtggattatgatttttcgtttttggatttgcttccgatgaatttcgggtttgattccttctccgacgacttctctggcttctccggtggtgatcgatttacagagattttacccatcgaagattacggaggagagagtttattagatgaatctttgattctttgggatttttga

SEQ ID NO: 94 - protein, 195 aa, Arabidopsis thaliana
MVKQAMKEEEKKRNTAMQSKYKGVRKRKWGKWVSEIRLPHSRERIWLGSYDTPEKAARAFDAAQFCLRGGDANFNFPNNPPSISVEKSLTPPEIQEAAARFANTFQDIVKGEEESGLVPGSEIRPESPSTSASVATSTVDYDFSFLDLLPMNFGFDSFSDDFSGFSGGDRFTEILPIEDYGGESLLDESLILWDF

SEQ ID NO: 95 - DNA, 1290 bp, Arabidopsis thaliana
  CDS (81)..(1088)
aacaaatctctctgtttctcccgctcttgctctgttttctcaaagacaaaagaggacatcgtcgttgactcctcttctctatggctactgctaagaacaagggaaaatcaatcagggtccttggtaccagtgaagcagagaaaaaggatgagatggagttggaggaggagttccagtttagtagcggcaagtataaagattcgggtcctggctcggacatgtggttaggagatgcttcctctacgtctccaagaagtcttaggaagactagaacctttgaccgacataatccctatctcgtatcttcttatgctactcctcagccgccaacaacaactacatgctctgtctcttttcccttttacctccctccagcgattcaaaatcaacaacgatttttacacccgaatgacccttcaggacaaagacagcaacaaatgatctcgtttgatcctcaacaacaggtgcaaccatatgttgcacaacagcagcaacaacaacaacatctattgcagtactggagagacattctgaagctgagtccgagcggaagaatgatgatgatgaacatgttaagacaagaaagcgatctgccactgacgaggccaccggttcaacccttcagcgccaccaagctatatagaggtgtcaggcaacgccactggggaaaatgggttgccgagatccgtaagccacgaaacaggacacgtctctggctagggacattcgatacagcagaagaagccgccatggcctacgaccgcgaggccttcaagttgaggggagagaccgctaggctcaatttccctgaactttttctcaataaacaagagccaactcccgtgcatcagaaacaatgtgagacggggactactagtgaagactcaagcagaagaggagaggatgattcgagcacggcattggcagtaggaggggtgagtgaggagacgggttgggctgaggcatggttcaatgcaattccagaggaatggggacctggaagccctctatgggatgattaccactttcccatttctaaccataaggacgatcttgacgccacacaaaactcttcttctgatacaatttaggacctttgttagaatatagatatgcttagttgtatgactgatctagcttgtgttttttttttgggtggagacagtttttgtcatcttccacattttagattctattttcgaccatcatttttttcttgatcggtgactatgaatctaatggggtcaatcattttcacatataaaacttaagtatttggtgtttgtactt

SEQ ID NO: 96 - protein, 336 aa, Arabidopsis thaliana
MATAKNKGKSIRVLGTSEAEKKDEMELEEEFQFSSGKYKDSGPGSDMWLGDASSTSPRSLRKTRTFDRHNPYLVSSYATPQPPTTTTCSVSFPFYLPPAIQNQQRFLHPNDPSGQRQQQMISFDPQQQVQPYVAQQQQQQQHLLQYWRDILKLSPSGRMMMMNMLRQESDLPLTRPPVQPFSATKLYRGVRQRHWGKWVAEIRKPRNRTRLWLGTFDTAEEAAMAYDREAFKLRGETARLNFPELFLNKQEPTPVHQKQCETGTTSEDSSRRGEDDSSTALAVGGVSEETGWAEAWFNAIPEEWGPGSPLWDDYHFPISNHKDDLDATQNSSSDTI

SEQ ID NO: 97 - DNA, 879 bp, Arabidopsis thaliana
  CDS (1)..(876)
atggactttgacgaggagctaaatctttgtattacgaaaggtaaaaatgttgatcattcttttggaggagaagcttcttccacgtccccaagatctatgaagaaaatgaagagtcctagtcgtcctaaaccctatttccaatcctcttcttctccttattcgttagaggctttccctttttctctcgatccaacacttcagaatcagcaacaacaactcggatcatacgttccggtacttgagcaacgacaagacccgacaatgcaaggccagaagcaaatgatctcctttagtcctcaacaacaacaacagcagcagcagtatatggcccagtactggagtgacacattgaatctgagtccaagaggaagaatgatgatgatgatgagccaagaagctgttcaaccttacatcgcaacgaagctgtacagaggagtgagacaacgtcaatggggaaaatgggtcgcagagatccgtaagccacgaagcagggcacgtctttggcttggtacctttgatacagctgaagaagctgccatggcctacgaccgccaagccttcaaattacgaggccacagcgcaacactgaatttcccggagcattttgtgaataaggaaagcgagctgcatgattcaaactcgtcggatcagaaagaacctgaaacgccacagccaagcgaggttaacttggagagcaaggaactaccggtgattgatgttgggagagaggaaggtatggctgaggcatggtacaatgccattacatcgggatggggtcctgaaagtcctctttgggatgatttggatagttctcatcagttttcatcagaaagctcatcttcttctcctctctcttgtcctatgaggcctttcttttga

SEQ ID NO: 98 - protein, 292 aa, Arabidopsis thaliana
MDFDEELNLCITKGKNVDHSFGGEASSTSPRSMKKMKSPSRPKPYFQSSSSPYSLEAFPFSLDPTLQNQQQQLGSYVPVLEQRQDPTMQGQKQMISFSPQQQQQQQQYMAQYWSDTLNLSPRGRMMMMMSQEAVQPYIATKLYRGVRQRQWGKWVAEIRKPRSRARLWLGTFDTAEEAAMAYDRQAFKLRGHSATLNFPEHFVNKESELHDSNSSDQKEPETPQPSEVNLESKELPVIDVGREEGMAEAWYNAITSGWGPESPLWDDLDSSHQFSSESSSSSPLSCPMRPFF

SEQ ID NO: 99 - DNA, 1499 bp, Arabidopsis thaliana
  CDS (644)..(1222)
tggtatcggtgaggctgagagttattcacttacaaaaaaaaaaaaaaacttgagtgtaaccaaaaaaaaaaagttgatatactttctggttttctccttaacttttattctttacaaatccatcccccttagatctgtttatttcccgctactttgattcatttctgttagtaatctgtctttcgtatagaagaaaactgatttcttggtttgtattttcttaaagagatcaatctttttttatttttgatcttcttgtgtttttttttctttgtagaattaatcgtttgtgagggtatttttttaattccctcctctcagaaatctacacagaggttttttattttataaacctcttttttcgattttcttgaaaacaaaaaatcctgttctttactttttttacaagaacaagggaaaaaaatttctttttattagaaatgacaacttctatggatttttacagtaacaaaacgtttcaacaatctgatccattcggtggtgaattaatggaagcgctttaccttttatcaaaagcccttccaacgattcatccgcgtttgcgttctctctacccgctccaatttcatacgggtcggatctccactcattttctcaccatcttagtcctaaaccggtctcaatgaaacaaaccggtacttccgcggctaaaccgacgaagctatacagaggagtgagacaacgtcactggggaaaatgggtggctgagattcgtttaccgaggaatcgaactcgactttggctcggaacattcgacacggcggaggaagctgctttagcttatgacaaggcggcgtataagctccgaggagattttgcgcggcttaatttccctgatctccgtcataacgacgagtatcaacctcttcaatcatcagtcgacgctaagcttgaagctatttgtcaaaacttagctgagacgacgcagaaacaggtgagatcaacgaagaagtcttcttctcggaaacgttcatcaaccgtcgcagtgaaactaccggaggaggactactctagcgccggatcttcgccgctgttaacggagagttatggatctggtggatcttcttcgccgttgtcggagctgacgtttggtgatacggaggaggagattcagccgccgtggaacgagaacgcgttggagaagtatccgtcgtacgagatcgattgggattcgattcttcagtgttcgagtcttgtaaattagatgttgccataggggtattttagggactttagagctctctgcgatggagtttttggtcattgcagagattttattattattaagggggtttgttatgttaatatcaaataagtttatctactttgatgttaattagtgttaatctctgcgtcggtccaagctgtttttttttggcatgcttcgaccgtgtgagatttcttatgtaatttttgtagttccttgattttcttagttcaagttaaattggcacaaaagagcaaaaaaaaaaaaaaaa

SEQ ID NO: 100 - protein, 193 aa, Arabidopsis thaliana
MKQTGTSAAKPTKLYRGVRQRHWGKWVAEIRLPRNRTRLWLGTFDTAEEAALAYDKAAYKLRGDFARLNFPDLRHNDEYQPLQSSVDAKLEAICQNLAETTQKQVRSTKKSSSRKRSSTVAVKLPEEDYSSAGSSPLLTESYGSGGSSSPLSELTFGDTEEEIQPPWNENALEKYPSYEIDWDSILQCSSLVN

SEQ ID NO: 101 - DNA, 962 bp, Arabidopsis thaliana
  CDS (77)..(880)
acaccggacattttgaaatctcaacaagaaccaaaccaaacaacaaaaaaacattcttaataattatctttctgttatgtcgatgacggcggattctcaatctgattatgcttttcttgagtccatacgacgacacttactaggagaatcggagccgatactcagtgagtcgacagcgagttcggttactcaatcttgtgtaaccggtcagagcattaaaccggtgtacggacgaaaccctagctttagcaaactgtatccttgcttcaccgagagctggggagatttgccgttgaaagaaaacgattctgaggatatgttagtttacggtatcctcaacgacgcctttcacggcggttgggagccgtcttcttcgtcttccgacgaagatcgtagctctttcccgagtgttaagatcgagactccggagagtttcgcggcggtggattctgttccggtcaagaaggagaagacgagtcctgtttcggcggcggtgacggcggcgaagggaaagcattatagaggagtgagacaaaggccgtgggggaaatttgcggcggagattagagacccggcgaagaacggagctagggtttggttaggaacgtttgagacggcggaggacgcggcgttggcttacgacagagctgctttcaggatgcgtggttcccgcgctttgttgaattttccgttgagagttaattcaggagaacccgacccggttcgaatcaagtccaagagatcttctttttcttcttctaacgagaacggagctccgaagaagaggagaacggtggccgccggtggtggaatggataagggattgacggtgaagtgcgaggttgttgaagtggcacgtggcgatcgtttattggttttataattttgatttttctttgttggatgattatatgattcttcaaaaaagaagaacgttaataaaaaaattcgtttattattgt

SEQ ID NO: 102 - protein, 268 aa, Arabidopsis thaliana
MSMTADSQSDYAFLESIRRHLLGESEPILSESTASSVTQSCVTGQSIKPVYGRNPSFSKLYPCFTESWGDLPLKENDSEDMLVYGILNDAFHGGWEPSSSSSDEDRSSFPSVKIETPESFAAVDSVPVKKEKTSPVSAAVTAAKGKHYRGVRQRPWGKFAAEIRDPAKNGARVWLGTFETAEDAALAYDRAAFRMRGSRALLNFPLRVNSGEPDPVRIKSKRSSFSSSNENGAPKKRRTVAAGGGMDKGLTVKCEVVEVARGDRLLVL

SEQ ID NO: 103 - DNA, 834 bp, Arabidopsis thaliana
  CDS (171)..(746)
aacacaatttgattacactgagcaacacaaaactggcgaaccaacgtgactctaacgaagaaaccggcaatggccagtatcactacaatgccgaagaaataacaagaatcataaacgagccagaatattatcccccgggttacaacttgtctaccaccgcaatttcaaacatggtgtctatgctgactaatgttgtctctggtgagaccgaaccctcggcatctgcgacatggacgatgggtcataagagagaaagagaagagttttctttgcctcctcaaccattgattaccggttcagctgtgactaaagaatgtgaaagctcaatgtccttggagaggccaaaaaaatatagaggagtaaggcaacgaccatggggaaaatgggcggcggagattcgagacccacacaaggcgacacgtgtatggcttgggacattcgagacagccgaggccgccgcaagagcctatgatgcggcagcacttcgctttagaggaagcaaagcaaagcttaatttccccgaaaatgttggaactcagacgattcaacgaaattctcatttcttgcaaaactctatgcaaccttctctgacatacatcgatcaatgtccaactctattatcttactctcgatgtatggagcaacaacaaccattagtaggcatgttgcagccaacagaagaggaaaatcactttttcgaaaaaccatggaccgaatatgatcaatacaattactcctcttttggttaactaacatattgtcaacgctttgtatttctacttattcgatctaccaattttttctctcccaatacaacttcagtctgattattgc

SEQ ID NO: 104 - protein, 192 aa, Arabidopsis thaliana
MVSMLTNVVSGETEPSASATWTMGHKREREEFSLPPQPLITGSAVTKECESSMSLERPKKYRGVRQRPWGKWAAEIRDPHKATRVWLGTFETAEAAARAYDAAALRFRGSKAKLNFPENVGTQTIQRNSHFLQNSMQPSLTYIDQCPTLLSYSRCMEQQQPLVGMLQPTEEENHFFEKPWTEYDQYNYSSFG

SEQ ID NO: 105 - DNA, 1085 bp, Arabidopsis thaliana
  CDS (120)..(953)
acttcactctctaatttccttctctctatctctcaccatattcgcgattaaaaactctcaacttttctctcaaatttctgatcctttgatccaacagttagaagaagattcatctgatcatggccctcgaagcgatgaacactccaacttcttctttcaccagaatcgaaacgaaagaagatttgatgaacgacgccgttttcattgagccgtggcttaaacgcaaacgctccaaacgtcagcgttctcacagcccttcttcgtcttcttcctcaccgcctcgatctcgacccaaatcccagaatcaagatcttacggaagaagagtatctcgctctttgtctcctcatgctcgctaaagatcaaccgtcgcaaacgcgatttcatcaacagtcgcaatcgttaacgccgccgccagaatcaaagaaccttccgtacaagtgtaacgtctgtgaaaaagcgtttccttcctatcaggctttaggcggtcacaaagcaagtcaccgaatcaaaccaccaaccgtaatctcaacaaccgccgatgattcaacagctccgaccatctccatcgtcgccggagaaaaacatccgattgctgcctccggaaagatccacgagtgttcaatctgtcataaagtgtttccgacgggtcaagctttaggcggtcacaaacgttgtcactacgaaggcaacctcggcggcggaggaggaggaggaagcaaatcaatcagtcacagtggaagcgtgtcgagcacggtatcggaagaaaggagccaccgtggattcatcgatctaaacctaccggcgttacctgaactcagccttcatcacaatccaatcgtcgacgaagagatcttgagtccgttgaccggtaaaaaaaccgcttttgttgaccgatcacgaccaagtcatcaagaaagaagatttatctttaaaaatctaatactcgactattaattcttgtgtgatttttttcgttacaaccatagtttcattttcatttttttagttacaaatttttaattgttctgatttggattgaatattggtatattgttaggggttgatacaaaaaaaaaaaaaaaa

SEQ ID NO: 106 - protein, 278 aa, Arabidopsis thaliana
MALEAMNTPTSSFTRIETKEDLMNDAVFIEPWLKRKRSKRQRSHSPSSSSSSPPRSRPKSQNQDLTEEEYLALCLLMLAKDQPSQTRFHQQSQSLTPPPESKNLPYKCNVCEKAFPSYQALGGHKASHRIKPPTVISTTADDSTAPTISIVAGEKHPIAASGKIHECSICHKVFPTGQALGGHKRCHYEGNLGGGGGGGSKSISHSGSVSSTVSEERSHRGFIDLNLPALPELSLHHNPIVDEEILSPLTGKKTAFVDRSRPSHQERRFIFKNLILDY

SEQ ID NO: 107 - DNA, 1783 bp, Arabidopsis thaliana
  CDS (162)..(1529)
attgttaaaagctctcacacaaccaccgttctccgtcacggtggcgctttattctctcatcggagcgcctcaccggtggcagacggtgtcgttgttcctctcctaaaacctccatcaatcaccatctctttacacagagctctaaccaaaatcttcgagagatggggcagagtatgagctgtggaagtcgaccggagcacggaatattcgcctctgtacagtgcggcgatatcatcactatccgtcgtgtgatggcgacggagcctagtctgttgaatcaaactactccttatgatcgtcactctgttcttcatgtcgctgctgctaatggtcagatcgagattttgtcattgcttttggaacgatttacgaatccagatttgttgaatcgtcacaagcagactccgttaatgttggctgcgatgtatggaagaatctcttgtgtgaagaagctagctgaagttggagctaatattttgatgtttgattctgtgaatcgaagaacatgtttgcattacgctgcttattatggacatgctaattgtgttcaagctattctctctgctgctcaatcaagtcctgttgctgtccattggggatatgcgagatttgtgaacataagagatgataaaggagcgactccgttgcatttagctgctcgacagagacgacctgaatgtgtgaatgttttgttggatagtggttctcttgtttgtgcatctactagtgtatatggttctccaggaagcacacctcttcatttagcagctagaagtggatctatagattgtgtcagaaagttgcttgcttggggtgctgatcgtcttcaacgagacgcttctgggagaataccttatgtggttgcgatgaagcataagcatggagcatgtggagccttacttaatccgtcctctgcagagccacttgtttggccatcaccattaaagttcatcagtgagcttaatgacgaggcgaaacttctcttagagcaggctttaatggaggctaacagggagagagagaaaaccatcctcaaaggaacagcttattccttaccatcaccctctttctctgacacggatgataacatgtccgaggtgagtgatacggaactgtgctgcatttgctttgagcaagtatgtacaattgaagttaaagactgtggtcaccaaatgtgtgcacaatgcacacttgcactgtgctgtcacaacaaaccaaacccaacgacctcaaccgtgactccaccggtctgtccgttctgtagaagcaccattgcatgtttagtcgtcgcccagaacaacaacaacaacaacgaaaagagcaaaagcctagatgatgttgttgttgttgatcgtgaggcaggtgatgttagctcctccaaattcagaaaacatagaagatcaataaaccttggcgaagaaagcagcagcttcatgggactatcaactattggatcattcggtaggataaccggccgtggctcgggaaggatcgcagccgaaaacgagctgatggacaaaccaatattgtgagggatcgattccgttttaagggacattttggggcatgggggagcaataaaaaagatgaggggatgaaattgtgagaatgtataaaatatagatgaatttatgttagatcttttgttgaagggaggaagattgaaataaggaaaaagatgtggggaggtgtgtaatgcaaggatttgttgtttctttgattaagtttggccaaaattgtttgttgttgttattatttggttacttgatatgaaagggaaacc

SEQ ID NO: 108 - protein, 456 aa, Arabidopsis thaliana
MGQSMSCGSRPEHGIFASVQCGDIITIRRVMATEPSLLNQTTPYDRHSVLHVAAANGQIEILSLLLERFTNPDLLNRHKQTPLMLAAMYGRISCVKKLAEVGANILMFDSVNRRTCLHYAAYYGHANCVQAILSAAQSSPVAVHWGYARFVNIRDDKGATPLHLAARQRRPECVNVLLDSGSLVCASTSVYGSPGSTPLHLAARSGSIDCVRKLLAWGADRLQRDASGRIPYVVAMKHKHGACGALLNPSSAEPLVWPSPLKFISELNDEAKLLLEQALMEANREREKTILKGTAYSLPSPSFSDTDDNMSEVSDTELCCICFEQVCTIEVKDCGHQMCAQCTLALCCHNKPNPTTSTVTPPVCPFCRSTIACLVVAQNNNNNNEKSKSLDDVVVVDREAGDVSSSKFRKHRRSINLGEESSSFMGLSTIGSFGRITGRGSGRIAAENELMDKPIL

SEQ ID NO: 109 - DNA, 714 bp, Arabidopsis thaliana
  CDS (1)..(711)
atgaagatacaatgtgatgtgtgtgagaaagctccggccacgcttatatgttgtgctgatgaagctgctctctgcgctaaatgtgacgttgaggttcatgctgctaataaactcgctagcaaacaccaacgcctttttcttgactctctctcaactaaattccctccctgcgacatctgccttgagaaggcagctttcatattctgtgtagaggatagggctctgctctgcagagattgcgatgaggcgacccatgcgccaaatactcgctctgctaatcaccagaggttcttagccactggaatccgagttgctcttagttccactagttgcaatcaagaagtggaaaagaatcactttgacccatctaatcagcagagtctctctaaaccgccaactcagcaacccgctgctccatctcctttgtgggctaccgatgaattcttcagctactctgatcttgactgcagtaataagaaagagcaactcgatctcggggagctggattggcttgcagagatgggtctgtttggtgaccagcctgatcaagaggctctaccggtagccgaagttcccgagctttccttttcacatttggctcatgctcattcctacaacagacctatgaagtccaatgtacccaacaagaagcagaggcttgagtaccggtatgatgatgaagaagagcacttcctagtccccgacctaggctaa

SEQ ID NO: 110 - protein, 237 aa, Arabidopsis thaliana
MKIQCDVCEKAPATLICCADEAALCAKCDVEVHAANKLASKHQRLFLDSLSTKFPPCDICLEKAAFIFCVEDRALLCRDCDEATHAPNTRSANHQRFLATGIRVALSSTSCNQEVEKNHFDPSNQQSLSKPPTQQPAAPSPLWATDEFFSYSDLDCSNKKEQLDLGELDWLAEMGLFGDQPDQEALPVAEVPELSFSHLAHAHSYNRPMKSNVPNKKQRLEYRYDDEEEHFLVPDLG

SEQ ID NO: 111 - DNA, 829 bp, Arabidopsis thaliana
  CDS (99)..(584)
atcacaactactatcacaccaaactcaaaaaacacaaaccacaagaggatcatttcattttttattgtttcgttttaatcatcatcatcagaagaaaaatggttgcgatatcggagatcaagtcgacggtggatgtcacggcggcgaattgtttgatgcttttatctagagttggacaagaaaacgttgacggtggcgatcaaaaacgcgttttcacatgtaaaacgtgtttgaagcagtttcattcgttccaagccttaggaggtcaccgtgcgagtcacaagaagcctaacaacgacgctttgtcgtctggattgatgaagaaggtgaaaacgtcgtcgcatccttgtcccatatgtggagtggagtttccgatgggacaagctttgggaggacacatgaggagacacaggaacgagagtggggctgctggtggcgcgttggttacacgcgctttgttgccggagcccacggtgactacgttgaagaaatctagcagtgggaagagagtggcttgtttggatctgagtctagggatggtggacaatttgaatctcaagttggagcttggaagaacagtttattgattttatttattttccttaaattttctgaatatatttgtttctctcattctttgaatttttcttaatattctagattatacatacatccgcagatttaggaaactttcatagagtgtaatcttttctttctgtaaaaatatattttacttgtagcattggagatttgttatgagattatcttacttagcatttagtgaataatctattagcctattttgccgacgcgaaaaaaaaaaaaaaaa

SEQ ID NO: 112 - protein, 162 aa, Arabidopsis thaliana
MVAISEIKSTVDVTAANCLMLLSRVGQENVDGGDQKRVFTCKTCLKQFHSFQALGGHRASHKKPNNDALSSGLMKKVKTSSHPCPICGVEFPMGQALGGHMRRHRNESGAAGGALVTRALLPEPTVTTLKKSSSGKRVACLDLSLGMVDNLNLKLELGRTVY

SEQ ID NO: 113 - DNA, 881 bp, Arabidopsis thaliana
  CDS (89)..(769)
agcaattaaacaatttcttcactgcaattcacaagcaaccttcaaactaaaactcgagagacaagaaatcctcagaatctttaacttaatggcgctcgaggctcttacatcaccaagattagcttctccgattcctcctttgttcgaagattcttcagtcttccatggagtcgagcactggacaaagggtaagcgatctaagagatcaagatccgatttccaccaccaaaacctcactgaggaagagtatctagctttttgcctcatgcttctcgctcgcgacaaccgtcagcctcctcctcctccggcggtggagaagttgagctacaagtgtagcgtctgcgacaagacgttctcttcttaccaagctctcggtggtcacaaggcaagccaccgtaagaacttatcacagactctctccggcggaggagatgatcattcaacctcgtcggcgacaaccacatccgccgtgactactggaagtgggaaatcacacgtttgcaccatctgtaacaagtcttttccttccggtcaagctctcggcggacacaagcggtgccactacgaaggaaacaacaacatcaacactagtagcgtgtccaactccgaaggtgcggggtccactagccacgttagcagtagccaccgtgggtttgacctcaacatccctccgatccctgaattctcgatggtcaacggagacgacgaagtcatgagccctatgccggcgaagaagcctcggtttgactttccggtcaaacttcaactttaaggaaatttacttagacgataagatttcgtttgtatactgttgagagttgtgtaggaatttgttgactgtacataccaaattggactttgactcaaaaaaaaaaaaaaaa

SEQ ID NO: 114 - protein, 227 aa, Arabidopsis thaliana
MALEALTSPRLASPIPPLFEDSSVFHGVEHWTKGKRSKRSRSDFHHQNLTEEEYLAFCLMLLARDNRQPPPPPAVEKLSYKCSVCDKTFSSYQALGGHKASHRKNLSQTLSGGGDDHSTSSATTTSAVTTGSGKSHVCTICNKSFPSGQALGGHKRCHYEGNNNINTSSVSNSEGAGSTSHVSSSHRGFDLNIPPIPEFSMVNGDDEVMSPMPAKKPRFDFPVKLQL

SEQ ID NO: 115 - DNA, 1107 bp, Arabidopsis thaliana
  CDS (139)..(957)
aactcggtccgagtcgactcggtccgacaagcgattttcgtgactccgtcgtcaccacgcctatgttaagtgagtaaatccgactcttctccgtagagatttctcctactttcaggctctcttgtgaatttggaagacatgagctttgttttccggggaagtagaggagatttagaaagcggattctcgggtggttttctacccgaaagacgagctatgcgtgttcatggagctcgaccagttaattctaattccctcgcttttctggttacagttcttttgctgtttatgattctcaattcgcatcagatgcctcctaatttcctgctgtggcttgtgcttggggtgtttttgatggcaacgacgcttaggatgtatgctacttgccaacaacttcaagctcatgctcaggctcaggctgcagcagcaagtggcctctttagccatactgagctgaggttgcatgtgcctccttccattgctcttgctacgagagggcgtcttcagggacttaggctccagctggctcttcttgatcgggaatttgatgacttagattatgaaactttaagagcacttgattctgataatgtttccacaacttctatgagcgaggaagagataaatgcacttccagttcacaagtacaaggtgttggatcctgaaaatggttgctctttggcaaagcaagcgtcaacctcatcctcagctgagaagatgctagattctgccaatgaaagtaaaaaaggaacagaagatgagctcacatgtagtgtttgcctagaacaagttaccgtaggggaaatcgttcgcaccttaccttgtttgcatcagtttcatgcaggatgtatcgatccatggttgagacagcaaggaacatgtcctgtctgtaaatttagagctcattcaggatggcaagaacaagatgagattgatgatgatgcttccgacatggtttgaaaagtttggtttcgaacacttgtttatgttattaatgtgcgtgcgaattaaaccccaaacaaatcatgtaatggtcaataactcaatatgatgtaactgatgactctccctttccaaaagtttgatttagattcatcatgaaaaagt

SEQ ID NO: 116 - protein, 273 aa, Arabidopsis thaliana
MSFVFRGSRGDLESGFSGGFLPERRAMRVHGARPVNSNSLAFLVTVLLLFMILNSHQMPPNFLLWLVLGVFLMATTLRMYATCQQLQAHAQAQAAAASGLFSHTELRLHVPPSIALATRGRLQGLRLQLALLDREFDDLDYETLRALDSDNVSTTSMSEEEINALPVHKYKVLDPENGCSLAKQASTSSSAEKMLDSANESKKGTEDELTCSVCLEQVTVGEIVRTLPCLHQFHAGCIDPWLRQQGTCPVCKFRAHSGWQEQDEIDDDASDMV

SEQ ID NO: 117 - DNA, 531 bp, Arabidopsis thaliana
  CDS (1)..(528)
atgggatcggaacaaaacgatagcacaagcttcacgcaatcgcaagcttcagagccaaagctatgtgttaaaggatgtggtttctttggatcaccatcaaacatggatctctgttctaaatgttacagaggcatttgtgctgaggaagctcaaacagcagttgctaaagctgctgttgaaaaatctttcaagccttctcctcctcgtagtctcttcatagcagaacctcctgctgttgttgtggaacccaaacccgaaaaggcggcagttgttgttgtctcggccgagccatcttcctcggcggttcctgaggcgaacgagccatcgagacctgcacgaaccaaccggtgtttgtgttgtaacaagaaggttgggatcatggggtttaagtgcaaatgcgggagcactttctgcggcgaacatcggtacccggagactcatgattgcagctttgatttcaaagaagttggacgtggagagattgccaaagctaatcctgtggttaaggctgataaaattcaaaggttctga

SEQ ID NO: 118 - protein, 176 aa, Arabidopsis thaliana
MGSEQNDSTSFTQSQASEPKLCVKGCGFFGSPSNMDLCSKCYRGICAEEAQTAVAKAAVEKSFKPSPPRSLFIAEPPAVVVEPKPEKAAVVVVSAEPSSSAVPEANEPSRPARTNRCLCCNKKVGIMGFKCKCGSTFCGEHRYPETHDCSFDFKEVGRGEIAKANPVVKADKIQRF

SEQ ID NO: 119 - DNA, 1068 bp, Arabidopsis thaliana
  CDS (1)..(1065)
atgttgaaagtagagagtaactgggcacaagcctgtgatacatgccgatcagccgcctgcaccgtgtactgccgggctgattctgcctacttgtgctccagttgtgatgctcaagttcatgctgccaatcgtcttgcttcccgccatgaacgtgttcgagtctgtcaatcatgtgagcgagccccggctgcctttttctgcaaggcagatgctgcatctctatgcacaacctgtgattcagagattcattccgcaaacccacttgctagacgccatcaacgagttccaattctgcccatttctgagtactcttacagttccacggccactaaccattcatgtgagacaacagtgacagatccagagaacagacttgtgcttggtcaagaagaagaggatgaagatgaagcagaggcggcttcatggttgttgcctaattcagggaaaaacagtggtaacaacaatggcttctcgattggggatgagtttctgaaccttgttgattatagttcgagtgataagcaattcacagatcaatccaatcagtatcaactagactgcaacgtacctcagaggagctatggggaagatggagttgttccacttcaaattgaagtatcaaagggcatgtaccaagagcaacagaactttcagctgagtatcaactgtggctcctggggagctcttcgaagctccaatggttccctcagtcatatggtgaatgtttcatctatggacctgggagttgtgccggagtcaacaacgagtgacgcaacagtatcaaacccaagatcgcccaaagcggtaacagaccaaccaccttaccctccagctcagatgctcagtccaagggacagagaagctagagtcctgagatacagagagaagaagaagatgaggaaatttgagaagacgataagatatgcttcaaggaaagcgtatgcagagaaaagaccacggatcaagggccggtttgcaaagaagaaagatgtcgatgaagaggcaaaccaagctttctccacaatgataacatttgacaccggatatggaattgttccatcattctga

SEQ ID NO: 120 - protein, 355 aa, Arabidopsis thaliana
MLKVESNWAQACDTCRSAACTVYCRADSAYLCSSCDAQVHAANRLASRHERVRVCQSCERAPAAFFCKADAASLCTTCDSEIHSANPLARRHQRVPILPISEYSYSSTATNHSCETTVTDPENRLVLGQEEEDEDEAEAASWLLPNSGKNSGNNNGFSIGDEFLNLVDYSSSDKQFTDQSNQYQLDCNVPQRSYGEDGVVPLQIEVSKGMYQEQQNFQLSINCGSWGALRSSNGSLSHMVNVSSMDLGVVPESTTSDATVSNPRSPKAVTDQPPYPPAQMLSPRDREARVLRYREKKKMRKFEKTIRYASRKAYAEKRPRIKGRFAKKKDVDEEANQAFSTMITFDTGYGIVPSF

SEQ ID NO: 121 - DNA, 1116 bp, Arabidopsis thaliana
  CDS (191)..(934)
atcccacctacttgttccccacaaaacactctctccctctttgttctttcatcttctctaagctctttctctgaacctacgcttctgctaagctattctaagagaagccagactagcaataaacccttcattttaagcattctgtttccttcttgagaaacctagatattttggtttcttgtatccggtgatgaagatacagtgtgatgtgtgtgagaaagctccggcgacggtgatttgttgcgccgacgaagctgctctctgtcctcaatgcgacatcgagattcacgccgctaacaaactcgctagcaagcaccaacgtcttcatcttaattccctctccaccaaattccctcgttgcgatatctgccaagagaaggcagctttcattttctgtgtagaggatagagctctgctttgcagggactgcgatgaatccatccacgtggctaattctcgatctgctaatcaccagaggttcttagccactgggatcaaagtagctctgacctcaactatatgtagtaaagaaattgagaagaatcaacctgagccttccaacaaccaacagaaggctaatcagattcctgctaaatccacaagccagcagcaacaacaaccttcttctgctactccacttccctgggctgttgacgatttctttcacttctctgatattgaatccaccgacaagaaaggacagcttgatcttggggcaggggagttggattggttttcagacatgggattcttcggtgatcagattaatgacaaggctcttcctgcagctgaagttcctgagctttctgtttcgcatttaggtcatgttcattcatacaaacctatgaagtcaaatgtttcacacaagaagccgaggtttgagaccagatatgatgatgatgatgaggaacacttcattgtccctgatcttggctaaaaagctatatgtaatctatgtgtagacattcttcaatgtaaaagaacaaacaagaaacctatctgcatgtgtggagttaatgtcatatacattttagttttgtcttaagttgtgtaagatatgttgagagcttataacaaatgtctgtgtttgagttttgttcaaaaaaaaaaaaaaaa

SEQ ID NO: 122 - protein, 248 aa, Arabidopsis thaliana
MKIQCDVCEKAPATVICCADEAALCPQCDIEIHAANKLASKHQRLHLNSLSTKFPRCDICQEKAAFIFCVEDRALLCRDCDESIHVANSRSANHQRFLATGIKVALTSTICSKEIEKNQPEPSNNQQKANQIPAKSTSQQQQQPSSATPLPWAVDDFFHFSDIESTDKKGQLDLGAGELDWFSDMGFFGDQINDKALPAAEVPELSVSHLGHVHSYKPMKSNVSHKKPRFETRYDDDDEEHFIVPDLG

SEQ ID NO: 123 - DNA, 909 bp, Arabidopsis thaliana
  CDS (1)..(906)
atggatcagtactcatcctctttggtcgatacttcattagatctcactattggcgttactcgtatgcgagttgaagaagatccaccgacaagtgctttggtggaagaattaaaccgagttagtgctgagaacaagaagctctcggagatgctaactttgatgtgtgacaactacaacgtcttgaggaagcaacttatggaatatgttaacaagagcaacataaccgagagggatcaaatcagccctcccaagaaacgcaaatccccggcgagagaggacgcattcagctgcgcggttattggcggagtgtcggagagtagctcaacggatcaagatgagtatttgtgtaagaagcagagagaagagactgtcgtgaaggagaaagtctcaagggtctattacaagaccgaagcttctgacactaccctcgttgtgaaagatgggtatcaatggaggaaatatggacagaaagtgactagagacaatccatctccaagagcttacttcaaatgtgcttgtgctccaagctgttctgtcaaaaagaaggttcagagaagtgtggaggatcagtccgtgttagttgcaacttatgagggtgaacacaaccatccaatgccatcgcagatcgattcaaacaatggcttaaaccgccacatctctcatggtggttcagcttcaacacccgttgcagcaaacagaagaagtagcttgactgtgccggtgactaccgtagatatgattgaatcgaagaaagtgacgagcccaacgtcaagaatcgattttccccaagttcagaaacttttggtggagcaaatggcttcttccttaaccaaagatcctaactttacagcagctttagcagcagctgttaccggaaaattgtatcaacagaatcataccgagaaatag

SEQ ID NO: 124 - protein, 302 aa, Arabidopsis thaliana
MDQYSSSLVDTSLDLTIGVTRMRVEEDPPTSALVEELNRVSAENKKLSEMLTLMCDNYNVLRKQLMEYVNKSNITERDQISPPKKRKSPAREDAFSCAVIGGVSESSSTDQDEYLCKKQREETVVKEKVSRVYYKTEASDTTLVVKDGYQWRKYGQKVTRDNPSPRAYFKCACAPSCSVKKKVQRSVEDQSVLVATYEGEHNHPMPSQIDSNNGLNRHISHGGSASTPVAANRRSSLTVPVTTVDMIESKKVTSPTSRIDFPQVQKLLVEQMASSLTKDPNFTAALAAAVTGKLYQQNHTEK

SEQ ID NO: 125 - DNA, 1462 bp, Arabidopsis thaliana
  CDS (382)..(1161)
accgaccttcttggttcttccggcgttgactgttacgaagatgatgaagacttgagagtttctgggtcgagttttggtgggtactatccagagagaaccgggtctggtttacctaagttcaagacggctcaaccaccacctcttccgatttcacaatcttctcataacttcactttctccgattaccttgattctcctctgcttctcagctcctcacacagtttgatatctccaacaacaggaacgtttccattgcaaggctttaatggaacaacaaacaatcactcagattttccctggcagctacaatctcaaccatcaaacgcttcttctgctttgcaagaaacatatggtgttcaagatcacgagaagaagcaggagatgattcctaatgagattgcaacacaaaacaacaatcaaagttttggaacagaacgtcagataaagataccagcatacatggtgagtaggaactctaatgatggttatggttggagaaaatacggtcagaaacaagtgaagaagagcgaaaaccctaggagttacttcaagtgtacgtatcctgattgtgtttccaagaagattgttgagacggcttctgatggacagatcactgagatcatttataaaggtggtcataatcatcctaagcctgagttcaccaagagaccatctcaatcttcattaccatcatcggttaatgggaggcgcttgtttaatcctgcttctgttgttagtgaacctcatgatcaatcagagaactcttcgatttcgtttgactatagtgatcttgagcagaaaagttttaaatcagagtatggtgagatagatgaagaggaggaacaacctgagatgaagaggatgaaaagagaaggtgaagatgaagggatgtctatagaagtaagcaaaggagttaaagagccaagagttgtggttcagacaataagtgatattgatgttcttatagatggctttagatggaggaaatatggtcaaaaagttgtcaaaggaaatactaatccaaggagctactacaagtgcacattccaaggttgtggagtgaagaagcaagtggaaagatccgcagcagacgagagagcagttctcactacctatgaaggaagacacaatcacgatatcccaaccgcgctacgtcgctcgtgaaattattgggacttagtcactagtaatatgatttaggctttctaaaaacaaaaaatcttactatggcttatcttttgtgctcattcacagtttgtttatttgtttgttacacagtcaatactttgttttgtacagagtggtgcttagtagtgtttttattattatcttggccttatagaataacctctcttctcatctgtgtgactttaaacacttgagagtccattttatagttcttgtgtattggtcttttgtttgatttatgtacatttttaatattcgaaaaaaaaaaaaaaaa

SEQ ID NO: 126 - protein, 260 aa, Arabidopsis thaliana
MIPNEIATQNNNQSFGTERQIKIPAYMVSRNSNDGYGWRKYGQKQVKKSENPRSYFKCTYPDCVSKKIVETASDGQITEIIYKGGHNHPKPEFTKRPSQSSLPSSVNGRRLFNPASVVSEPHDQSENSSISFDYSDLEQKSFKSEYGEIDEEEEQPEMKRMKREGEDEGMSIEVSKGVKEPRVVVQTISDIDVLIDGFRWRKYGQKVVKGNTNPRSYYKCTFQGCGVKKQVERSAADERAVLTTYEGRHNHDIPTALRRS

SEQ ID NO: 127 - DNA, 438 bp, Arabidopsis thaliana
  CDS (1)..(435)
atggagggatatgataatgggtcgttgtatgctccttttttgtcgttgaaatctcattcgaaaccagagctgcatcaaggcgaagaagagagctcaaaggttagatcagaaggttgttcgaaaagcgtggagtcgtcgaaaaagaaggggaagaaacaaaggtatgcgtttcaaacaaggagccaagtggatattcttgatgatggttatcgatggaggaaatatggccaaaaggccgtcaagaacaacaagttccctaggagttactataggtgtacatatggaggatgcaatgtgaagaagcaagtgcaaagattaacagtggaccaagaagtggtcgtgacaacctacgaaggagtgcattcgcatcccatcgagaaatccaccgaaaacttcgagcatattctcactcaaatgcaaatctactcttctttctag

SEQ ID NO: 128 - protein, 145 aa, Arabidopsis thaliana
MEGYDNGSLYAPFLSLKSHSKPELHQGEEESSKVRSEGCSKSVESSKKKGKKQRYAFQTRSQVDILDDGYRWRKYGQKAVKNNKFPRSYYRCTYGGCNVKKQVQRLTVDQEVVVTTYEGVHSHPIEKSTENFEHILTQMQIYSSF

SEQ ID NO: 129 - DNA, 1254 bp, Arabidopsis thaliana
  CDS (137)..(1090)
aaaagtccaagcaccaatctagacctcttaggaaaaaaacctaaaaacctaatccccaaacctaaaaggcttatctcatctcttcttctttgtcttctttactctttttttacctctctcttcattgttcttcaccatgtctaatgaaaccagagatctctacaactaccaatacccttcatcgttttcgttgcacgaaatgatgaatctgcctacttcaaatccatcttcttatggaaacctcccatcacaaaacggttttaatccatctacttattccttcaccgattgtctccaaagttctccagcagcgtatgaatctctacttcagaaaacttttggtctttctccctcttcctcagaggttttcaattcttcgatcgatcaagaaccgaaccgtgatgttactaatgacgtaatcaatggtggtgcatgcaacgagactgaaactagggtttctccttctaattcttcctctagtgaggctgatcaccccggtgaagattccggtaagagccggaggaaacgagagttagtcggtgaagaagatcaaatttccaaaaaagttgggaaaacgaaaaagactgaggtgaagaaacaaagagagccacgagtctcgtttatgactaaaagtgaagttgatcatcttgaagatggttatagatggagaaaatacggccaaaaggctgtaaaaaatagcccttatccaaggagttactatagatgtacaacacaaaagtgcaacgtgaagaaacgagtggagagatcgttccaagatccaacggttgtgattacaacttacgagggtcaacacaaccacccgattccgactaatcttcgaggaagttctgccgcggctgctatgttctccgcagacctcatgactccaagaagctttgcacatgatatgtttaggacggcagcttatactaacggcggttctgtggcggcggctttggattatggatatggacaaagtggttatggtagtgtgaattcaaaccctagttctcaccaagtgtatcatcaagggggtgagtatgagctcttgagggagatttttccttcaattttctttaagcaagagccttgatcgatcattgttataactacatatattatatatattgagagagagaggtagagaaaaaaaaaacttatatgtaacttaagatcttattttgtctctcttatttgcatgtacatattttttcatgaaagaatgagacagttgggcttgcttaaaaaaaaaat

SEQ ID NO: 130 - protein, 318 aa, Arabidopsis thaliana
MSNETRDLYNYQYPSSFSLHEMMNLPTSNPSSYGNLPSQNGFNPSTYSFTDCLQSSPAAYESLLQKTFGLSPSSSEVFNSSIDQEPNRDVTNDVINGGACNETETRVSPSNSSSSEADHPGEDSGKSRRKRELVGEEDQISKKVGKTKKTEVKKQREPRVSFMTKSEVDHLEDGYRWRKYGQKAVKNSPYPRSYYRCTTQKCNVKKRVERSFQDPTVVITTYEGQHNHPIPTNLRGSSAAAAMFSADLMTPRSFAHDMFRTAAYTNGGSVAAALDYGYGQSGYGSVNSNPSSHQVYHQGGEYELLREIFPSIFFKQEP

SEQ ID NO: 131 - DNA, 1148 bp, Arabidopsis thaliana
  CDS (104)..(1075)
acgtctctctctttctctctactctctgtttcctcataattcaatcactatatttttttaaaaacatttgacttcatcgatcggttaacaattaatcaaaaagatgggacgatcaccatgttgtgagaagaagaatggtctcaagaaaggaccatggactcctgaggaggatcaaaagctcattgattatatcaatatacatggttatggaaattggagaactcttcccaagaatgctgggttacaaagatgtggtaagagttgtcgtctccggtggaccaactatctccgaccagatattaagcgtggaagattctcttttgaagaagaagaaaccattattcaacttcacagcatcatgggaaacaagtggtctgcgattgcggctcgtttgcctggaagaacagacaacgagatcaaaaactattggaacactcacatcagaaaaagacttctaaagatgggaatcgacccggttacacacactccacgtcttgatcttctcgatatctcctccattctcagctcatctatctacaactcttcgcatcatcatcatcatcatcatcaacaacatatgaacatgtcgaggctcatgatgagtgatggtaatcatcaaccattggttaaccccgagatactcaaactcgcaacctctctcttttcaaaccaaaaccaccccaacaacacacacgagaacaacacggttaaccaaaccgaagtaaaccaataccaaaccggttacaacatgcctggtaatgaagaattacaatcttggttccctatcatggatcaattcacgaatttccaagacctcatgccaatgaagacgacggtccaaaattcattgtcatacgatgatgattgttcgaagtccaattttgtattagaaccttattactccgactttgcttcagtcttgaccacaccttcttcaagcccgactccgttaaactcaagttcctcaacttacatcaatagtagcacttgcagcaccgaggatgaaaaagagagttattacagtgataatatcactaattattcgtttgatgttaatggttttctccaattccaataaacaaaacgccattggaatagagttatgtaaacatgcaatcattgtatttgttatatagattttgttacat

SEQ ID NO: 132 - protein, 324 aa, Arabidopsis thaliana
MGRSPCCEKKNGLKKGPWTPEEDQKLIDYINIHGYGNWRTLPKNAGLQRCGKSCRLRWTNYLRPDIKRGRFSFEEEETIIQLHSIMGNKWSAIAARLPGRTDNEIKNYWNTHIRKRLLKMGIDPVTHTPRLDLLDISSILSSSIYNSSHHHHHHHQQHMNMSRLMMSDGNHQPLVNPEILKLATSLFSNQNHPNNTHENNTVNQTEVNQYQTGYNMPGNEELQSWFPIMDQFTNFQDLMPMKTTVQNSLSYDDDCSKSNFVLEPYYSDFASVLTTPSSSPTPLNSSSSTYINSSTCSTEDEKESYYSDNITNYSFDVNGFLQFQ

SEQ ID NO: 133 - DNA, 2684 bp, Arabidopsis thaliana
  CDS (898)..(2475)
acttcttcttcttcttcttctcgatttcttactgttttcttatccaacgaaatctggaattaaaaatggaatctttatcgaatccaagctgattttgtttctttcattgaatcatctctctaaagtggaattttgtaaagagaagatctgaagttgtgtagaggagcttagtgatggagacaaattcgtctggagaagatctggttattaagactcggaagccatatacgataacaaagcaacgtgaaaggtggactgaggaagaacataatagattcattgaagctttgaggctttatggtagagcatggcagaagattgaagaacatgtagcaacaaaaactgctgtccagataagaagtcacgctcagaaatttttctccaaggtaaaatcggttaattttgaaatgatgttctcatcttcattggcttaatgcttaagacttattgaaagccaggcaagttttctgcttcttttgcttcttagtcaggagatagatagattacgtttttagagtttagtaatgagcaataagtcttaaaatagttggagaaatgacgagatgtaatcgttttcttttgtttatgcctatatcttgttaatccacaaacatgtacatagattcttcagaagaatgttagtttctttagattcttcagataaacttgtgtcttcttaccgattctgaggtagtggcaaaagtgggctgagtgctagaaatttttgaatgttccttgtgataagccatagaggtaaaccatttttgattttccagttctgtcatttaaacttgttagtgtcattagatttttgtttgtttacgtttgtttagagggtaacaaaactactctcatctctctcaggtagagaaagaggctgaagctaaaggtgtagctatgggtcaagcgctagacatagctattcctcctccacggcctaagcgtaaaccaaacaatccttatcctcgaaagacgggaagtggaacgatccttatgtcaaaaacgggtgtgaatgatggaaaagagtcccttggatcagaaaaagtgtcgcatcctgagatggccaatgaagatcgacaacaatcaaagcctgaagagaaaactctgcaggaagacaactgttcagattgtttcactcatcagtatctctctgctgcatcctccatgaataaaagttgtatagagacatcaaacgcaagcactttccgcgagttcttgccttcacgggaagagggaagtcagaataacagggtaagaaaggagtcaaactcagatttgaatgcaaaatctctggaaaacggtaatgagcaaggacctcagacttatccgatgcatatccctgtgctagtgccattggggagctcaataacaagttctctatcacatcctccttcagagccagatagtcatccccacacagttgcaggagattatcagtcgtttcctaatcatataatgtcaacccttttacaaacaccggctctttatactgccgcaactttcgcctcatcattttggcctcccgattctagtggtggctcacctgttccagggaactcacctccgaatctggctgccatggccgcagccactgttgcagctgctagtgcttggtgggctgccaatggattattacctttatgtgctcctcttagttcaggtggtttcactagtcatcctccatctacttttggaccatcatgtgatgtagagtacacaaaagcaagcactttacaacatggttctgtgcagagccgagagcaagaacacttcgaggcatcaaaggctcgatcttcactggactcagaggatgttgaaaataagagtaaaccagtttgtcatgagcagccttctgcaacacctgagagtgatgcaaagggttcagatggagcaggagacagaaaacaagttgaccggtcctcgtgtggctcaaacactccgtcgagtagtgatgatgttgaggcggatgcatcagaaaggcaagaggatggcaccaatggtgaggtgaaagaaacgaatgaagacactaataaacctcaaacttcagagtccaatgcacgccgcagtagaatcagctccaatataaccgatccatggaagtctgtgtctgacgagggtcgaattgccttccaagctctcttctccagagaggtattgccgcaaagttttacatatcgagaagaacacagagaggaagaacaacaacaacaagaacaaagatatccaatggcacttgatcttaacttcacagctcagttaacaccagttgatgatcaagaggagaagagaaacacaggatttcttggaatcggattagatgcttcaaagctaatgagtagaggaagaacaggttttaaaccatacaaaagatgttccatggaagccaaagaaagtagaatcctcaacaacaatcctatcattcatgtggaacagaaagatcccaaacggatgcggttggaaactcaagcttccacatgagactctattttcatctgatctgttgtttgtactctgtttttaagttttcaagaccactgctacattttctttttcttttgaggcctttgtatttgtttccttgtccatagtcttcctgtaacatttgactctgtattattcaacaaatcataaactgtttaatctttttttttccagaaaaaaaaaaaagaaaaaaaaaaaaaaaa

SEQ ID NO: 134 - protein, 526 aa, Arabidopsis thaliana
MGQALDIAIPPPRPKRKPNNPYPRKTGSGTILMSKTGVNDGKESLGSEKVSHPEMANEDRQQSKPEEKTLQEDNCSDCFTHQYLSAASSMNKSCIETSNASTFREFLPSREEGSQNNRVRKESNSDLNAKSLENGNEQGPQTYPMHIPVLVPLGSSITSSLSHPPSEPDSHPHTVAGDYQSFPNHIMSTLLQTPALYTAATFASSFWPPDSSGGSPVPGNSPPNLAAMAAATVAAASAWWAANGLLPLCAPLSSGGFTSHPPSTFGPSCDVEYTKASTLQHGSVQSREQEHFEASKARSSLDSEDVENKSKPVCHEQPSATPESDAKGSDGAGDRKQVDRSSCGSNTPSSSDDVEADASERQEDGTNGEVKETNEDTNKPQTSESNARRSRISSNITDPWKSVSDEGRIAFQALFSREVLPQSFTYREEHREEEQQQQEQRYPMALDLNFTAQLTPVDDQEEKRNTGFLGIGLDASKLMSRGRTGFKPYKRCSMEAKESRILNNNPIIHVEQKDPKRMRLETQAST

SEQ ID NO: 135 - DNA, 1938 bp, Arabidopsis thaliana
  CDS (1)..(1935)
atggatactaatacatctggagaagaattattagctaaggcaagaaagccatatacaataacaaagcagcgagagcgatggactgaggatgagcatgagaggtttctagaagccttgaggctttatggaagagcttggcaacgaattgaagaacatattgggacaaagactgctgttcagatcagaagtcatgcacaaaagttcttcacaaagttggagaaagaggctgaagttaaaggcatccctgtttgccaagctttggacatagaaattccgcctcctcgtcctaaacgaaaacccaatactccttatcctcgaaagcctgggaacaacggtacatcttcctctcaagtatcatcagcaaaagatgcaaaacttgtttcatcggcctcttcttcacagttgaatcaggcgttcttggatttggaaaaaatgccgttctctgagaaaacatcaactggaaaagaaaatcaagatgagaattgctcgggtgtttctactgtgaacaagtatcccttaccaacgaaacaggtaagtggcgacattgaaacaagtaagacctcaactgtggacaacgcggttcaagatgttcccaagaagaacaaagacaaagatggtaacgatggtactactgtgcacagcatgcaaaactacccttggcatttccacgcagatattgtgaacgggaatatagcaaaatgccctcaaaatcatccctcaggtatggtatctcaagacttcatgtttcatcctatgagagaagaaactcacgggcacgcaaatcttcaagctacaacagcatctgctactactacagcttctcatcaagcgtttccagcttgtcattcacaggatgattaccgttcgtttctccagatatcatctactttctccaatcttattatgtcaactctcctacagaatcctgcagctcatgctgcagctacattcgctgcttcggtctggccttatgcgagtgtcgggaattctggtgattcatcaaccccaatgagctcttctcctccaagtataactgccattgccgctgctacagtagctgctgcaactgcttggtgggcttctcatggacttcttcctgtatgcgctccagctccaataacatgtgttccattctcaactgttgcagttccaactccagcaatgactgaaatggataccgttgaaaatactcaaccgtttgagaaacaaaacacagctctgcaagatcaaaacttggcttcgaaatctccagcttcatcatctgatgattcagatgagactggagtaaccaagctaaatgccgactcaaaaaccaatgatgataaaattgaggaggttgttgttactgccgctgtgcatgactcaaacactgcccagaagaaaaatcttgtggaccgctcatcctgtggctcaaatacaccttcagggagtgacgcagaaactgatgcattagataaaatggagaaagataaagaggatgtgaaggagacagatgagaatcagccagatgttattgagttaaataaccgtaagattaaaatgagagacaacaacagcaacaacaatgcaactactgattcgtggaaggaagtctccgaagagggtcgtatagcgtttcaggctctctttgcaagagaaagattgcctcaaagcttttcgcctcctcaagtggcagagaatgtgaatagaaaacaaagtgacacgtcaatgccattggctcctaatttcaaaagccaggattcttgtgctgcagaccaagaaggagtagtaatgatcggtgttggaacatgcaagagtcttaaaacgagacagacaggatttaagccatacaagagatgttcaatggaagtgaaagagagccaagttgggaacataaacaatcaaagtgatgaaaaagtctgcaaaaggcttcgattggaaggagaagcttctacatga

SEQ ID NO: 136 - protein, 645 aa, Arabidopsis thaliana
MDTNTSGEELLAKARKPYTITKQRERWTEDEHERFLEALRLYGRAWQRIEEHIGTKTAVQIRSHAQKFFTKLEKEAEVKGIPVCQALDIEIPPPRPKRKPNTPYPRKPGNNGTSSSQVSSAKDAKLVSSASSSQLNQAFLDLEKMPFSEKTSTGKENQDENCSGVSTVNKYPLPTKQVSGDIETSKTSTVDNAVQDVPKKNKDKDGNDGTTVHSMQNYPWHFHADIVNGNIAKCPQNHPSGMVSQDFMFHPMREETHGHANLQATTASATTTASHQAFPACHSQDDYRSFLQISSTFSNLIMSTLLQNPAAHAAATFAASVWPYASVGNSGDSSTPMSSSPPSITAIAAATVAAATAWWASHGLLPVCAPAPITCVPFSTVAVPTPAMTEMDTVENTQPFEKQNTALQDQNLASKSPASSSDDSDETGVTKLNADSKTNDDKIEEVVVTAAVHDSNTAQKKNLVDRSSCGSNTPSGSDAETDALDKMEKDKEDVKETDENQPDVIELNNRKIKMRDNNSNNNATTDSWKEVSEEGRIAFQALFARERLPQSFSPPQVAENVNRKQSDTSMPLAPNFKSQDSCAADQEGVVMIGVGTCKSLKTRQTGFKPYKRCSMEVKESQVGNINNQSDEKVCKRLRLEGEAST

SEQ ID NO: 137 - DNA, 1989 bp, Arabidopsis thaliana
  CDS (1)..(1986)
atgaatatgagtgatttaggttgggatgatgaagataaatcggtggttagtgctgttttagggcatttagcttctgattttcttcgagcaaactctaattcgaatcagaatctctttcttgttatgggaactgatgatactctgaataagaagctctctagtctcgttgattggccaaactcggagaatttcagctggaactacgctattttctggcaacaaaccatgtctagatccggacaacaagtcttaggttggggagatgggtgttgtcgagagcctaatgaggaagaggaatcaaaagttgttaggtcttataattttaacaacatgggggcagaggaagagacatggcaagatatgaggaagagagtgttgcagaagcttcataggttgtttggtggatctgatgaagacaattatgctttgagcttagagaaagttactgctactgagattttcttcttagcttccatgtatttcttcttcaatcacggtgaaggcggtcctgggaggtgttattcttcagggaaacatgtgtggctctctgatgcagttaactctgagtctgactattgtttcaggtcttttatggcgaaatctgcgggaatcagaacgatcgttatggttcctactgatgctggtgttcttgagcttggttctgtttggtctttgcctgaaaacattggcttggttaagtctgttcaagctttgttcatgaggagagttacgcaaccagtaatggtgacttcaaacactaacatgactggagggattcacaagcttttcgggcaggatttgagtggagctcacgcgtatcctaagaagctcgaagtgagaagaaacttggatgagagattcactcctcaaagttgggaaggctataataacaataaaggtccaacatttggttacacacctcagagggatgatgtgaaagtgctagagaatgtgaatatggttgtagataataacaattacaagacgcagattgagtttgcgggatcatcagttgctgcttcttcgaatccatctacaaacactcagcaagaaaaatcagaatcttgtacagagaaaagaccagtgagcttgttagcaggagcaggaatagtttctgttgttgatgagaagagaccgagaaagagagggagaaagcctgcaaacggaagagaagagccattgaaccatgtggaagctgagaggcagagacgcgagaagcttaaccaaagattctacgctttacgatcagttgttccaaacatttctaaaatggacaaggcttctctacttggagacgcaatttcttacatcaaagagcttcaagagaaagtcaagataatggaagatgaaagagtaggaacagataagagcttatcagaatcaaacacaataacagtagaagaaagtccagaagttgacattcaagctatgaatgaagaggttgttgtaagagtaatctcgcctttggattcacatccagcttcaagaatcatacaagcaatgagaaactcaaatgttagtctaatggaggctaagttatcattagctgaagacacaatgtttcacacttttgtgataaagtctaacaacgggtcggatccattgacgaaagagaagcttatagcagcgttttaccccgagaccagctcgacgcaaccgccattgccttcttctagttcacaggtctctgggtttgtttctccgatcagatccaatttcaatccttctccgtcagattatctcctcggccacgatgtctctccgccgcactatcctcgatctccaccgtcaaacccagagagccactctctcaaagtctctacctttctcgatgagtcacatctcctccgcatccgccaccgccgccgtgagaaacccactgggaagagacctctcttcaatccccttcgcacaagcccagaaactgaaaccagattcaaccaatctcgtcaccgatcgctcgatttcgtcttcaattgggcaatctga

SEQ ID NO: 138 - protein, 662 aa, Arabidopsis thaliana
MNMSDLGWDDEDKSVVSAVLGHLASDFLRANSNSNQNLFLVMGTDDTLNKKLSSLVDWPNSENFSWNYAIFWQQTMSRSGQQVLGWGDGCCREPNEEEESKVVRSYNFNNMGAEEETWQDMRKRVLQKLHRLFGGSDEDNYALSLEKVTATEIFFLASMYFFFNHGEGGPGRCYSSGKHVWLSDAVNSESDYCFRSFMAKSAGIRTIVMVPTDAGVLELGSVWSLPENIGLVKSVQALFMRRVTQPVMVTSNTNMTGGIHKLFGQDLSGAHAYPKKLEVRRNLDERFTPQSWEGYNNNKGPTFGYTPQRDDVKVLENVNMVVDNNNYKTQIEFAGSSVAASSNPSTNTQQEKSESCTEKRPVSLLAGAGIVSVVDEKRPRKRGRKPANGREEPLNHVEAERQRREKLNQRFYALRSVVPNISKMDKASLLGDAISYIKELQEKVKIMEDERVGTDKSLSESNTITVEESPEVDIQAMNEEVVVRVISPLDSHPASRIIQAMRNSNVSLMEAKLSLAEDTMFHTFVIKSNNGSDPLTKEKLIAAFYPETSSTQPPLPSSSSQVSGFVSPIRSNFNPSPSDYLLGHDVSPPHYPRSPPSNPESHSLKVSTFLDESHLLRIRHRRREKPTGKRPLFNPLRTSPETETRFNQSRHRSLDFVFNWAI

SEQ ID NO: 139 - DNA, 870 bp, Arabidopsis thaliana
  CDS (1)..(867)
atgtcagaattattacagttgcctccaggtttccgatttcaccctaccgatgaagagcttgtcatgcactatctctgccgcaaatgtgcctctcagtccatcgccgttccgatcatcgctgagatcgatctctacaaatacgatccatgggagcttcctggtttagccttgtatggtgagaaggaatggtacttcttctctcccagggacagaaaatatcccaacggttcgcgtcctaaccggtccgctggttctggttactggaaagctaccggagctgataaaccgatcggactacctaaaccggtcggaattaagaaagctcttgttttctacgccggcaaagctccaaagggagagaaaaccaattggatcatgcacgagtaccgtctcgccgacgttgaccggtccgttcgcaagaagaagaatagtctcaggctggatgattgggttctctgccggatttacaacaaaaaaggagctaccgagaggcggggaccaccgcctccggttgtttacggcgacgaaatcatggaggagaagccgaaggtgacggagatggttatgcctccgccgccgcaacagacaagtgagttcgcgtatttcgacacgtcggattcggtgccgaagctgcatactacggattcgagttgctcggagcaggtggtgtcgccggagttcacgagcgaggttcagagcgagcccaagtggaaagattggtcggccgtaagtaatgacaataacaatacccttgattttgggtttaattacattgatgccaccgtggataacgcgtttggaggaggagggagtagtaatcagatgtttccgctacaggatatgttcatgtacatgcagaagccttactag

SEQ ID NO: 140 - protein, 289 aa, Arabidopsis thaliana
MSELLQLPPGFRFHPTDEELVMHYLCRKCASQSIAVPIIAEIDLYKYDPWELPGLALYGEKEWYFFSPRDRKYPNGSRPNRSAGSGYWKATGADKPIGLPKPVGIKKALVFYAGKAPKGEKTNWIMHEYRLADVDRSVRKKKNSLRLDDWVLCRIYNKKGATERRGPPPPVVYGDEIMEEKPKVTEMVMPPPPQQTSEFAYFDTSDSVPKLHTTDSSCSEQVVSPEFTSEVQSEPKWKDWSAVSNDNNNTLDFGFNYIDATVDNAFGGGGSSNQMFPLQDMFMYMQKPY

SEQ ID NO: 141 - DNA, 1202 bp, Arabidopsis thaliana
  CDS (112)..(918)
tcctcgatttcaatctttgagataaaccacaaagatcctccgattcgaaggtttataaaaactcaaaatcgaatcttatccacaagaaaacaacaaggtacttttccaaaaatgaaggcggagttgaatttgccggcgggattccgatttcatccgacggacgaagagcttgtcaagttctatctttgccggagatgtgcgtcagaaccgattaacgttccggttatcgcagagattgacttgtacaaattcaatccaagggagcttccagaaatggcgttgtacggtgagaaagaatggtacttcttctcgcatagagaccggaaatacccaaacgggtcgagaccaaaccgggcagctggaaccggttattggaaagcgactggagctgataaaccgatcggaaaaccgaagacgttagggattaagaaagcactcgtcttctacgcaggaaaagctccgaaagggattaaaacgaattggattatgcacgagtatcgtctcgctaatgtcgatcgatctgcttctaccaacaagaagaacaacttaagacttgatgattgggttttgtgtcggatatacaataagaaaggaacaatggagaagtatttaccggcggcggctgagaaaccgacggaaaagatgagtacgtcggactcaagatgctcaagtcacgtgatttcaccggacgtcacgtgttctgataactgggaggttgagagtgagcccaaatggattaatctggaagacgcgttagaggcatttaatgatgacacgtccatgtttagttccattggtttgttgcaaaatgacgcctttgttcctcagtttcagtaccagtcctccgatttcgtcgattcgtttcaggacccgttcgagcagaaaccgttcttgaattggaattttgctcctcaagggtaaaaataatcggcaaaaagttgaagcttttcagagtcttcgatcaccggcattgtgtcggatcctgacccggagaccaagtcgggtcatacgattacataatcgggttattgagatttccacatttggatttccgagactaaccaacttaacggattctggggtaattggggggttttgcacaggtgaatcacactgagtcagcaagtttcgattttttggttttgttttgtaatgattgattaaatgtctaaagatatcacgaagttaaaaaaaaaaaaaaa

SEQ ID NO: 142 - protein, 269 aa, Arabidopsis thaliana
MKAELNLPAGFRFHPTDEELVKFYLCRRCASEPINVPVIAEIDLYKFNPRELPEMALYGEKEWYFFSHRDRKYPNGSRPNRAAGTGYWKATGADKPIGKPKTLGIKKALVFYAGKAPKGIKTNWIMHEYRLANVDRSASTNKKNNLRLDDWVLCRIYNKKGTMEKYLPAAAEKPTEKMSTSDSRCSSHVISPDVTCSDNWEVESEPKWINLEDALEAFNDDTSMFSSIGLLQNDAFVPQFQYQSSDFVDSFQDPFEQKPFLNWNFAPQG

SEQ ID NO: 143 - DNA, 1188 bp, Arabidopsis thaliana
  CDS (124)..(978)
aaaaacctcaactttcttctctcttctcaaaaacccttccctcttcgtctccaaacaacaacaaacacaacaacaacaaaaatcttacaagaagatcatttttagaaaccctattaggataaaatggattacgaggcatcaagaatcgtcgaaatggtagaagatgaagaacatatagatctaccaccaggattcagatttcaccctactgatgaagaactcataactcactacctcaaaccaaaggttttcaacactttcttctctgctactgccattggtgaagttgatctcaacaagattgagccttgggacttaccatggaaggctaagatgggagaaaaagaatggtatttcttctgtgtgagagaccggaaatacccgaccggtttaaggacaaaccgggcgacagaagccggttattggaaagccacaggaaaagacaaagagatattcaagggaaaatcacttgtgggtatgaagaaaactttggttttctataaaggaagagctcctaaaggagttaaaaccaattgggttatgcatgaatatcgtttagaaggcaaatattgtattgaaaatcttccccaaacagctaagaacgaatgggttatatgtcgtgttttccaaaaacgtgccgatggtacaaaggttccaatgtcaatgcttgatccacacattaaccgaatggaaccagccggtttaccttcgttaatggattgttctcaacgagactccttcaccggttcgtcgtctcacgtgacctgcttctccgaccaagaaaccgaagacaaaagacttgtccacgagtccaaagacggttttggttctctgttttactcggatcctctgtttttacaagacaattattcgctaatgaagctgttgcttgacggtcaagaaactcaattctccggcaaacctttcgacggtcgtgattcgtccggtacagaagaattggattgcgtttggaatttctgagttgtataagttatgttgtagacttgtagtagtcatgtgttcgtgtgtgtgaatgaatattcttgttacatttttttgtaaaaaaggagaaaaaaatatgctagaaagtcaattgcttttgttatgtagcattagtgttttttatgtactcaatagacttcctaattaaataaaaatcttaatttatttgcaaaaaaaaaaaaaaaa

SEQ ID NO: 144 - protein, 285 aa, Arabidopsis thaliana
MDYEASRIVEMVEDEEHIDLPPGFRFHPTDEELITHYLKPKVFNTFFSATAIGEVDLNKIEPWDLPWKAKMGEKEWYFFCVRDRKYPTGLRTNRATEAGYWKATGKDKEIFKGKSLVGMKKTLVFYKGRAPKGVKTNWVMHEYRLEGKYCIENLPQTAKNEWVICRVFQKRADGTKVPMSMLDPHINRMEPAGLPSLMDCSQRDSFTGSSSHVTCFSDQETEDKRLVHESKDGFGSLFYSDPLFLQDNYSLMKLLLDGQETQFSGKPFDGRDSSGTEELDCVWNF

SEQ ID NO: 145 - DNA, 894 bp, Arabidopsis thaliana
  CDS (1)..(891)
atgggtgttagagagaaagatccgttagcccagttgagtttgccaccaggttttagattttatccgacagatgaagagcttcttgttcagtatctatgtcggaaagttgcaggctatcatttctctctccaggtcatcggagacatcgatctctacaagttcgatccttgggatttgccaagtaaggctttgtttggagagaaggaatggtatttctttagcccaagagatcggaaatatccgaacgggtcaagacccaatagagtagccgggtcgggttattggaaagcaacgggtactgacaaaattatcacggcggatggtcgtcgtgtcgggattaaaaaagctctggtcttttacgccggaaaagctcccaaaggcactaaaaccaactggattatgcacgagtatcgcttaatagaacattctcgtagccatggaagctccaagttggatgattgggtgttgtgtcgaatttacaagaaaacatctggatctcagagacaagctgttactcctgttcaagcttgtcgtgaagagcatagcacgaatgggtcgtcatcgtcttcttcatcacagcttgacgacgttcttgattcgttcccggagataaaagaccagtcttttaatcttcctcggatgaattcgctcaggacgattcttaacgggaactttgattgggctagcttggcaggtcttaatccaattccagagctagctccgaccaatggattaccgagttacggtggttacgatgcgtttcgagcggcggaaggtgaggcggagagtgggcatgtgaatcggcagcagaactcgagcgggttgactcagagtttcgggtacagctcgagtgggtttggtgtttcgggtcaaacattcgagtttaggcaatga

SEQ ID NO: 146 - protein, 297 aa, Arabidopsis thaliana
MGVREKDPLAQLSLPPGFRFYPTDEELLVQYLCRKVAGYHFSLQVIGDIDLYKFDPWDLPSKALFGEKEWYFFSPRDRKYPNGSRPNRVAGSGYWKATGTDKIITADGRRVGIKKALVFYAGKAPKGTKTNWIMHEYRLIEHSRSHGSSKLDDWVLCRIYKKTSGSQRQAVTPVQACREEHSTNGSSSSSSSQLDDVLDSFPEIKDQSFNLPRMNSLRTILNGNFDWASLAGLNPIPELAPTNGLPSYGGYDAFRAAEGEAESGHVNRQQNSSGLTQSFGYSSSGFGVSGQTFEFRQ

SEQ ID NO: 147 - DNA, 1920 bp, Arabidopsis thaliana
  CDS (62)..(1645)
acagaaaaatcgaaactttttagggttttttttttttgtgataacgagagagaaaaaagtgatggacttgtcggttgagaacggaggtttagctccaggttttaggtttcatccgacggacgaagaacttgtcgtctattatctcaaaagaaagatccgtcggaaaaaactcagagtcgaagcaatcggcgagactgatgtctataagtttgatcctgaggaattacctgagaaagcgttgtataagactagagatcgtcaatggttctttttcagcttaagggataggaaacatggaagtaggtcaagtagagctactgaacgtggctattggaaagcaacagggaaggatagagtcattcattgtgattcgagacccgttggagagaagaagactcttgttttccatagaggcagggcacctaatggcgaacggactaattgggtgatgcatgagtatacattgcacaaagaggagctcaagaggtgtggtggtgaagatgttaaggatgcttatgttctttacaagatttataagaaaagtgggtctggtcctaagaatggtgagcaatatggagctccttttattgaagaagaatgggctgaagatgatgatgatgatgttgatgagcctgctaatcagctcgttgtttcggctagtgttgataatagtttatgggggaaagggcttaaccaatctgaattggatgataatgatattgaagagctgatgagtcaggttagagatcagtctggtccaacattgcagcagaatggggtgtctggactgaactctcatgtagacacgtataatctggagaacctggaggaagatatgtatttggaaatcaatgatcttatggaacctgaacctggaccaacttctgtggaagtcatggagaataactggaacgaggatggttctggtctcctgaatgatgatgatttcgttggtgctgattcatatttccttgatttgggagtgacaaatcctcagttagattttgttagtggtgatttgaaaaatgggtttgcacaaagtcttcaggtgaatacttctttaatgacttaccaggccaataataaccagttccagcagcaatcagggaagaaccaagctagtaactggccactccgtaacagctataccagacagataaacaatggatcatcgtgggtgcaggagctaaacaatgacggacttaccgttacccggtttggtgaggcgcctggtacaggtgattcatctgaattcctaaaccctgttccttctggtataagtacaactaatgaagatgacccgtcaaaagacgagtctagtaagtttgcttctagtgtatggactttcctggaatccattcctgcaaagccagcatatgcatcagagaatccatttgtgaagctgaaccttgttagaatgtcaaccagtggtggtcgtttcaggtttacttctaaaagcacaggtaataatgttgttgttatggatagtgactcagcagtgaagaggaacaagtctggaggaaacaacgataagaagaagaagaagaacaaaggtttcttttgcttatcgatcattggggctttatgtgctttgttttgggtgatcataggaacaatgggaggttcagggaggcctttgttatggtgagaaccgaaaaatccaagaggttaagagacataaaggcttggttttgtgtgaaccattagagagtcaagtcattgtaattattctcttggattattagattcagaagctgtttagtatcacagtttatgcttggaagttttctctggtattgttaaaaaagtaccaatagaaataagcaaaagaattttcttatcttttaggatatttgaacaaatgatgttacttaactagattattaacttggaggttgttgtacaaaaaaaaaaaaaaaa

SEQ ID NO: 148 - protein, 528 aa, Arabidopsis thaliana
MDLSVENGGLAPGFRFHPTDEELVVYYLKRKIRRKKLRVEAIGETDVYKFDPEELPEKALYKTRDRQWFFFSLRDRKHGSRSSRATERGYWKATGKDRVIHCDSRPVGEKKTLVFHRGRAPNGERTNWVMHEYTLHKEELKRCGGEDVKDAYVLYKIYKKSGSGPKNGEQYGAPFIEEEWAEDDDDDVDEPANQLVVSASVDNSLWGKGLNQSELDDNDIEELMSQVRDQSGPTLQQNGVSGLNSHVDTYNLENLEEDMYLEINDLMEPEPGPTSVEVMENNWNEDGSGLLNDDDFVGADSYFLDLGVTNPQLDFVSGDLKNGFAQSLQVNTSLMTYQANNNQFQQQSGKNQASNWPLRNSYTRQINNGSSWVQELNNDGLTVTRFGEAPGTGDSSEFLNPVPSGISTTNEDDPSKDESSKFASSVWTFLESIPAKPAYASENPFVKLNLVRMSTSGGRFRFTSKSTGNNVVVMDSDSAVKRNKSGGNNDKKKKKNKGFFCLSIIGALCALFWVIIGTMGGSGRPLLW

SEQ ID NO: 149 - DNA, 1219 bp, Arabidopsis thaliana
  CDS (177)..(950)
atgctaaggagccctcccaaaaaagaacaacaaatcacatttttatataactgttaacataataatctcagcctcatcaacacacatatatagatagccaacatcacacaaacatagagattccaaaaaataaaaataaagaaaacataaatcctctgaggaaaaattccgatgagatgacagaaggtggagaatattctccggcgatgatgtcagcagagccattcttgaccatgaagaagatgaagaagagcaaccacaacaagaacaatcagagaaggtttagcgacgagcagatcaagtcactggagatgatgtttgagtctgagacaaggcttgagccaaggaagaaggttcaattagctagagagctagggttgcagccgaggcaagtggctatatggtttcagaacaagagggctcgttggaaatccaagcagctcgagactgagtacaacattctcagacaaaactacgacaacttggcttctcagttcgagtccttaaagaaagaaaaacaagctttagtctctgagttgcagaggctaaaagaggcgacgcaaaagaagacacaggaggaggaaaggcagtgtagtggagatcaagcggtggttgctctaagcagcacacatcatgaatcagaaaacgaagagaaccggaggcgtaaaccggaagaggttagaccggagatggagatgaaagatgataagggtcatcatggggttatgtgtgatcatcatgattatgaagatgatgataatggttatagtaacaacatcaagagagagtattttggtgggtttgaggaagaaccagatcacttaatgaacattgttgaaccagctgatagttgtttgacatcatctgatgattggagaggtttcaaatcagatactactactctcttggaccaatccagcaacaattacccttggcgggatttttggtcatgaaaacaataaactctaaacaagaagatgaaacagattgagactaaagattggatatatacatattcaaatcgaaatttaccggtctacatcgcatgaaccgagccacggatatagagatattcggtccagcaaatgactcgtttctcagcgagaattttgcaggattttgagctgaaattgtatggttttgtctgtataaatgatgtgtttagaaagacgtatattctcaaaaaaaaaaaaaaaaaaaaagaaaaaaaaaaaaaaaa

SEQ ID NO: 150 - protein, 258 aa, Arabidopsis thaliana
MTEGGEYSPAMMSAEPFLTMKKMKKSNHNKNNQRRFSDEQIKSLEMMFESETRLEPRKKVQLARELGLQPRQVAIWFQNKRARWKSKQLETEYNILRQNYDNLASQFESLKKEKQALVSELQRLKEATQKKTQEEERQCSGDQAVVALSSTHHESENEENRRRKPEEVRPEMEMKDDKGHHGVMCDHHDYEDDDNGYSNNIKREYFGGFEEEPDHLMNIVEPADSCLTSSDDWRGFKSDTTTLLDQSSNNYPWRDFWS

SEQ ID NO: 151 - DNA, 978 bp, Arabidopsis thaliana
  CDS (89)..(793)
aaagaaagaaagaaaaaaaagaaacaaataattccaaaaccttctctcttaatcaaaatcaagaaacttacaagatctggtgaaaaccatggaagaaggagattttttcaactgctgtttcagcgagattagtagtggcatgaccatgaataagaagaagatgaagaagagcaataaccaaaagaggtttagcgaggaacagatcaagtcacttgagcttatatttgagtctgagacgaggcttgagccgaggaagaaggttcaggtagctagagagctagggctgcaaccaagacaagtggctatatggtttcaaaacaagagggctcgatggaaaactaagcaacttgagaaagagtataacactcttagagccaattacaacaatttggcttcacaatttgaaatcatgaagaaagaaaagcaatctctggtctctgagctgcagagactaaacgaagagatgcaaaggcctaaagaagaaaagcatcatgagtgttgtggtgatcaaggactggctctaagcagcagcacagagtcgcataatggaaagagtgagccagaagggaggttagaccaagggagtgttctatgtaatgatggtgattacaacaacaacattaaaacagagtattttgggttcgaggaagagactgatcatgagctgatgaacattgtggagaaagctgatgatagttgcttgacatcttctgagaattggggaggtttcaattctgattctctcttagaccaatctagcagcaattaccctaactggtgggagttttggtcataaaagcatataagaaaaaaacagaacataagcgaagagaaagagtgtgaatagtttgtaaattatgtgttaagaaaaataaatttagtttagtttaaatcttgtttcgatctatgtatctactatgttcaatactctttgtagctaattagtagcttataatgagactagaaaagttttgaagc

SEQ ID NO: 152 - protein, 235 aa, Arabidopsis thaliana
MEEGDFFNCCFSEISSGMTMNKKKMKKSNNQKRFSEEQIKSLELIFESETRLEPRKKVQVARELGLQPRQVAIWFQNKRARWKTKQLEKEYNTLRANYNNLASQFEIMKKEKQSLVSELQRLNEEMQRPKEEKHHECCGDQGLALSSSTESHNGKSEPEGRLDQGSVLCNDGDYNNNIKTEYFGFEEETDHELMNIVEKADDSCLTSSENWGGFNSDSLLDQSSSNYPNWWEFWS

SEQ ID NO: 153 - DNA, 2043 bp, Arabidopsis thaliana
  CDS (1)..(2040)
atggctgcttactttcacggaaacccaccggagatctctgccggatccgacggtggtcttcaaacgttgatcctcatgaatccaactacttacgttcagtacacccaacaagacaacgactcgaacaacaacaacaacagcaacaatagcaacaacaacaacacaaacacaaacacaaacaacaacaacagtagtttcgttttcctcgattcccacgcgccgcagccaaacgcgagccagcagttcgtcggaataccactctcaggtcacgaagctgcttccattacagccgccgacaacatctccgtacttcacggttatcctccgcgcgtgcagtacagtctctacggtagccaccaagtggatcccactcaccagcaagccgcgtgtgagacgccacgcgcgcagcaaggcctctctttaaccctctcgtctcaacagcagcagcaacagcaacatcatcaacaacaccagcctattcacgtcggattcgggtccggacatggagaagatatccgggtcgggtctggctctacaggatcgggggtaacaaacggtatagctaatcttgttagctccaagtacttgaaggcagcacaagagcttcttgacgaagtagtcaacgctgattccgatgacatgaacgctaaatcccaactattctcatcgaaaaagggtagttgcggaaatgataaacctgtcggagaatcatcggccggcgctggaggagaaggttccggtggcggagcagaagcagccgggaaacgtccggtggagctaggcacggcagagagacaagaaatacagatgaagaaagcaaaacttagtaacatgcttcatgaggtggagcagagatatagacagtaccaccagcagatgcagatggtgatctcttcgttcgagcaagcggcagggataggatcagcgaagtcatacacgtcgctagcattgaaaaccatatcaagacagttccgttgcttgaaagaggcgatcgctggtcagataaaagcggccaacaagagtcttggggaggaagattcagtgtctggtgttgggaggtttgaggggtcgaggctcaagttcgtggaccaccacttgagacagcaaagagctcttcaacaactgggaatgattcaacatccttccaataatgcttggagacctcaacgtggtctcccagaacgagccgtctcagttctccgtgcttggctcttcgaacactttcttcatccataccctaaggattcggacaagcacatgctagctaagcaaacaggactcactcgtagccaggtgtcgaactggtttataaacgcgagagttcggttatggaaaccaatggtggaggagatgtacatggaggaaatgaaggagcaggcaaagaacatgggatccatggaaaagactcctttggatcaaagcaacgaagattctgcttcaaagtcaacaagtaaccaagaaaagagcccaatggcggacactaattaccatatgaatcccaatcacaacggtgacctagaaggcgtcactggaatgcaaggaagccccaagagactaagaaccagcgacgagacaatgatgcagccaataaatgcggatttcagctccaacgagaagctcacgatgaagattctagaagaacggcaagggataagatcagatggtggctaccctttcatgggtaatttcgggcaataccaaatggatgagatgtcaagatttgatgtagtctcagaccaggagctcatggcgcaaaggtactcaggaaacaacaatggcgtgtccctcacgttaggtttacctcattgtgatagcttgtcgtccacgcaccatcagggtttcatgcagacccaccatgggattcctatagggagaagagtgaaaataggagaaacagaggaatatggacccgccaccatcaatggtggtagctcgaccacaaccgcacattcatcagcggcagctgccgcggcttacaatgggatgaacatacagaaccagaagagatatgtggctcagttattgcccgacttcgttgcataa

SEQ ID NO: 154 - protein, 680 aa, Arabidopsis thaliana
MAAYFHGNPPEISAGSDGGLQTLILMNPTTYVQYTQQDNDSNNNNNSNNSNNNNTNTNTNNNNSSFVFLDSHAPQPNASQQFVGIPLSGHEAASITAADNISVLHGYPPRVQYSLYGSHQVDPTHQQAACETPRAQQGLSLTLSSQQQQQQQHHQQHQPIHVGFGSGHGEDIRVGSGSTGSGVTNGIANLVSSKYLKAAQELLDEVVNADSDDMNAKSQLFSSKKGSCGNDKPVGESSAGAGGEGSGGGAEAAGKRPVELGTAERQEIQMKKAKLSNMLHEVEQRYRQYHQQMQMVISSFEQAAGIGSAKSYTSLALKTISRQFRCLKEAIAGQIKAANKSLGEEDSVSGVGRFEGSRLKFVDHHLRQQRALQQLGMIQHPSNNAWRPQRGLPERAVSVLRAWLFEHFLHPYPKDSDKHMLAKQTGLTRSQVSNWFINARVRLWKPMVEEMYMEEMKEQAKNMGSMEKTPLDQSNEDSASKSTSNQEKSPMADTNYHMNPNHNGDLEGVTGMQGSPKRLRTSDETMMQPINADFSSNEKLTMKILEERQGIRSDGGYPFMGNFGQYQMDEMSRFDVVSDQELMAQRYSGNNNGVSLTLGLPHCDSLSSTHHQGFMQTHHGIPIGRRVKIGETEEYGPATINGGSSTTTAHSSAAAAAAYNGMNIQNQKRYVAQLLPDFVA

SEQ ID NO: 155 - DNA, 1162 bp, Arabidopsis thaliana
  CDS (49)..(933)
aaattcgcttttttttttcttctttgtatattttttttttttttgaccatggcggaggaatttggaagcatagatttactcggagatgaagatttcttcttcgatttcgatccttcaatcgtaattgattctcttccggcggaggattttcttcagtcttcaccggattcatggatcggagaaatcgagaatcaattgatgaacgatgagaatcatcaagaggagagttttgtggaattggatcagcaatcggtttcagatttcatagcggatctactcgttgattatccaactagcgattctggctccgttgatttggcggctgataaagttctaaccgtcgattctcccgccgccgctgatgattccgggaaggagaattcggatttggttgttgagaagaagtctaatgattctggtagcgagattcatgatgatgatgacgaagaaggagacgatgatgctgtggctaaaaaacgaagaaggagagtaagaaatagagatgcggcggttagatcgagagagaggaagaaggaatatgtacaagatttagagaagaagagtaagtatctcgaaagagaatgcttgagactaggacgtatgcttgagtgcttcgttgctgaaaaccagtctctacgttactgtttgcaaaagggtaatggcaataatactaccatgatgtcgaagcaggagtctgctgtgctcttgttggaatccctgctgttgggttccctgctttggcttctgggagtaaacttcatttgcctattcccttatatgtcccacacaaagtgttgcctcctacgtccagaaccagaaaagctggttctaaacgggctcgggagtagtagcaaaccgtcttataccggcgttagtcggagatgtaagggttcgaggcctaggatgaaataccaaatcttaacccttgcggcgtgacaacgccttttttaactgcttcttttgcgcattttgagttgtagatgagtgtcttttagttttctctctcttgttttgtatttcgctgttgaaagttttctgtctaatatcgataagttaacagtgaatgtgggtcttatggttatggatgatatctatctaataatgcttctgcctttaaaatgttgattttgaggcataacttcaggcaaaaaaaaaaaagaaa

SEQ ID NO: 156 - protein, 295 aa, Arabidopsis thaliana
MAEEFGSIDLLGDEDFFFDFDPSIVIDSLPAEDFLQSSPDSWIGEIENQLMNDENHQEESFVELDQQSVSDFIADLLVDYPTSDSGSVDLAADKVLTVDSPAAADDSGKENSDLVVEKKSNDSGSEIHDDDDEEGDDDAVAKKRRRRVRNRDAAVRSRERKKEYVQDLEKKSKYLERECLRLGRMLECFVAENQSLRYCLQKGNGNNTTMMSKQESAVLLLESLLLGSLLWLLGVNFICLFPYMSHTKCCLLRPEPEKLVLNGLGSSSKPSYTGVSRRCKGSRPRMKYQILTLAA

SEQ ID NO: 157 - DNA, 438 bp, Arabidopsis thaliana
  CDS (1)..(435)
atggcaaacgcagagaagacaagttcaggttccgacatagatgagaagaaaagaaaacgcaagttatcaaaccgcgaatctgcaaggaggtcgcgtttgaagaaacagaagttaatggaagacacgattcatgagatctccagtcttgaacgacgaatcaaagagaacagtgagagatgtcgagctgtaaaacagaggcttgactcggtcgaaacggagaacgcgggtcttagatcggagaagatttggctctcgagttacgttagcgatttagagaatatgattgctacgacgagtttaacgctgacgcagagtggtggtggcgattgtgtcgacgatcagaacgcaaacgcgggaatagcggttggagattgtagacgtacaccgtggaaattgagttgtggttctctacaaccaatggcgtcctttaagacatga

SEQ ID NO: 158 - protein, 145 aa, Arabidopsis thaliana
MANAEKTSSGSDIDEKKRKRKLSNRESARRSRLKKQKLMEDTIHEISSLERRIKENSERCRAVKQRLDSVETENAGLRSEKIWLSSYVSDLENMIATTSLTLTQSGGGDCVDDQNANAGIAVGDCRRTPWKLSCGSLQPMASFKT

SEQ ID NO: 159 - DNA, 1697 bp, Arabidopsis thaliana
  CDS (74)..(1435)
ccaaagaaaaaaaataaattcgaaggtaaatatccagaagcttgatcctcctagttgtacgaaagcttgagtaatggggtctagattaaacttcaagagctttgttgatggtgtgagtgagcagcagccaacggtggggactagtcttccattgactaggcagaactctgtgttctcgttaacctttgatgagtttcagaactcatggggtggtggaattgggaaagattttgggtctatgaacatggatgagctcttgaagaacatttggactgcagaggaaagtcattcaatgatgggaaacaataccagttacaccaacatcagcaatggtaatagtggaaacactgttattaacggcggtggtaacaacattggtgggttagctgttggtgtgggaggagaaagtggtggttttttcactggtgggagtttgcagagacaaggttcacttaccttgcctcggacgattagtcagaaaagggttgatgatgtctggaaggagctgatgaaggaggatgacattggaaatggtgttgttaatggtgggacaagcggaattccgcagaggcaacaaacgctgggagagatgactttggaggagtttttggtcagggctggtgtggttagggaagaacctcaaccggtggagagtgtaactaacttcaatggcggattctatggatttggcagtaatggaggtcttgggacagctagtaatgggtttgttgcaaaccaacctcaagatttgtcaggaaatggagtagcggtgagacaggatctgctgactgctcaaactcagccactacagatgcagcagccacagatggtgcagcagccacagatggtgcagcagccgcaacaactgatacagacgcaggagaggccttttcccaaacagaccactatagcattttccaacactgttgatgtggttaaccgttctcaacctgcaacacagtgccaggaagtgaagccttcaatacttggaattcataaccatcctatgaacaacaatctactgcaagctgtcgattttaaaacaggagtaacggttgcagcagtatctcctggaagccagatgtcacctgatctgactccaaagagcgccctggatgcatctttgtcccctgttccttacatgtttgggcgagtgagaaaaacaggtgcagttctggagaaagtgattgagagaaggcaaaaaaggatgataaagaatagggaatcagctgcaagatcccgcgctcgcaagcaagcttatacgatggaactggaagcagaaattgcgcaactcaaagaattgaatgaagagttgcagaagaaacaagttgaaatcatggaaaagcagaaaaatcagcttctggagcctctgcgccagccatggggaatgggatgcaaaaggcaatgcttgcgaaggacattgacgggtccctggtagagcttataatggcgtctaaggaacccaacaaagcgccgaagttatagaacaactcagaagatagaaagctagctttgtacgtagtttaggcaggttctgtgggtgattgtaaatcttgaagtgtggcggatttgacagagatagataaacacatatctgttctattttcctaaatcttttggttttatcttcctgatgtaatggatctttatcatttgtcttgaacatctttgtgacttaaccagagtgaatttatctt

SEQ ID NO: 160 - protein, 454 aa, Arabidopsis thaliana
MGSRLNFKSFVDGVSEQQPTVGTSLPLTRQNSVFSLTFDEFQNSWGGGIGKDFGSMNMDELLKNIWTAEESHSMMGNNTSYTNISNGNSGNTVINGGGNNIGGLAVGVGGESGGFFTGGSLQRQGSLTLPRTISQKRVDDVWKELMKEDDIGNGVVNGGTSGIPQRQQTLGEMTLEEFLVRAGVVREEPQPVESVTNFNGGFYGFGSNGGLGTASNGFVANQPQDLSGNGVAVRQDLLTAQTQPLQMQQPQMVQQPQMVQQPQQLIQTQERPFPKQTTIAFSNTVDVVNRSQPATQCQEVKPSILGIHNHPMNNNLLQAVDFKTGVTVAAVSPGSQMSPDLTPKSALDASLSPVPYMFGRVRKTGAVLEKVIERRQKRMIKNRESAARSRARKQAYTMELEAEIAQLKELNEELQKKQVEIMEKQKNQLLEPLRQPWGMGCKRQCLRRTLTGPW

SEQ ID NO: 161 - DNA, 1717 bp, Arabidopsis thaliana
  CDS (234)..(1379)
aaaaaaaaaaaaaactgaactctttttcgctctggtttttttagagagagagaaagatgaaaatgcgtttaattgctgtttaggtttcgaattcgcgatttaaatttctgggtttctctctgtttaagcttcttcttcttcatcttctgcttacgtttcttcttcaaggagctttcggattcttgtagaaagagtcattgttctcttgagtgggaaaccttgaaaccattcctatgggaaatagcagcgaggaaccaaagcctcctaccaaatcagataaaccatcttcacccccggtggatcaaacaaatgttcatgtctaccctgattgggcagctatgcaggcatattatggtccaagagtagcaatgcctccttattacaattcagctatggctgcatctggtcatcctcctcctccttacatgtggaatcctcagcatatgatgtcaccatatggagcaccctatgctgctgtttatcctcatggaggaggagtttacgctcatcccggtattcccatgggatcactgcctcaaggtcaaaaggatccacctttaacaactccggggacgcttttgagcatcgacactcctactaaatctacagggaacacagacaatggattgatgaagaagctgaaagagtttgatgggcttgctatgtctctaggaaatgggaatcctgaaaatggtgcagatgaacataaacgatcacggaacagctcagaaactgatggttctactgatggaagtgatgggaatacaactggggcagatgaaccgaaacttaaaagaagtcgagagggaactccaacaaaagatgggaaacaattggttcaagctagctcatttcattctgtttctccgtcaagtggtgataccggcgtaaaactcattcaaggatctggagctatactctctcctggtgtaagtgcaaattccaaccccttcatgtcacaatctttagccatggttcctcctgaaacttggcttcagaacgagagagaactgaaacgggagcgaaggaaacagtctaatagagaatctgctagaaggtcaagattaaggaaacaggccgagacagaagaacttgctaggaaagtggaagccttgacagccgaaaacatggcattaagatctgaactaaaccaacttaatgagaaatctgataaactaagaggagcaaatgcaaccttgttggacaaactgaaatgctcggaacccgaaaagagagtccccgcaaatatgttgtctagagttaagaactcaggagctggagataagaacaagaaccaaggagacaatgattctaactctacaagcaaattgcatcaactgctcgatacgaagcctcgagctaaagcagtagctgcaggctgatcgatggtaattcatgtcgatttctacttaatttgtcgacataaacaaagaaaataagtgctactaatttcagaaaaacttgatagatagtatagtagagagagagagagagagagaggtgtgatgattattgatctataaattttcggagagagagagggagaaagagaaacttttcctccagatgaaaatttggtgttatggtttgttactgttaatatagagaggcttttctttttttataaaatggcttcctttgttgcatttccttgttttagacctgatgtaattttatgaaatcggtgttattgctttgcgtaaaaaaaaaaaaaaaa

SEQ ID NO: 162 - protein, 382 aa, Arabidopsis thaliana
MGNSSEEPKPPTKSDKPSSPPVDQTNVHVYPDWAAMQAYYGPRVAMPPYYNSAMAASGHPPPPYMWNPQHMMSPYGAPYAAVYPHGGGVYAHPGIPMGSLPQGQKDPPLTTPGTLLSIDTPTKSTGNTDNGLMKKLKEFDGLAMSLGNGNPENGADEHKRSRNSSETDGSTDGSDGNTTGADEPKLKRSREGTPTKDGKQLVQASSFHSVSPSSGDTGVKLIQGSGAILSPGVSANSNPFMSQSLAMVPPETWLQNERELKRERRKQSNRESARRSRLRKQAETEELARKVEALTAENMALRSELNQLNEKSDKLRGANATLLDKLKCSEPEKRVPANMLSRVKNSGAGDKNKNQGDNDSNSTSKLHQLLDTKPRAKAVAAG

SEQ ID NO: 163 - DNA, 1554 bp, Arabidopsis thaliana
  CDS (162)..(1379)
acaaaatatctctccctctatctgcaaattttccaaagttgcatcctttcaatttccactcctctctaatataattcacattttcccactattgctgattcatttttttttgtgaattatttcaaacccacataaaaaaatctttgtttaaatttaaaaccatggatccttcatttaggttcattaaagaggagtttcctgctggattcagtgattctccatcaccaccatcttcttcttcatacctttattcatcttccatggctgaagcagccataaatgatccaacaacattgagctatccacaaccattagaaggtctccatgaatcagggccacctccatttttgacaaagacatatgacttggtggaagattcaagaaccaatcatgtcgtgtcttggagcaaatccaataacagcttcattgtctgggatccacaggccttttctgtaactctccttcccagattcttcaagcacaataacttctccagttttgtccgccagctcaacacatatggtttcagaaaggtgaatccggatcggtgggagtttgcaaacgaagggtttcttagagggcaaaagcatctcctcaagaacataaggagaagaaaaacaagtaataatagtaatcaaatgcaacaacctcaaagttctgaacaacaatctctagacaatttttgcatagaagtgggtaggtacggtctagatggagagatggacagcctaaggcgagacaagcaagtgttgatgatggagctagtgagactaagacagcaacaacaaagcaccaaaatgtatctcacattgattgaagagaagctcaagaagaccgagtcaaaacaaaaacaaatgatgagcttccttgcccgcgcaatgcagaatccagattttattcagcagctagtagagcagaaggaaaagaggaaagagatcgaagaggcgatcagcaagaagagacaaagaccgatcgatcaaggaaaaagaaatgtggaagattatggtgatgaaagtggttatgggaatgatgttgcagcctcatcctcagcattgattggtatgagtcaggaatatacatatggaaacatgtctgaattcgagatgtcggagttggacaaacttgctatgcacattcaaggacttggagataattccagtgctagggaagaagtcttgaatgtggaaaaaggaaatgatgaggaagaagtagaagatcaacaacaagggtaccataaggagaacaatgagatttatggtgaaggtttttgggaagatttgttaaatgaaggtcaaaattttgattttgaaggagatcaagaaaatgttgatgtgttaattcagcaacttggttatttgggttctagttcacacactaattaagaagaaattgaaatgatgactactttaagcatttgaatcaacttgtttcctattagtaatttggctttgtttcaatcaagtgagtcgtggactaacttattgaatttgggggttaaatccgtttcttatttttggaaataaaattgctttttgtttaaaaaaaaaaaaaaaa

SEQ ID NO: 164 - protein, 406 aa, Arabidopsis thaliana
MDPSFRFIKEEFPAGFSDSPSPPSSSSYLYSSSMAEAAINDPTTLSYPQPLEGLHESGPPPFLTKTYDLVEDSRTNHVVSWSKSNNSFIVWDPQAFSVTLLPRFFKHNNFSSFVRQLNTYGFRKVNPDRWEFANEGFLRGQKHLLKNIRRRKTSNNSNQMQQPQSSEQQSLDNFCIEVGRYGLDGEMDSLRRDKQVLMMELVRLRQQQQSTKMYLTLIEEKLKKTESKQKQMMSFLARAMQNPDFIQQLVEQKEKRKEIEEAISKKRQRPIDQGKRNVEDYGDESGYGNDVAASSSALIGMSQEYTYGNMSEFEMSELDKLAMHIQGLGDNSSAREEVLNVEKGNDEEEVEDQQQGYHKENNEIYGEGFWEDLLNEGQNFDFEGDQENVDVLIQQLGYLGSSSHTN

SEQ ID NO: 165 - DNA, 23 bp, Artificial Sequence
  Description of Artificial Sequenceprimer
cgccagggttttcccagtcacga

SEQ ID NO: 166 - DNA, 24 bp, Artificial Sequence
  Description of Artificial Sequenceprimer
agcggataacaatttcacacagga

(19) United States
(12) Patent Application Publication
(10) Pub. No.: US 2005/0009187 A1
(43) Pub. Date: Jan. 13, 2005

(54) Environmental stress-responsive promoter and genes encoding transcriptional factor

(76) Inventors: Kazuo Shinozaki, Ibaraki (JP); Motoaki Seki, Kanagawa (JP); Miki Fujita, Ibaraki (JP)

Correspondence Address: BIRCH STEWART KOLASCH & BIRCH, PO BOX 747, FALLS CHURCH, VA, 22040-0747, US

(21) Appl. No.: 10/495,918

(22) PCT Filed: Nov. 15, 2002
(86) PCT No.: PCT/JP02/11955

(30) Foreign Application Priority Data
Nov. 19, 2001 (JP) 2001-353038

(51) Int. Cl. C12N 15/82; C07H 21/04; C12N 15/87; C12N 15/09; C12N 15/63

(52) U.S. Cl. 435/468; 536/23.6; 435/320.1; 800/278; 536/24.1

(57) ABSTRACT

The present invention provides a stress responsive promoter. The environmental stress responsive promoter of the present invention comprises DNA of the following (a), (b) or (c):(a) DNA consisting of any nucleotide sequence selected from SEQ ID NOS: 1 to 90; (b) DNA consisting of a nucleotide sequence comprising a deletion, substitution or addition of one or more nucleotides relative to any nucleotide sequence selected from SEQ ID NOS: 1 to 90, and functioning as an environmental stress responsive promoter; and (c) DNA hybridizing under stringent conditions to DNA consisting of any nucleotide sequence selected from SEQ ID NOS: 1 to 90, and functioning as an environmental stress responsive promoter.

### TECHNICAL FIELD

The present invention relates to an environmental stress-responsive promoter and a gene encoding environmental stress-responsive transcriptional factor.

### BACKGROUND ART

Large quantities of genomic and cDNA sequences have been determined with respect to a number of organisms by gene sequencing projects. In a plant model, Arabidopsis thaliana, the complete genomic sequences of two chromosomes have been determined (Lin, X. et al., (1999), Nature 402, 761-768; and Mayer, K. et al., (1999), Nature 402, 769-777).

The expressed sequence tag (EST) project also has greatly contributed to the discovery of expression genes (Hofte, H. et al., (1993), Plant J. 4, 1051-1061; Newman, T. et al., (1994), Plant Physiol. 106, 1241-1255; and Cooke, R. et al., (1996), Plant J. 9, 101-124; and Asamizu, E. et al., (2000), DNA Res. 7, 175-180). For example, the database of EST (dbEST) of the National Center for Biotechnology Information(NCBI) includes partial cDNA sequences, in which more than half (about 28,000 genes) of the total genes are reproduced, (as estimated from the gene content of Arabidopsis thaliana chromosome 2 completely sequenced [Lin, X. et al., (1999), Nature 402, 761-768]).

Recently, microarray (DNA chip) technology has become a useful tool for analyzing genome-scale gene expression (Schena, M. et al., (1995), Science 270, 467-470; Eisen, M. B. and Brown, P. O. (1999), Methods Enzymol. 303, 179-205). In the technology using a DNA chip, cDNA sequences are arrayed on a slide glass in a density of not smaller than 1,000 genes/cm ². The cDNA sequences thus arrayed are hybridized simultaneously with a pair of cDNA probes tagged with two color fluorescent labels, which have been prepared from RNA samples of different types of cells or tissues. In this manner, a large amount of genes can be directly analyzed and compared for gene expression. This technology was demonstrated for the first time by analyzing 48 Arabidopsis genes for differential expression in root and shoots (Schena, M. et al., (1995), Science 270, 467-470). Furthermore, a microarray was used in investigating 1,000 clones randomly taken from a human cDNA library in order to identify a novel gene responsive to heat shock and protein kinase C activation (Schena, M. et al., (1996), Proc. Natl. Acad. Sci. USA, 93, 10614-10619).

In another method, a DNA chip is used in analyzing the expression profile of an inflammatory-disease associated gene under various induction conditions (Heller, R. A. et al., (1997), Proc. Natl. Acad. Sci. USA, 94, 2150-2155). Furthermore, using a microarray, a yeast genome having more than 6,000 coding sequences has been analyzed for dynamic expression (DeRisi, J. L. et al., (1997) Science 278, 680-686; and Wodicka, L. et al., (1997), Nature Biotechnol. 15, 1359-1367).

However, in the field of plant science, only a few reports have been made on microarray analysis (Schena, M. et al., (1995), Science 270, 467-470; Ruan, Y. et al., (1998), Plant J. 15, 821-833; Aharoni. A. et al., (2000), Plant Cell 12, 647-661; and Reymond, P. et al., (2000), Plant Cell 12, 707-719).

The growth of plants is significantly affected by environmental stresses such as drought, high salinity and low temperature. Of the stresses, drought or water deficiency is the most critical factor that limits growth of plants and production of crops. Such a drought stress causes various biochemical and physiological responses in plants.

To survive under these conditions of stress, plants acquire responsivity and adaptability to the stresses. Recently, several types of genes responsive to drought at a transcriptional level have been reported (Bohnert, H. J. et al., (1995), Plant Cell 7, 1099-1111; Ingram, J., and Bartels, D. (1996), Plant Mol. Biol. 47, 377-403; Bray, E. A. (1997), Trends Plant Sci. 2, 48-54; Shinozaki, K., and Yamaguchi-Shinozaki, K. (1997), Plant Physiol. 115, 327-334; Shinozaki, K., and Yamaguchi-Shinozaki, K. (1999), “Molecular responses to drought stress. Molecular responses to cold, drought, heat and salt stress in higher plants”, edited by Shinozaki, K. and Yamaguchi-Shinozaki, K. R. G. Landes Company; and Shinozaki, K., and Yamaguchi-Shinozaki, K. (2000), Curr. Opin. Plant Biol. 3, 217-223).

On the other hand, in an attempt to improve stress resistance of plants by introducing a gene, stress-inducible genes have been used (Holmberg, N., and Bulow, L. (1998), Trends Plant Sci. 3, 61-66; and Bajaj, S. et al., (1999), Mol. Breed. 5, 493-503). Not only to further clarify the mechanism of stress resistance and stress responsivity of a higher plant at a molecular level but also to improve the stress resistance of a crop by gene manipulation, it is important to analyze the function of a stress-inducible gene.

Dehydration responsive element and C-repeat sequence (DRE/CRT) has been identified as an important cis-acting element when drought, high salt and cold stress-responsive genes are expressed in an ABA independent manner, where ABA refers to abscisic acid, a kind of plant hormone and serves as a signal transmission factor of seed dormancy and environmental stress (Yamaguchi-Shinozaki, K., and Shinozaki, K. (1994), Plant Cell 6, 251-264; Thomashow, M. F. et al., (1999), Plant Mol. Biol. 50, 571-599; and Shinozaki, K., and Yamaguchi-Shinozaki, K. (2000), Curr. Opin. Plant Biol. 3, 217-223). Furthermore, a transcriptional factor (DREB/CBF) involved in DRE/CRT responsive gene expression has been cloned (Stockinger. E. J. et al., (1997), Proc. Natl. Acad. Sci. USA 94, 1035-1040; Liu, Q. et al., (1998), Plant Cell 10, 1391-1406; Shinwari, Z. K. et al., (1998), Biochem. Biophys. Res. Commun. 250, 161-170; and Gilmour, S. J. et al., (1998), Plant J. 16, 433-443). DREB1/CBF is considered to function in cold-responsive gene expression, whereas DREB2 is involved in drought-responsive gene expression. Strong resistance to freezing stress was observed in a transgenic Arabidopis plant that overexpresses CBF1 (DREB1B) CDNA under the control of a cauliflower mosaic virus (CaMV) 35S promoter (Jaglo-Ottosen, K. R. et al., (1998), Science 280, 104-106).

The present inventors have reported that when DREB1A (CBF3) cDNA molecules are overexpressed in transgenic plants under the control of a CaMV 35S promoter or a stress-inducible rd29A promoter, strong constitutive expression of stress-inducible DREB1A target genes are induced to improve resistance to freezing, drought and salt stresses (Liu, Q. et al., (1998), Plant Cell 10, 1391-1406; and Kasuga, M. et al., (1999), Nature Biotechnol. 17, 287-291). Furthermore, the present inventors have already identified six DREB1A target genes such as rd29A/lti78/cor78, kin1, kin2/cor6.6, cor15a, rd17/cor47, and erd10 (Kasuga, M. et al., (1999), Nature Biotechnol. 17, 287-291). However, it has not yet been sufficiently elucidated how the overexpressed DREB1A cDNA improves stress resistance to freezing, drought and salt in a transgenic plant. To investigate the molecular mechanisms of drought and freezing resistance, it is important to identify and analyze as many genes controlled by DREB1A as possible.

### DISCLASURE OF THE INVENTION

The present invention is directed to providing an environmental stress-responsive promoter and a gene encoding an environmental stress-responsive transcriptional factor.

The present inventors have intensively studied to solve the aforementioned problems. As a result, they succeeded in identifying novel genes responsive to cold, drought and salt stresses and isolating promoter regions thereof by using CDNA microarray analysis, thereby accomplishing the present invention.

More specifically, the present invention is directed to an environmental stress-responsive promoter comprising DNA of the following (a), (b) or (c):
(a) DNA consisting of any nucleotide sequence selected from SEQ ID NOS: 1 to 90;
(b) DNA consisting of a nucleotide sequence comprising a deletion, substitution or addition of one or more nucleotides relative to any nucleotide sequence selected from SEQ ID NOS: 1 to 90, and functioning as an environmental stress responsive promoter; and
(c) DNA hybridizing under stringent conditions to DNA consisting of any nucleotide sequence selected from SEQ ID NOS: 1 to 90, and functioning as an environmental stress responsive promoter.

Examples of environmental stress include at least one selected from the group consisting of cold stress, drought stress, and salt stress.

The present invention is also directed to an expression vector comprising the promoter mentioned above, or an expression vector having an arbitrary gene integrated therein.

Furthermore, the present invention is directed to a transformant comprising the expression vector.

Moreover, the present invention is directed to a transgenic plant, such as a plant body, plant organ, plant tissue or plant culture cell, comprising the expression vector.

The present invention is still further directed to a method for producing a stress-resistant plant, comprising culturing or cultivating the transgenic plant.

On the other hand, the present inventors identified novel genes encoding cold, drought and salt stress-responsive transcriptional factors by use of cDNA microarray analysis, thereby accomplishing the present invention.

More specifically, the present invention is directed to a gene encoding an environmental stress-responsive transcriptional factor comprising an amino acid of the following (a) or (b):
(a) any amino acid sequence selected from SEQ ID NOS: 2n (n is an integer from 47 to 82);
(b) an amino acid sequence comprising a deletion, substitution or addition of one or more amino acids relative to any amino acid sequence selected from SEQ ID NOS: 2n (n is an integer from 47 to 82), functioning as an environmental stress-responsive transcriptional factor.

Also, the present invention is directed to a gene according to claim 1, comprising DNA of the following (a), (b) or (c):
(a) DNA consisting of any nucleotide sequence selected from SEQ ID NOS: 2n−1 (n is an integer from 47 to 82);
(b) DNA consisting of a nucleotide sequence comprising a deletion, substitution or addition of one or more nucleotides relative to any nucleotide sequence selected from SEQ ID NOS: 2n−1 (n is an integer from 47 to 82), and encoding an environmental stress-responsive transcriptional factor; and
(c) DNA hybridizing under stringent conditions to DNA consisting of any nucleotide sequence selected from SEQ ID NOS: 2n−1 (n is an integer from 47 to 82), and encoding an environmental stress-responsive transcriptional factor.

In the present invention, examples of environmental stress include at least one selected from the group consisting of cold stress, drought stress, and salt stress.

The present invention is also directed to an expression vector containing the gene, a transformant containing the expression vector, and a transgenic plant containing the expression vector.

Furthermore, the present invention is directed to a transgenic plant, such as a plant body, plant organ, plant tissue or plant culture cell.

Moreover, the present invention is directed to a method for producing a stress-resistant plant, comprising culturing or cultivating the transgenic plant.

Hereinafter, the present invention will be described in detail.

The present inventors constructed full-length cDNA libraries from Arabidopsis plants placed under different conditions, such as dehydration-treated plants and cold-treated plants (Seki. M. et al., (1998), Plant J. 15, 707-720), by the biotinylated CAP trapper method (Carninci. P. et al., (1996), Genomics, 37, 327-336). Then, Arabidopsis full-length cDNA microarrays were respectively prepared using about 1,300 full-length cDNA molecules and about 7,000 full-length cDNA molecules both containing stress-inducible genes. Besides using these dehydration and cold-inducible full-length cDNA molecules, another cDNA microarray was prepared using a DREB1A target gene, a transcriptional regulator for controlling expression of a stress-responsive gene. Thereafter, expression patterns of genes under drought and cold stress were monitored to exhaustively analyze stress-responsive genes. As a result, from the full-length cDNA microarray containing about 1,300 of full-length cDNA molecules, novel environmental stress-responsive genes, that is, 44 drought-inducible genes and 19 cold-inducible genes were isolated. 30 out of the 44 drought-inducible genes, and 10 out of the 19 cold-inducible genes were novel stress-inducible genes. Moreover, it was found that 12 stress-inducible genes were DREB1A target genes and 6 out of the 12 stress-inducible genes were novel genes. As a result of the analysis, 301 drought-inducible genes, 54 cold-inducible genes and 211 high salt-stress inducible genes were isolated from a cDNA microarray containing about 7,000 full-length cDNA molecules.

Thereafter, not only promoter regions but also environmental genes encoding environmental stress-responsive transcriptional factors were successfully isolated from these environmental stress-responsive genes.

As described above, a full-length cDNA microarray is useful tool for analyzing the expression manner of Arabidopsis thaliana drought- and cold-stress inducible genes and analyzing the target gene of a stress associated transcriptional regulator.

1. Isolation of Promoter

The promoter of the present invention contains a cis-element which is present upstream of a gene encoding a stress-responsive protein expressed by an environmental stress such as a cold, drought, or high salt stress and which activates the transcription of a gene present downstream thereof by binding of a transcriptional factor. Examples of such a cis-element include a dehydration responsive element (DRE), an abscisic acid responsive element (ABRE), and a cold-stress responsive element. Examples of genes encoding proteins binding to these elements include a DRE binding protein 1A gene (referred to also as a “DREB1A gene”), DRE binding protein 1C gene (referred to also as a “DREB1C gene”), DRE binding protein 2A gene (referred to also as a “DREB2A gene”), and DRE binding protein 2B gene (referred to also as a “DREB2B gene”).

In isolating a promoter of the present invention, first, stress-responsive genes are isolated by using a microarray. In constructing a microarray, use may be made of about 1,300 cDNA molecules in total including genes isolated from Arabidopsis full-length cDNA libraries, responsive to dehydration (RD) genes, early responsive to dehydration (ERD) genes, kin1 genes, kin2 genes, and cor15a genes; and furthermore, α-tubulin genes as an internal standard; and moreover, mouse nicotinic acetylcholine receptor epsilon subunit (nAChRE) genes and mouse glucocorticoid receptor homologous genes, as negative controls.

As a microarray used in isolating the promoter of the present invention, use may be made of about 7,000 cDNA molecules in total including genes isolated from an Arabidopsis full-length cDNA library, responsive to dehydration (RD) genes, early responsive to dehydration(ERD) genes, and PCR amplification fragments as an internal standard obtained from λ control template DNA fragments (TX803, manufactured by Takara Shuzo); and mouse nicotinic acetylcholine receptor epsilon subunit (nAChRE) genes and mouse glucocorticoid receptor homologous genes, as negative controls.

A plasmid DNA extracted with a plasmid preparation device (manufactured by Kurabo) is sequenced by sequence analysis using a DNA sequencer (ABI PRISM 3700, PE Applied Biosystems, CA, USA). Based on the GenBank/EMBL database, the obtained sequence is screened for homology by using the BLAST program.

After poly A selection is performed, reverse transcription is carried out to synthesize double-stranded DNA molecules and a cDNA molecule is inserted into a vector.

The cDNA molecule inserted into a vector for constructing cDNA libraries is amplified by PCR using complementary primers to the sequences of vectors on both sides of the cDNA molecule. Examples of such vectors include λZAPII and λPS.

A microarray can be prepared according to a conventional method, which is not particularly limited. For example, using a gene tip microarray stamp machine GTMASS SYSTEM (manufactured by Nippon Laser & Electronics Lab.), the above obtained PCR product is loaded from a microtiter plate and spotted on a microslide glass at predetermined intervals. Then, to prevent a non-specific signal from being expressed, the slide is immersed into a blocking solution.

Examples of plant materials include a plant strain obtained by destroying specific genes as well as wild type plants. A transgenic plant having cDNA of DREB1A introduced therein may be used. Examples of plant species include Arabidopsis thaliana, tobacco and rice. Of them, Arabidopsis thaliana is preferable.

Dehydration- and cold-stress treatments can be carried out according to a known method (Yamaguchi-Shinozaki, K., and Shinozaki, K. (1994), Plant Cell 6, 251-264).

After plant bodies (wild type plants and DREB1A overexpression transformants) are exposed to stress, they are sampled and stored in cryogenic conditions with liquid nitrogen. The wild type and DREB1A overexpression transformants are used in an experiment to identify a DREB1A target gene. From plant bodies, mRNA is isolated and purified by a known method or a kit.

In the presence of Cy3 dUTP or Cy5 dUTP for labeling (Amersham Pharmacia), each of mRNA samples is subjected to reverse transcription and then used in hybridization.

After the hybridization, the microarray is scanned with a scanning laser microscope or the like. As a program for analyzing data of a microarray, Imagene Ver 2.0 (BioDiscovery) and QuantArray (GSI Lumonics) etc. may be used.

After the scanning, a plasmid having a target gene is prepared. In this way, the target genes are isolated.

A promoter region is determined by analyzing the nucleotide sequence of the gene isolated above and using a gene analysis program based on the genomic information of database (GenBank/EMBL, ABRC). The isolated genes can be classified into ones inducible by both dehydration and cold stress, ones inducible specifically by drought stress, and one inducible specifically by cold stress. According to the gene analysis program, from the genes mentioned above, 90 types of genes below can be identified.

(FL03-07-F12, FL04-12-F24, FL04-14-N10, FL04-14-P24, FL04-17-I03, FL04-17-M08, FL04-17-M22, FL05-05-A17, FL05-05-F20, FL05-05-G20, FL05-09-N09, FL05-10-J09, FL05-10-M08, FL05-11-H09, FL05-12-H13, FL05-13-I20, FL05-14-E15, FL05-14-E16, FL05-16-F03, FL05-16-H23, FL05-18-M07, FL05-18-O21, FL05-19-F21, FL05-19-O22, FL05-21-K17, FL06-10-F03, FL06-12-H12, FL07-12-I23, FL08-08-H23, FL08-08-O14, FL08-09-M05, FL08-10-K08, FL08-11-P07, FL08-13-F10, FL08-19-D04, FL08-19-G15, FL09-06-B11, FL09-07-G17, FL09-10-A12, FL09-13-P15, FL02-05-I05, FL04-12-N15, FL04-16-P21, FL04-17-N22, FL04-20-P19, FL02-09-H01, FL05-01-D08, FL05-02-G08, FL05-02-O17, FL05-07-L13, FL05-08-B14, FL05-09-N10, FL05-11-L01, FL05-12-J09, FL05-14-D24, FL05-14-F20, FL05-14-I08, FL05-15-C04, FL05-15-E19, FL05-18-A06, FL05-18-H15, FL05-19-C02, FL05-20-M16, FL05-20-N18, FL05-21-E06, FL05-21-L12, FL06-07-B08, FL06-08-H20, FL06-09-N04, FL06-11-K21, FL07-07-G15, FL07-12-D17, FL08-11-C23, FL08-13-G20, FL08-15-M21, FL08-18-N19, FL08-19-C07, FL08-19-P05, FL09-07-G09, FL09-07-G15, FL09-10-J18, FL09-11-I12, FL09-12-B03, FL09-16-I11, FL09-16-M04, FL11-01-J18, FL11-07-D13, FL11-07-F02, FL11-07-N15 and FL11-10-D10). The promoter regions of these genes are represented by SEQ ID NOS: 1 to 90, respectively.

As long as a promoter of the present invention acts as an environmental stress-responsive promoter, use may be made of any promoter having a nucleotide sequence selected from SEQ ID NOS: 1 to 90 wherein one or more nucleotides, preferably one or several nucleotides (for example 1 to 10, preferably 1 to 5) may be deleted, substituted or added. Furthermore, DNA hybridizing with the DNA comprising any nucleotide sequence selected from SEQ ID NOS: 1 to 90 under stringent conditions and acting as an environmental stress-responsive promoter is also included in the promoter of the present invention.

Once the nucleotide sequence of a promoter according to the present invention is determined, the promoter can be obtained by chemical synthesis, PCR using a cloned probe as a template, or hybridization using a DNA fragment having the nucleotide sequence as a probe. Furthermore, a mutant of the promoter of the present invention, which has the same functions as those of a non-mutated promoter, can be also synthesized by a site-specific mutagenesis or the like.

To introduce a mutation into a promoter sequence, a known method such as the Kunkel method, Gapped duplex method or an equivalent method may be employed. A mutation may be introduced by using a mutation-introducing kit (for example, Mutant-K manufactured by Takara or Mutant-G manufactured by Takara) which uses a site-specific mutagenesis or by using the LA PCR in vitro mutagenesis series kit (manufactured by Takara).

The term “functioning as an environmental stress-responsive promoter” used herein refers to a function of activating transcription caused by binding RNA polymerase to the promoter when the promoter is exposed to a predetermined environmental stress condition.

The term “environmental stress” used herein generally refers to an abiotic stress such as drought stress, cold stress, high salt stress, or intensive light stress. The term “drought” used herein refers to a state of water deficiency, and the term “cold” used herein refers to a state where an object is exposed to a lower temperature than the optimum living temperature for each organism (e.g., in the case of Arabidopsis thaliana, it is exposed to a temperature of −20 to +21° C. continuously for one hour to several weeks). The term “high salt” used herein refers to a state where a plant is treated with NaCl of 50 mM to 600 mM in concentration continuously for 0.5 hours to several weeks. The term “intensive light stress” used herein refers to a state where too intensive light to use for photosynthesis is applied to a plant, and corresponds to a case where, for example, light of 5,000 to 10,000 Lx or more is applied. These environmental stresses may be applied singly or in combination.

The plant promoter of the present invention includes a promoter having a nucleotide sequence represented by SEQ ID NOS: 1 to 90 wherein a nucleotide sequence may be added to the 3′ end in order to increase transcriptional efficiency or a nucleotide sequence may be deleted from the 5′ end to the extent not to lose the activity of a promoter.

Furthermore, the promoter of the present invention includes DNA which hybridizes with DNA consisting of any nucleotide sequence selected from SEQ ID NOS: 1 to 90 under stringent conditions and acts as an environmental stress-responsive promoter. The term “stringent conditions” used herein refers to the conditions of sodium concentration of 25 to 500 mM, preferably 25 to 300 mM, and a temperature of 42 to 68° C., preferably 42 to 65° C.; more preferably, conditions of 5×SSC (83 mM NaCl, 83 mM sodium citrate) and a temperature of 42° C.

2. Construction of Expression Vector

An expression vector of the present invention can be obtained by ligating (inserting) a promoter according to the present invention to an appropriate vector. The vector into which a promoter of the present invention is to be inserted is not particularly limited as long as it can be replicated in a host. Examples of such a vector include a plasmid, shuttle vector and helper plasmid.

Examples of such a plasmid DNA include plasmids derived from Escherichia coli (e.g., pBR322, pBR325, pUC118, pUC119, pUC18, pUC19, and pBluescript); plasmids derived from Bacillus subtilis (e.g., pUB110 and pTP5); and plasmids derived from yeasts (e.g., YEp13 and YCp50). Examples of a phage DNA include λ phages (Charon4A, Charon21A EMBL3, EMBL4, λgt10, λgt11, and λZAP). Further animal virus vectors such as retrovirus and a vaccinia virus and insect virus vectors such as a baculovirus can be also used.

To insert a promoter according to the present invention into a vector, use may be made of a method of digesting a purified DNA with appropriate restriction enzymes, inserting the obtained DNA fragment into the restriction site of a suitable vector DNA or a multi-cloning site, and ligating it to the vector.

In the present invention, to express an arbitrary gene, the arbitrary gene can be further inserted into the aforementioned expression vector. The technique inserting an arbitrary gene is the same as the method inserting a promoter into a vector. An arbitrary gene is not particularly limited. Examples of the gene include genes shown in Table 2 and known genes other than those.

In a case where a reporter gene, for example, a GUS gene, widely used in plants is linked to the 3′ end of a promoter of the present invention, the strength of the promoter can be easily evaluated by checking GUS activity. As such a reporter gene other than the GUS gene, luciferase and a green fluorescent protein can be used.

As described above, various types of vectors can be used in the present invention. Further, a desired gene is ligated to the promoter of the present invention in a sense or antisense direction and then, the construction can be inserted into a vector such as pBI101 (Clonetech) called a binary vector.

3. Isolation of Transcriptional Factor

A transcriptional factor binds to a cis element which is present upstream of a gene and activates the transcription of the gene present downstream thereof. The transcriptional factors isolated in the present invention are induced by environmental stresses such as a low temperature, dehydration, and high salt concentration.

Environmental stress-responsive transcriptional factors are roughly divided into those belonging to a DREB family, ERF family, zinc finger family, WRKY family, MYB family, bHLH family, NAC family, homeo domain family and bZIP family.

In isolating a transcriptional factor, first, stress responsive genes are isolated by using a microarray. As a microarray, use may be made of about 7,000 cDNA molecules in total including genes isolated from Arabidopsis full-length cDNA libraries, responsive to dehydration (RD) genes, early responsive to dehydration (ERD) genes; PCR amplification fragments obtained from a λ control template DNA fragment (TX803, manufactured by Takara Shuzo), as an internal standard; and mouse nicotinic acetylcholine receptor epsilon subunit (nAChRE) genes and mouse glucocorticoid receptor homologous genes, as negative controls.

A plasmid DNA extracted by a plasmid preparation device (manufactured by Kurabo) is sequenced by sequence analysis using a DNA sequencer (ABI PRISM 3700, PE Applied Biosystems, CA, USA). Based on the GenBank/EMBL database, the obtained sequence is screened for homology by using the BLAST program.

After poly A selection is performed, reverse transcription is carried out to synthesize a double-stranded DNA molecule and a cDNA molecule is inserted into a vector.

The cDNA molecule inserted into a vector for constructing cDNA libraries is amplified by PCR using complementary primers to the sequences of vectors on both sides of the cDNA molecule. Examples of such vectors include λZAPII and λPS.

A microarray can be prepared according to a conventional method, which is not particularly limited. For example, using a gene tip microarray stamp machine GTMASS SYSTEM (manufactured by Nippon Laser & Electronics Lab.), the above obtained PCR product is loaded from the microtiter plate and spotted on a microslide glass at predetermined intervals. Then, to prevent a non-specific signal from being expressed, the slide is immersed into a blocking solution.

Examples of plant materials include a plant strain obtained by destroying a specific gene as well as wild type plants. A transgenic plant having a cDNA of DREB1A introduced therein may be used. Examples of plant species include Arabidopsis thaliana, tobacco and rice. Of them, Arabidopsis thaliana is preferable.

Dehydration- and cold-stress treatments can be carried out according to a known method (Yamaguchi-Shinozaki, K., and Shinozaki, K. (1994), Plant Cell 6, 251-264).

After plant bodies (wild type plants and DREB1A overexpression transformants) are exposed to stress, they are sampled and stored in cryogenic conditions with liquid nitrogen. The wild type and DREB1A overexpression transformants are used in an experiment to identify a DREB1A target gene. From plant bodies, mRNA is isolated and purified by a known method or a kit.

In the presence of Cy3 dUTP or Cy5 dUTP for labeling (Amersham Pharmacia), each of mRNA samples is subjected to reverse transcription and then used in hybridization.

After hybridization, the microarray is scanned with a scanning laser microscope or the like. As a program for analyzing data of a microarray, Imagene Ver 2.0 (BioDiscovery) and QuantArray (GSI Lumonics) etc., may be used.

After the scanning, a plasmid having a target gene is prepared. In this way, the target genes are isolated.

A transcriptional factor is determined by analyzing the nucleotide sequence of the gene isolated above and using a gene analysis program based on the genomic information of database (GenBank/EMBL, ABRC). The isolated genes can be classified into ones inducible by both drought and cold stress, ones inducible specifically by drought stress, and one inducible specifically by cold stress. According to the gene analysis program, from the genes mentioned above, genes encoding 36 types of transcriptional factors can be identified. The nucleotide sequences of the genes encoding 36 types of transcriptional factors are represented by SEQ ID NOS: 2n−1 (n is an integer of 47 to 82) and amino acid sequences of the transcriptional factors are represented by SEQ ID NOS: 2n (n is an integer of 47 to 82). Sequence ID numbers and the names of genes encoding transcriptional factors are shown in Table 1.

TABLE 1

| Name of gene | SEQ ID NO: |
|---|---|
| RAFL05-11-M11 | SEQ ID NO: 93 |
| RAFL06-11-K21 | SEQ ID NO: 95 |
| RAFL05-16-H23 | SEQ ID NO: 97 |
| RAFL08-16-D06 | SEQ ID NO: 99 |
| RAFL08-16-G17 | SEQ ID NO: 101 |
| RAFL06-08-H20 | SEQ ID NO: 103 |
| RAFL07-10-G04 | SEQ ID NO: 105 |
| RAFL04-17-D16 | SEQ ID NO: 107 |
| RAFL05-19-M20 | SEQ ID NO: 109 |
| RAFL08-11-M13 | SEQ ID NO: 111 |
| RAFL04-15-K19 | SEQ ID NO: 113 |
| RAFL05-11-L01 | SEQ ID NO: 115 |
| RAFL05-14-C11 | SEQ ID NO: 117 |
| RAFL05-19-G24 | SEQ ID NO: 119 |
| RAFL05-20-N02 | SEQ ID NO: 121 |
| RAFL05-18-H12 | SEQ ID NO: 123 |
| RAFL06-10-D22 | SEQ ID NO: 127 |
| RAFL06-12-M01 | SEQ ID NO: 129 |
| RAFL05-14-D24 | SEQ ID NO: 131 |
| RAFL05-20-N17 | SEQ ID NO: 133 |
| RAFL04-17-F21 | SEQ ID NO: 135 |
| RAFL09-12-N16 | SEQ ID NO: 137 |
| RAFL05-19-I05 | SEQ ID NO: 139 |
| RAFL05-21-I22 | SEQ ID NO: 141 |
| RAFL08-11-H20 | SEQ ID NO: 143 |
| RAFL05-21-C17 | SEQ ID NO: 145 |
| RAFL05-08-D06 | SEQ ID NO: 147 |
| RAFL05-20-M16 | SEQ ID NO: 149 |
| RAFL11-01-J18 | SEQ ID NO: 151 |
| RAFL11-09-C20 | SEQ ID NO: 153 |
| RAFL05-18-N16 | SEQ ID NO: 155 |
| RAFL11-10-D10 | SEQ ID NO: 157 |
| RAFL04-17-N22 | SEQ ID NO: 159 |
| RAFL05-09-G15 | SEQ ID NO: 161 |
| RAFL05-21-L12 | SEQ ID NO: 163 |

Note that as long as a transcriptional factor of the present invention functions as an environmental stress-responsive transcriptional factor, use may be made of any transcriptional factor having a nucleotide sequence selected from SEQ ID NOS: 2n−1 (n is an integer of 47 to 82) wherein one or more nucleotides, preferably one or several nucleotides (for example 1 to 10, preferably 1 to 5) have been deleted, substituted or added. Furthermore, DNA hybridizing with the DNA comprising any nucleotide sequence selected from SEQ. ID NOS. 2n−1 (n is an integer of 47 to 82) under stringent conditions and encoding an environmental stress-responsive transcriptional factor is also included in the transcriptional factor of the present invention. The term “stringent conditions” used herein refers to the conditions of sodium concentration of 25 to 500 mM, preferably 25 to 300 mM, and a temperature of 42 to 68° C., preferably 42 to 65° C.; more preferably, conditions of 5×SSC (83 mM NaCl, 83 mM sodium citrate) and a temperature of 42° C.

36 types of transcriptional factors isolated in the present invention may be classified as follows.
(1) DREB family: RAFL05-11-M11, RAFL06-11-K21, RAFL05-16-H23, RAFL08-16-D16;
(2) ERF family: RAFL08-16-G17, RAFL06-08-H20;
(3) Zinc finger family: RAFL07-10-G04, RAFL04-17-D16, RAFL05-19-M20, RAFL08-11-M13, RAFL04-15-K19, RAFL05-11-L01, RAFL05-14-C11, RAFL05-19-G24, RAFL05-20-N02;
(4) WRKY family: RAFL05-18-H12, RAFL05-19-E19, RAFL06-10-D22, RAFL06-12-M01;
(5) MYB family: RAFL05-14-D24, RAFL05-20-N17, RAFL04-17-F21;
(6) bHLH family: RAFL09-12-N16;
(7) NAC family: RAFL05-19-I05, RAFL05-21-I22, RAFL08-11-H20, RAFL05-21-C17, RAFL05-08-D06;
(8) Homeo domain family: RAFL05-20-M16, RAFL11-01-J18, RAFL11-09-C20; and
(9) bZIP family: RAFL05-18-N16, RAFL11-10-D10, RAFL04-17-N22, RAFL05-09-G15.

Note that RAFL05-21-L12 cannot be classified into (1) to (9).

Once the nucleotide sequence of a gene encoding a transcriptional factor according to the present invention is determined, the gene encoding a transcriptional factor according to the present invention can be obtained by chemical synthesis, PCR using a cloned probe as a template, or hybridizing a DNA fragment having the nucleotide sequence as a probe. Furthermore, a mutant of the gene encoding a transcriptional factor according to the present invention, and having the same functions as those of a non-mutated transcriptional factor, can be also synthesized by a site-specific mutagenesis or the like.

To introduce a mutation into a nucleotide sequence of a gene encoding a transcriptional factor, a known method such as the Kunkel method, Gapped duplex method, or an equivalent method may be employed. A mutation may be introduced by using a mutation-introducing kit (for example, Mutant-K manufactured by Takara and Mutant-G manufactured by Takara) which uses a site-specific mutagenesis or by using the LA PCR in vitro mutagenesis series kit (manufactured by Takara).

The term “environmental stress” used herein generally refers to an abiotic stress such as drought stress, cold stress, high salt stress, or intensive light stress. The term “drought” used herein refers to a state of water deficiency, the term “cold” used herein refers to a state where an object is exposed to a lower temperature than the optimum living temperature of each organism (e.g., in the case of Arabidopsis thaliana, e.g., in the case of Arabidopsis thaliana, it is exposed to a temperature of −20 to +21° C. continuously for one hour to several weeks). The term “high salt” used herein refers to a state where a plant is treated with NaCl of 50 mM to 600 mM in concentration continuously for 0.5 hours to several weeks. The term “intensive light stress” used herein refers to a state where too intensive light to use for photosynthesis is applied to a plant, and corresponds to a case where, for example, light of 5,000 to 10,000 Lx or more is applied. These environmental stresses may be applied singly or in combination.

4. Construction of Expression Vector

The expression vector of the present invention can be obtained by ligating (inserting) a gene encoding a transcriptional factor according to the present invention to an appropriate vector. The vector into which a gene encoding a transcriptional factor of the present invention is inserted is not particularly limited as long as it can be replicated in a host. Examples of such a vector include a plasmid, shuttle vector and helper plasmid.

Examples of such a plasmid DNA include plasmids derived from Escherichia coli (e.g., pBR322, pBR325, pUC118, pUC119, pUC18, pUC19, and pBluescript), plasmids derived from Bacillus subtilis (e.g., pUB110 and pTP5); and plasmids derived from yeasts (e.g., YEp13 and YCp50). Examples of a phage DNA include λ phages (Charon4A, Charon21A EMBL3, EMBL4, λgt10, λgt11, and λZAP). Further animal virus vectors such as retrovirus and a vaccinia virus and insect virus vectors such as a baculovirus can be also used.

To insert a transcriptional factor of the present invention into a vector, use may be made of a method of digesting a purified DNA with appropriate restriction enzymes, inserting the obtained DNA fragment into the restriction site of a suitable vector DNA or a multi-cloning site, and ligating it to the vector.

In a case where a reporter gene, for example, a GUS gene, widely used in plants is linked to the 3′ end of the gene encoding a transcriptional factor of the present invention, the strength of the gene expression can be easily evaluated by checking GUS activity. As such a reporter gene other than the GUS gene, luciferase and a green fluorescent protein can be used.

5. Preparation of Transformant

A transformant of the present invention can be obtained by introducing an expression vector of the present invention into a host. The host used herein is not particularly limited as long as it can express a promoter, a gene of interest, or an environmental stress-responsive transcriptional factor. Of them, a plant is preferable. In a case of a plant host, a transformant plant (transgenic plant) can be obtained as follows.

A plant to be transformed in the present invention refers to an entire plant, a plant organ (such as leaf, petal, stem, root, or seed), a plant tissue (such as the epidermis, phloem, parenchyma, xylem, or vascular bundle), or a plant culture cell. Examples of plants used for transformation include plants belonging to the Brassicaceae, Graniineae, Solanaceae and Leguminosae (see below); however they are not limited to these plants.

Brassicaceae: Arabidopsis thaliana

Gramineae: Nicotiana tabacum

Solanaceae: Zea mays, Oryza sativa

Leguminosae: Glycine max

The aforementioned recombinant vector can be introduced into a plant by a conventional transformation method such as electroporation, Agrobacterium method, particle gun method, or PEG method.

For example, where electroporation is used, a gene is introduced into a host by treating a vector by an electroporation device equipped with a pulse controller under conditions: a voltage of 500 to 1,600 V, 25 to 1,000 μF, and 20 to 30 msec.

When a particle gun method is used, a plant body, organ and tissue may be directly used. Alternatively, they may be used after they are sectioned to pieces or after protoplasts of them are prepared. The samples thus prepared may be processed by a gene-introduction device (for example, PDS-1000/He manufactured by Bio-Rad). Processing conditions vary depending upon a plant or sample. Generally, processing is performed at a pressure of about 1,000 to 1800 psi and a distance of about 5 to 6 cm.

Furthermore, a gene of interest can be introduced into a plant by using a plant virus as a vector. Examples of available plant viruses include a cauliflower mosaic virus. More specifically, a virus genome is inserted into a vector derived from Escherichia coli to prepare a recombinant and then such a gene of interest is inserted into the virus genome. The virus genome thus modified is excised out from the recombinant with restriction enzymes and inoculated into a plant host. In this manner the gene of interest can be introduced into the plant host.

In the method using a Ti plasmid of the Agrobacterium, when bacteria belonging to the Agrobacterium are transfected to a plant, a portion of plasmid DNA of the bacteria is transferred into a plant genome. Using such a characteristic, a gene of interest is introduced into a plant host. Of bacteria belonging to the Agrobacterium, Agrobacterium tumefaciens, when it is introduced into a plant by transfection, produces a tumor called a crown gall. Also, a plant when it is transfected with Agrobacterium rhizogenes, it produces hairy roots. These phenomena are caused by transferring a region called a T-DNA region (transferred DNA region) present in a plasmid such as a Ti plasmid or Ri plasmid present in each bacterium into a plant and incorporating the region into a plant genome at a time of transfection.

By inserting desired DNA, which is to be incorporated into a plant genome, into the T-DNA region on a Ti or Ri plasmid, the desired DNA can be incorporated into a plant genome, when the host is transfected with Agrobacterium bacteria.

Tumoral tissues, shoots and hairy roots obtained as a result of transformation can be directly used in cell culture, tissue culture, or organ culture. Also, when a plant hormone such as auxin, cytokinin, gibberellin, abscisic acid, ethylene, or brassinoride, is administered to them in an appropriate concentration by using a conventional plant tissue culture method, a plant body can be regenerated from them.

A vector according to the present invention can be not only incorporated into the plant hosts mentioned above but also introduced into bacteria belonging to the Escherichia such as Escherichia coli, the Bacillus such as Bacillus subtilis and the Pseudomonas such as Pseudomonas putida; yeasts such as Saccharomyces cerevisiae and Schizosaccharomyces pombe; animal cells such as COS cells and CHO cells; and insect cells such as Sf9 cells, to obtain a transformant. Where a bacterium such as Escherichia coli or yeast is used as a host, it is preferable that a recombinant vector according to the present invention can be self-replicated in the bacterium and, at the same time, is comprised of a promoter of the present invention, a ribosome binding sequence, a gene of interest and a transcription termination sequence. Furthermore, a gene regulating the promoter may be included in the bacterium.

A method for introducing a recombinant vector into bacteria is not particularly limited as long as it is a method which can introduce DNA into bacteria. Examples of such a method include a method of using calcium ions and an electroporation method.

When a yeast is used as a host, Saccharomyces cerevisiae and Schizosaccharomyces pombe may be used. A method for introducing a recombinant vector is not particularly limited as long as it is a method for introducing DNA into a yeast. Examples of such a method include electroporation, spheroplast method, and lithium acetate method.

Where an animal cell is used as a host, a monkey COS-7 cell, Vero, Chinese hamster ovary cell (CHO cell), and mouse L cell etc. are used. Examples of methods for introducing a recombinant vector into an animal cell include electroporation, calcium phosphate method, and lipofection method.

When an insect cell is used as a host, a Sf9 cell and the like may be used. Examples of method for introducing a recombinant vector into an insect cell include a calcium phosphate method, lipofection method, and electroporation method.

Whether a gene is incorporated into a host or not is confirmed by a PCR method, Southern hybridization, Northern hybridization method or the like. For example, PCR is performed by preparing DNA from a transformant, and designing DNA specific primers. PCR is carried out under the same conditions as used for preparing the plasmid mentioned above. Thereafter, the obtained amplified product is subjected to agarose gel electrophoresis, polyacrylamide gel electrophoresis or capillary electrophoresis, and stained with ethidium bromide, or SYBR Green solution, etc. If the amplified product is found as a single band, it is confined that a transformant is obtained. Alternatively, the amplified product can be also detected by PCR using primers previously stained with a fluorescent dye or the like. Furthermore, there may be employed a method in which the amplified product is bound to a solid phase such as a microplate and confirmed by fluorescent or an enzymatic reaction.

4. Production of Plant

In the present invention, a transformed plant body can be regenerated from the above transformed plant cell or the like. As a regeneration method, use is made of one in which callus-form transformed cells are transferred to a medium having a different hormone in a different concentration and cultured to form an adventitious embryo, from which an entire plant body is obtained. Examples of the medium to be used herein include an LS medium and an MS medium.

The “method for producing a plant body” of the present invention comprises steps of: introducing a plant expression vector, into which the above plant promoter or a gene encoding an environmental stress-responsive transcriptional factor is inserted, into a host cell to obtain a transformed plant cell; regenerating a transformed plant body from the transformed plant cell; obtaining a plant seed from the transformed plant body; and producing a plant body from the plant seed.

To obtain plant seeds from a transformed plant body, for example, the transformed plant body is collected from a rooting medium and transferred to a pot having soil containing water placed therein. Then, the transformed plant body is grown at constant temperature to form flowers. Finally seeds are obtained. To produce a plant body from a seed, for example, when a seed formed on a transformed plant body has matured, the seed is isolated and seeded in soil containing water, followed by growing at constant temperature under illumination. The plant thus bred becomes an environmental stress-resistant plant exhibiting the stress resistance corresponding to the responsivity of a promoter introduced therein or a gene encoding the environmental stress-responsive transcriptional factor introduced therein.

### BRIEF DESCRIPTION OF THE DRAWINGS

FIG. 1 is a characteristic graph showing the relationship between dehydration treatment time and expression ratio regarding FL03-07-F12;

FIG. 2 is a characteristic graph showing the relationship between cold treatment time and expression ratio regarding FL04-12-F24;

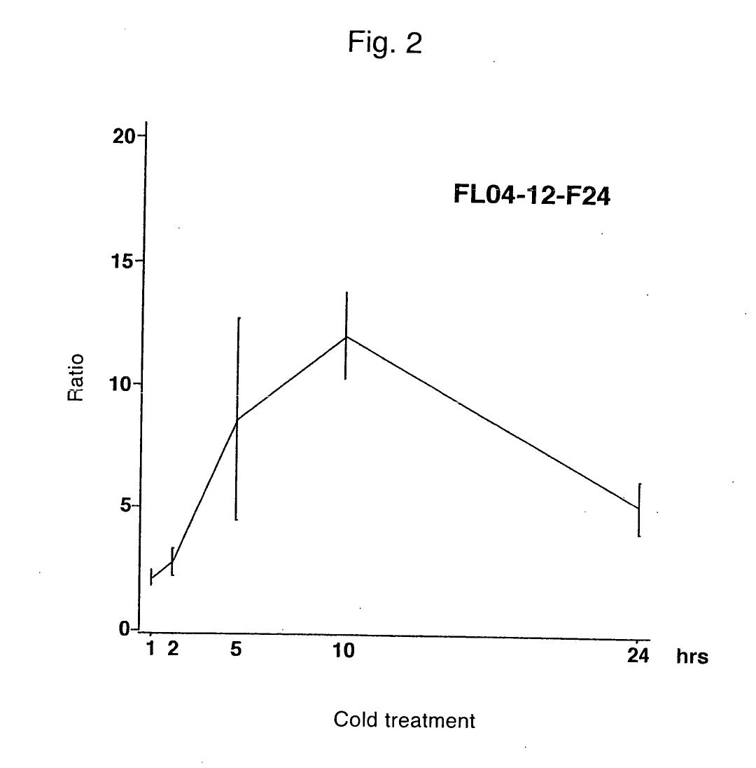

FIG. 3 is a characteristic graph showing the relationship between dehydration treatment time and expression ratio regarding FL04-14-N10;

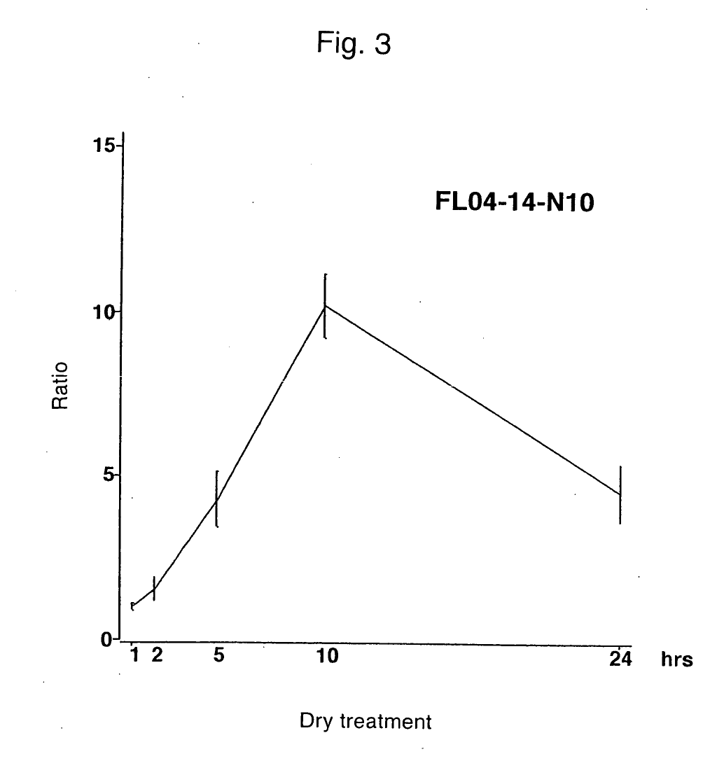

FIG. 4 is a characteristic graph showing the relationship between dehydration treatment time and expression ratio regarding FL04-14-P24;

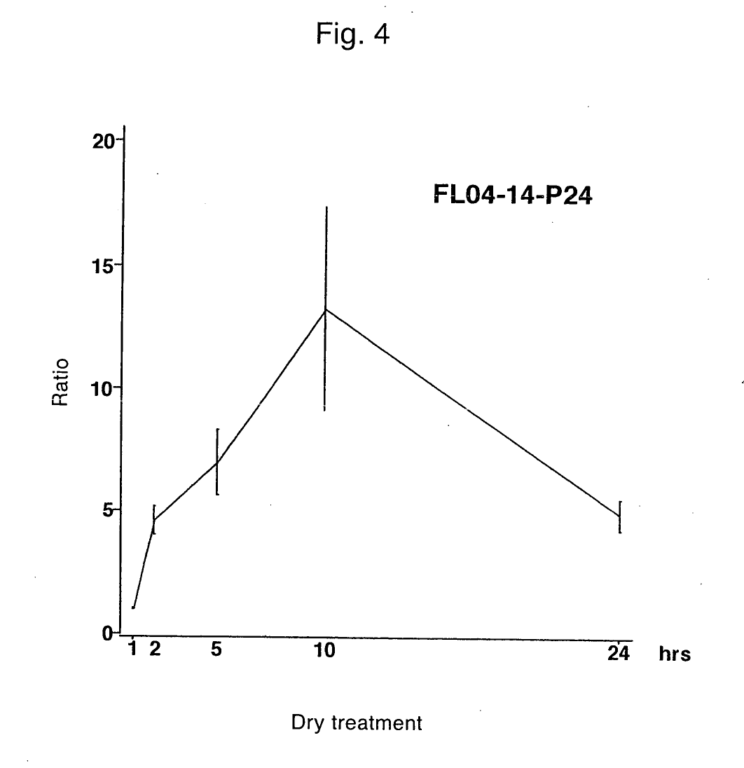

FIG. 5 is a characteristic graph showing the relationship between dehydration treatment time and expression ratio regarding FL04-17-I03;

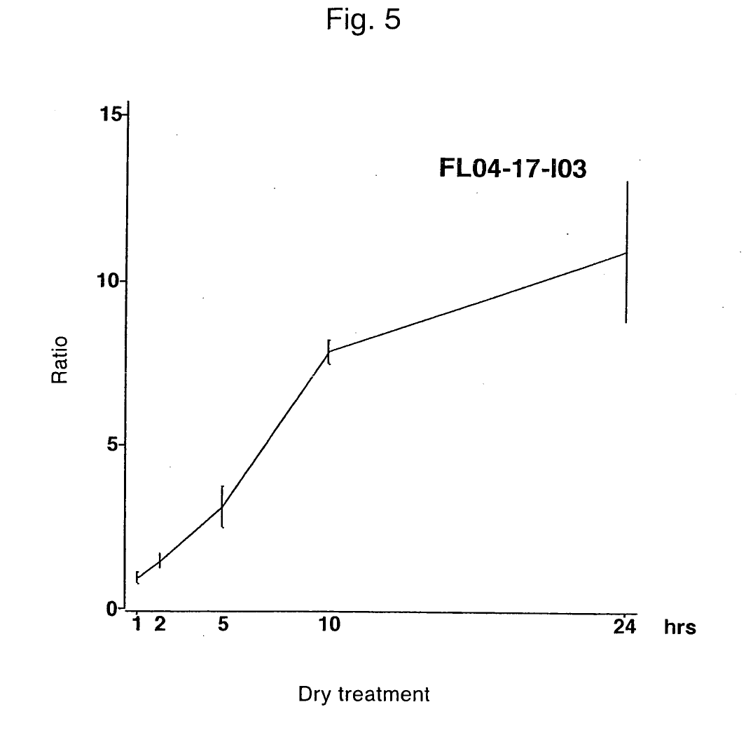

FIG. 6 is a characteristic graph showing the relationship between high salt treatment time and expression ratio regarding FL04-17-I03;

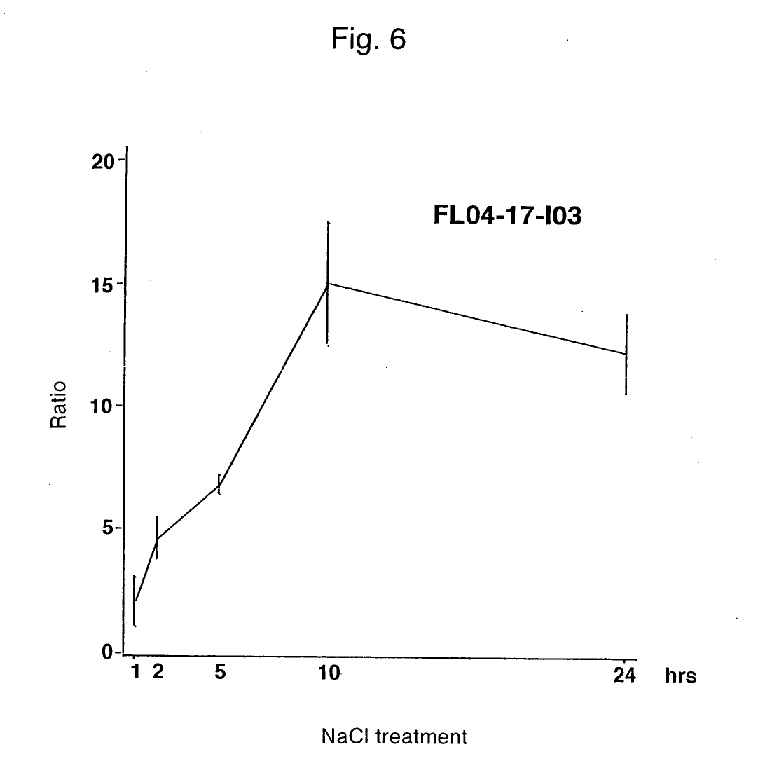

FIG. 7 is a characteristic graph showing the relationship between high salt treatment time and expression ratio regarding FL04-17-M08;

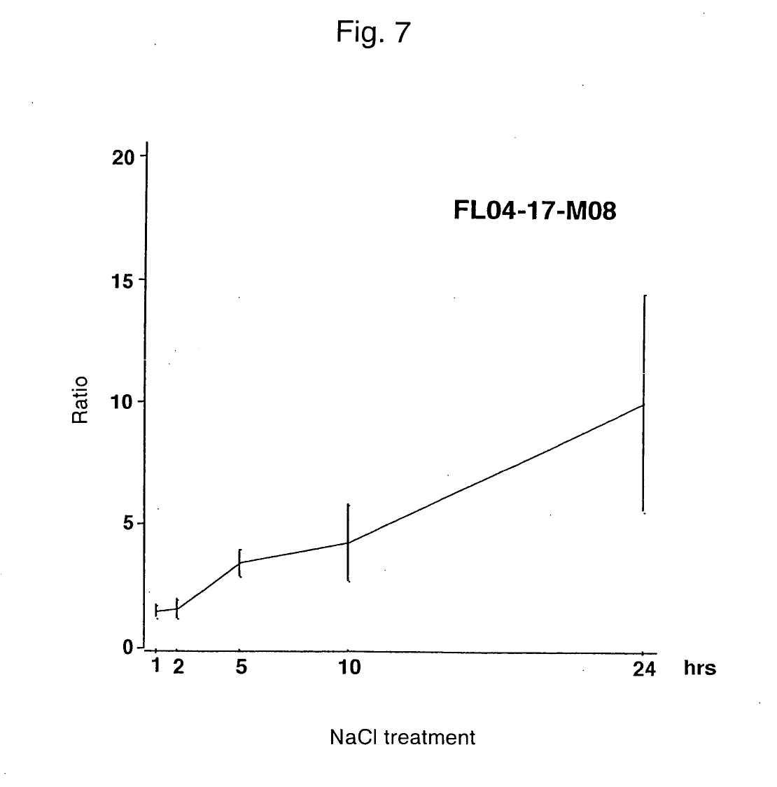

FIG. 8 is a characteristic graph showing the relationship between dehydration treatment time and expression ratio regarding FL04-17-M22;

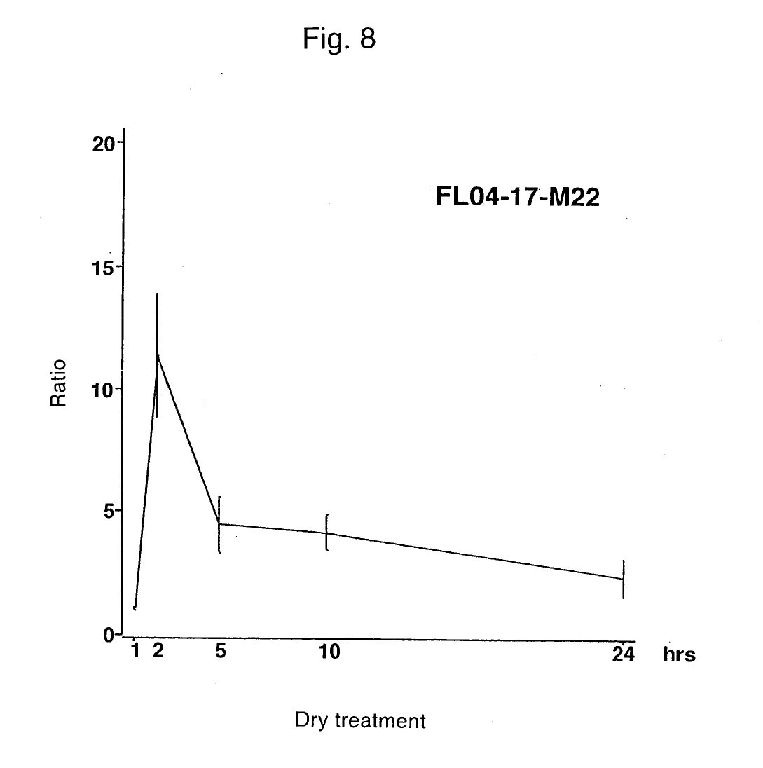

FIG. 9 is a characteristic graph showing the relationship between dehydration treatment time and expression ratio regarding FL05-05-A17;

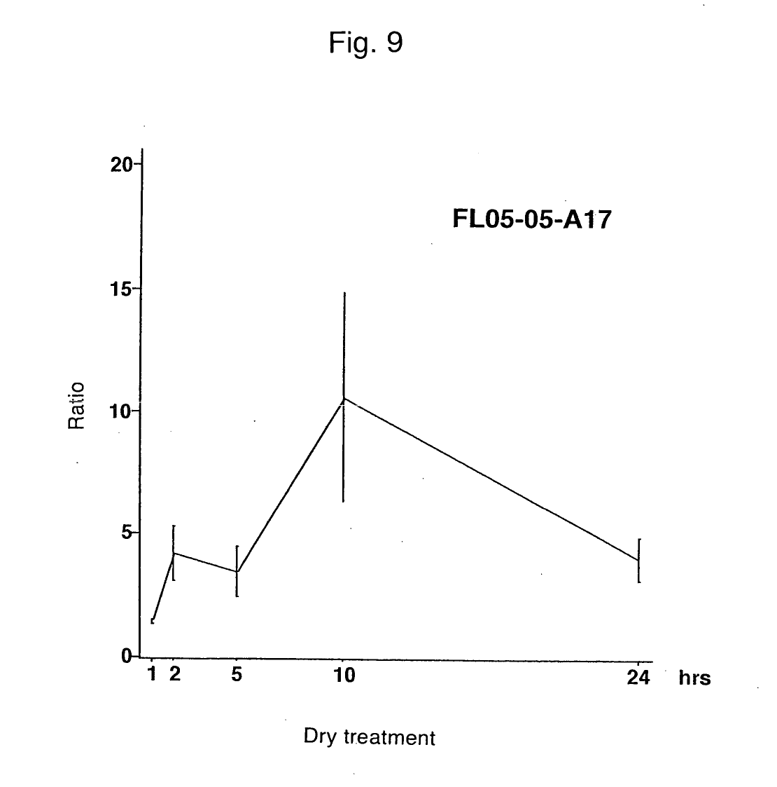

FIG. 10 is a characteristic graph showing the relationship between dehydration treatment time and expression ratio regarding FL05-05-F20;

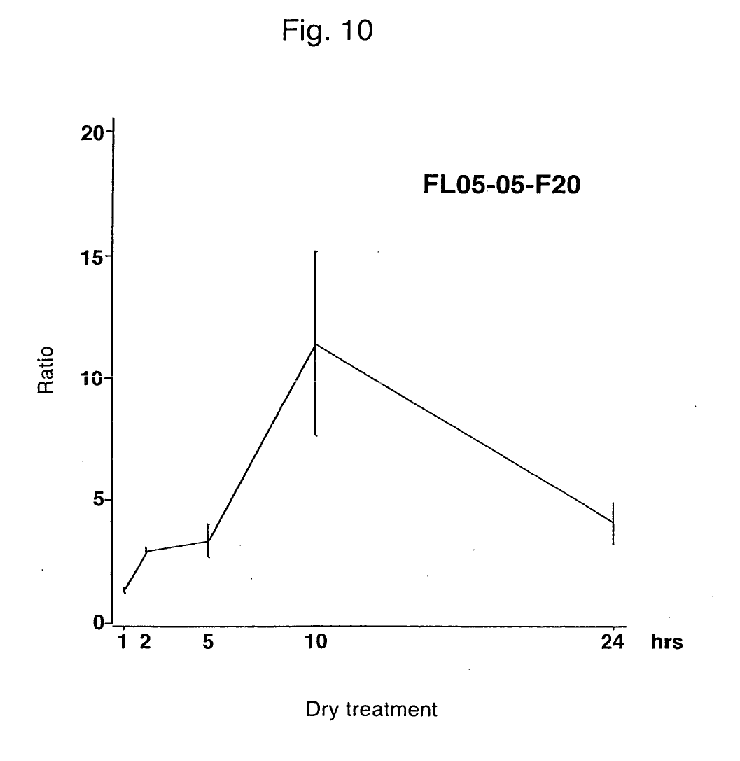

FIG. 11 is a characteristic graph showing the relationship between dehydration treatment time and expression ratio regarding FL05-05-G20;

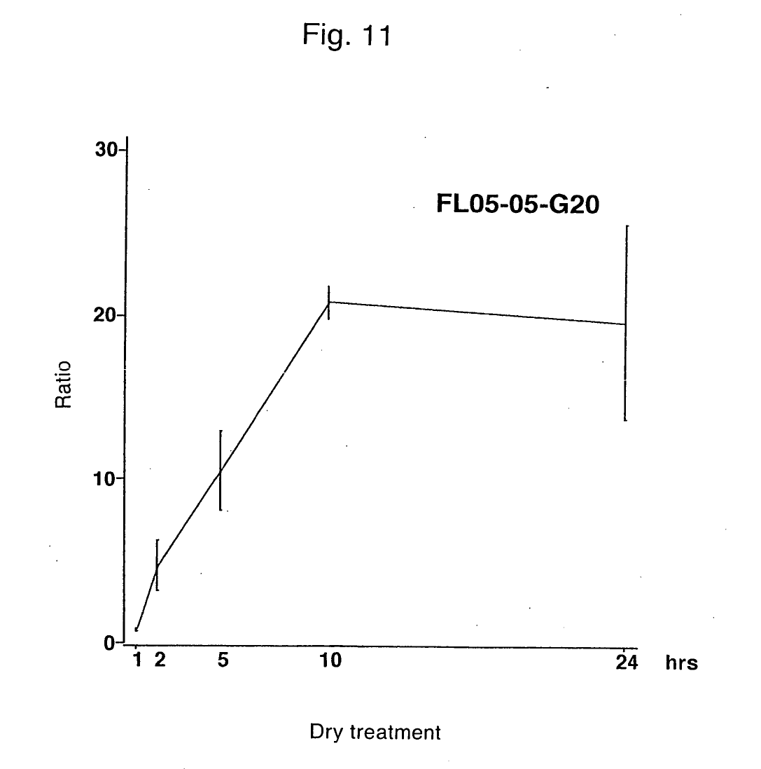

FIG. 12 is a characteristic graph showing the relationship between dehydration treatment time and expression ratio regarding FL05-09-N09;

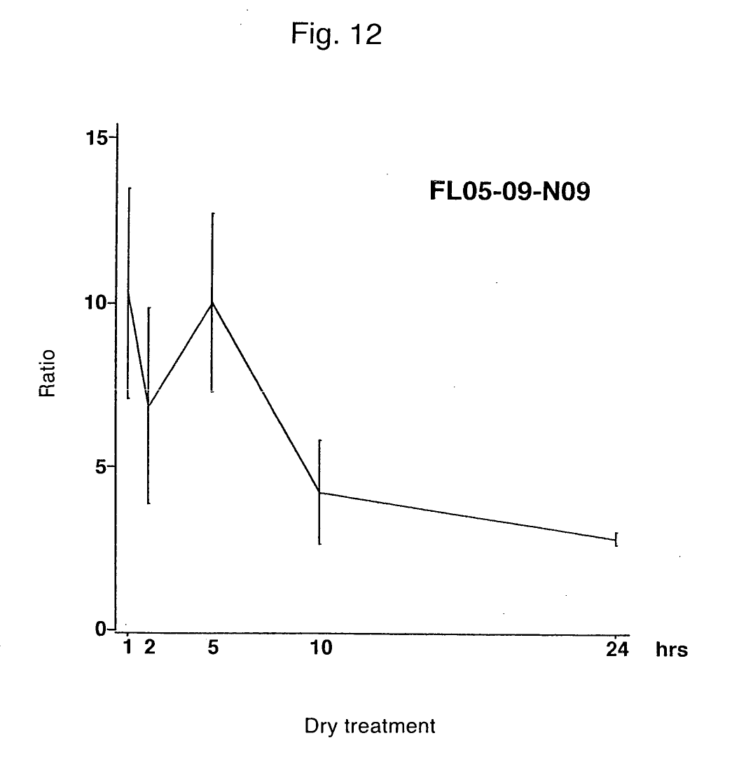

FIG. 13 is a characteristic graph showing the relationship between dehydration treatment time and expression ratio regarding FL05-10-J09;

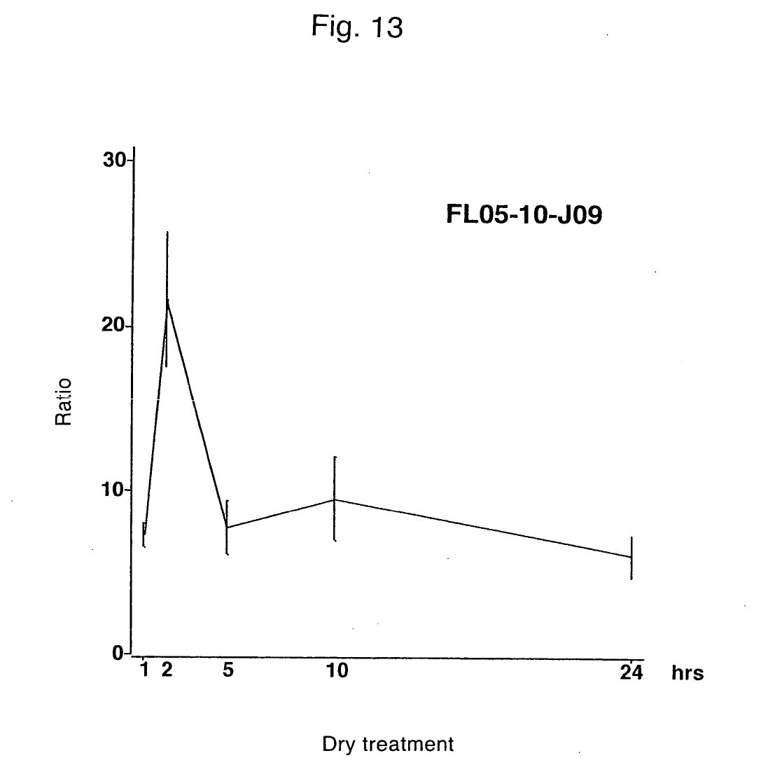

FIG. 14 is a characteristic graph showing the relationship between high salt treatment time and expression ratio regarding FL05-10-J09;

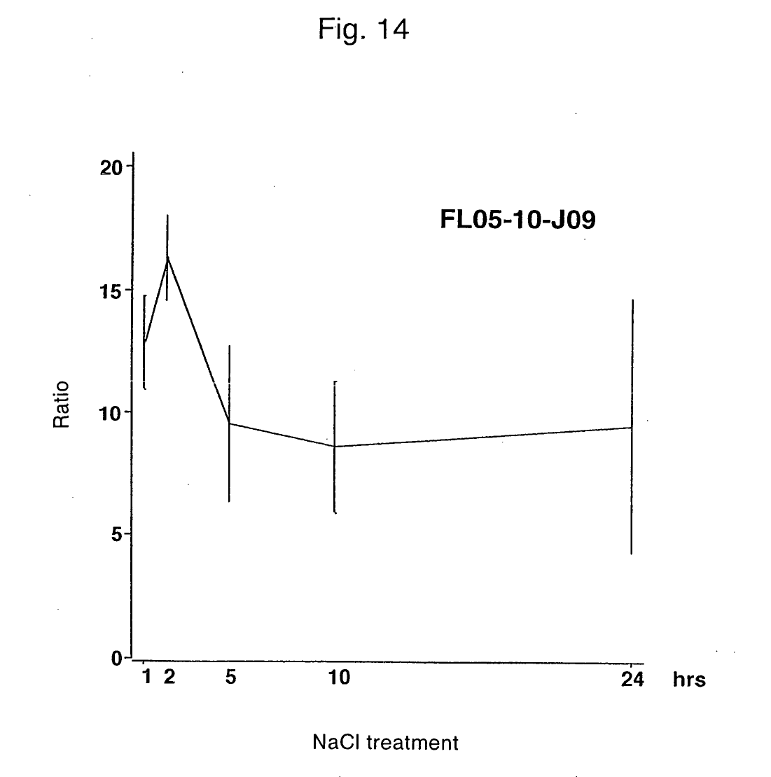

FIG. 15 is a characteristic graph showing the relationship between high salt treatment time and expression ratio regarding FL05-10-M08;

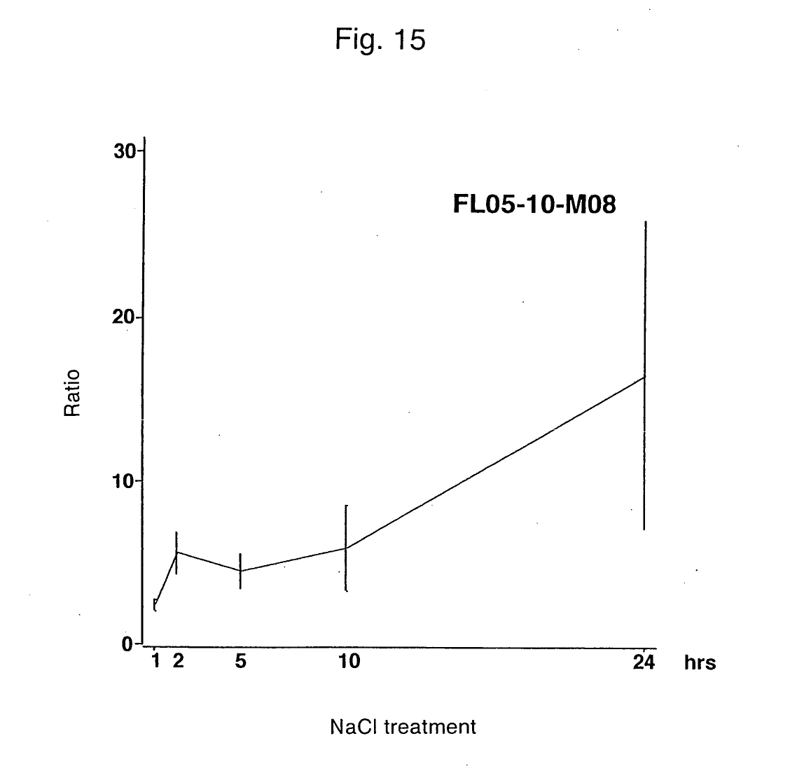

FIG. 16 is a characteristic graph showing the relationship between high salt treatment time and expression ratio regarding FL05-11-H09;

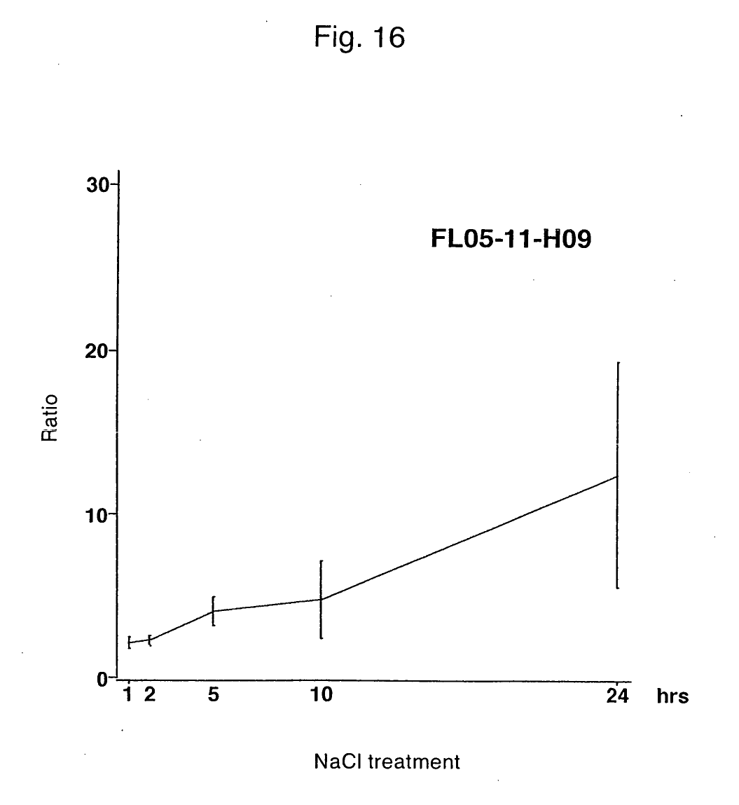

FIG. 17 is a characteristic graph showing the relationship between dehydration treatment time and expression ratio regarding FL05-12-H13;

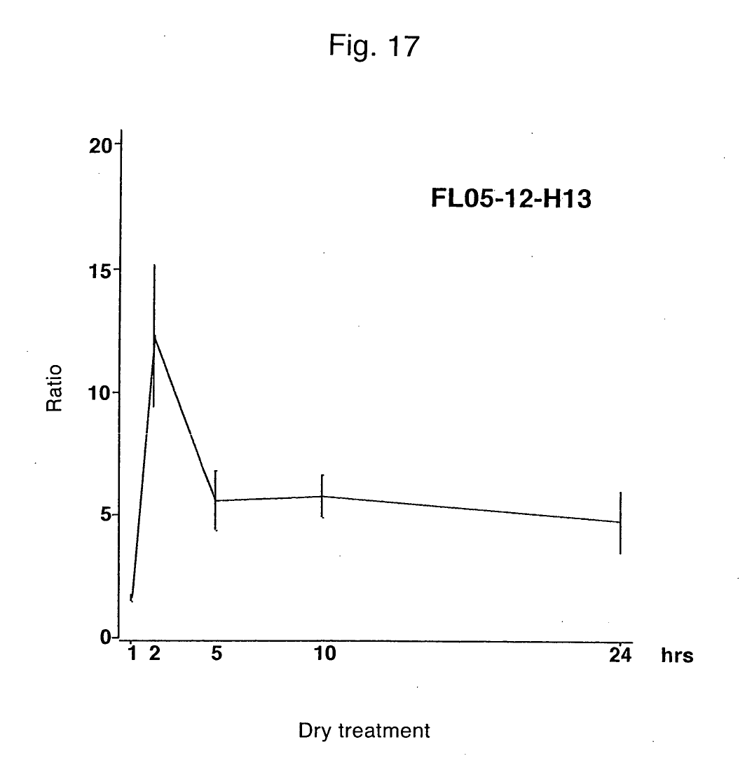

FIG. 18 is a characteristic graph showing the relationship between high salt treatment time and expression ratio regarding FL05-12-H13;

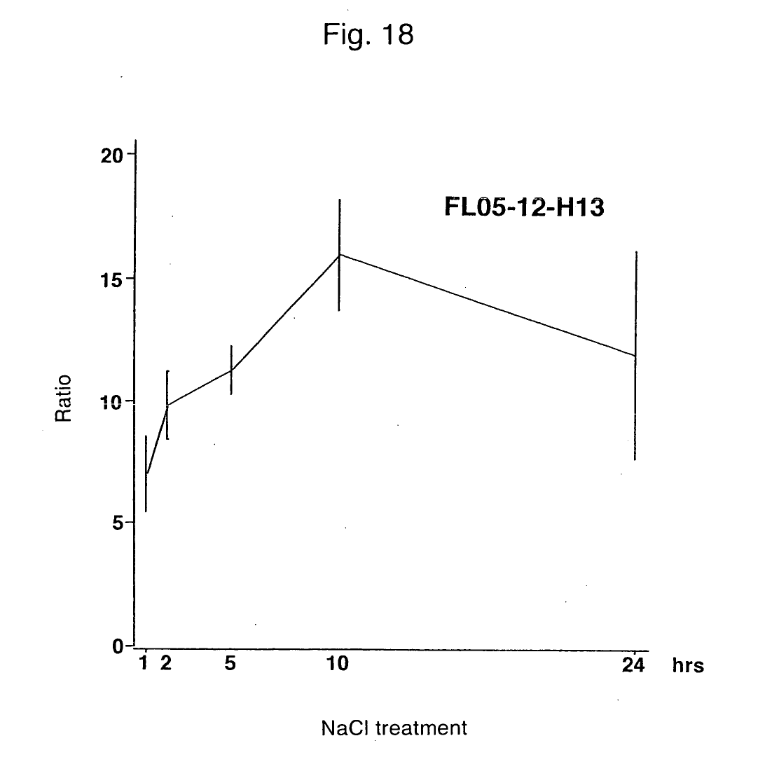

FIG. 19 is a characteristic graph showing the relationship between ABA treatment time and expression ratio regarding FL05-13-I20;

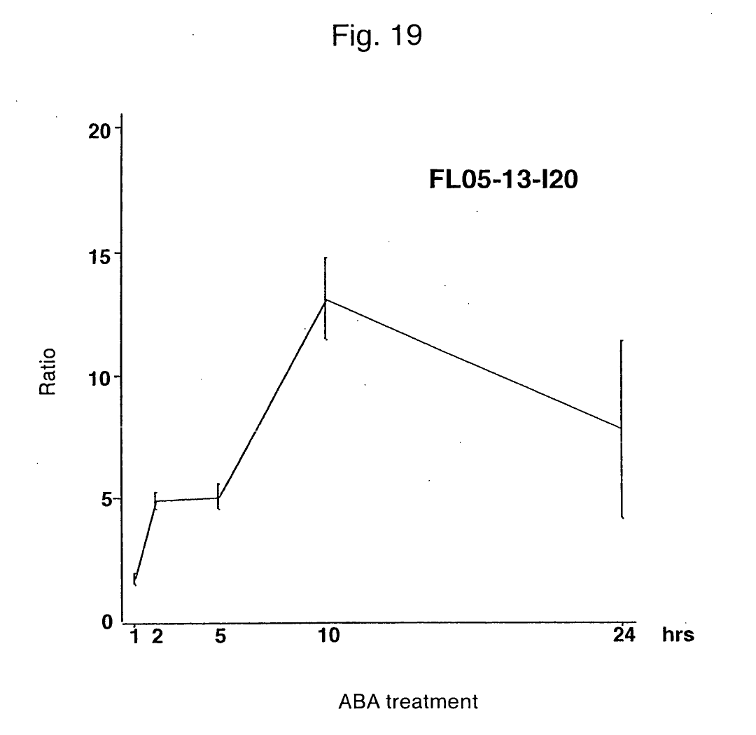

FIG. 20 is a characteristic graph showing the relationship between dehydration treatment time and expression ratio regarding FL05-14-E15;

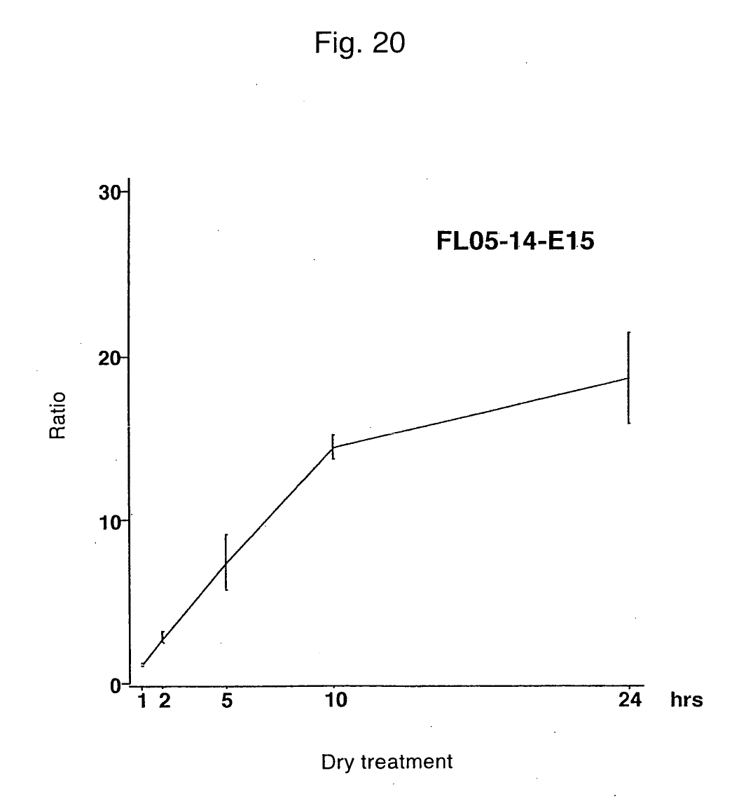

FIG. 21 is a characteristic graph showing the relationship between dehydration treatment time and expression ratio regarding FL05-14-E16;

FIG. 22 is a characteristic graph showing the relationship between cold treatment time and expression ratio regarding FL05-14-E16;

FIG. 23 is a characteristic graph showing the relationship between ABA treatment time and expression ratio regarding FL05-14-E16;

FIG. 24 is a characteristic graph showing the relationship between dehydration treatment time and expression ratio regarding FL05-16-F03;

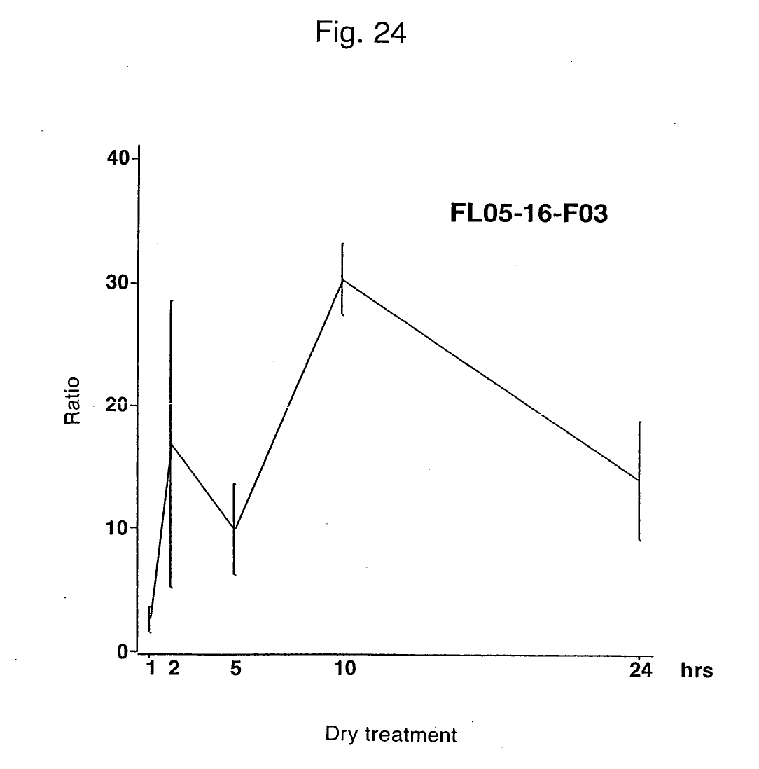

FIG. 25 is a characteristic graph showing the relationship between ABA treatment time and expression ratio regarding FL05-16-F03;

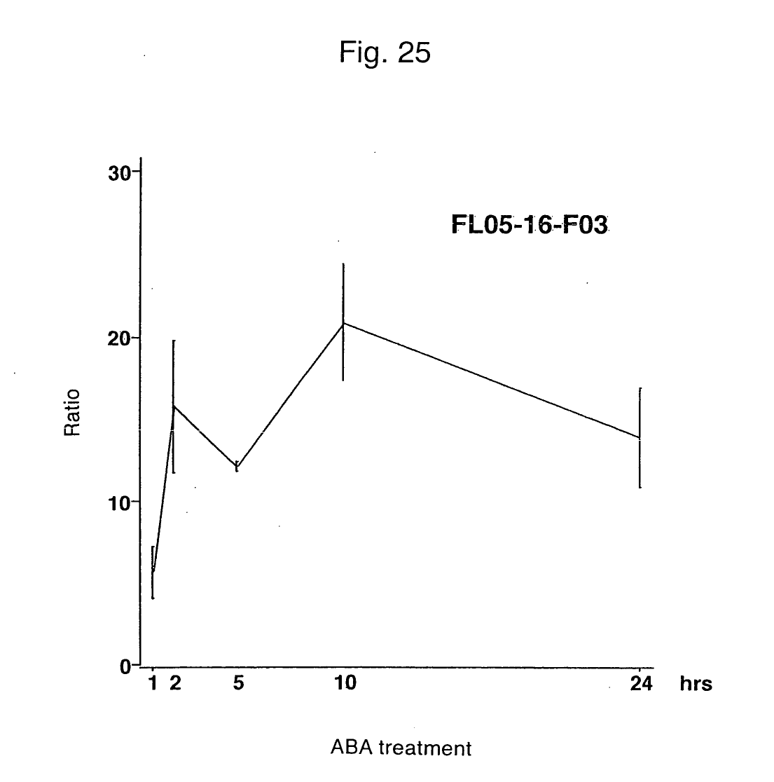

FIG. 26 is a characteristic graph showing the relationship between dehydration treatment time and expression ratio regarding FL05-16-H23;

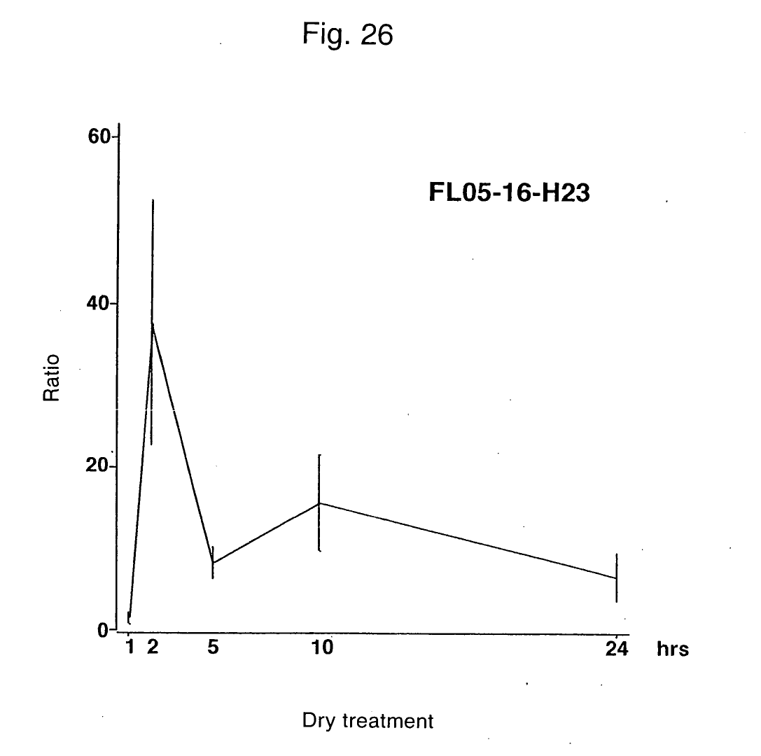

FIG. 27 is a characteristic graph showing the relationship between high salt treatment time and expression ratio regarding FL05-16-H23;

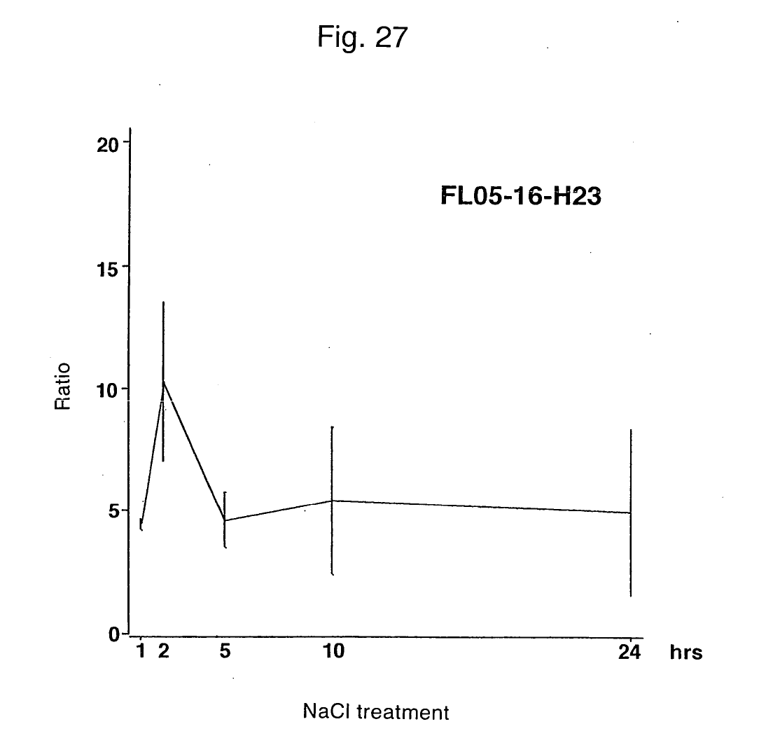

FIG. 28 is a characteristic graph showing the relationship between dehydration treatment time and expression ratio regarding FL05-18-M07;

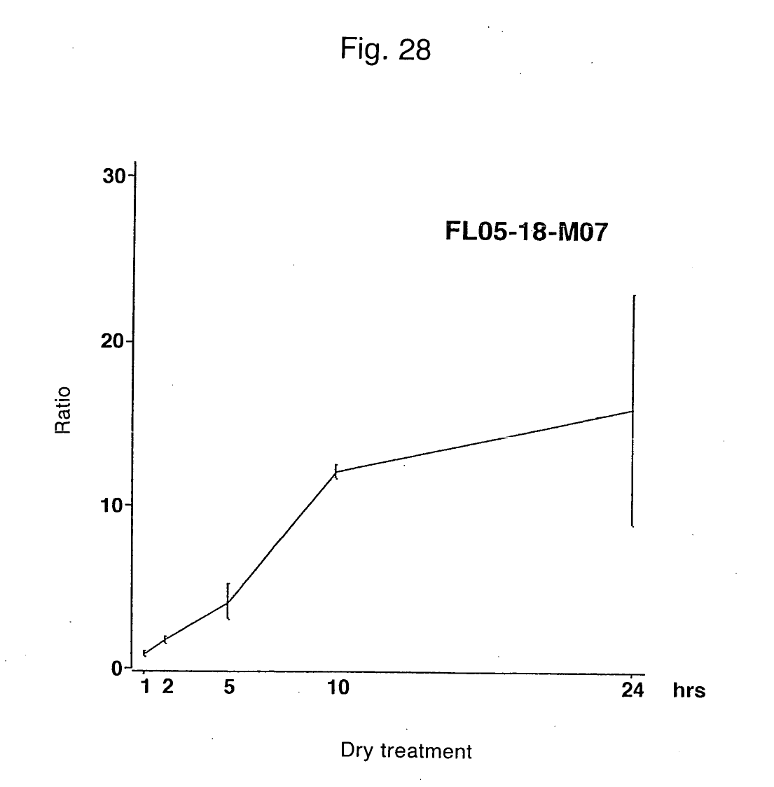

FIG. 29 is a characteristic graph showing the relationship between ABA treatment time and expression ratio regarding FL05-18-M07;

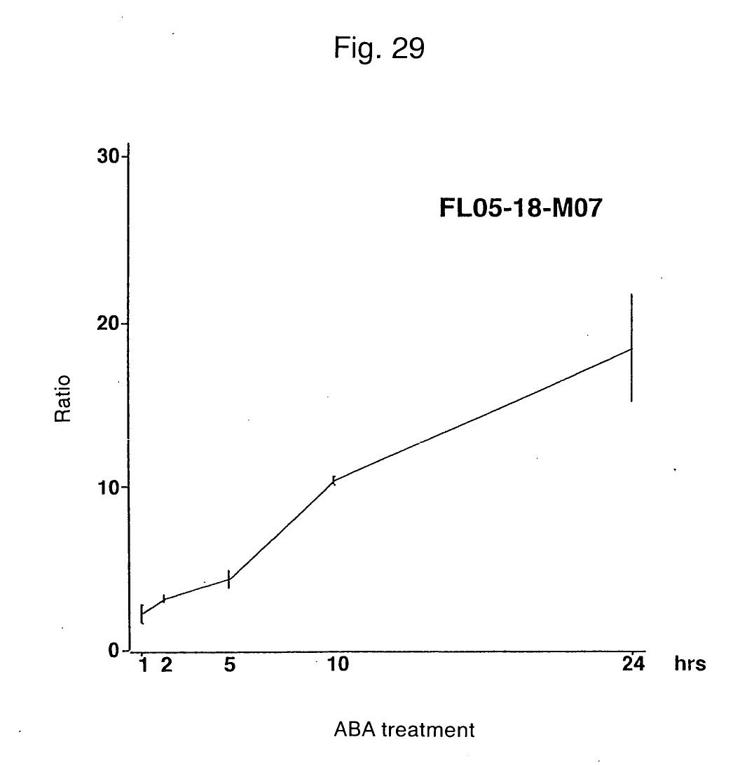

FIG. 30 is a characteristic graph showing the relationship between ABA treatment time and expression ratio regarding FL05-18-021;

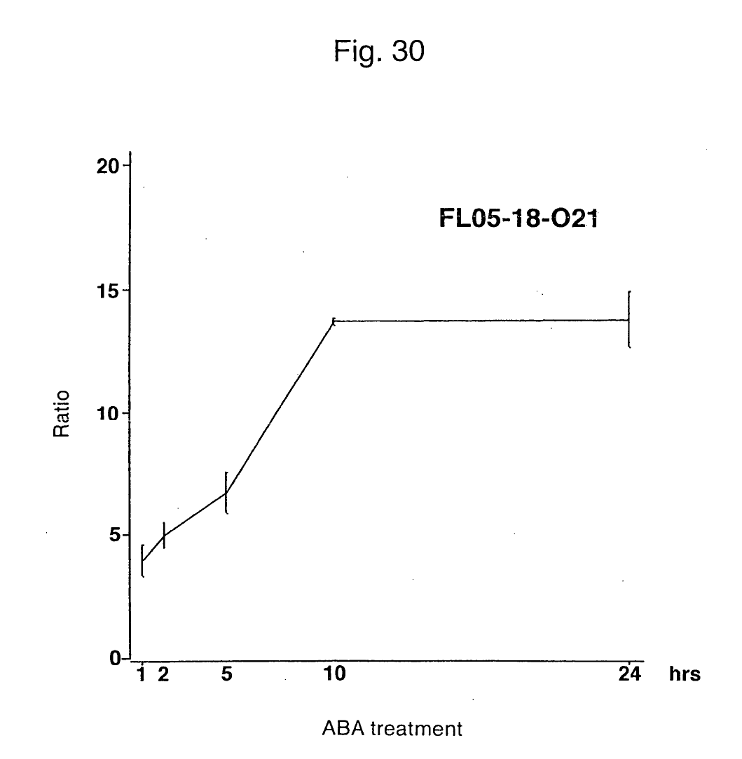

FIG. 31 is a characteristic graph showing the relationship between dehydration treatment time and expression ratio regarding FL05-19-F21;

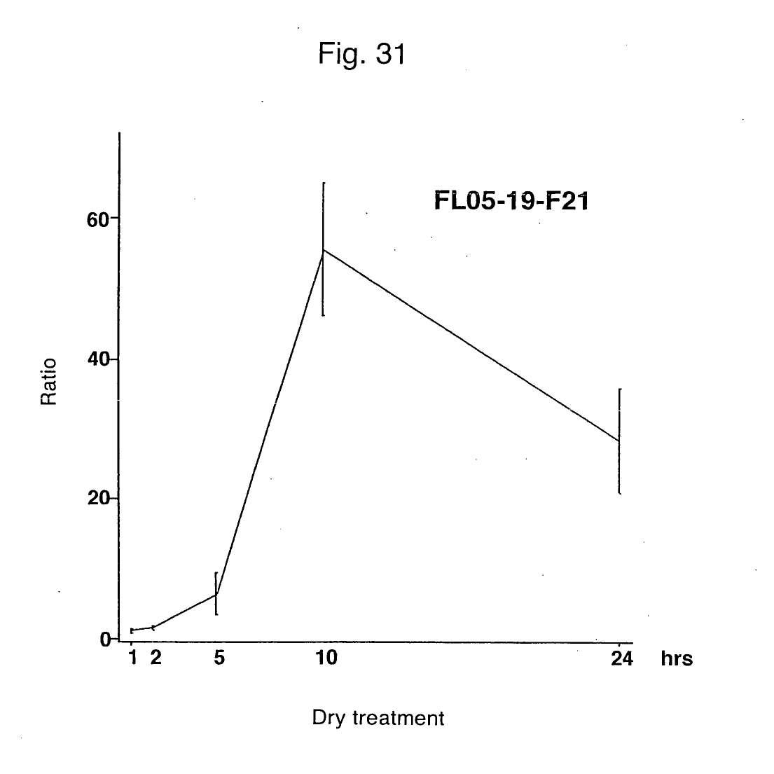

FIG. 32 is a characteristic graph showing the relationship between ABA treatment time and expression ratio regarding FL05-19-F21;

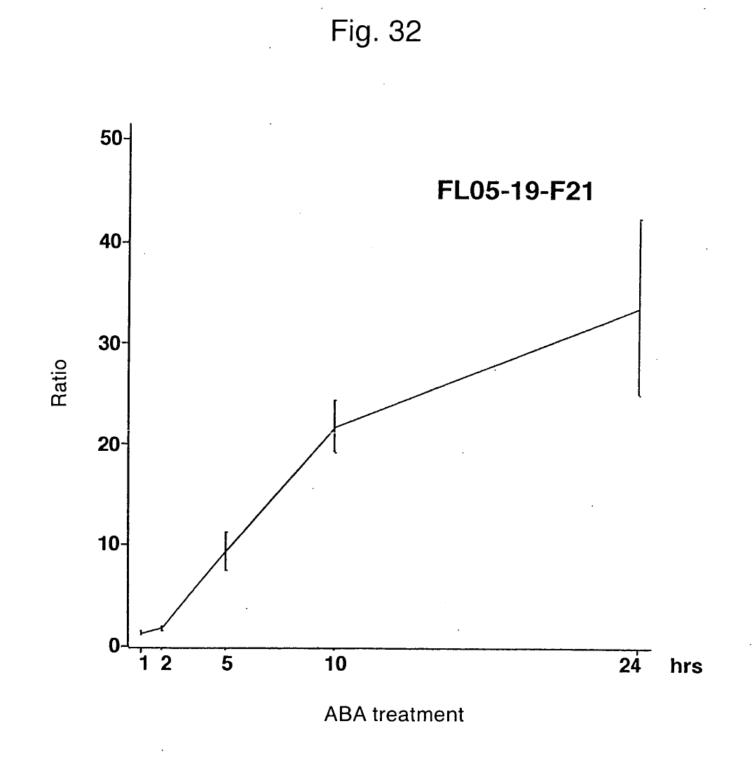

FIG. 33 is a characteristic graph showing the relationship between dehydration treatment time and expression ratio regarding FL05-19-O22;

FIG. 34 is a characteristic graph showing the relationship between high salt treatment time and expression ratio regarding FL05-19-O22;

FIG. 35 is a characteristic graph showing the relationship between ABA treatment time and expression ratio regarding FL05-19-O22;

FIG. 36 is a characteristic graph showing the relationship between high salt treatment time and expression ratio regarding FL05-21-K17;

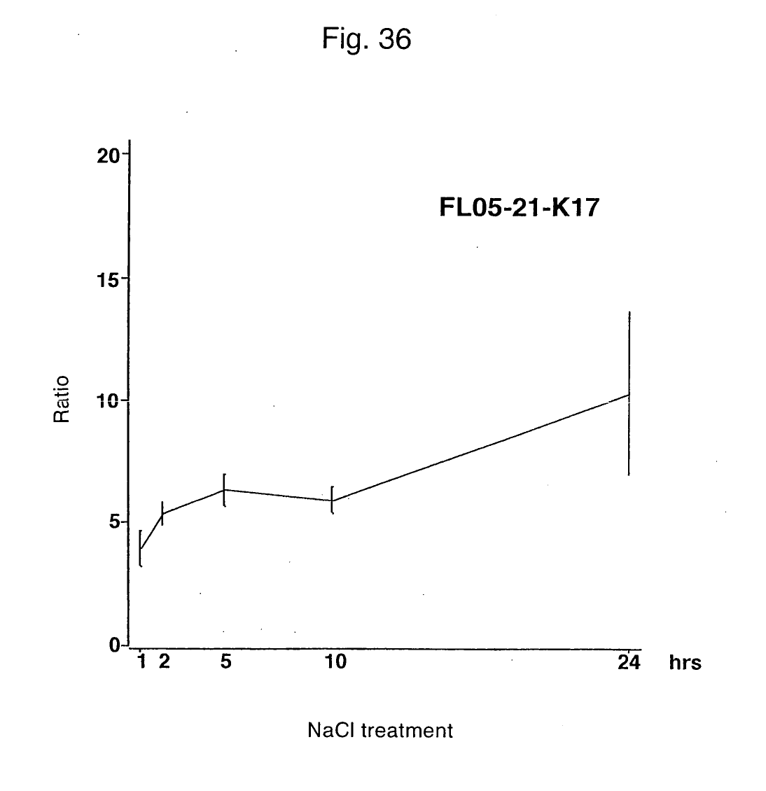

FIG. 37 is a characteristic graph showing the relationship between ABA treatment time and expression ratio regarding FL06-10-F03;

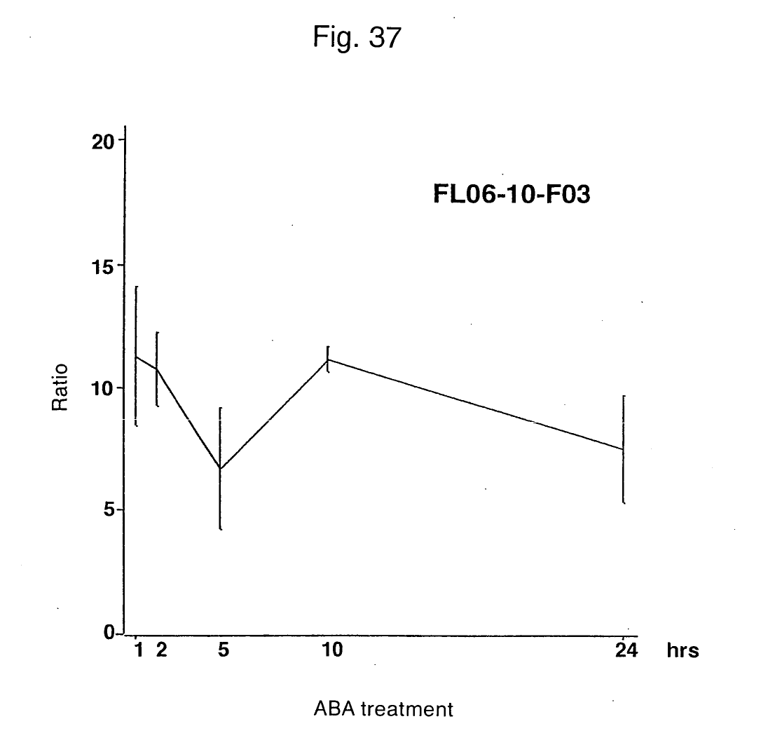

FIG. 38 is a characteristic graph showing the relationship between dehydration treatment time and expression ratio regarding FL06-12-H12;

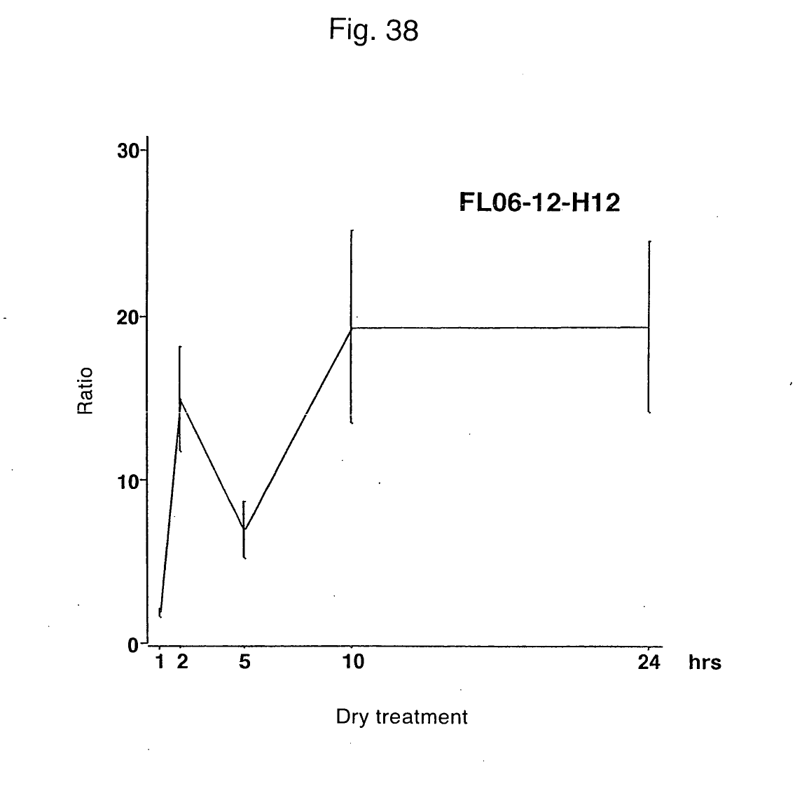

FIG. 39 is a characteristic graph showing the relationship between high salt treatment time and expression ratio regarding FL06-12-H12;

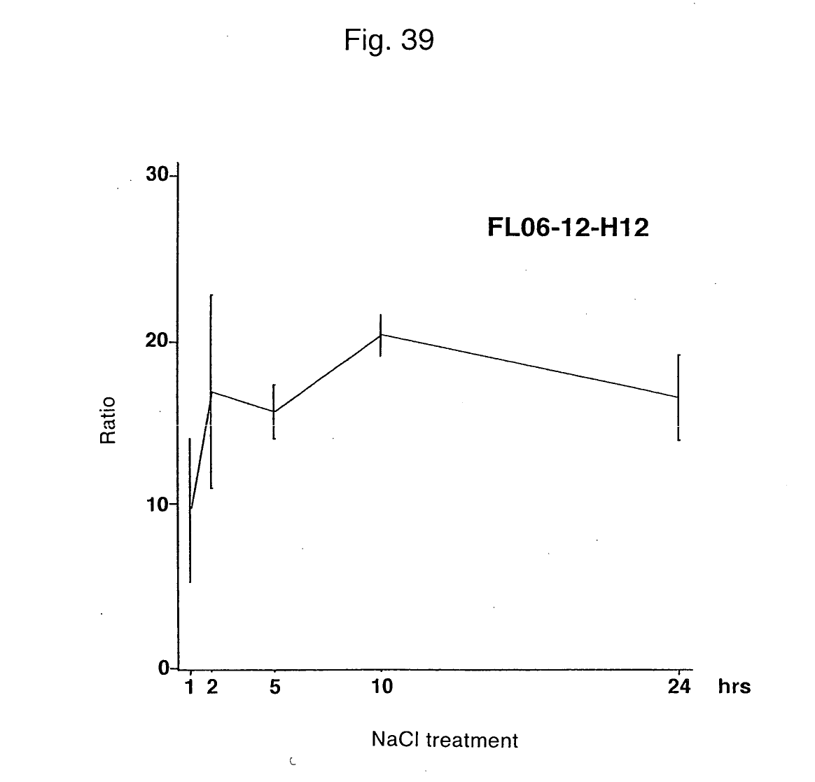

FIG. 40 is a characteristic graph showing the relationship between high salt treatment time and expression ratio regarding FL07-12-I23;

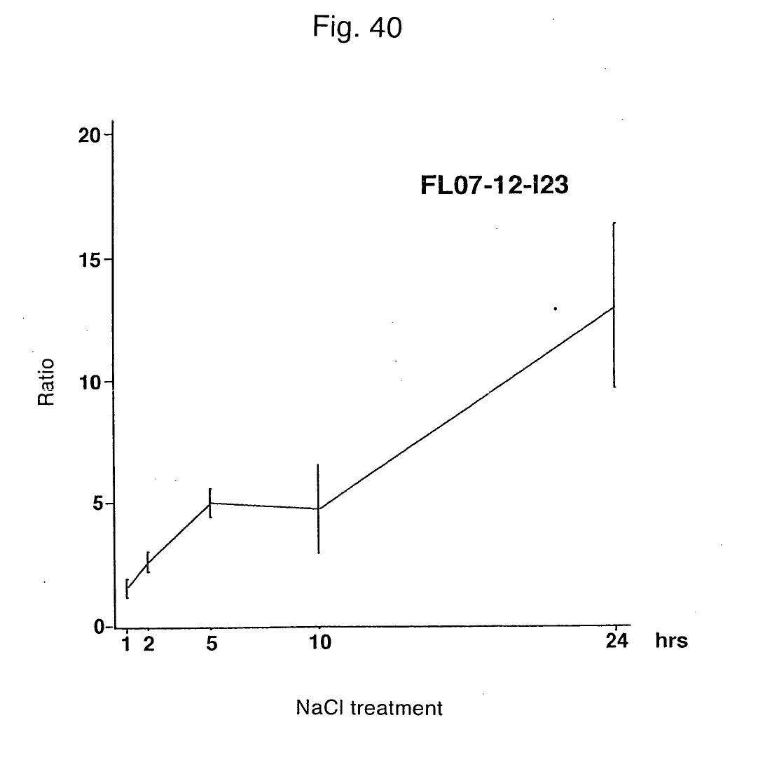

FIG. 41 is a characteristic graph showing the relationship between high salt treatment time and expression ratio regarding FL08-08-H23;

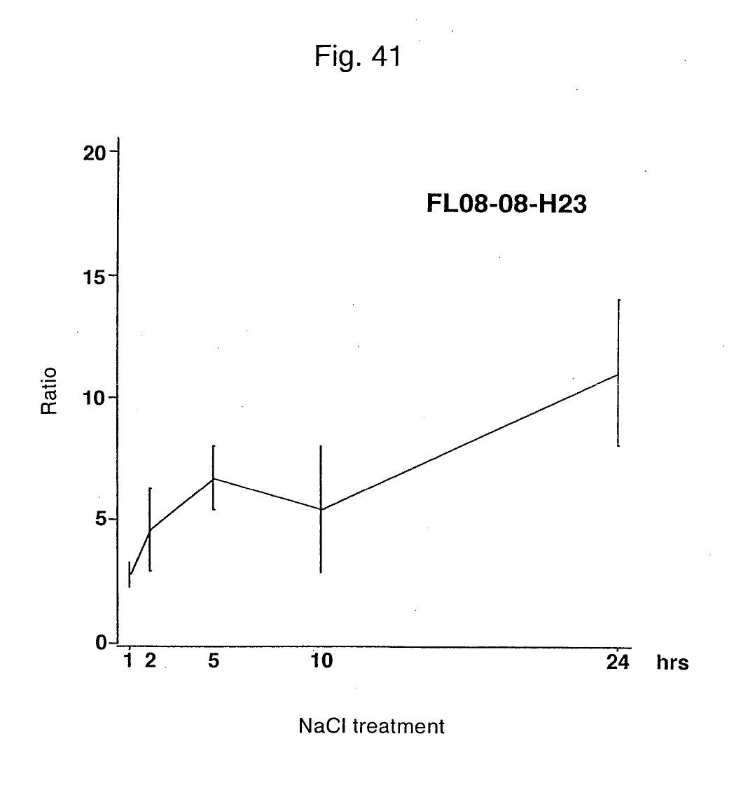

FIG. 42 is a characteristic graph showing the relationship between dehydration treatment time and expression ratio regarding FL08-08-O14;

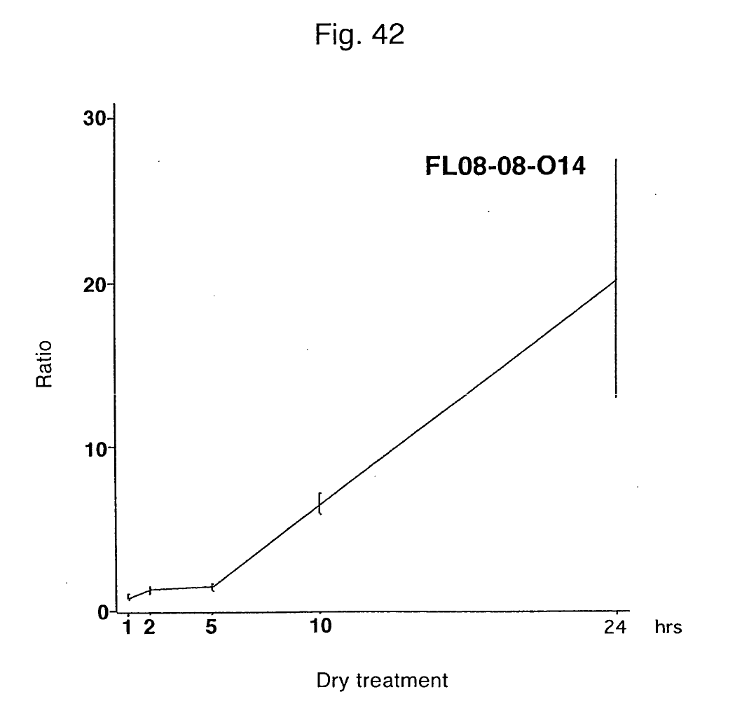

FIG. 43 is a characteristic graph showing the relationship between dehydration treatment time and expression ratio regarding FL08-09-M05;

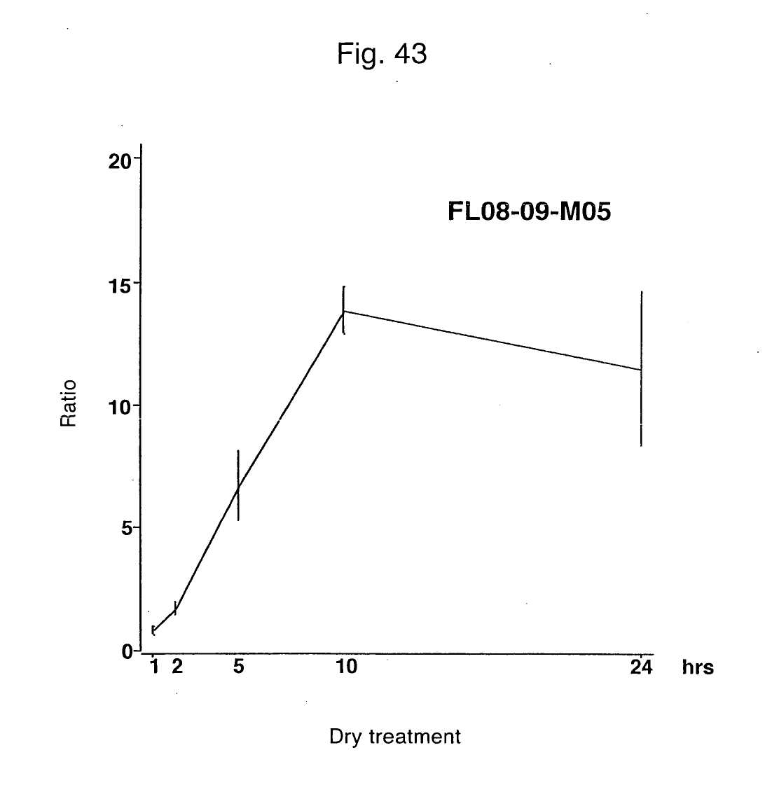

FIG. 44 is a characteristic graph showing the relationship between high salt treatment time and expression ratio regarding FL08-10-K08;

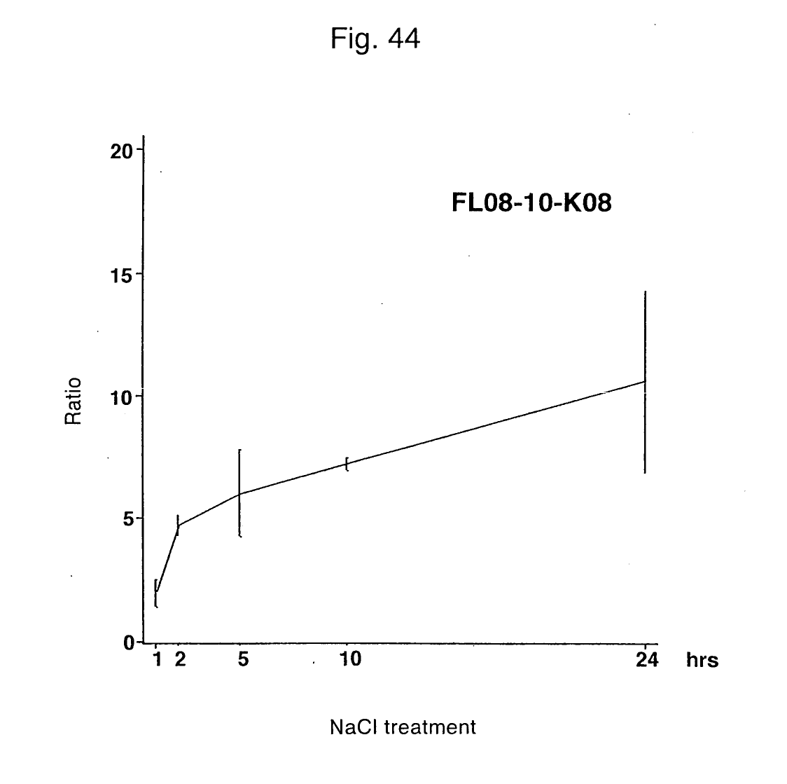

FIG. 45 is a characteristic graph showing the relationship between dehydration treatment time and expression ratio regarding FL08-11-P07;

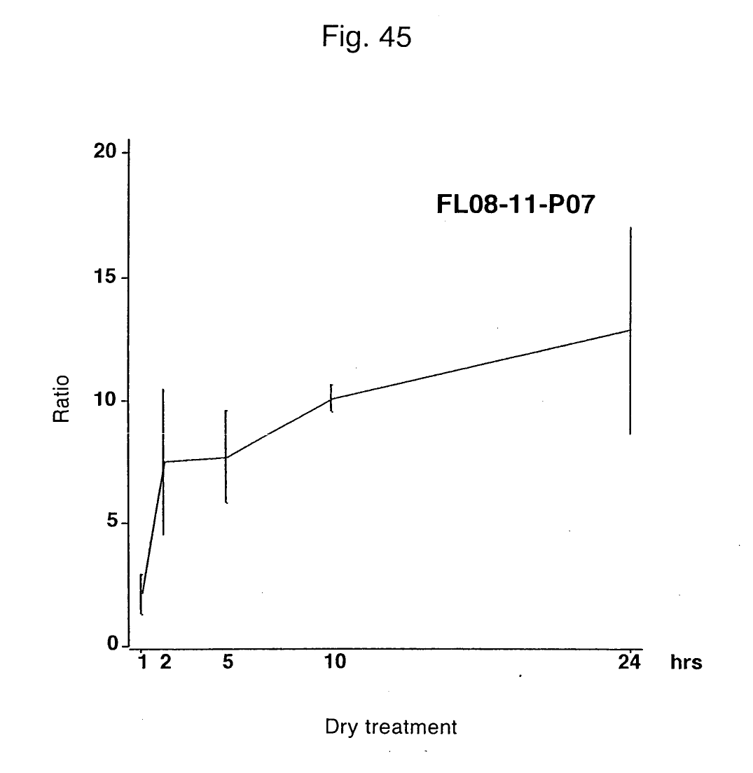

FIG. 46 is a characteristic graph showing the relationship between cold treatment time and expression ratio regarding FL08-11-P07;

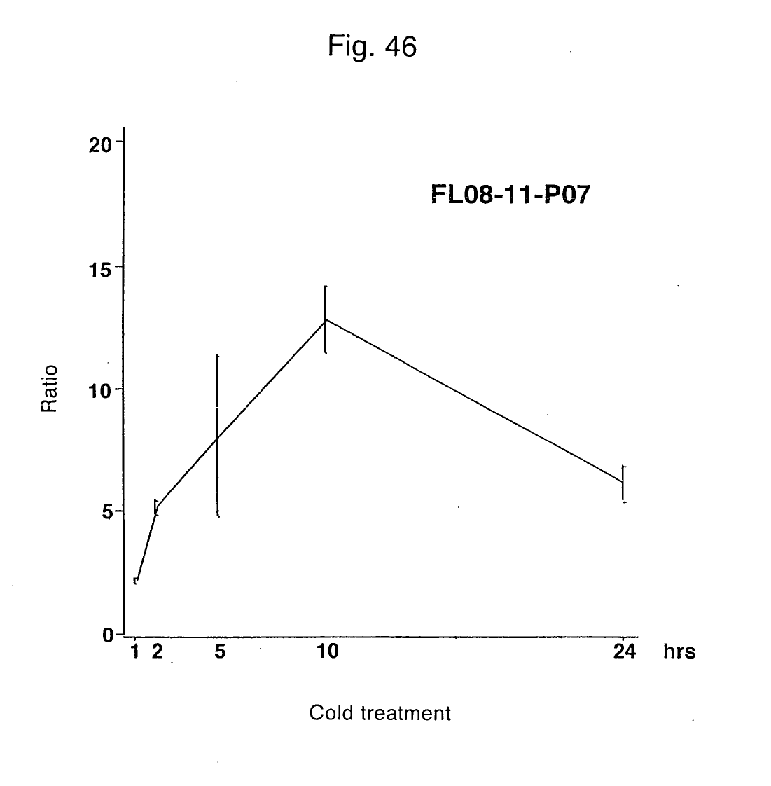

FIG. 47 is a characteristic graph showing the relationship between dehydration treatment time and expression ratio regarding FL08-13-F10;

FIG. 48 is a characteristic graph showing the relationship between high salt treatment time and expression ratio regarding FL08-13-F10;

FIG. 49 is a characteristic graph showing the relationship between ABA treatment time and expression ratio regarding FL08-13-F10;

FIG. 50 is a characteristic graph showing the relationship between dehydration treatment time and expression ratio regarding FL08-19-D04;

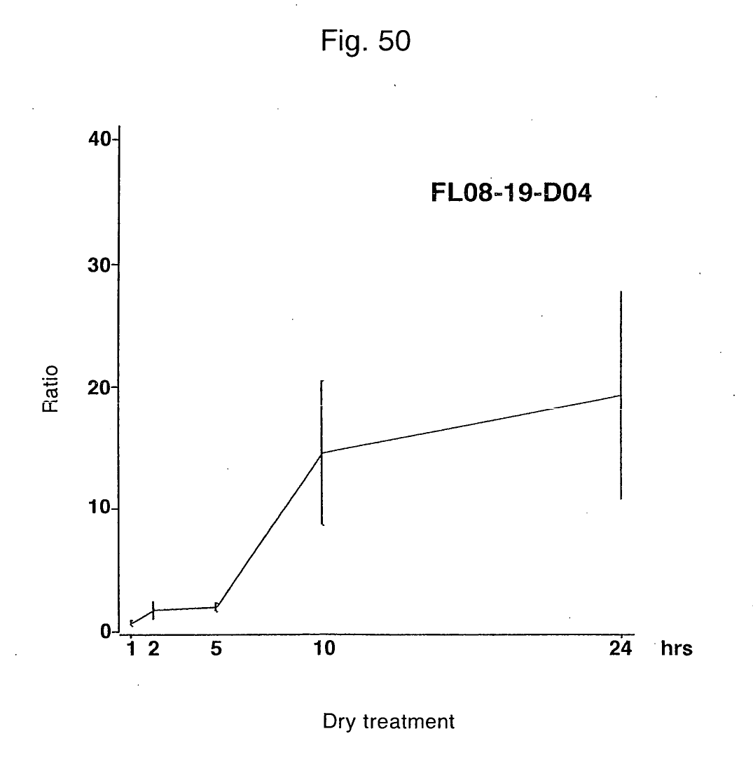

FIG. 51 is a characteristic graph showing the relationship between high salt treatment time and expression ratio regarding FL08-19-G15;

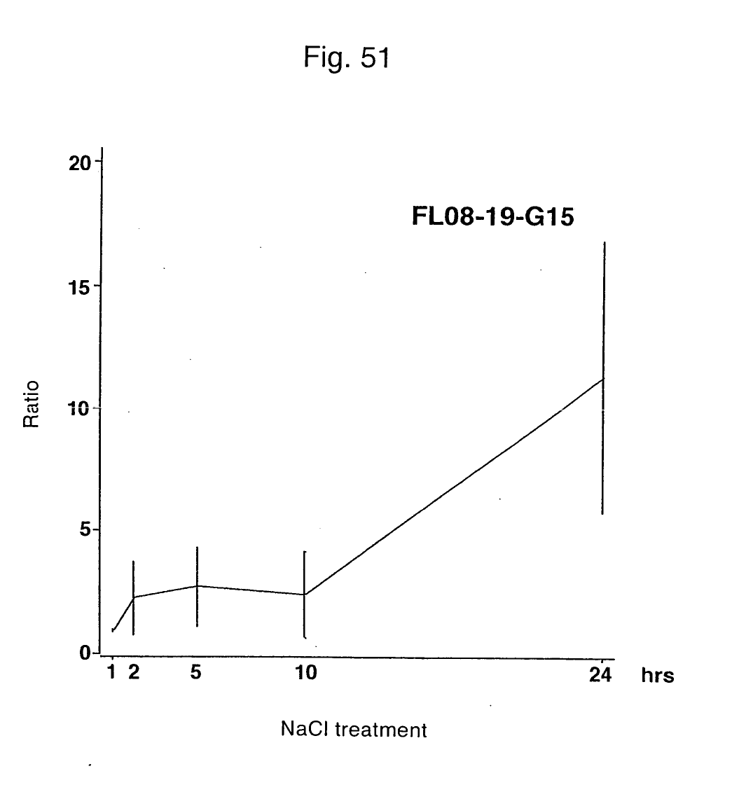

FIG. 52 is a characteristic graph showing the relationship between ABA treatment time and expression ratio regarding FL09-06-B11;

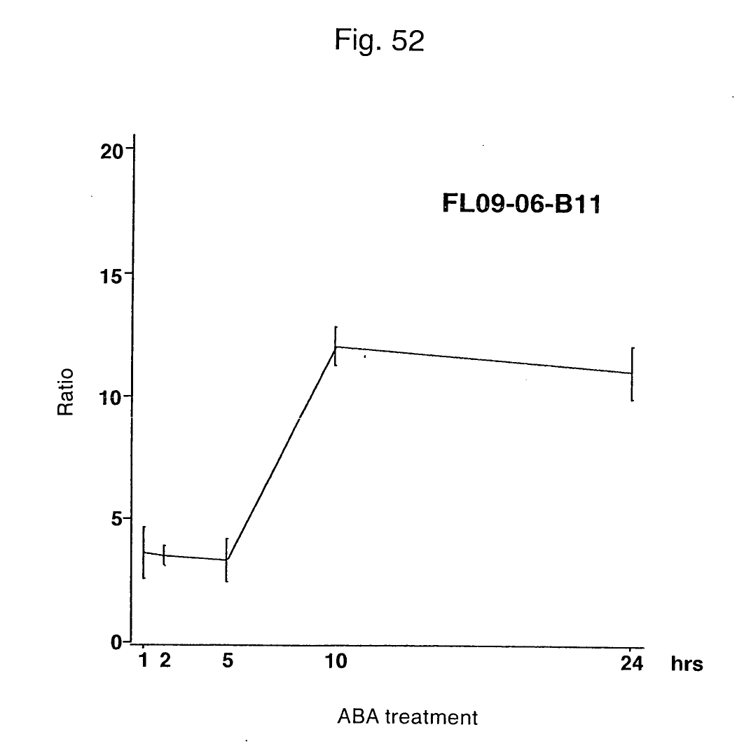

FIG. 53 is a characteristic graph showing the relationship between ABA treatment time and expression ratio regarding FL09-07-G17;

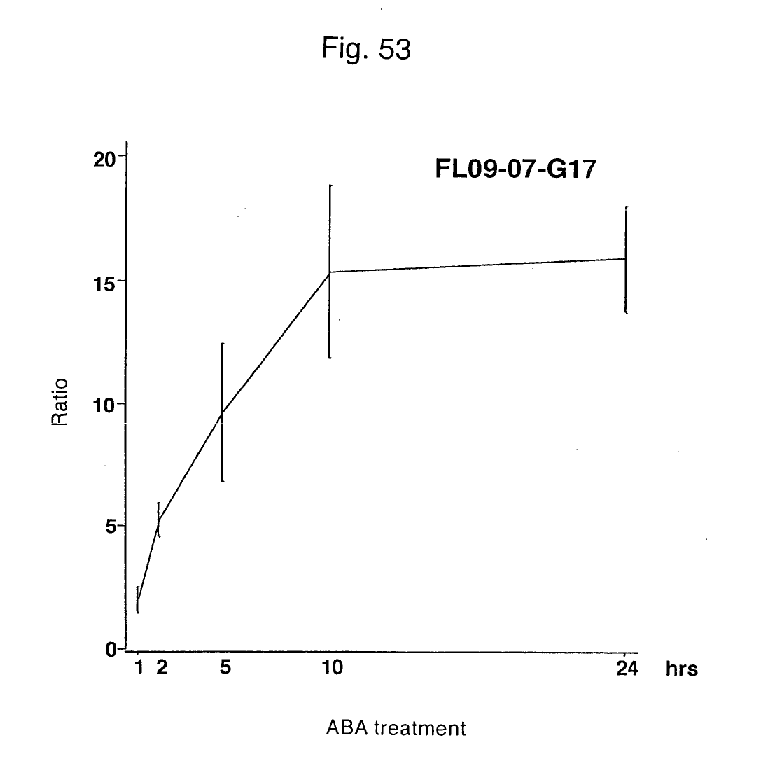

FIG. 54 is a characteristic graph showing the relationship between ABA treatment time and expression ratio regarding FL09-10-A12;

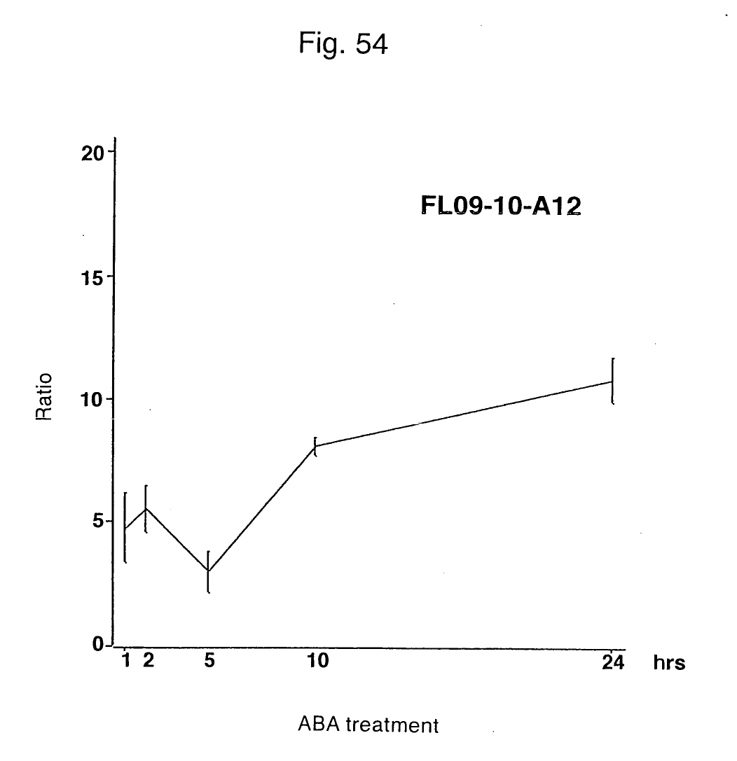

FIG. 55 is a characteristic graph showing the relationship between dehydration treatment time and expression ratio regarding FL09-13-P15;

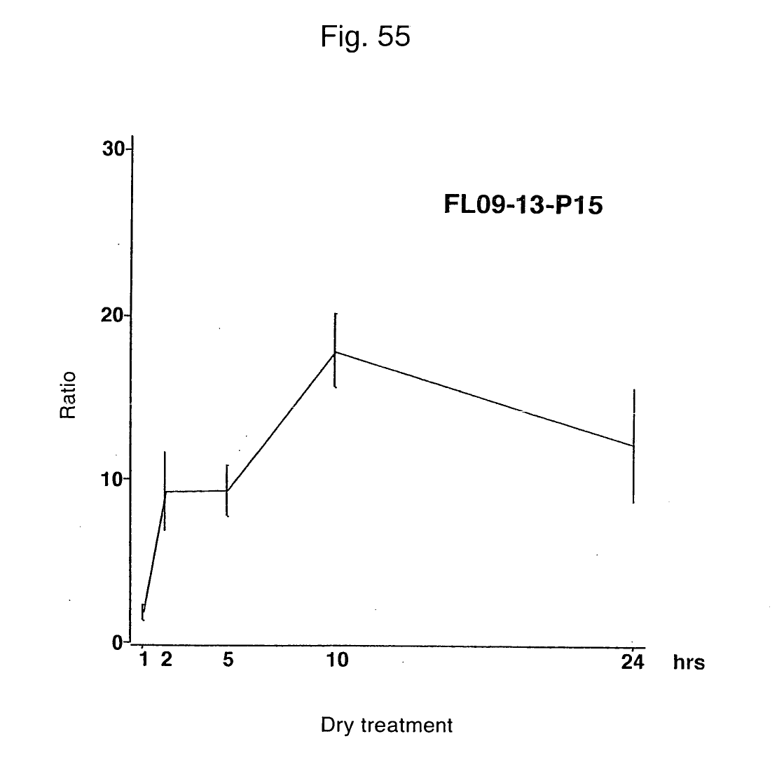

FIG. 56 is a characteristic graph showing the relationship between high salt treatment time and expression ratio regarding FL02-05-I05;

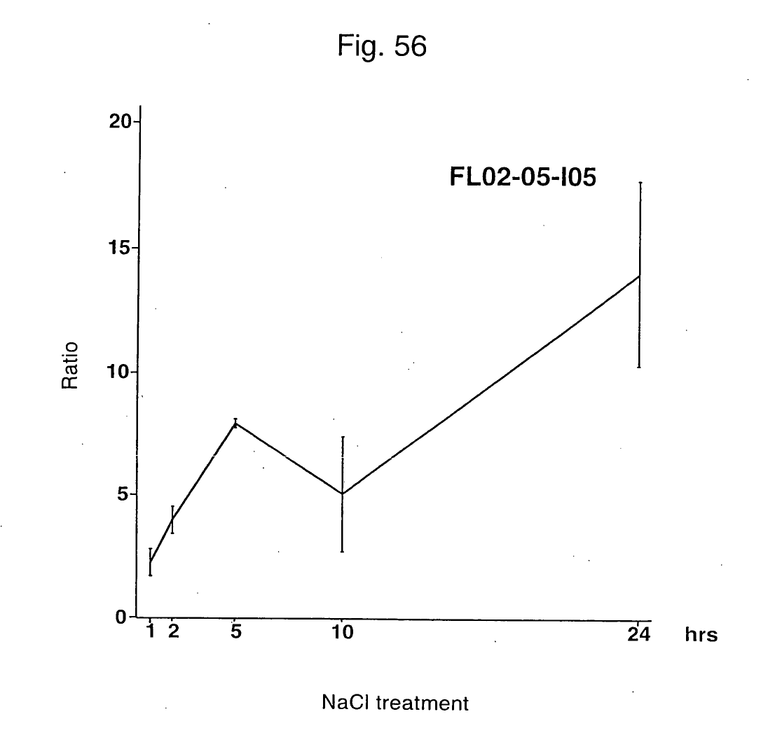

FIG. 57 is a characteristic graph showing the relationship between cold treatment time and expression ratio regarding FL04-12-N15;

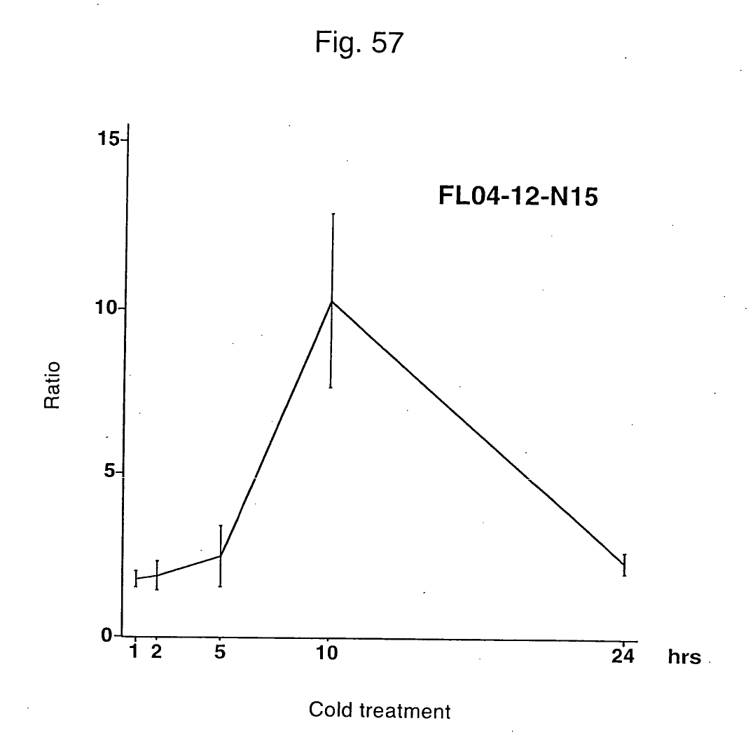

FIG. 58 is a characteristic graph showing the relationship between dehydration treatment time and expression ratio regarding FL04-16-P21;

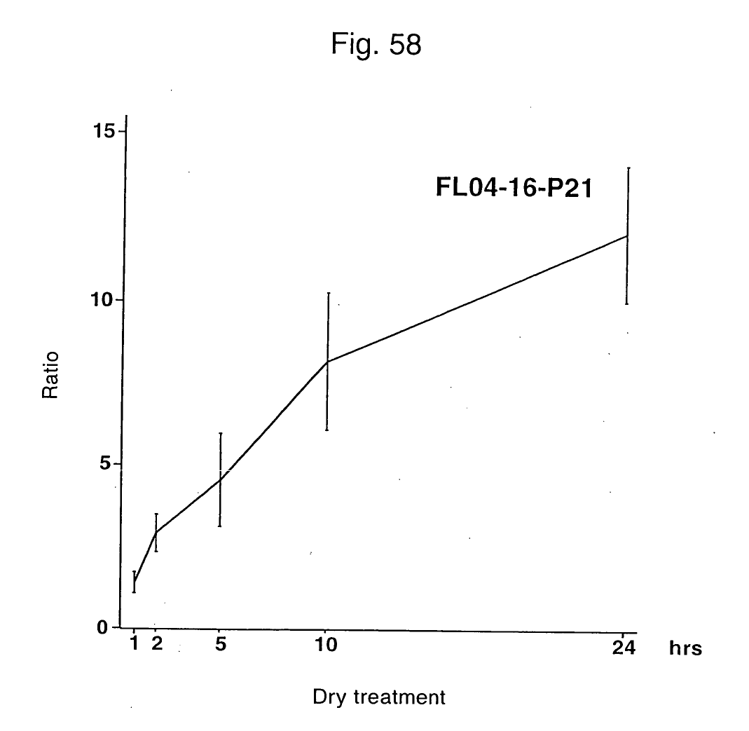

FIG. 59 is a characteristic graph showing the relationship between high salt treatment time and expression ratio regarding FL04-17-N22;

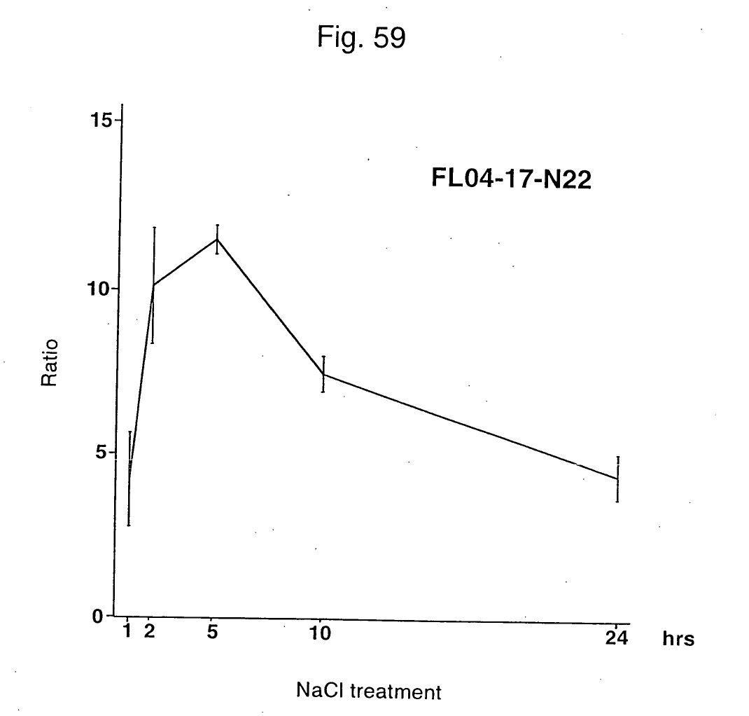

FIG. 60 is a characteristic graph showing the relationship between dehydration treatment time and expression ratio regarding FL04-20-P19;

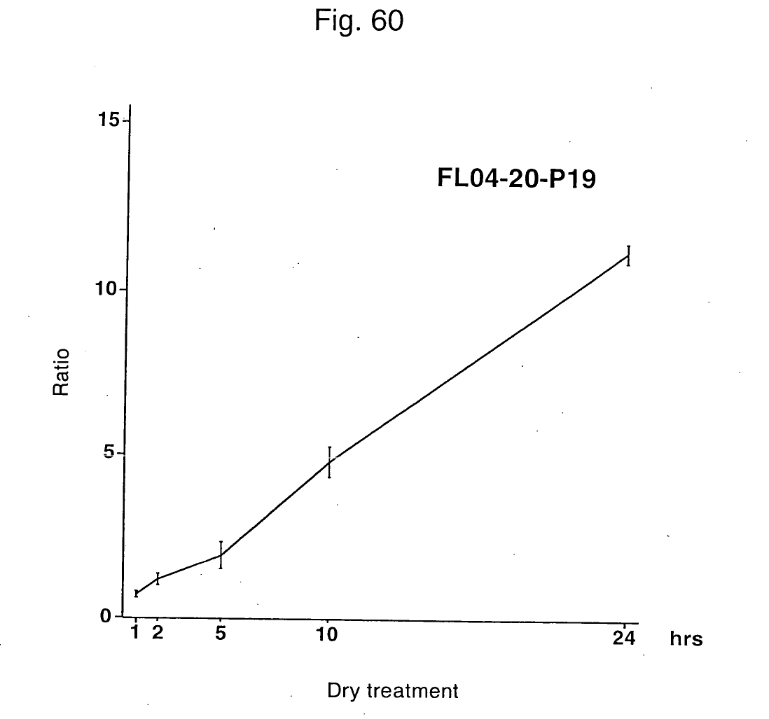

FIG. 61 is a characteristic graph showing the relationship between dehydration treatment time and expression ratio regarding FL02-09-H01;

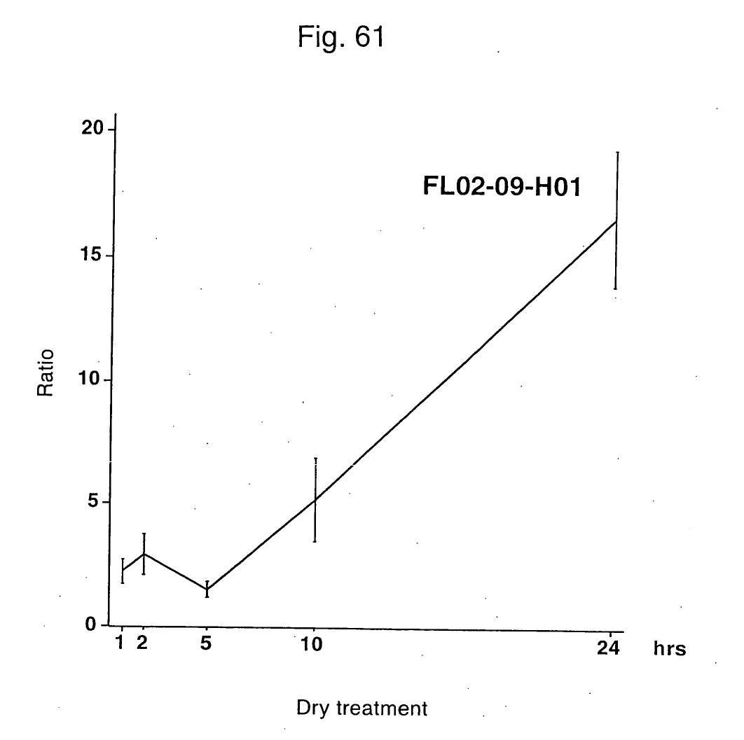

FIG. 62 is a characteristic graph showing the relationship between dehydration treatment time and expression ratio regarding FL05-01-D08;

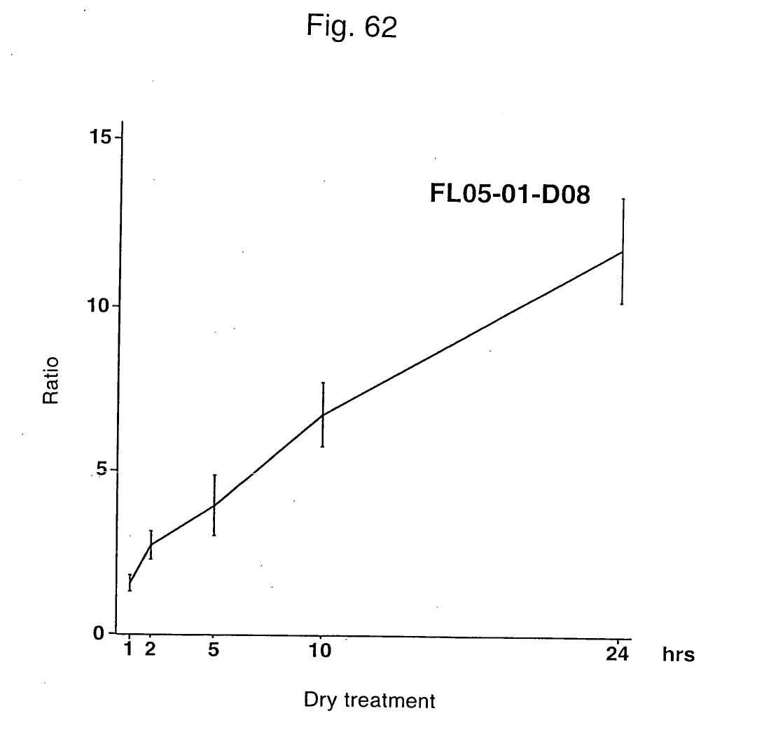

FIG. 63 is a characteristic graph showing the relationship between high salt treatment time and expression ratio regarding FL05-02-G08;

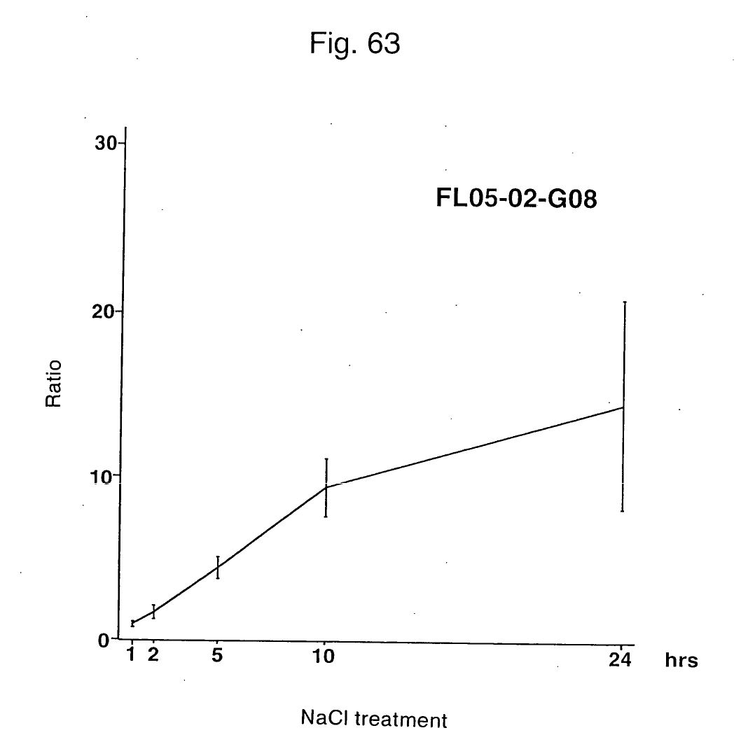

FIG. 64 is a characteristic graph showing the relationship between dehydration treatment time and expression ratio regarding FL05-02-O17;

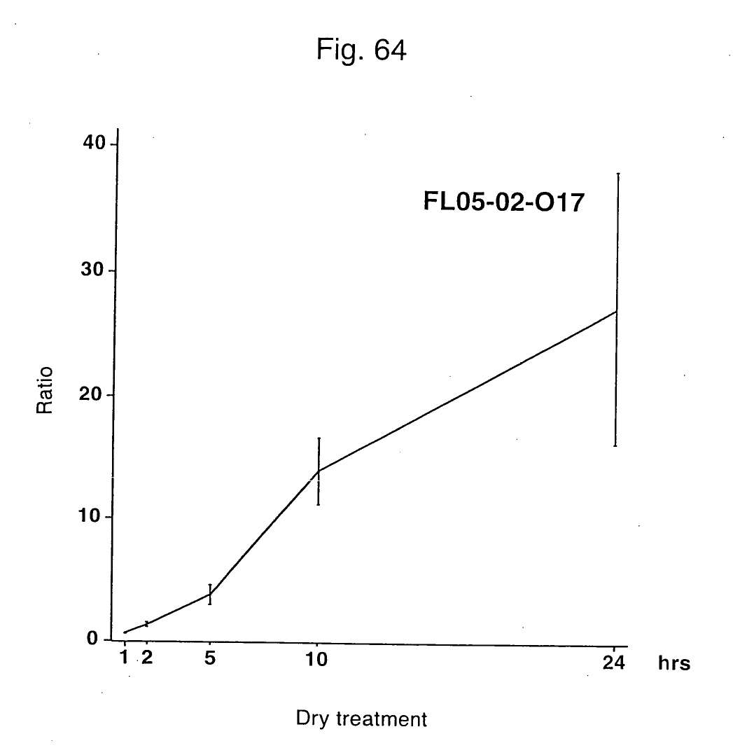

FIG. 65 is a characteristic graph showing the relationship between high salt treatment time and expression ratio regarding FL05-07-L13;

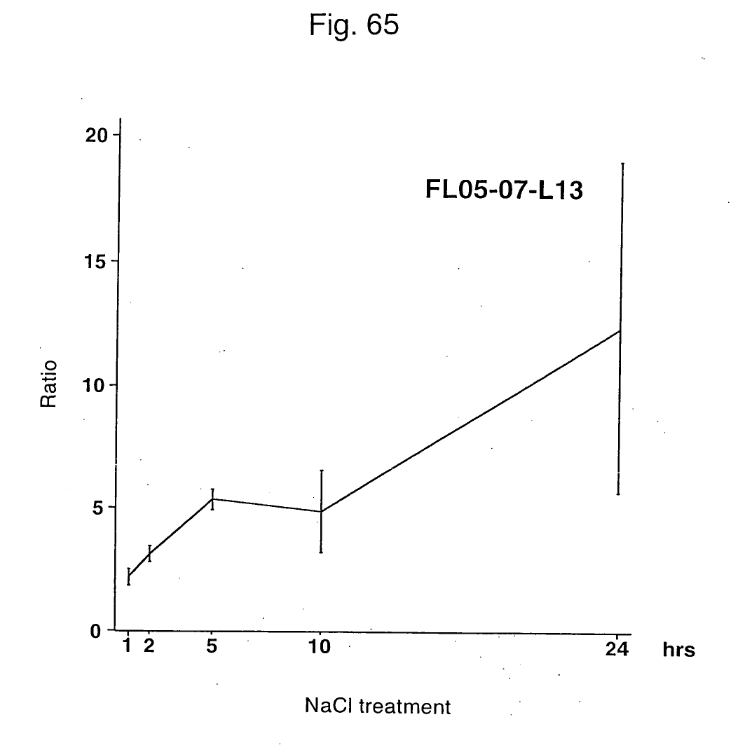

FIG. 66 is a characteristic graph showing the relationship between dehydration treatment time and expression ratio regarding FL05-08-B14;

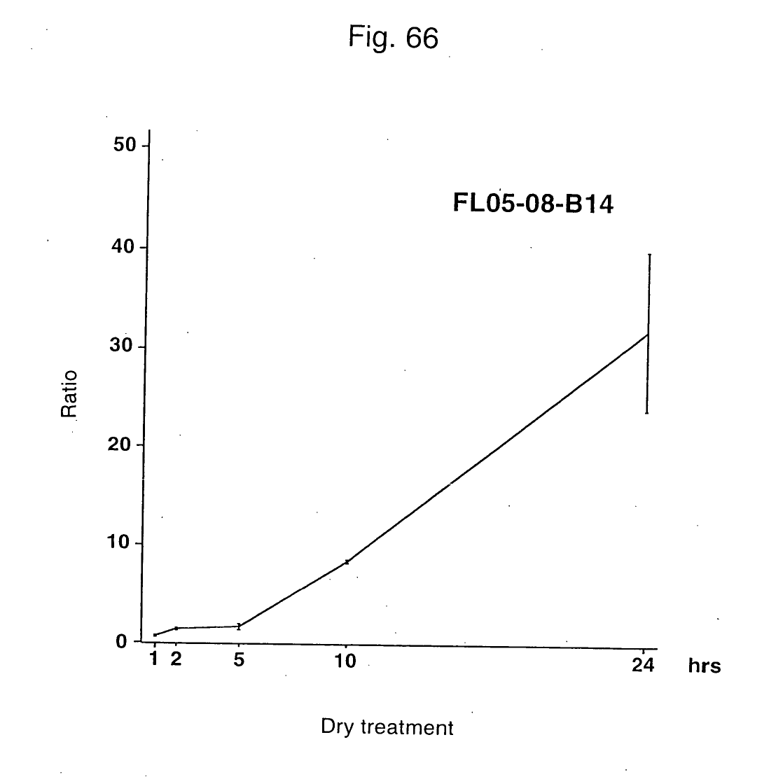

FIG. 67 is a characteristic graph showing the relationship between dehydration treatment time and expression ratio regarding FL05-09-N10;

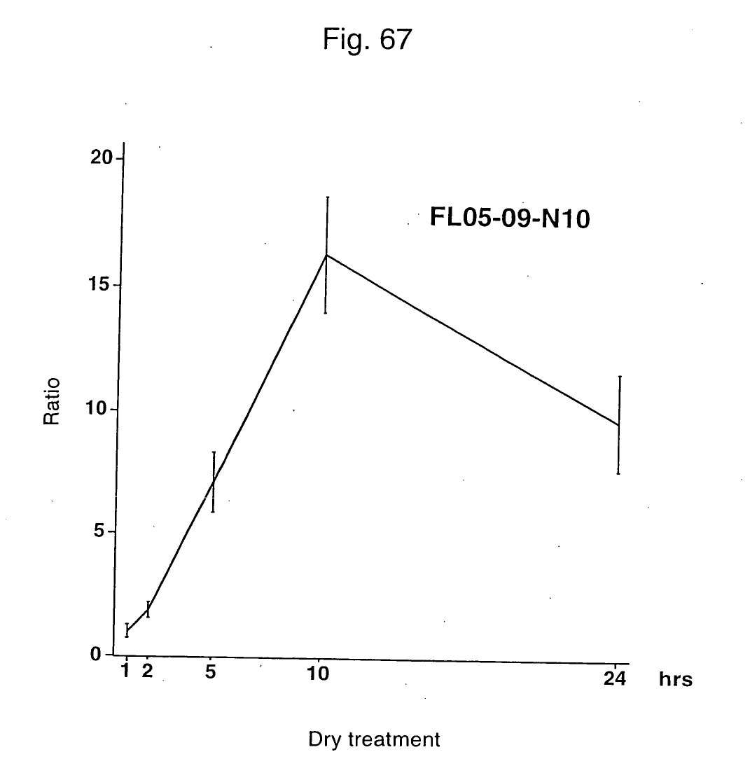

FIG. 68 is a characteristic graph showing the relationship between dehydration treatment time and expression ratio regarding FL05-11-L01;

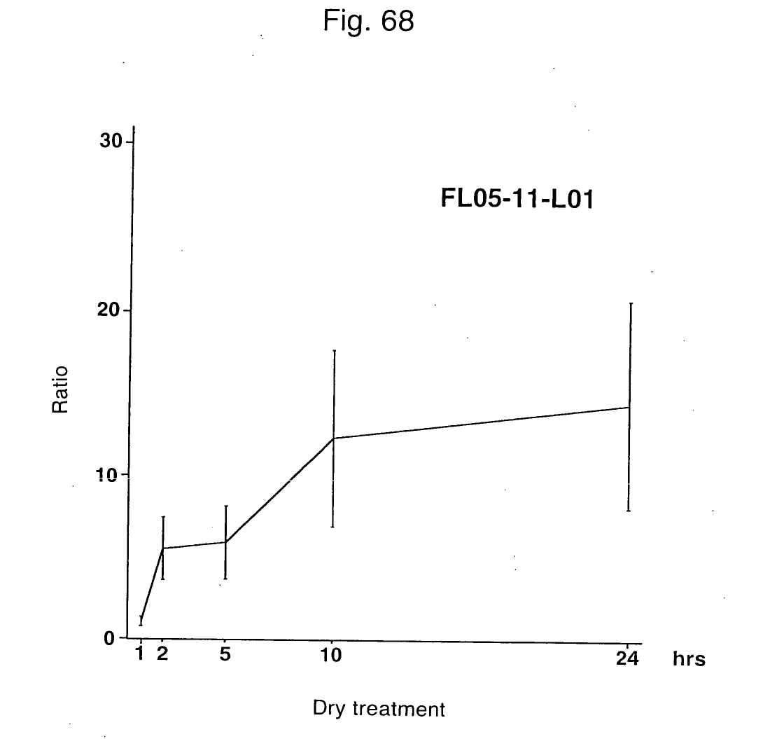

FIG. 69 is a characteristic graph showing the relationship between dehydration treatment time and expression ratio regarding FL05-12-J09;

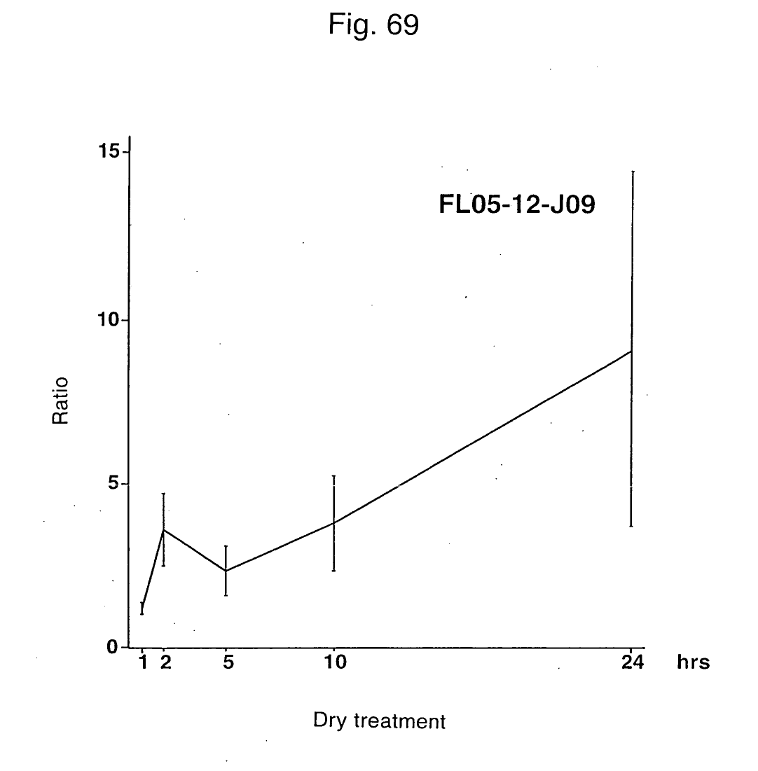

FIG. 70 is a characteristic graph showing the relationship between dehydration treatment time and expression ratio regarding FL05-14-D24;

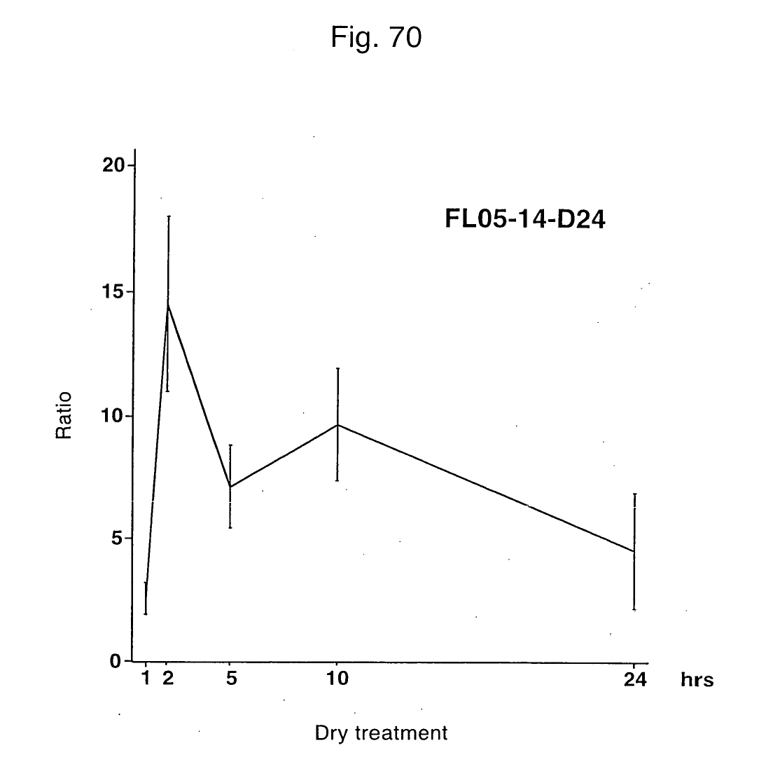

FIG. 71 is a characteristic graph showing the relationship between dehydration treatment time and expression ratio regarding FL05-14-F20;

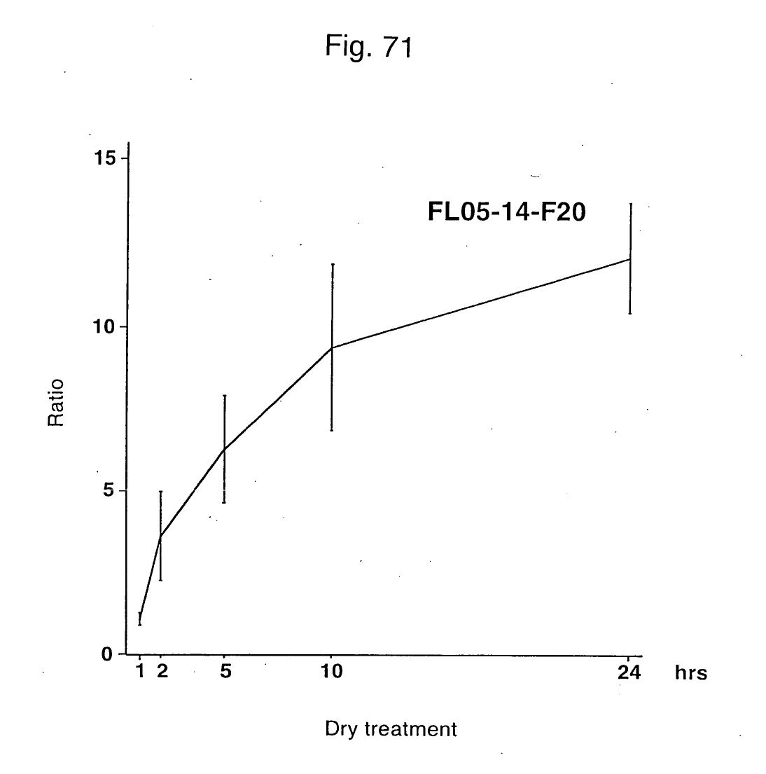

FIG. 72 is a characteristic graph showing the relationship between dehydration treatment time and expression ratio regarding FL05-14-I08;

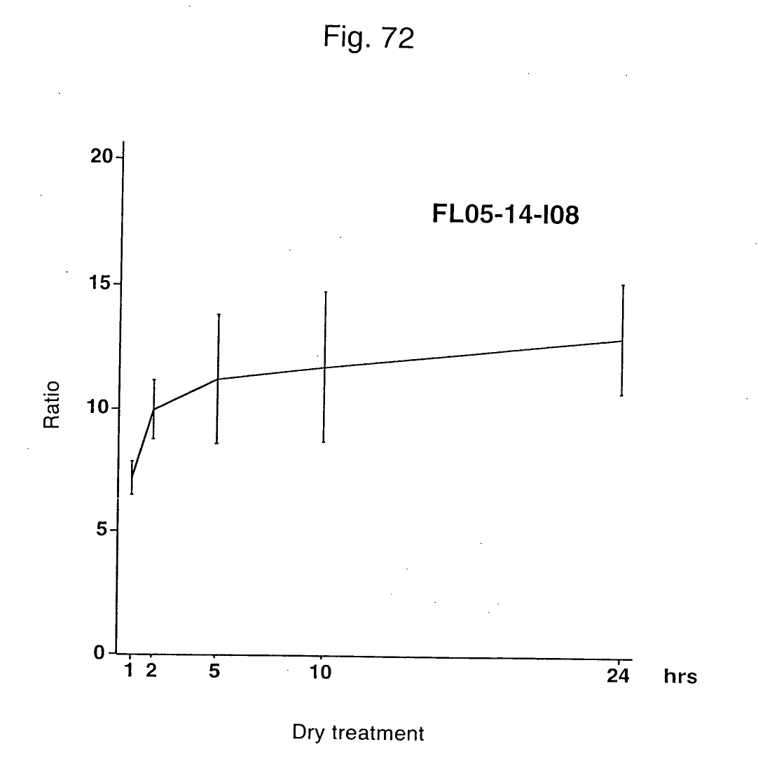

FIG. 73 is a characteristic graph showing the relationship between dehydration treatment time and expression ratio regarding FL05-15-C04;

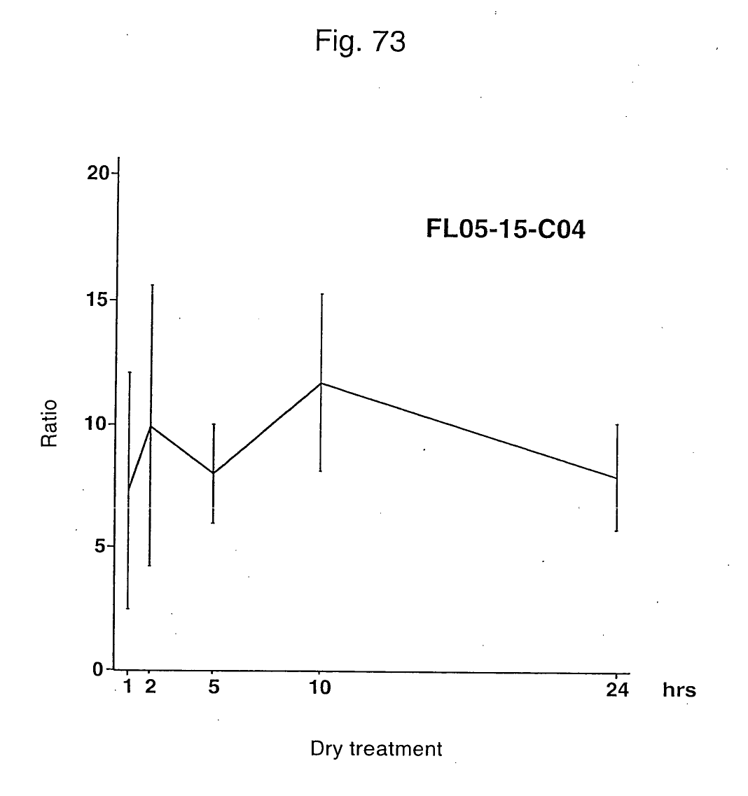

FIG. 74 is a characteristic graph showing the relationship between dehydration treatment time and expression ratio regarding FL05-15-E19;

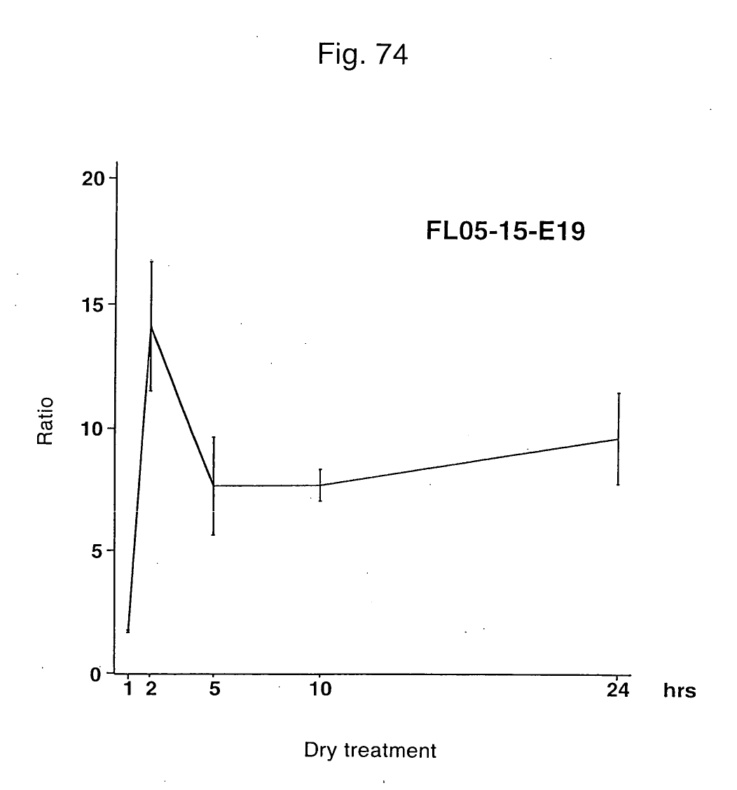

FIG. 75 is a characteristic graph showing the relationship between dehydration treatment time and expression ratio regarding FL05-18-A06;

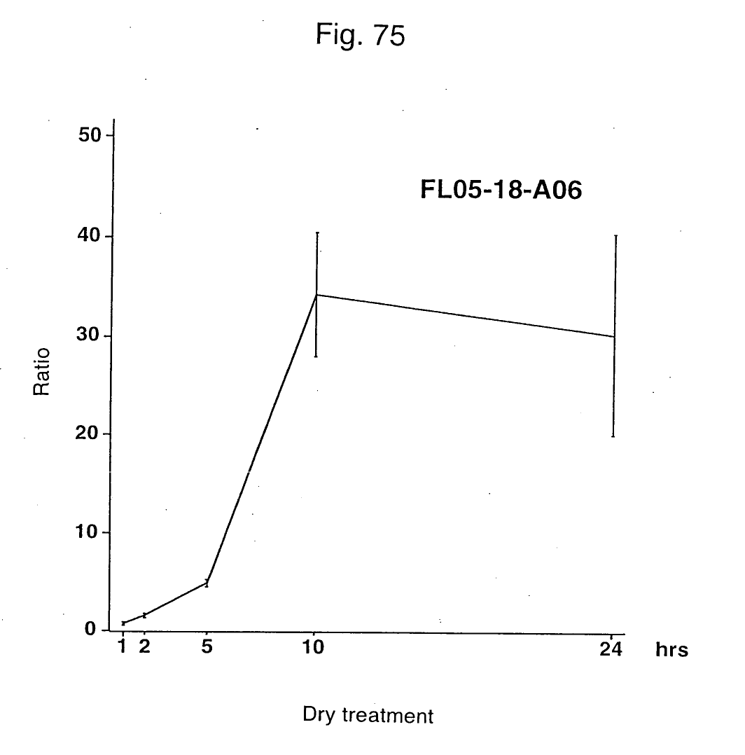

FIG. 76 is a characteristic graph showing the relationship between high salt treatment time and expression ratio regarding FL05-18-H 15;

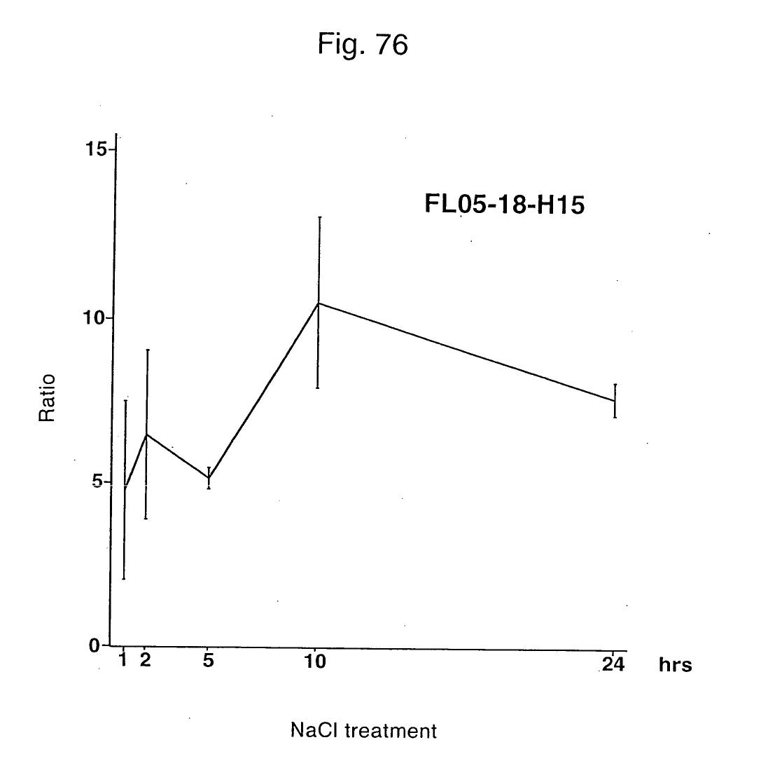

FIG. 77 is a characteristic graph showing the relationship between dehydration treatment time and expression ratio regarding FL05-19-C02;

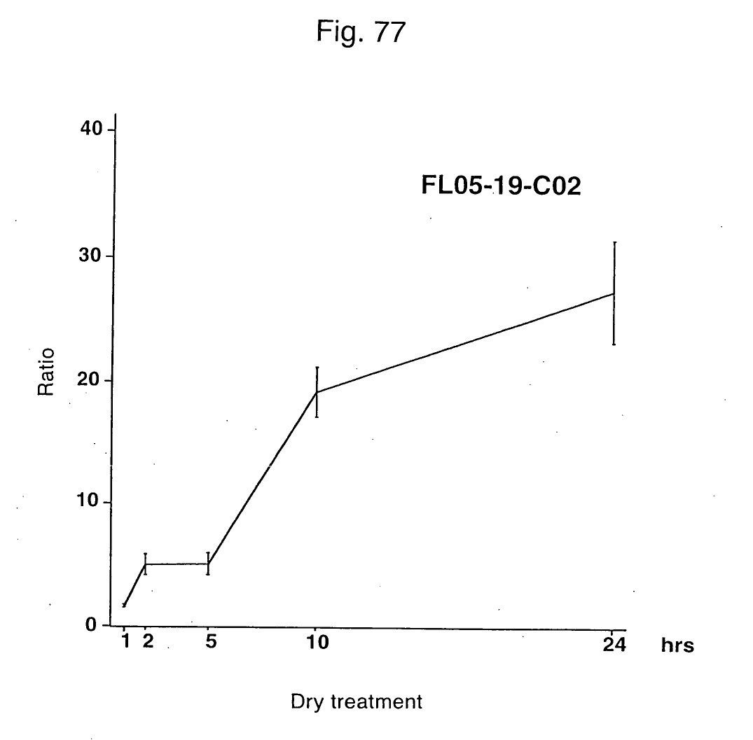

FIG. 78 is a characteristic graph showing the relationship between dehydration treatment time and expression ratio regarding FL05-20-M16;

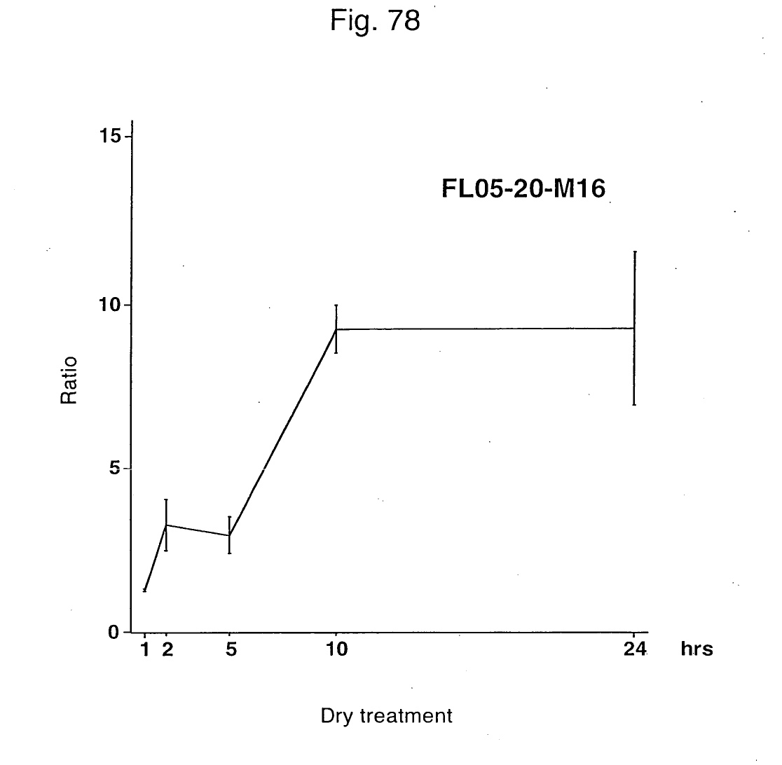

FIG. 79 is a characteristic graph showing the relationship between cold treatment time and expression ratio regarding FL05-20-N18;

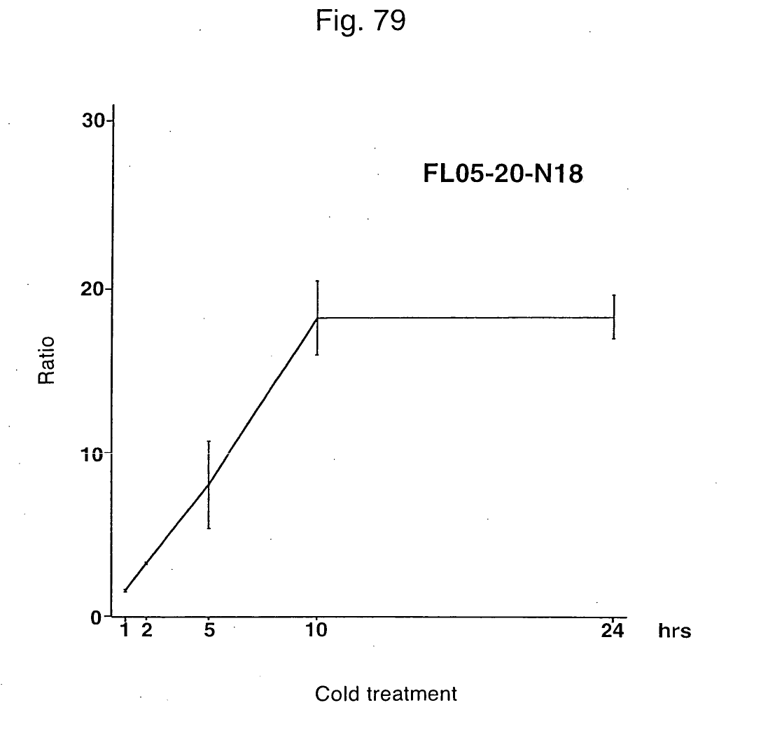

FIG. 80 is a characteristic graph showing the relationship between dehydration treatment time and expression ratio regarding FL05-21-E06;

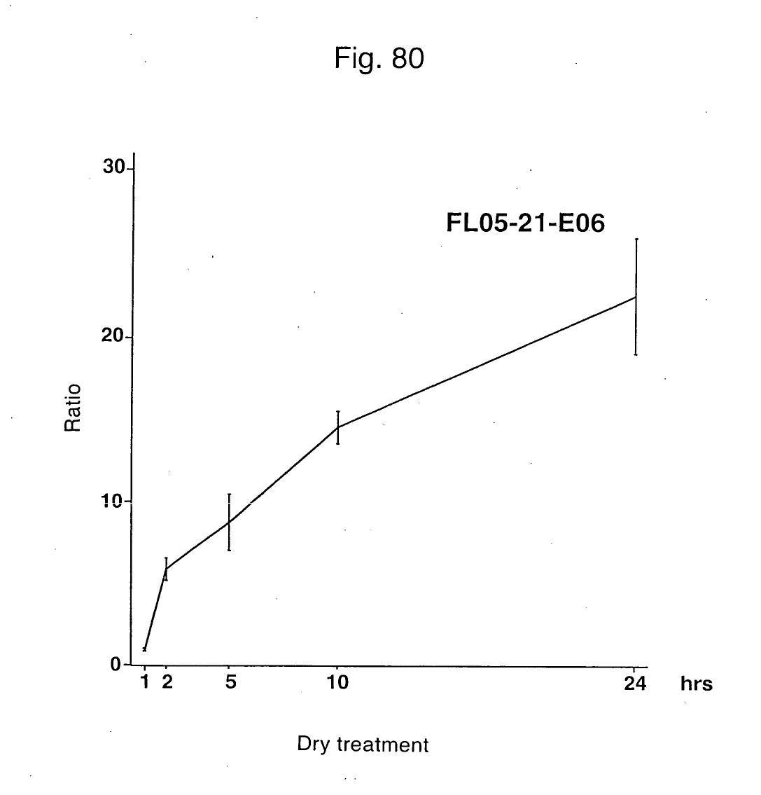

FIG. 81 is a characteristic graph showing the relationship between dehydration treatment time and expression ratio regarding FL05-21-L12;

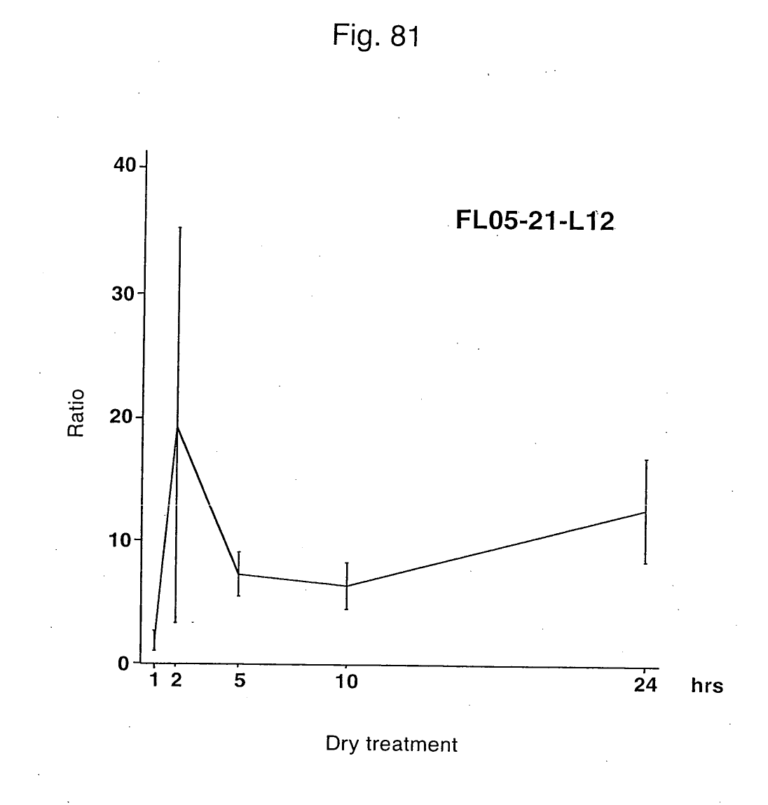

FIG. 82 is a characteristic graph showing the relationship between dehydration treatment time and expression ratio regarding FL06-07-B08;

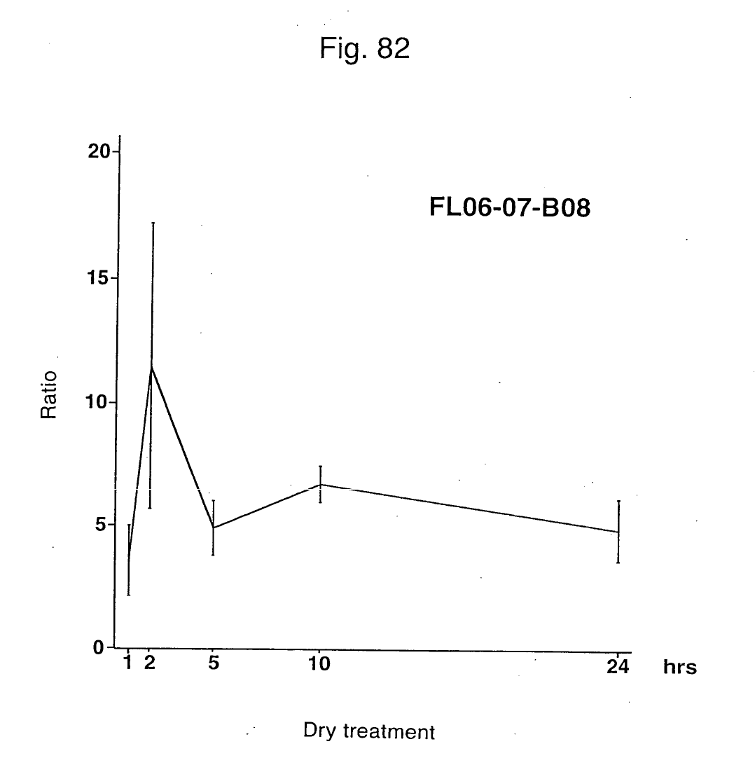

FIG. 83 is a characteristic graph showing the relationship between dehydration treatment time and expression ratio regarding FL06-08-H20;

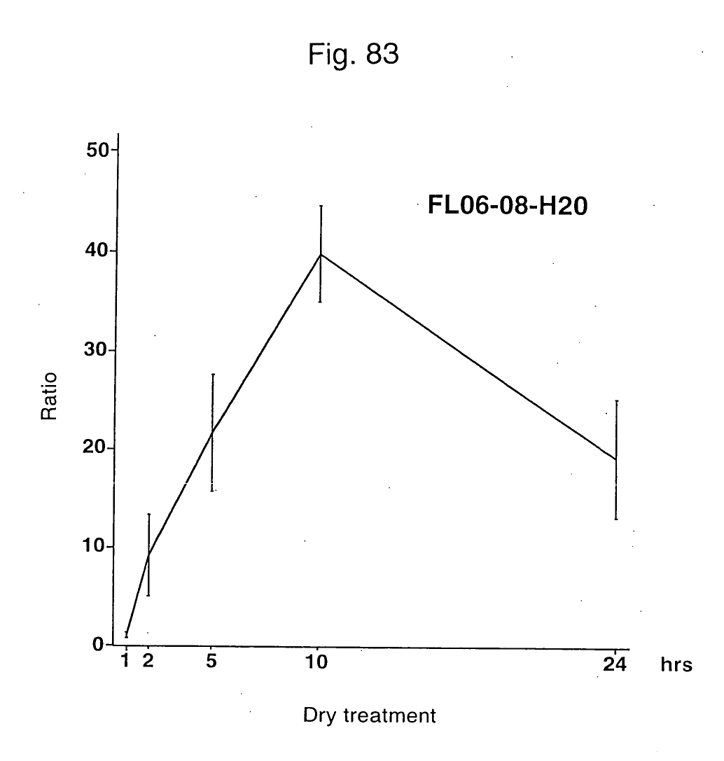

FIG. 84 is a characteristic graph showing the relationship between dehydration treatment time and expression ratio regarding FL06-09-N04;

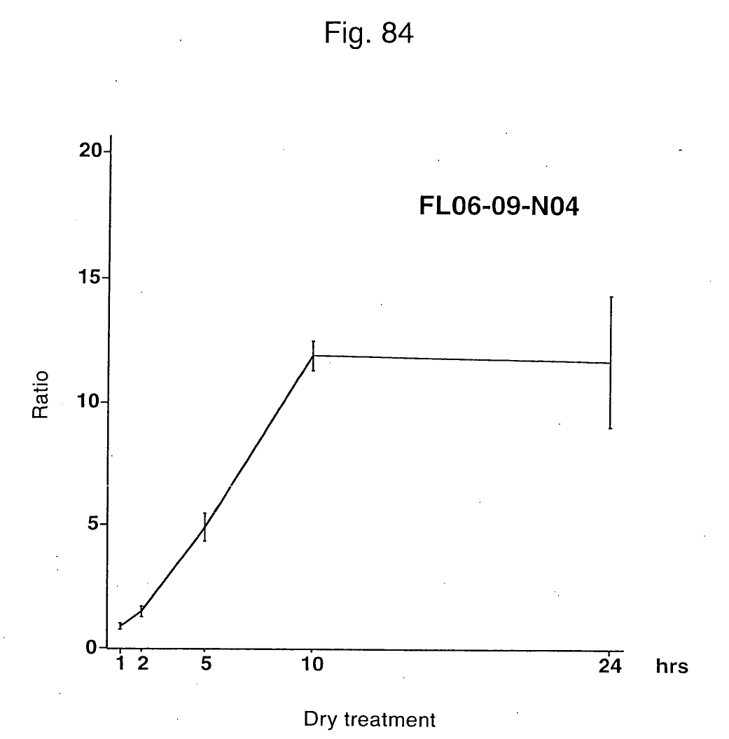

FIG. 85 is a characteristic graph showing the relationship between dehydration treatment time and expression ratio regarding FL06-11-K21;

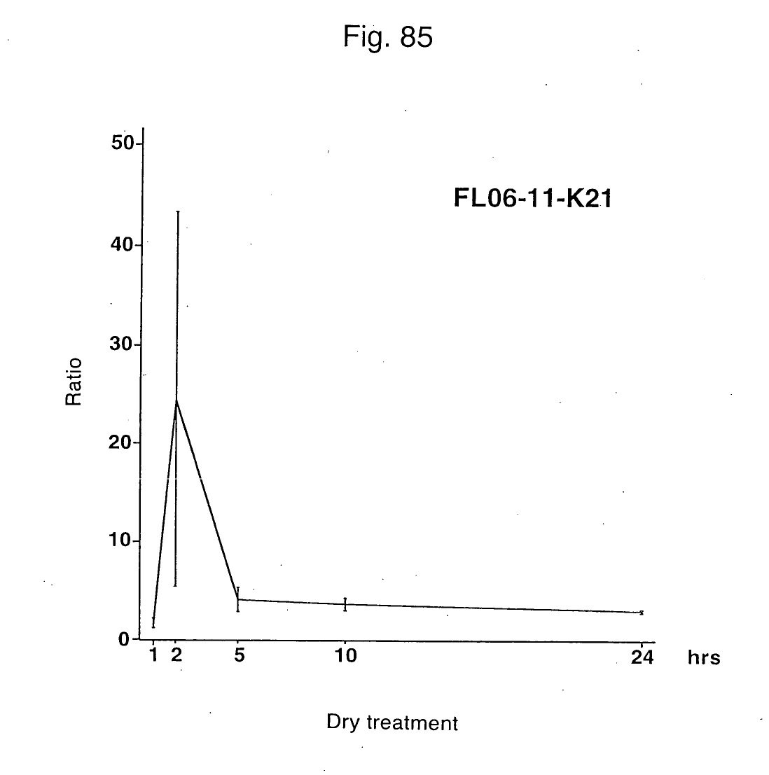

FIG. 86 is a characteristic graph showing the relationship between high salt treatment time and expression ratio regarding FL07-07-G15;

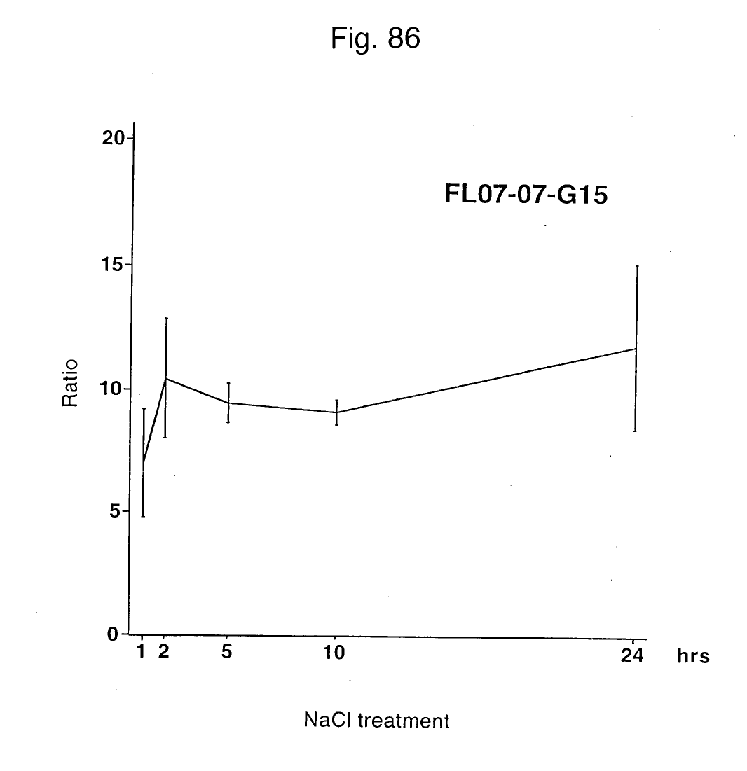

FIG. 87 is a characteristic graph showing the relationship between high salt treatment time and expression ratio regarding FL07-12-D17;

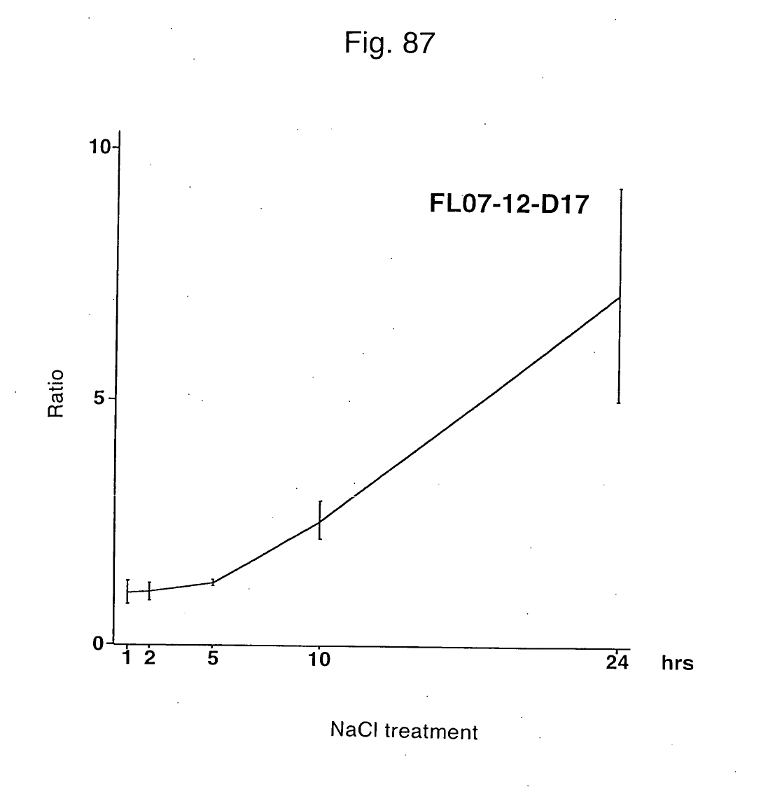

FIG. 88 is a characteristic graph showing the relationship between dehydration treatment time and expression ratio regarding FL08-11-C23;

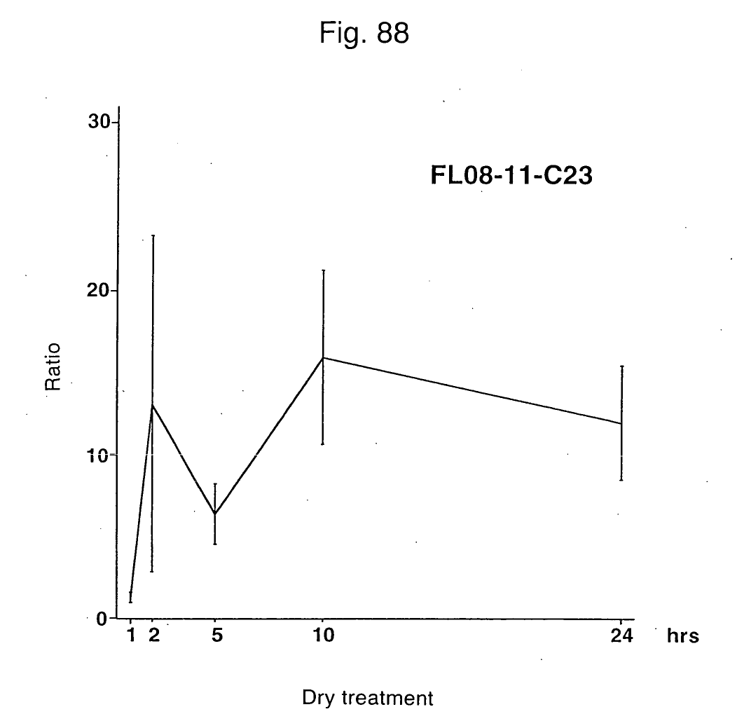

FIG. 89 is a characteristic graph showing the relationship between dehydration treatment time and expression ratio regarding FL08-13-G20;

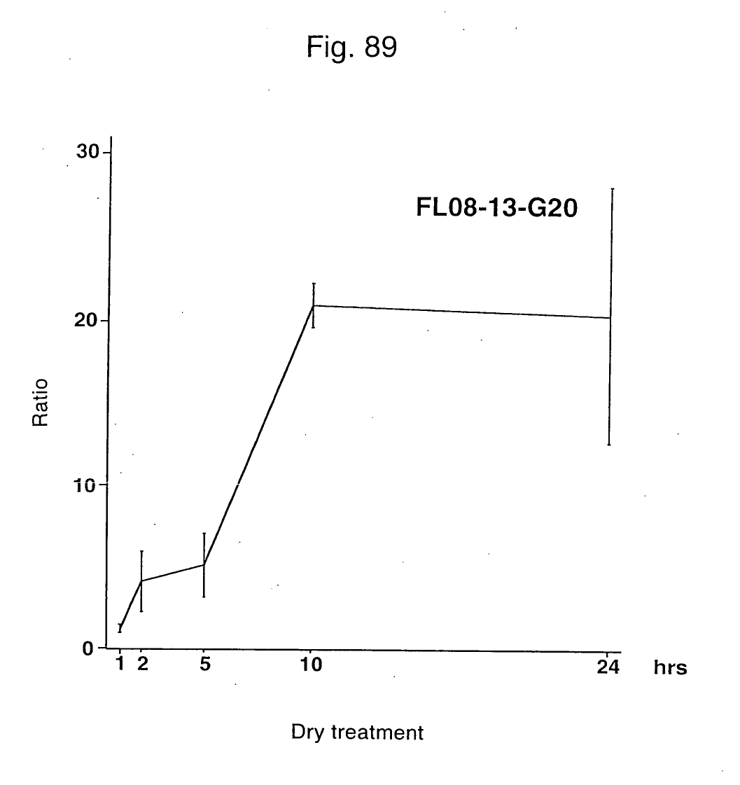

FIG. 90 is a characteristic graph showing the relationship between dehydration treatment time and expression ratio regarding FL08-15-M21;

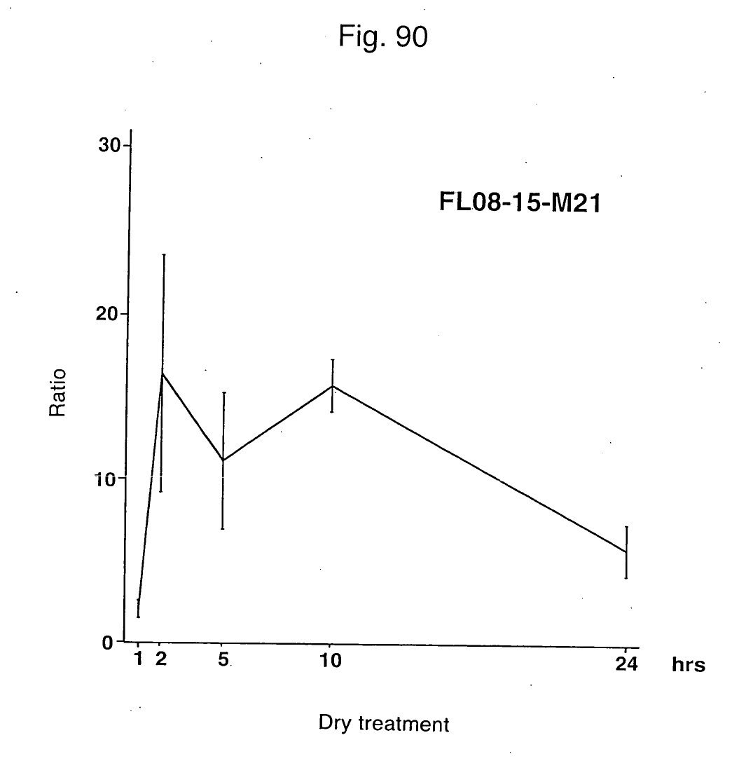

FIG. 91 is a characteristic graph showing the relationship between dehydration treatment time and expression ratio regarding FL08-18-N19;

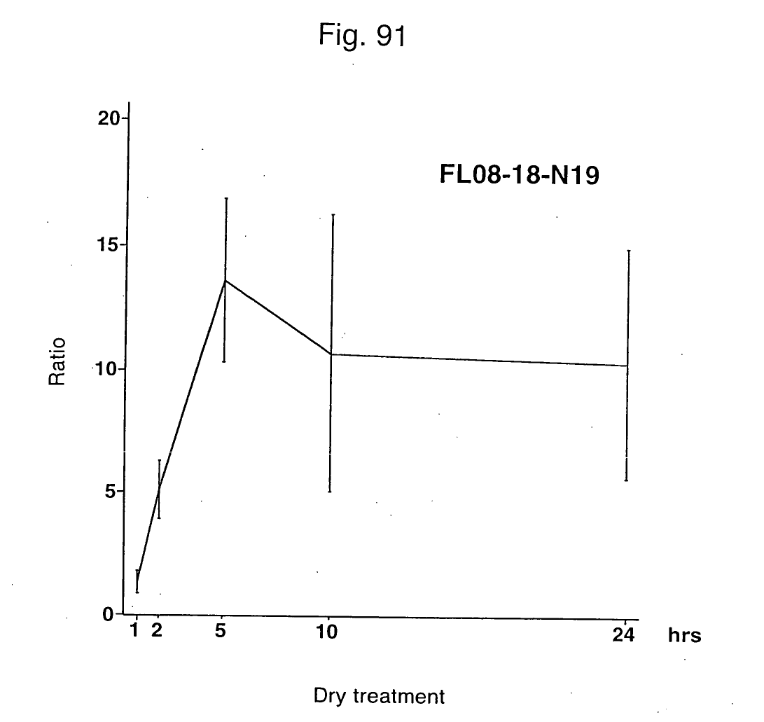

FIG. 92 is a characteristic graph showing the relationship between dehydration treatment time and expression ratio regarding FL08-19-C07;

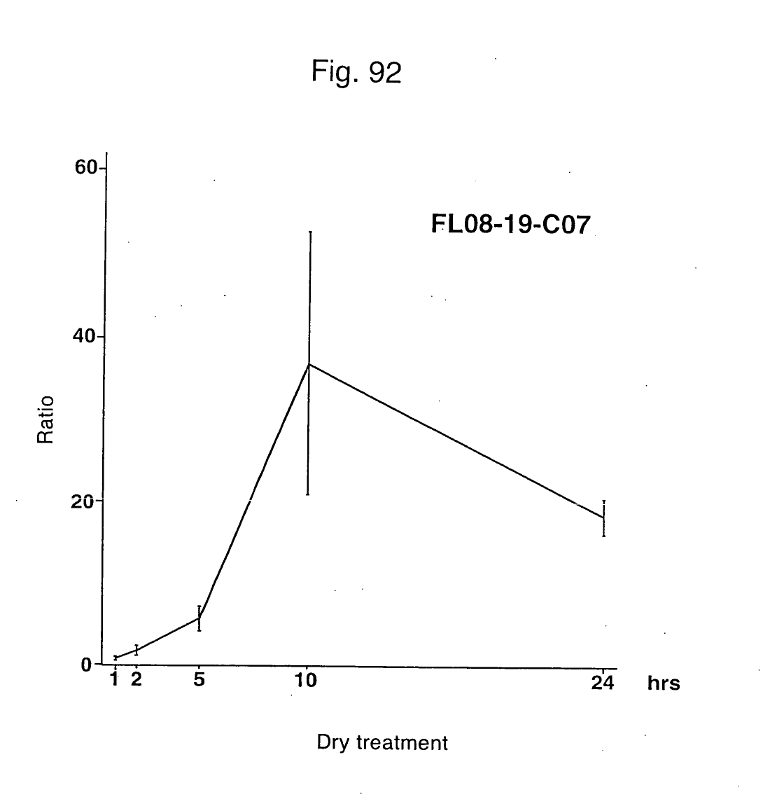

FIG. 93 is a characteristic graph showing the relationship between high salt treatment time and expression ratio regarding FL08-19-P05;

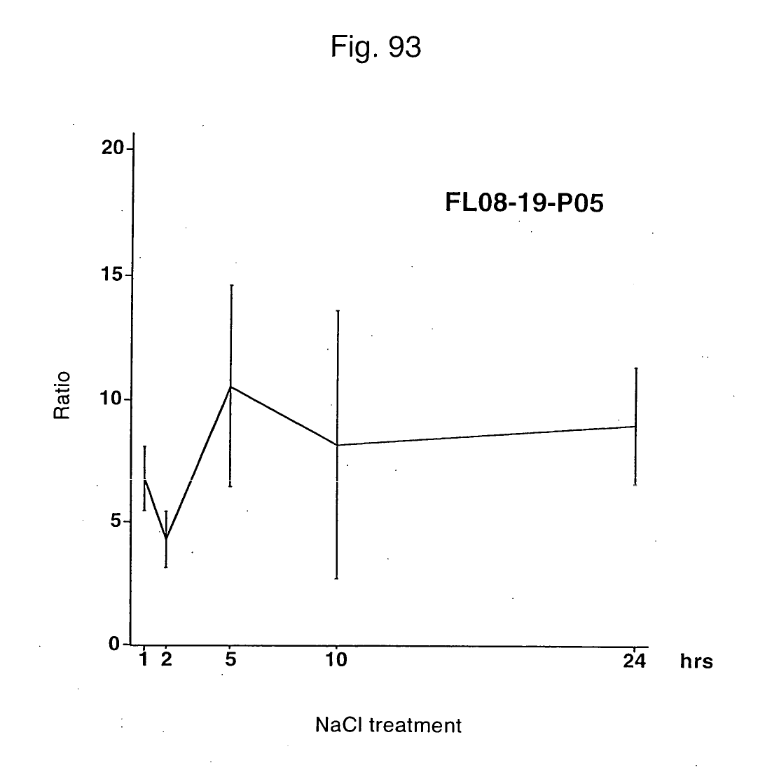

FIG. 94 is a characteristic graph showing the relationship between high salt treatment time and expression ratio regarding FL09-07-G09;

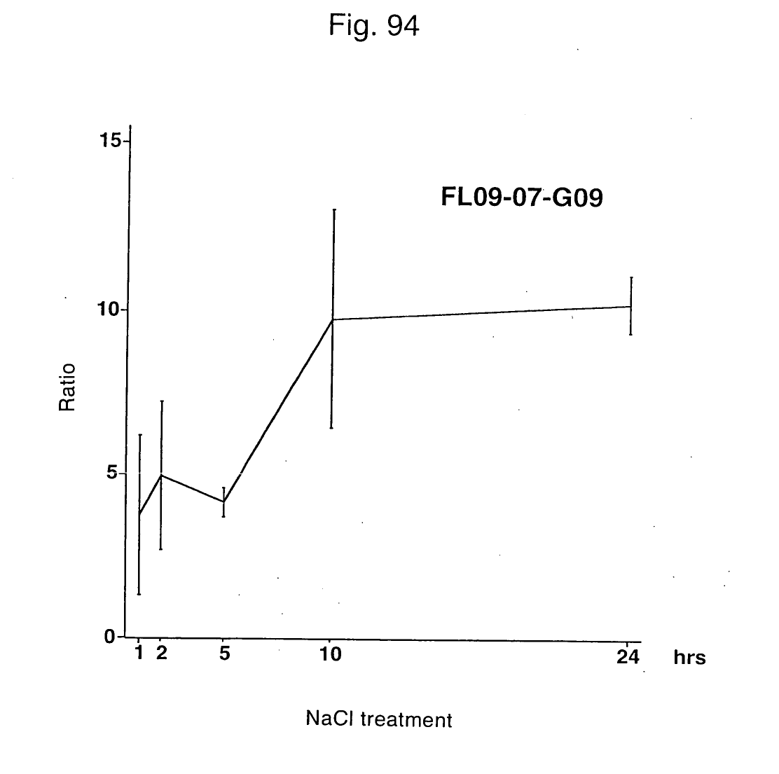

FIG. 95 is a characteristic graph showing the relationship between dehydration treatment time and expression ratio regarding FL09-07-G15;

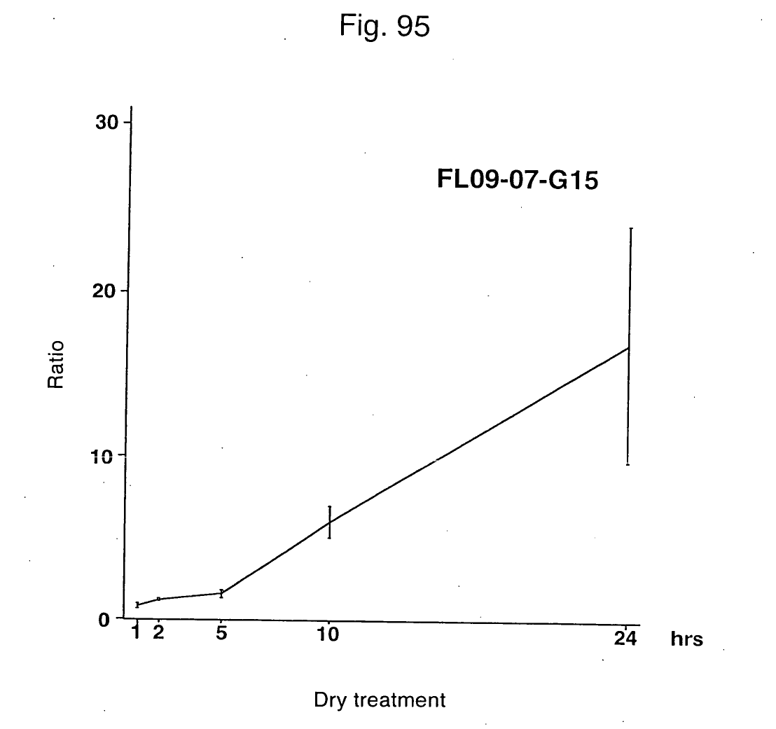

FIG. 96 is a characteristic graph showing the relationship between high salt treatment time and expression ratio regarding FL09-10-J18;

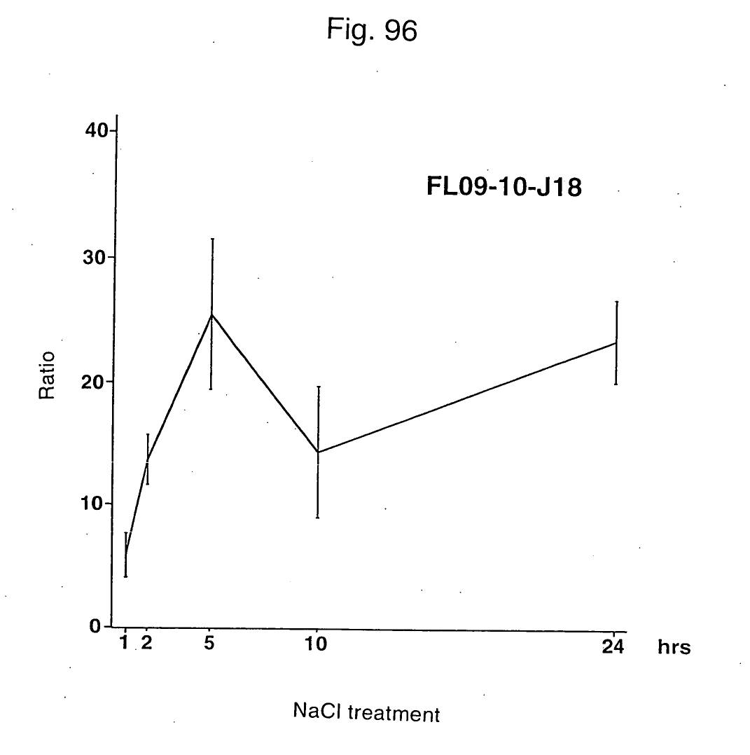

FIG. 97 is a characteristic graph showing the relationship between dehydration treatment time and expression ratio regarding FL09-11-I12;

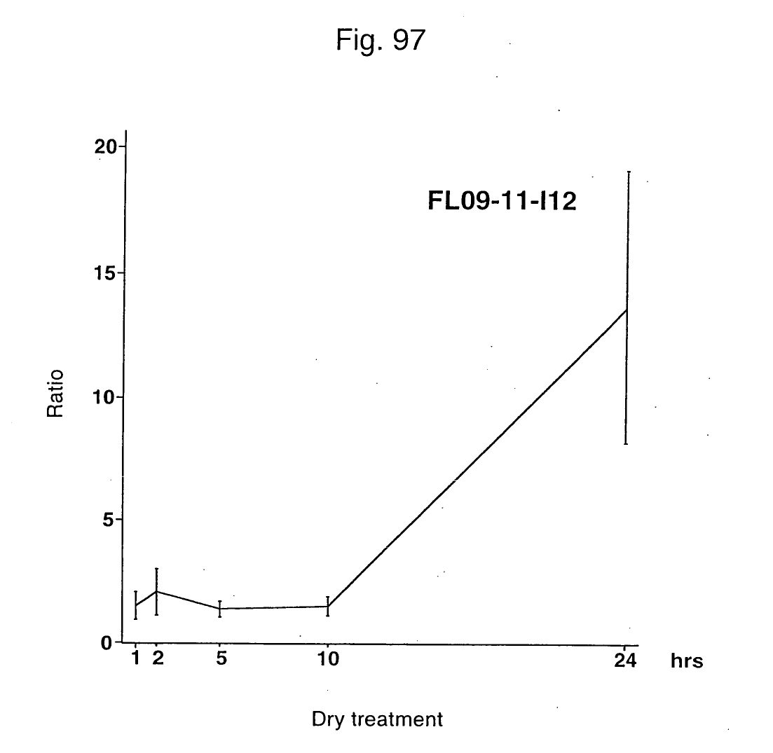

FIG. 98 is a characteristic graph showing the relationship between dehydration treatment time and expression ratio regarding FL09-12-B03;

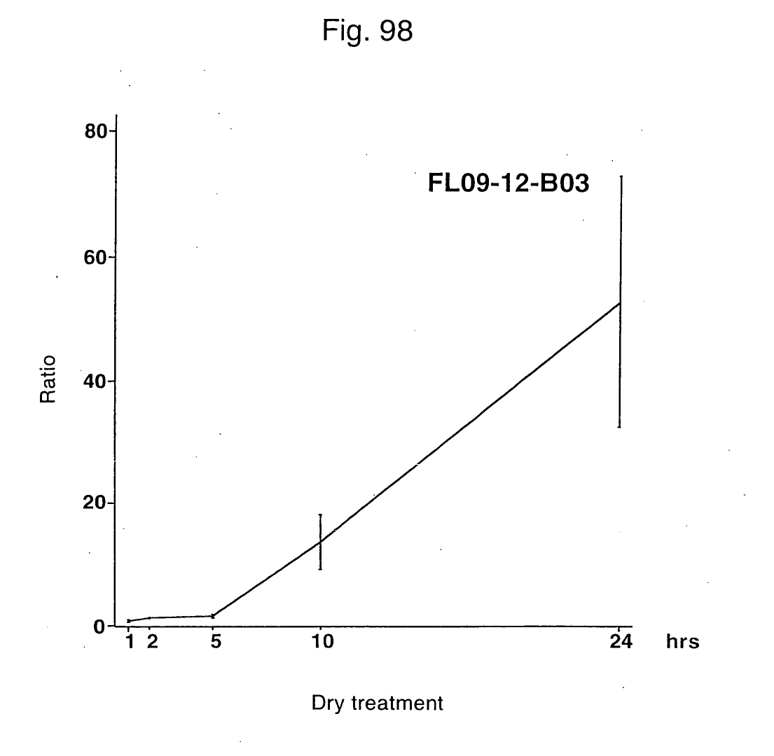

FIG. 99 is a characteristic graph showing the relationship between high salt treatment time and expression ratio regarding FL09-16-I11;

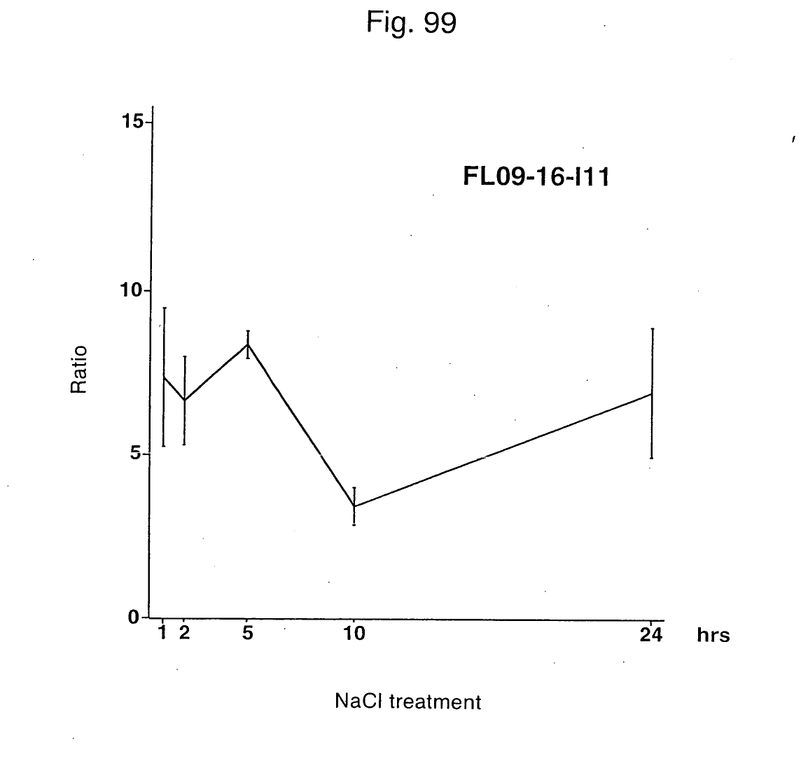

FIG. 100 is a characteristic graph showing the relationship between high salt treatment time and expression ratio regarding FL09-16-M04;

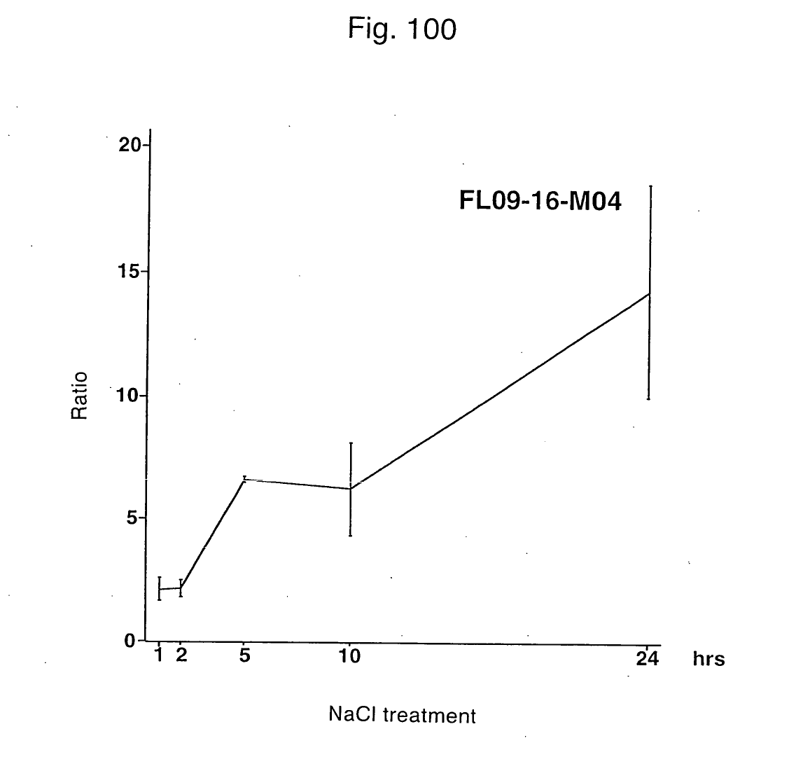

FIG. 101 is a characteristic graph showing the relationship between dehydration treatment time and expression ratio regarding FL11-01-J18;

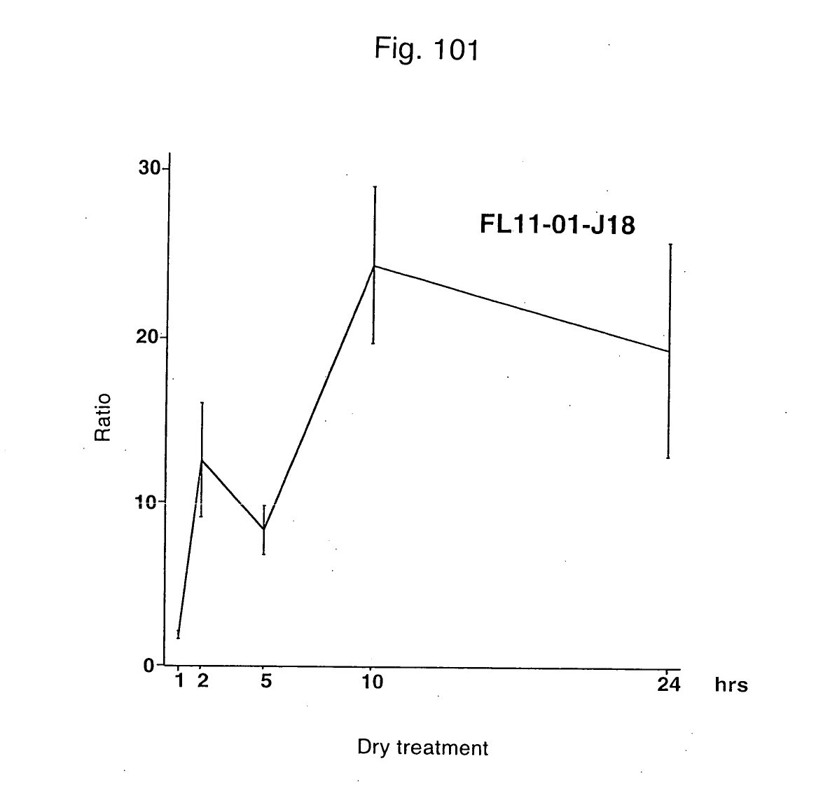

FIG. 102 is a characteristic graph showing the relationship between high salt treatment time and expression ratio regarding FL11-07-D13;

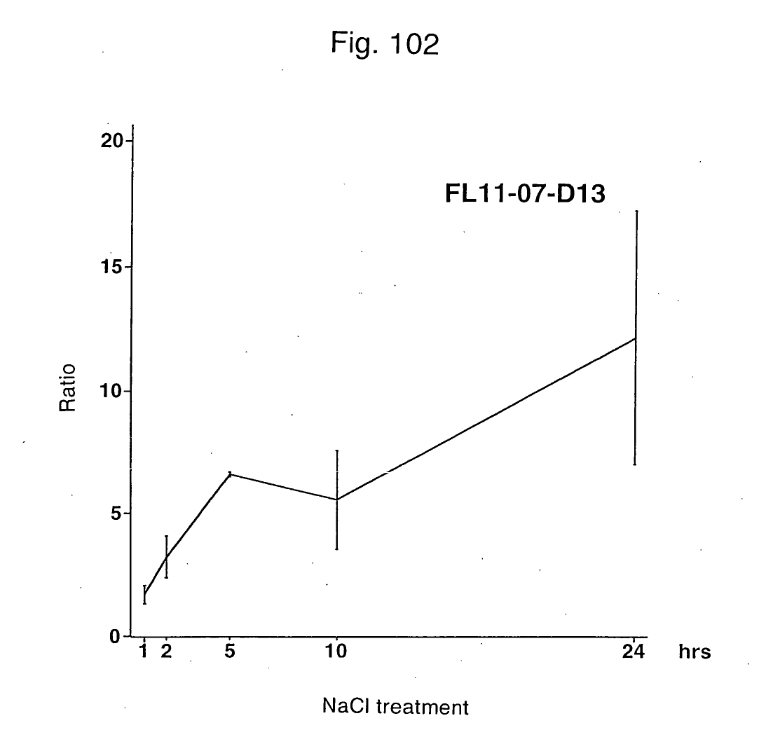

FIG. 103 is a characteristic graph showing the relationship between high salt treatment time and expression ratio regarding FL11-07-F02;

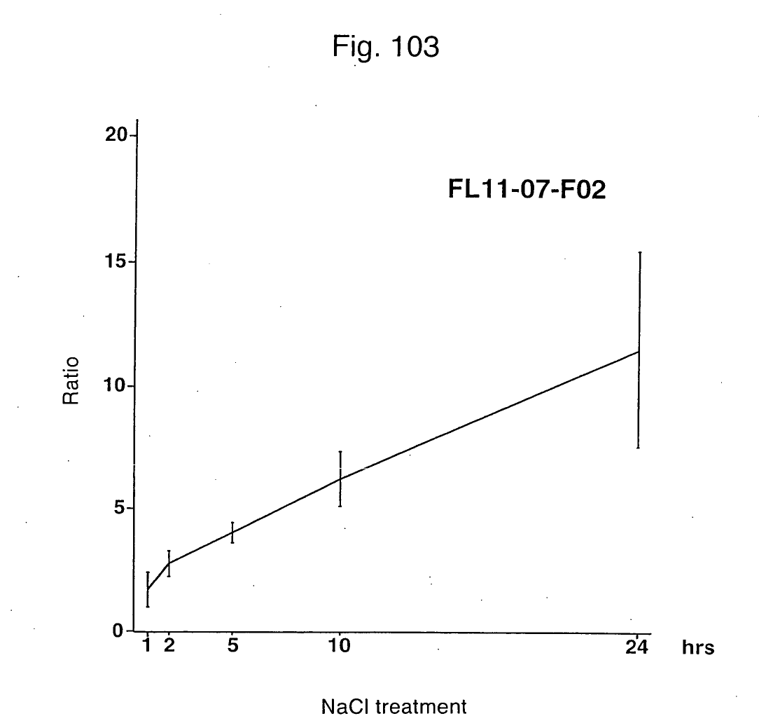

FIG. 104 is a characteristic graph showing the relationship between high salt treatment time and expression ratio regarding FL11-07-N15;

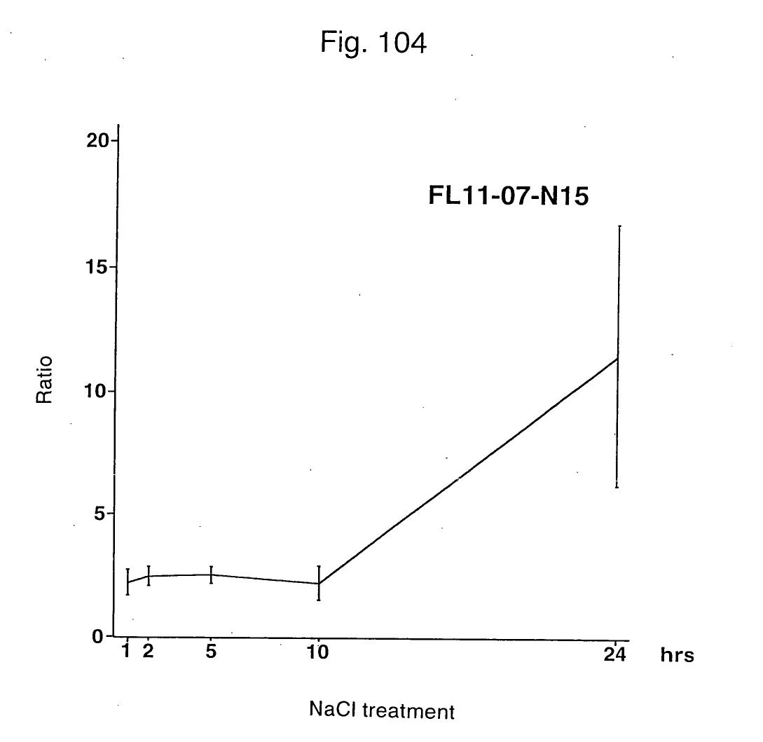

FIG. 105 is a characteristic graph showing the relationship between high salt treatment time and expression ratio regarding FL11-10-D10;

FIG. 106 is a characteristic graph showing the relationship between high salt stress and expression ratio regarding RAFL08-16-G17;

FIG. 107 is a characteristic graph showing the relationship between drought stress and expression ratio regarding RAFL05-11-M11;

FIG. 108 is a characteristic graph showing the relationship between high salt stress and expression ratio regarding RAFL05-11-M11;

FIG. 109 is a characteristic graph showing the relationship between high salt stress and expression ratio regarding RAFL06-11-K21;

FIG. 110 is a characteristic graph showing the relationship between drought stress and expression ratio regarding RAFL06-11-K21;

FIG. 111 is a characteristic graph showing the relationship between drought stress and expression ratio regarding RAFL06-08-H20;

FIG. 112 is a characteristic graph showing the relationship between high salt stress and expression ratio regarding RAFL06-08-H20;

FIG. 113 is a characteristic graph showing the relationship between high salt stress and expression ratio regarding RAFL05-16-H23;

FIG. 114 is a characteristic graph showing the relationship between drought stress and expression ratio regarding RAFL05-16-H23;

FIG. 115 is a characteristic graph showing the relationship between drought stress and expression ratio regarding RAFL08-16-D06;

FIG. 116 is a characteristic graph showing the relationship between drought stress and expression ratio regarding RAFL07-10-G04;

FIG. 117 is a characteristic graph showing the relationship between drought stress and expression ratio regarding RAFL04-17-D16;

FIG. 118 is a characteristic graph showing the relationship between drought stress and expression ratio regarding RAFL05-19-M20;

FIG. 119 is a characteristic graph showing the relationship between high salt stress and expression ratio regarding RAFL08-11-M13;

FIG. 120 is a characteristic graph showing the relationship between drought stress and expression ratio regarding RAFL04-15-K19;

FIG. 121 is a characteristic graph showing the relationship between cold stress and expression ratio regarding RAFL04-15-K19;

FIG. 122 is a characteristic graph showing the relationship between drought stress and expression ratio regarding RAFL05-11-L01;

FIG. 123 is a characteristic graph showing the relationship between high salt stress and expression ratio regarding RAFL05-11-L01;

FIG. 124 is a characteristic graph showing the relationship between drought stress and expression ratio regarding RAFL05-14-C11;

FIG. 125 is a characteristic graph showing the relationship between high salt stress and expression ratio regarding RAFL05-19-G24;

FIG. 126 is a characteristic graph showing the relationship between drought stress and expression ratio regarding RAFL05-19-G24;

FIG. 127 is a characteristic graph showing the relationship between cold stress and expression ratio regarding RAFL05-19-G24;

FIG. 128 is a characteristic graph showing the relationship between drought stress and expression ratio regarding RAFL05-20-N02;

FIG. 129 is a, characteristic graph showing the relationship between drought stress and expression ratio regarding RAFL05-18-H12;

FIG. 130 is a characteristic graph showing the relationship between high salt stress and expression ratio regarding RAFL05-18-H12;

FIG. 131 is a characteristic graph showing the relationship between high salt stress and expression ratio regarding RAFL05-19-E19;

FIG. 132 is a characteristic graph showing the relationship between high salt stress and expression ratio regarding RAFL06-10-D22;

FIG. 133 is a characteristic graph showing the relationship between high salt stress and expression ratio regarding RAFL06-12-M01;

FIG. 134 is a characteristic graph showing the relationship between drought stress and expression ratio regarding RAFL06-12-M01;

FIG. 135 is a characteristic graph showing the relationship between drought stress and expression ratio regarding RAFL05-14-D24;

FIG. 136 is a characteristic graph showing the relationship between high salt stress and expression ratio regarding RAFL05-14-D24;

FIG. 137 is a characteristic graph showing the relationship between cold stress and expression ratio regarding RAFL05-20-N17;

FIG. 138 is a characteristic graph showing the relationship between drought stress and expression ratio regarding RAFL05-20-N17;

FIG. 139 is a characteristic graph showing the relationship between drought stress and expression ratio regarding RAFL04-17-F21;

FIG. 140 is a characteristic graph showing the relationship between drought stress and expression ratio regarding RAFL09-12-N16;

FIG. 141 is a characteristic graph showing the relationship between drought stress and expression ratio regarding AFL05-19-I05;

FIG. 142 is a characteristic graph showing the relationship between high salt stress and expression ratio regarding RAFL05-19-I05;

FIG. 143 is a characteristic graph showing the relationship between high salt stress and expression ratio regarding RAFL05-21-I22;

FIG. 144 is a characteristic graph showing the relationship between drought stress and expression ratio regarding RAFL08-11-H20;

FIG. 145 is a characteristic graph showing the relationship between high salt stress and expression ratio regarding RAFL08-11-H20;

FIG. 146 is a characteristic graph showing the relationship between high salt stress and expression ratio regarding RAFL05-21-C17;

FIG. 147 is a characteristic graph showing the relationship between drought stress and expression ratio regarding RAFL05-21-C17;

FIG. 148 is a characteristic graph showing the relationship between high salt stress and expression ratio regarding RAFL05-08-D06;

FIG. 149 is a characteristic graph showing the relationship between drought stress and expression ratio regarding RAFL05-20-M16;

FIG. 150 is a characteristic graph showing the relationship between high salt stress and expression ratio regarding RAFL05-20-M16;

FIG. 151 is a characteristic graph showing the relationship between drought stress and expression ratio regarding RAFL11-01-J18;

FIG. 152 is a characteristic graph showing the relationship between high salt stress and expression ratio regarding RAFL11-01-J18;

FIG. 153 is a characteristic graph showing the relationship between high salt stress and expression ratio regarding RAFL11-09-C20;

FIG. 154 is a characteristic graph showing the relationship between high salt stress and expression ratio regarding RAFL05-18-N16;

FIG. 155 is a characteristic graph showing the relationship between drought stress and expression ratio regarding RAFL11-10-D10;

FIG. 156 is a characteristic graph showing the relationship between high salt stress and expression ratio regarding RAFL11-10-D10;

FIG. 157 is a characteristic graph showing the relationship between drought stress and expression ratio regarding RAFL04-17-N22;

FIG. 158 is a characteristic graph showing the relationship between high salt stress and expression ratio regarding RAFL04-17-N22;

FIG. 159 is a characteristic graph showing the relationship between drought stress and expression ratio regarding RAFL05-09-G15;

FIG. 160 is a characteristic graph showing the relationship between high salt stress and expression ratio regarding RAFL05-09-G15;

FIG. 161 is a characteristic graph showing the relationship between drought stress and expression ratio regarding RAFL05-21-L12; and

FIG. 162 is a characteristic graph showing the relationship between high salt stress and expression ratio regarding RAFL05-21-L12.

### BEST MODE FOR CARRYING OUT THE INVENTION

Hereinafter, the present invention will be further explained in detail by way of examples, which should not be construed as limiting the scope of the present invention.

### EXAMPLE 1.

### Isolation of Promoter

1. Materials and Methods

(1) Arabidopsis cDNA Clone

A microarray was constructed by using about 7,000 cDNA molecules in total including genes isolated from an Arabidopsis full-length cDNA libraries, responsive-to-dehydration (RD) genes, early responsive-to-dehydration (ERD) genes, kin 1 genes, kin2 genes, and cor15a genes; α-tubulin genes as an internal standard; and mouse nicotinic acetylcholine receptor epsilon subunit (nAChRE) genes and mouse glucocorticoid receptor homologous genes, as negative controls.
Positive control: dehydration-inducible genes (responsive-to-dehydration genes: rd, and early responsive-to-dehydration genes: erd)
Internal standard: α-tubulin gene
Negative control: mouse nicotinic acetylcholine receptor epsilon subunit (nAChRE) genes and mouse glucocorticoid receptor homologous genes, which do not substantially have homology with any given sequence in an Arabidopsis database for analyzing non-specific hybridization.
   (2) Arabidopsis Full-Length cDNA Microarray

The present inventors have constructed full-length cDNA libraries from an Arabidopsis plant body under different conditions (e.g., dehydration treatment, cold treatment and non-treatment in different growth stages from budding to maturation of seeds) by the biotinylated CAP trapper method. From the full-length cDNA libraries, the present inventors isolated individually about 7,000 independent Arabidopsis full-length cDNA molecules. The cDNA fragments, which were amplified by PCR, were arranged on a slide glass in accordance with a known method (Eisen and Brown, 1999). The present inventors prepared a full-length cDNA microarray containing about 7,000 Arabidopsis full-length cDNA molecules, which contain the genes below.

(3) Isolation of Dehydration-, Cold-, High Salt-, and ABA-Inducible Genes Using cDNA Microarray

In this example, dehydration-, cold-, high salt-, and ABA-inducible genes were isolated by using a full-length cDNA microarray containing about 7,000 Arabidopsis full-length cDNA molecules.

Probes of a plant treated with different stresses and an untreated plant with stress and labeled with Cy3 and Cy5 fluorescent dyes were mixed. The probes were hybridized with the full-length cDNA microarray containing about 7,000 Arabidopsis full-length cDNA molecules. By such a double labeling of a pair of cDNA probes wherein one of the mRNA samples was labeled with Cy3-dUTP and the other was labeled with Cy5-dUTP, hybridization with DNA elements on a microarray can be performed simultaneously, with the result that quantitative determination of gene expression under two different conditions (that is, stressed and unstressed conditions) can be directly and easily performed. The hybridized microarray was scanned by two discrete laser channels for Cy3 and Cy5 emission from each of DNA elements. Subsequently, the intensity ratio between two fluorescent signals from each DNA element was determined. Based on the relative value of the intensity ratio, a change of differential expression of genes represented as a cDNA spot on the microarray was determined. In this example, an α-tubulin gene, whose expression level was almost equivalent under two different experimental conditions was used, as an internal control gene.

In the full-length cDNA microarray containing about 7,000 Arabidopsis full-length cDNA molecules, a procedure for identifying dehydration-, cold-, high salt-, and ABA-inducible genes will be explained.
1) Both mRNA molecules derived from a plant treated with one of the stresses mentioned above and mRNA molecules derived from a wild-type plant unstressed were used to prepare Cy3-labeled cDNA and Cy5-labeled cDNA probes, respectively. These cDNA probes were mixed and hybridized with the cDNA microarray. In this example, an α-tubulin gene, which exhibits almost the same expression level under two type conditions, was as used as an internal control gene. A gene that exhibits the expression ratio of dehydration:unstressed, cold:unstressed, or high salt:unstressed more than double of that of the α-tubulin gene was defined as an inducible gene by a stress given to the gene.
2) Both mRNA molecules derived from a 35S:DREB1A transgenic plant and mRNA molecules derived from a wild-type plant unstressed were used to prepare Cy3-labeled cDNA and Cy5-labeled cDNA probes, respectively. These cDNA probes were mixed and hybridized with a cDNA microarray. In this example, an α-tubulin gene exhibiting almost the same expression level under two type conditions was used as an internal control gene. A gene of 35S:DREB1A transgenic plant exhibiting an expression ratio more than double of that of a gene of the wild type plant unstressed was defined as a DREB1A target gene.

Both mRNA molecules derived from a plant treated with a stress and mRNA molecules derived from a wild-type plant unstressed were used to prepare Cy3-labeled cDNA and Cy5-labeled cDNA probes, respectively. These cDNA probes were mixed and hybridized with a cDNA microarray. The same experiment was repeated three times to evaluate the reproducibility of microarray analysis. When the same mRNA sample was hybridized with various microarrays, a good correlation was observed. A gene that exhibits an expression ratio (dehydration/unstressed, cold/unstressed) more than double of that of the α-tubulin gene was defined as an inducible gene by a stress given to the gene.

(4) Analysis of Sequence

Plasmid DNA extracted by a plasmid preparation device (NA 100) manufactured by Kurabo was sequenced to find homology of gene sequences. The DNA sequence was determined by a dye terminator cycle sequencing method using a DNA sequencer (ABI PRISM 3700. PE Applied Biosystems, CA, USA). Based on the GenBank/EMBL database, homology of sequences was found by using the BLAST program.

(5) Amplification of cDNA

λZAPII (Carninci et al., 1996) was used as a vector for constructing a cDNA library. The cDNA inserted in a vector for the library was amplified by PCR using complementary primers to the sequences of both sides of the cDNA.

The sequences of primers are as follows:

| | |
|---|---|
| FL forward 1224: | |
| 5′-CGCCAGGGTTTTCCCAGTCACGA | (SEQ ID NO: 91) |
| FL reverse 1233: | |
| 5′-AGCGGATAACAATTTCACACAGGA | (SEQ ID NO: 92) |

To 100 μl of a PCR solution mixture (0.25 mM dNTP, 0.2 μM PCR primer, 1×Ex Taq Buffer, and 1.25 U Ex Taq polymerase (manufactured by Takara Shuzo)), a plasmid (1 to 2 ng) was added as a template. PCR was performed under the following conditions: an initial reaction at 94° C. for 3 minutes, 35 cycles each consisting of 95° C. for one minute, 60° C. for 30 seconds and 72° C. for 3 minutes, and a final reaction at 72° C. for 3 minutes. After a PCR product was precipitated with ethanol, the precipitate was dissolved in 25 μl of 3×SSC and then subjected to electrophoresis using 0.7% agarose gel. The quality of the DNA obtained and amplification efficiency of PCR were confirmed.

(6) Construction of cDNA Microarray

Using a gene tip microarray stamp machine GTMASS SYSTEM (manufactured by Nippon Laser & Electronics Lab.), 0.5 μl of a PCR product (100 to 500 ng/ml) was loaded from a 384-well microtiter plate to form spots of the PCR product (5 nl for each) at intervals of 280 μm on 6 micro slide glasses (S7444, manufactured Matsunami) coated with poly-L lysine. To spot DNA in an equivalent amount, the slide after printing was placed in a beaker containing heated distilled water to moisten it and placed at 100° C. for 3 seconds to dry it. After the slide was placed on a slide rack, the rack was transferred into a glass chamber. To the glass chamber, a blocking solution (15 ml of 1M sodium borate salt (pH 8.0), 5.5 g succinic anhydrous compound (Wako), and 335 ml of 1-methyl-2-pyrrolidon (Wako)) was poured. After the glass chamber housing the slide rack was shaken up and down 5 times and gently shaking for 15 minutes, the slide rack was transferred to a glass chamber containing boiling water, shaken 5 times, and allowed to stand alone for 2 minutes. Thereafter, the slide rack was transferred to a glass chamber containing 95% ethanol, shaken 5 times, and centrifuged at 800 rpm for 30minutes.

(7) Plant Material and Isolation of RNA

As a plant material, use was made of a wild type Arabidopsis thaliana plant body which was seeded on an agar medium and grown for 3 weeks (Yamaguchi-Shinozaki and Shinozaki, 1994) and an Arabidopsis thaliana (Colombian species) plant body into which DREB1A cDNA(Kasuga et al., 1999) connected to a 35S promoter of a cauliflower mosaic virus was introduced. Dehydration- and cold-stress treatments were performed in accordance with the method of Yamaguchi-Shinozaki and Shinozaki (1994). More specifically, dehydration treatment was performed by pulling a plant body out of the agar medium, placing it on a filter, and dried at a temperature of 22° C. and a relative humidity of 60%. The cold treatment was performed by transferring a plant body grown at 22° C. to 4° C. High salt stress treatment was performed by growing a plant body at an aqueous solution containing 250 mM NaCl.

After wild type plant bodies were exposed to stress-treatment for 2 or 10 hours, a sample was taken from each of plant bodies and stored in cryogenic conditions with liquid nitrogen. Furthermore, wild type and DREB1A overexpresstion-type transformants cultured in an agar medium without kanamycin were subjected to an experiment for identifying a DREB1A target gene. The DREB1A overexpresstion-type transformant was not treated with stresses. The total RNA was isolated from a plant body by using ISOGEN (Nippon gene, Tokyo, Japan) and mRNA was isolated and purified by Oligotex-dT30 mRNA purification kit (Takara, Tokyo, Japan).

(8) Fluorescent Labeling of Probe

Each of the mRNA samples was subjected to a reverse transcription reaction in the presence of Cy3 dUTP or Cy5 dUTP (Amersham Pharmacia). The composition of the buffer (30 μl) used in the reverse transcription reaction is shown in Table 2.

TABLE 2

| | |
|---|---|
| poly(A)⁺ RNA with 6 μg oligo(dT) 18-mer | 1 μg |
| 10 mM DTT | |
| 500 μM dATP, dCTP and dGTP | |
| 200 μM dTTP | |
| 100 μM Cy3 dUTP or Cy5 dUTP | |
| 400 units of SuperScript II Reverse Transcriptase | |
| (Life technologies) | |
| 1X Superscript First Strand Synthesis Buffer | |
| (Life technologies) | |
| Total | 30 μL |

After reaction was performed at 42° C. for one hour, two samples (labeled with Cy3 and Cy5) were mixed to obtain a reaction mixture. To this reaction mixture, 15 μl of 0.1 M NaOH and 1.5 μl of 20 mM EDTA were added and treated at 70° C. for 10 minutes. Further, 15 μl of 0.1 M HCl was added to the reaction mixture, a sample was taken and transferred to a Micro con 30 micro concentrator (Amicon). 400 μl of TE buffer was added to the sample and centrifuged until the volume of the buffer reached 10 to 20 μl. The effulent was discarded. 400 μl of TE buffer and 20 μl of 1 mg/ml human Cot-1 DNA (Gibco BRL) were added to the resultant mixture and the mixture was again centrifuged. The labeled samples were centrifugally collected and several μl of distilled water was added thereto. The obtained probes, 2 μl of 10 μg/μl yeast tRNA, 2 μl of 1 μg/μl pd(A)₁₂₋₁₈ (Amersham Pharmacia), 3.4 ml of 20×SSC, and 0.6 μl of 10% SDS were added. Further, the samples were denatured at 100° C. for 1 minute and placed at room temperature for 30 minutes and thereafter used in hybridization.

(9) Microarray Hybridization and Scanning

A probe was subjected to high-speed centrifugation for one minute by a benchtop micro centrifuge. To avoid generation of bubbles, the probe was placed at the center of an array and a cover slip was placed thereon. Four drops of 5 μl of 3×SSC were dropped on a slide glass and a chamber was kept at a suitable humidity to prevent the probe from being dried during hybridization. After the slide glass was placed in a cassette for hybridization (THC-1, BM machine) and the cassette was sealed, hybridization treatment was performed at 65° C. for 12 to 16 hours. The slide glass was taken out from the cassette and placed on the slide rack. After the cover slip was carefully removed in solution 1 (2×SSC, 0.1% SDS), the rack was washed while shaking and transferred into solution 2 (1×SSC) to wash for 2 minutes. The rack was further transferred to solution 3 (0.2×SSC), allowed to stand for 2 minutes, and centrifuged at 800 rpm for 1 min to dry.

The microarray was scanned at a resolution of 10 μm per pixel by use of a scanning laser microscope (ScanArray 4000; GSI Lumonics, Watertown, Mass.). As a program for analyzing microarray data, Imagene Ver 2.0 (BioDiscovery) and QuantArray (GSI Lumonics) were used.

(10) Northern Analysis

Northern analysis was performed using total RNA, (Yamaguchi-Shinozaki and Shinozaki, 1994). DNA fragments were isolated from the Arabidopsis thaliana full-length cDNA library by a PCR method and used as probes for Northern hybridization.

(11) Determination of Promoter Region

Based on the genomic information of Arabidopsis thaliana in a data base (GenBank/EMBL, ABRC), a promoter region was analyzed by using the BLAST program for gene analysis.

2. Results

(1) Stress-Inducible Gene

Fluorescent-labeled cDNA was prepared by subjecting mRNA isolated from an unstressed Arabidopsis thaliana plant to a reverse transcription reaction in the presence of Cy5-dUTP. A second probe labeled with Cy3-dUTP was prepared from a plant treated at low temperature for 2 hours. Both probes were simultaneously hybridized with a cDNA microarray comprising about 7,000 Arabidopsis thaliana cDNA clones and then a pseudo color image was created.

Genes induced and suppressed by a stress are represented by a red signal and green signal, respectively. Genes expressed at virtually the same level in both treatments are represented by a yellow signal. The intensity of each spot corresponds to the absolute value of the expression level of each gene. It is shown that a cold-inducible gene (rd29A) is represented by a red signal whereas an α-tubulin gene (an internal control) is represented by a yellow signal.

(2) Identification of Promoter Region

As a result of identifying a promoter region, the promoter gene regions of 90 types of genes were obtained in a full-length cDNA microarray containing about 7,000 of Arabidopsis full-length cDNA molecules. The name of these 90 types of genes and their promoter sequences are summarized in Table 3

TABLE 3

| Name of gene | SEQ ID NO: |
|---|---|
| FL03-07-F12 | SEQ ID NO: 1 |
| FL04-12-F24 | SEQ ID NO: 2 |
| FL04-14-N10 | SEQ ID NO: 3 |
| FL04-14-P24 | SEQ ID NO: 4 |
| FL04-17-I03 | SEQ ID NO: 5 |
| FL04-17-M08 | SEQ ID NO: 6 |
| FL04-17-M22 | SEQ ID NO: 7 |
| FL05-05-A17 | SEQ ID NO: 8 |
| FL05-05-F20 | SEQ ID NO: 9 |
| FL05-05-G20 | SEQ ID NO: 10 |
| FL05-09-N09 | SEQ ID NO: 11 |
| FL05-10-J09 | SEQ ID NO: 12 |
| FL05-10-M08 | SEQ ID NO: 13 |
| FL05-11-H09 | SEQ ID NO: 14 |
| FL05-12-H13 | SEQ ID NO: 15 |
| FL05-13-I20 | SEQ ID NO: 16 |
| FL05-14-E15 | SEQ ID NO: 17 |
| FL05-14-E16 | SEQ ID NO: 18 |
| FL05-16-F03 | SEQ ID NO: 19 |
| FL05-16-H23 | SEQ ID NO: 20 |
| FL05-18-M07 | SEQ ID NO: 21 |
| FL05-18-O21 | SEQ ID NO: 22 |
| FL05-19-F21 | SEQ ID NO: 23 |
| FL05-19-O22 | SEQ ID NO: 24 |
| FL05-21-K17 | SEQ ID NO: 25 |
| FL06-10-F03 | SEQ ID NO: 26 |
| FL06-12-H12 | SEQ ID NO: 27 |
| FL07-12-I23 | SEQ ID NO: 28 |
| FL08-08-H23 | SEQ ID NO: 29 |
| FL08-08-O14 | SEQ ID NO: 30 |
| FL08-09-M05 | SEQ ID NO: 31 |
| FL08-10-K08 | SEQ ID NO: 32 |
| FL08-11-P07 | SEQ ID NO: 33 |
| FL08-13-F10 | SEQ ID NO: 34 |
| FL08-19-D04 | SEQ ID NO: 35 |
| FL08-19-G15 | SEQ ID NO: 36 |
| FL09-06-B11 | SEQ ID NO: 37 |
| FL09-07-G17 | SEQ ID NO: 38 |
| FL09-10-A12 | SEQ ID NO: 39 |
| FL09-13-P15 | SEQ ID NO: 40 |
| FL02-05-I05 | SEQ ID NO: 41 |
| FL04-12-N15 | SEQ ID NO: 42 |
| FL04-16-P21 | SEQ ID NO: 43 |
| FL04-17-N22 | SEQ ID NO: 44 |
| FL04-20-P19 | SEQ ID NO: 45 |
| FL02-09-H01 | SEQ ID NO: 46 |
| FL05-01-D08 | SEQ ID NO: 47 |
| FL05-02-G08 | SEQ ID NO: 48 |
| FL05-02-O17 | SEQ ID NO: 49 |
| FL05-07-L13 | SEQ ID NO: 50 |
| FL05-08-B14 | SEQ ID NO: 51 |
| FL05-09-N10 | SEQ ID NO: 52 |
| FL05-11-L01 | SEQ ID NO: 53 |
| FL05-12-J09 | SEQ ID NO: 54 |
| FL05-14-D24 | SEQ ID NO: 55 |
| FL05-14-F20 | SEQ ID NO: 56 |
| FL05-14-I08 | SEQ ID NO: 57 |
| FL05-15-C04 | SEQ ID NO: 58 |
| FL05-15-E19 | SEQ ID NO: 59 |
| FL05-18-A06 | SEQ ID NO: 60 |
| FL05-18-H15 | SEQ ID NO: 61 |
| FL05-19-C02 | SEQ ID NO: 62 |
| FL05-20-M16 | SEQ ID NO: 63 |
| FL05-20-N18 | SEQ ID NO: 64 |
| FL05-21-E06 | SEQ ID NO: 65 |
| F105-21-L12 | SEQ ID NO: 66 |
| FL06-07-B08 | SEQ ID NO: 67 |
| FL06-08-H20 | SEQ ID NO: 68 |
| FL06-09-N04 | SEQ ID NO: 69 |
| FL06-11-K21 | SEQ ID NO: 70 |
| FL07-07-G15 | SEQ ID NO: 71 |
| FL07-12-D17 | SEQ ID NO: 72 |
| FL08-11-C23 | SEQ ID NO: 73 |
| FL08-13-G20 | SEQ ID NO: 74 |
| FL08-15-M21 | SEQ ID NO: 75 |
| FL08-18-N19 | SEQ ID NO: 76 |
| FL08-19-C07 | SEQ ID NO: 77 |
| FL08-19-P05 | SEQ ID NO: 78 |
| FL09-07-G09 | SEQ ID NO: 79 |
| FL09-07-G15 | SEQ ID NO: 80 |
| FL09-10-J18 | SEQ ID NO: 81 |
| FL09-11-I12 | SEQ ID NO: 82 |
| FL09-12-B03 | SEQ ID NO: 83 |
| FL09-16-I11 | SEQ ID NO: 84 |
| FL09-16-M04 | SEQ ID NO: 85 |
| FL11-01-J18 | SEQ ID NO: 86 |
| FL11-07-D13 | SEQ ID NO: 87 |
| FL11-07-F02 | SEQ ID NO: 88 |
| FL11-07-N15 | SEQ ID NO: 89 |
| FL11-10-D10 | SEQ ID NO: 90 |

(3) The Relationship between Stress Treatment Time and Expression Ratio

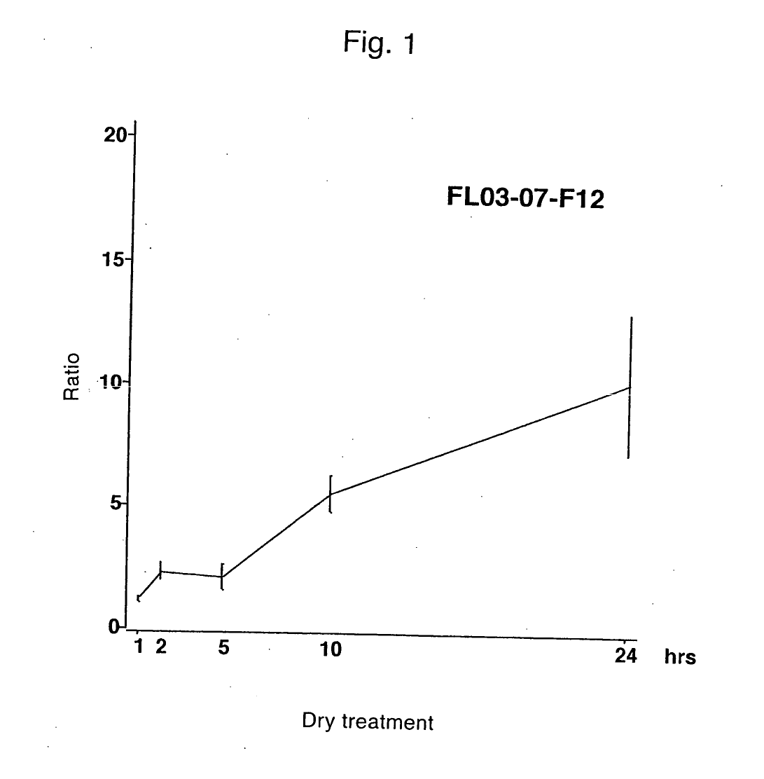

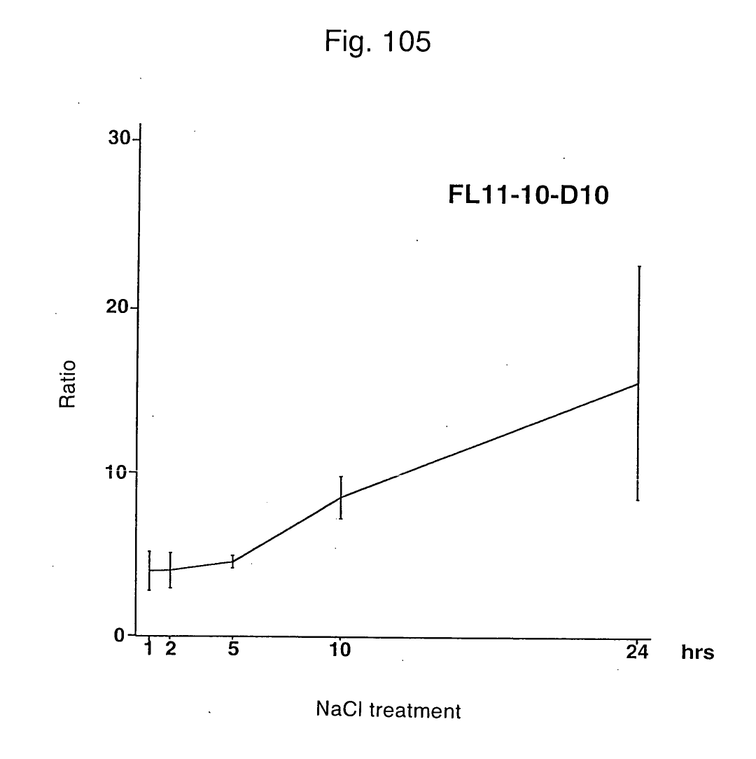

The 90 types of stress inducible genes isolated above were analyzed for the relationship between stress treatment time and expression ratio. The results are shown in FIGS. 1 to 105. The relationship between 90 types of genes and stress treatment are shown in Table 4.

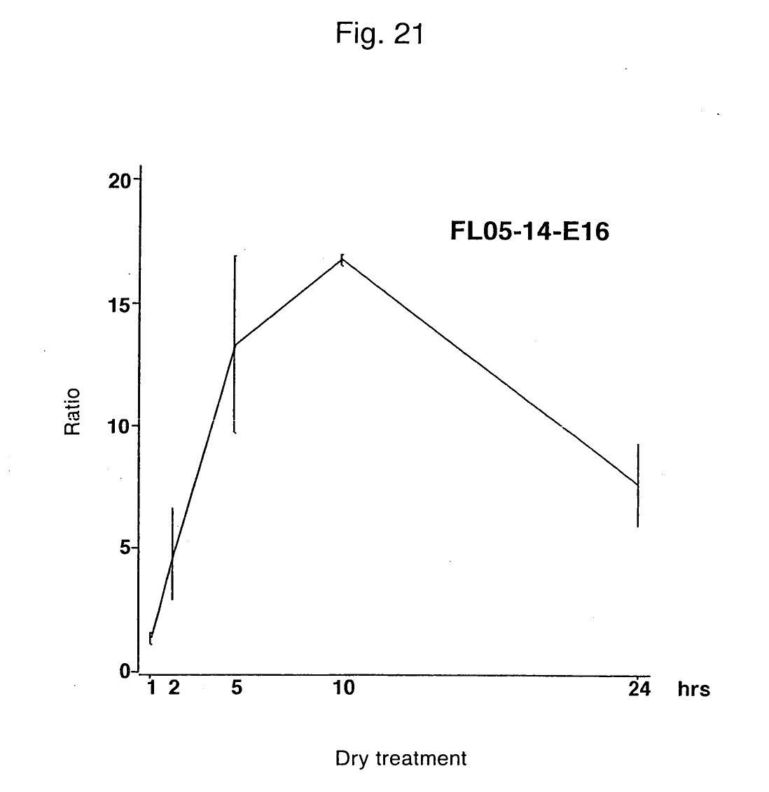

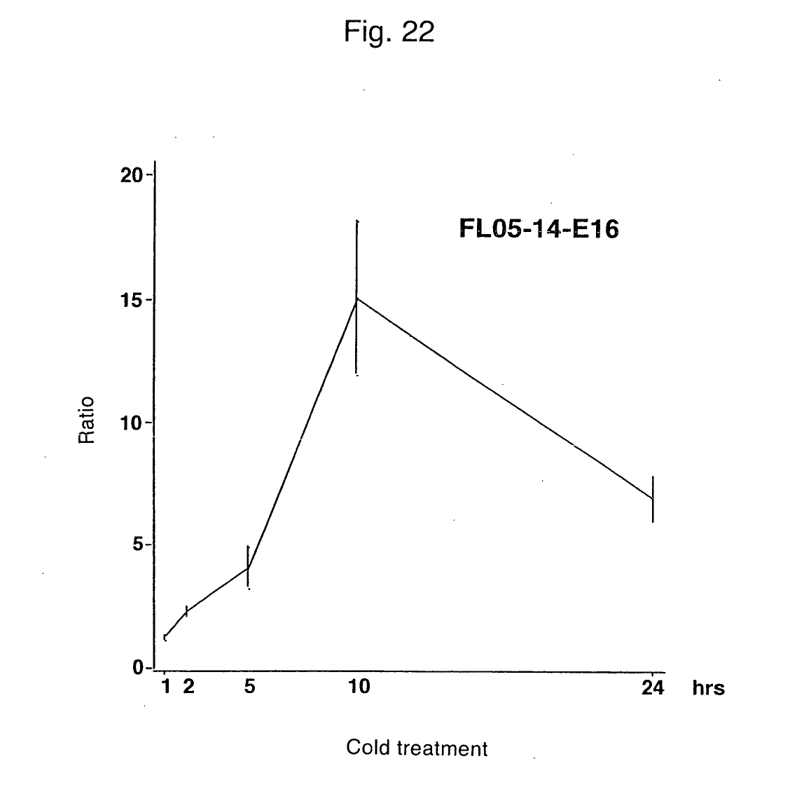

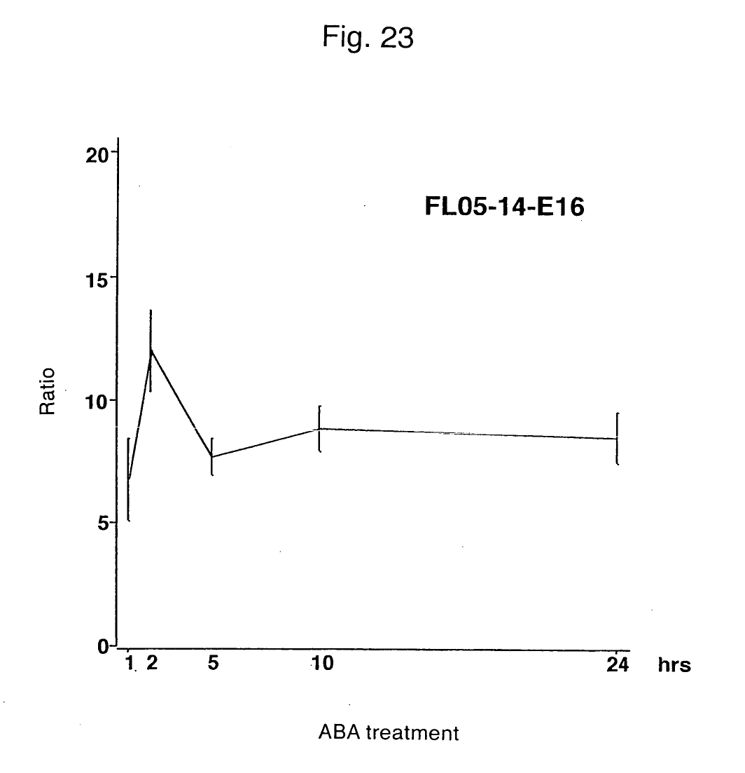

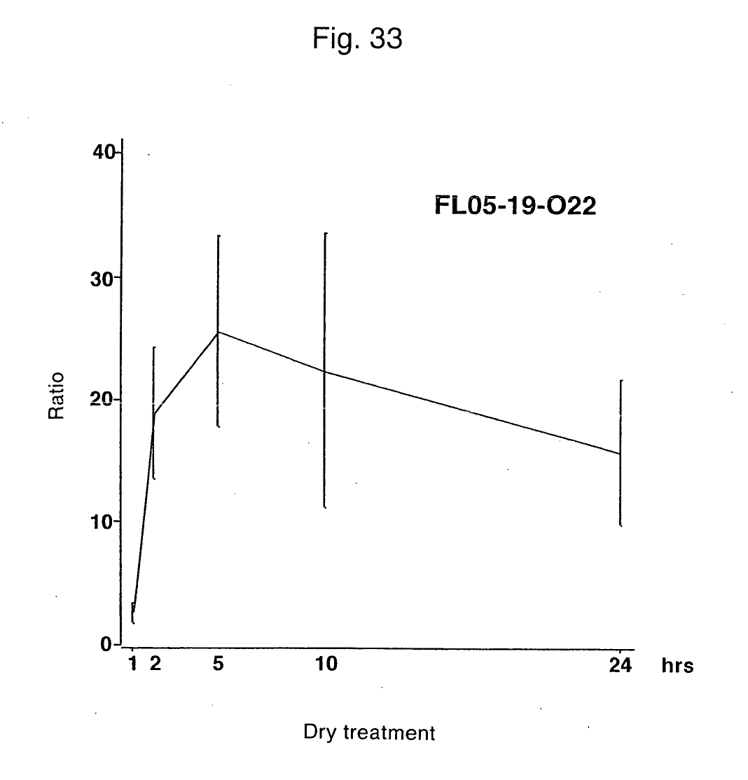

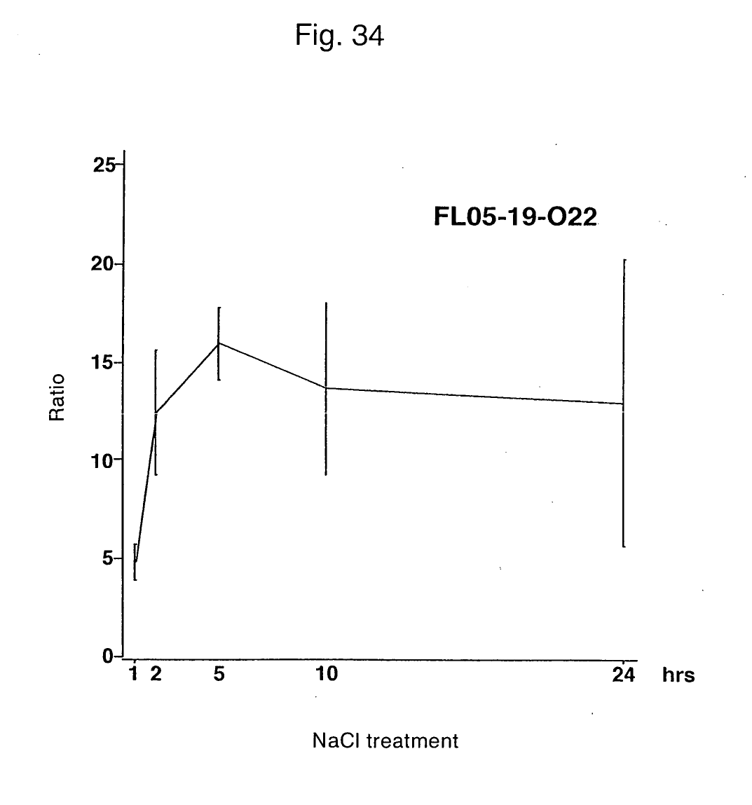

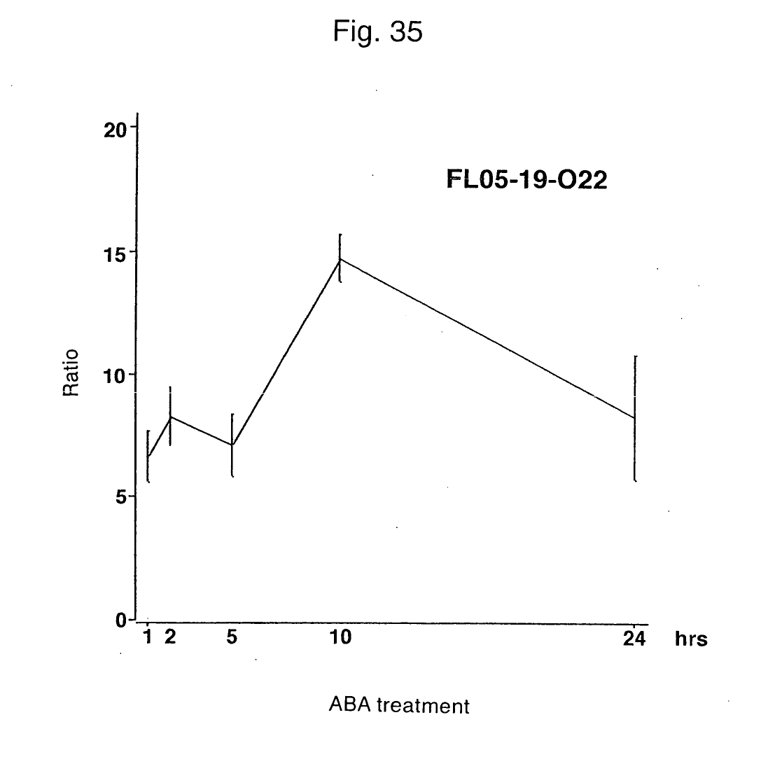

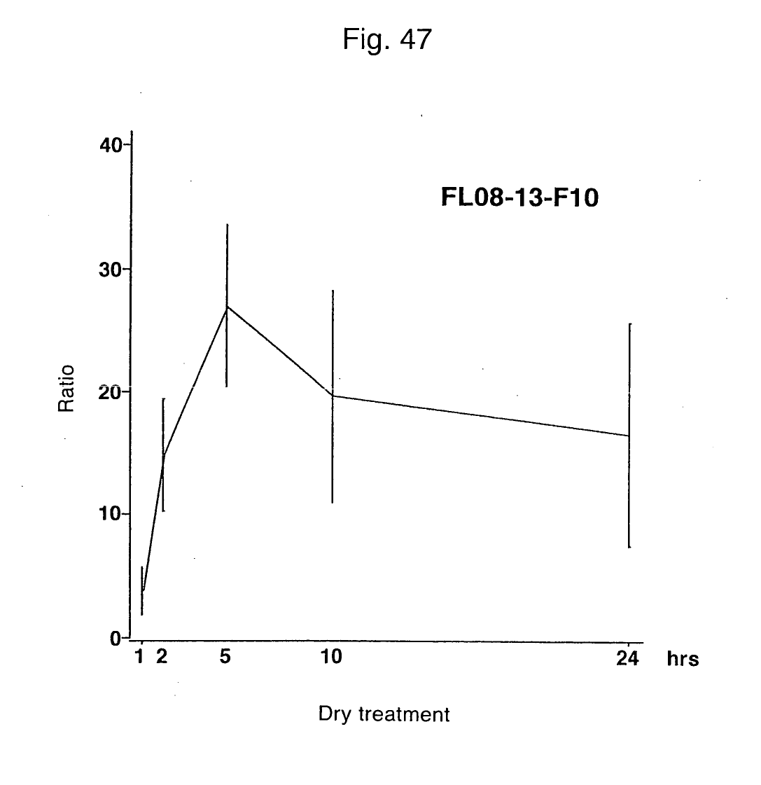

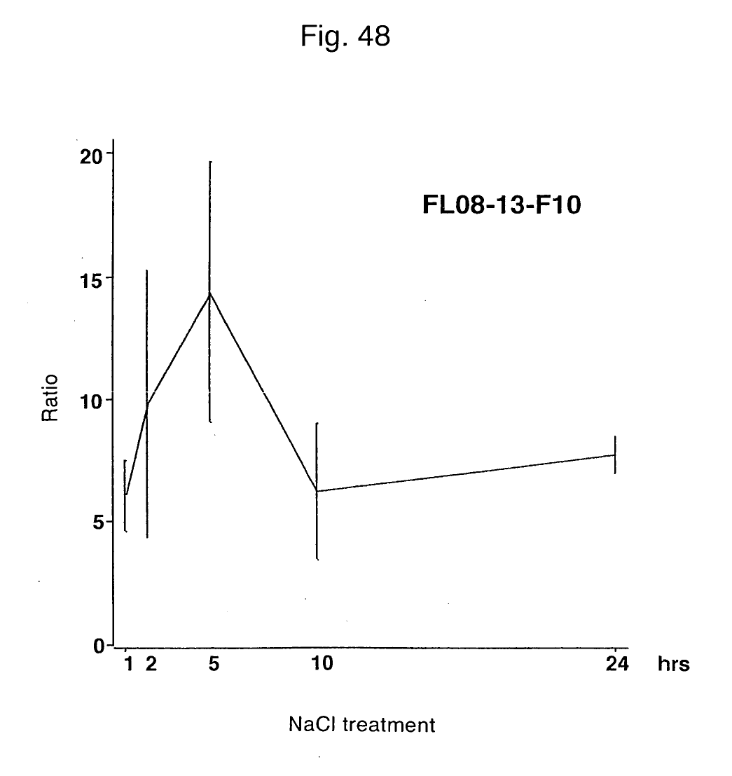

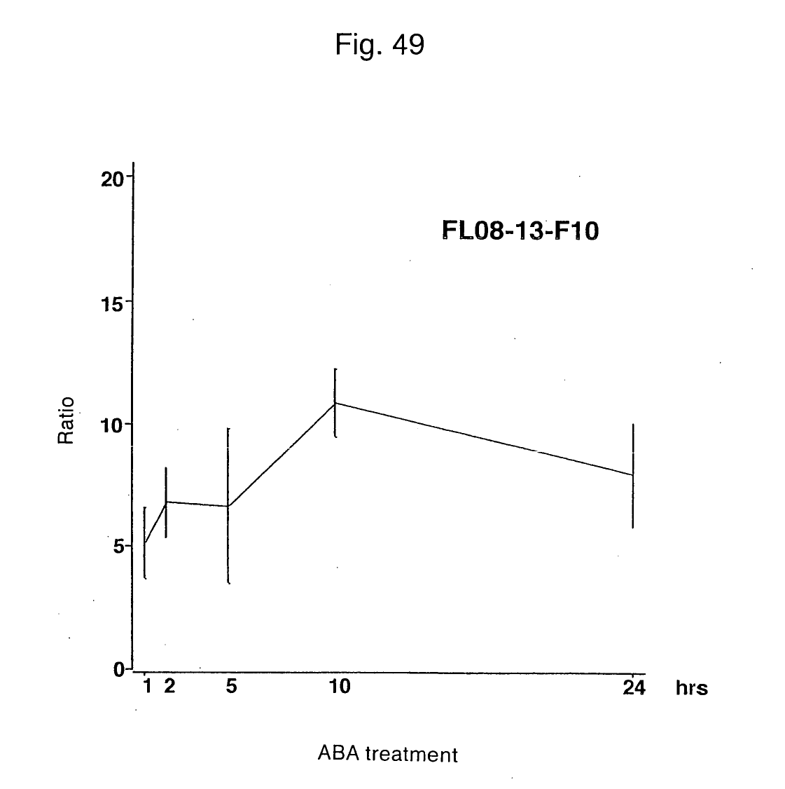

TABLE 4

| Name of gene | Type of applied stress | Drawing |
|---|---|---|
| FL03-07-F12 | Dehydration | |
| FL04-12-F24 | Exposure to cold | |
| FL04-14-N10 | Dehydration | |
| FL04-14-P24 | Dehydration | |
| FL04-17-I03 | Dehydration, Exposure to a high level salt solution | |
| FL04-17-M08 | Exposure to a high level salt solution | |
| FL04-17-M22 | Dehydration | |
| FL05-05-A17 | Dehydration | |
| FL05-05-F20 | Dehydration | |
| FL05-05-G20 | Dehydration | |
| FL05-09-N09 | Dehydration | |
| FL05-10-J09 | Dehydration, Exposure to a high level salt solution | |
| FL05-10-M08 | Exposure to a high level salt solution | |
| FL05-11-H09 | Exposure to a high level salt solution | |
| FL05-12-H13 | Dehydration, Exposure to a high level salt solution | |
| FL05-13-I20 | ABA treatment | |
| FL05-14-E15 | Dehydration | |
| FL05-14-E16 | Dehydration, Exposure to cold, ABA treatment | FIGS. 21-23 |
| FL05-16-F03 | Dehydration, ABA treatment | |
| FL05-16-H23 | Dehydration, Exposure to a high level salt solution | |
| FL05-18-M07 | Dehydration, ABA treatment | |
| FL05-18-O21 | ABA treatment | |
| FL05-19-F21 | Dehydration, ABA treatment | |
| FL05-19-O22 | Dehydration, Exposure to a high level salt solution, ABA | FIGS. 33-35 |
| | treatment | |
| FL05-21-K17 | Exposure to a high level salt solution | |
| FL06-10-F03 | ABA treatment | |
| FL06-12-H12 | Dehydration, Exposure to a high level salt solution | |
| FL07-12-I23 | Exposure to a high level salt solution | |
| FL08-08-H23 | Exposure to a high level salt solution | |
| FL08-08-O14 | Dehydration | |
| FL08-09-M05 | Dehydration | |
| FL08-10-K08 | Exposure to a high level salt solution | |
| FL08-11-P07 | Dehydration, Exposure to cold | |
| FL08-13-F10 | Dehydration, Exposure to a high level salt solution, ABA | FIGS. 47-49 |
| | treatment | |
| FL08-19-D04 | Dehydration | |
| FL08-19-G15 | Exposure to a high level salt solution | |
| FL09-06-B11 | ABA treatment | |
| FL09-07-G17 | ABA treatment | |
| FL09-10-A12 | ABA treatment | |
| FL09-13-P15 | Dehydration | |
| FL02-05-I05 | Exposure to a high level salt solution | |
| FL04-12-N15 | Exposure to cold | |
| FL04-16-P21 | Dehydration | |
| FL04-17-N22 | Exposure to a high level salt solution | |
| FL04-20-P19 | Dehydration | |
| FL02-09-H01 | Dehydration | |
| FL05-01-D08 | Dehydration | |
| FL05-02-G08 | Exposure to a high level salt solution | |
| FL05-02-O17 | Dehydration | |
| FL05-07-L13 | Exposure to a high level salt solution | |
| FL05-08-B14 | Dehydration | |
| FL05-09-N10 | Dehydration | |
| FL05-11-L01 | Dehydration | |
| FL05-12-J09 | Dehydration | |
| FL05-14-D24 | Dehydration | |
| FL05-14-F20 | Dehydration | |
| FL05-14-I08 | Dehydration | |
| FL05-15-C04 | Dehydration | |
| FL05-15-E19 | Dehydration | |
| FL05-18-A06 | Dehydration | |
| FL05-18-H15 | Exposure to a high level salt solution | |
| FL05-19-C02 | Dehydration | |
| FL05-20-M16 | Dehydration | |
| FL05-20-N18 | Exposure to cold | |
| FL05-21-E06 | Dehydration | |
| FL05-21-L12 | Dehydration | |
| FL06-07-B08 | Dehydration | |
| FL06-08-H20 | Dehydration | |
| FL06-09-N04 | Dehydration | |
| FL06-11-K21 | Dehydration | |
| FL07-07-G15 | Exposure to a high level salt solution | |
| FL07-12-D17 | Exposure to a high level salt solution | |
| FL08-11-C23 | Dehydration | |
| FL08-13-G20 | Dehydration | |
| FL08-15-M21 | Dehydration | |
| FL08-18-N19 | Dehydration | |
| FL08-19-C07 | Dehydration | |
| FL08-19-P05 | Exposure to a high level salt solution | |
| FL09-07-G09 | Exposure to a high level salt solution | |
| FL09-07-G15 | Dehydration | |
| FL09-10-J18 | Exposure to a high level salt solution | |
| FL09-11-I12 | Dehydration | |
| FL09-12-B03 | Dehydration | |
| FL09-16-I11 | Exposure to a high level salt solution | |
| FL09-16-M04 | Exposure to a high level salt solution | |
| FL11-01-J18 | Dehydration | |
| FL11-07-D13 | Exposure to a high level salt solution | |
| FL11-07-F02 | Exposure to a high level salt solution | |
| FL11-07-N15 | Exposure to a high level salt solution | |
| FL11-10-D10 | Exposure to a high level salt solution | |

In FIGS. 1 to 105, the vertical axis shows the expression ratio of a gene, which is calculated as follows:
Expression ratio=[(FI of a cDNA molecule under stress)/(FI of a cDNA molecule under no stress)]÷[(FI of α-tubulin under stress)/(FI of α-tubulin under no stress)]
where FI is the intensity of fluorescence.

As shown in FIGS. 1 to 105, the stress inducible genes isolated by a method according to the present invention exhibit different profiles; however, it is found that expression is induced by adding each stress. From this, it is demonstrated that the nucleotide sequences positioned upstream of these 90 types of genes and represented by SEQ ID NO: 1 to 90 serve as stress responsive promoters.

### EXAMPLE 2.

### Isolation of Gene Encoding Environmental Stress Responsive Transcriptional Factor

1. Materials and Methods

(1) Arabidopsis cDNA Clone

A microarray was constructed by using about 7,000 cDNA molecules in total including genes isolated from Arabidopsis full-length cDNA libraries, responsive to dehydration (RD) genes, early responsive to dehydration (ERD) genes, kin 1 genes, kin2 genes, and cor15a genes; fragments amplified from λ control template DNA by PCR as an internal standard; and mouse nicotinic acetylcholine receptor epsilon subunit (nAChRE) genes and mouse glucocorticoid receptor homologous genes, as negative controls.
Positive control: dehydration-inducible genes (responsive to dehydration genes: rd, and early responsive to dehydration genes: erd);
Internal standard: fragments amplified from λ control template DNA by PCR (TX803, manufactured by Takara Shuzo, hereinafter referred to as a “control fragment”);
Negative control: mouse nicotinic acetylcholine receptor epsilon subunit (nAChRE) genes and mouse glucocorticoid receptor homologous genes, as negative controls, which do not substantially have homology with any given sequence in an Arabidopsis database for analyzing non-specific hybridization.
   (2) Arabidopsis Full-Length cDNA Microarray

The present inventors have constructed full-length cDNA libraries from an Arabidopsis plant body under different conditions (e.g., dehydration treatment, cold treatment and non-treatment in different growth stages from budding to maturation of seeds) by the biotinylated CAP trapper method. From the full-length cDNA libraries, the present inventors isolated individually about 7,000 independent Arabidopsis full-length cDNA molecules. The cDNA fragments, which were amplified by PCR, were arranged on a slide glass in accordance with a known method (Eisen and Brown, 1999). The present inventors prepared a full-length cDNA microarray containing about 7,000 Arabidopsis full-length cDNA molecules, which contain the genes below.

(3) Dehydration-, Cold-, and High Salt-Inducible Genes Using cDNA Microarray

In this example, dehydration-, cold- and high salt-inducible genes were isolated by using a full length cDNA microarray containing about 7,000 Arabidopsis full-length cDNA molecules.

Probes of plants treated with different stresses and an untreated plant with stress and labeled with Cy3 and Cy5 fluorescent dyes were mixed. The probes were hybridized with the full-length cDNA microarray containing about 7,000 Arabidopsis full-length cDNA molecules. By such a double labeling of a pair of cDNA probes wherein one of the mRNA samples was labeled with Cy3-dUTP and the other was labeled with Cy5-dUTP, hybridization with DNA elements on a microarray can be performed simultaneously, with the result that quantitative determination of gene expression under two different conditions (that is, stressed and unstressed conditions) can be directly and easily performed. The hybridized microarray was scanned by two discrete laser channels for Cy3 and Cy5 emission from each of DNA elements. Subsequently, the intensity ratio between two fluorescent signals from each DNA element was determined. Based on the relative value of the intensity ratio, a change of differential expression of genes represented as a cDNA spot on the microarray was determined. In this example, an α-tubulin gene, whose expression level was almost equivalent under two different experimental conditions, was used as an internal control gene.

In the full-length cDNA microarray containing about 7,000 Arabidopsis full-length cDNA molecules, a procedure for identifying dehydration-, cold-, and high salt-inducible genes will be explained.

Both mRNA molecules derived from a plant treated with one of the stresses mentioned above and mRNA molecules derived from a wild-type plant unstressed were used to prepare Cy3-labeled cDNA and Cy5-labeled cDNA probes, respectively. These cDNA probes were mixed and hybridized with a cDNA microarray. In this example, a control fragment, which exhibits almost the same expression level under two type conditions, was used as an internal control gene. A gene that exhibits the expression ratio (dehydration/unstressed, cold/unstressed or high salt/unstressed) more than 5 times of that of the control fragment was defined as an inducible gene by a stress given to the gene.

(4) Analysis of Sequence

Plasmid DNA extracted by a DNA extraction device (model Biomek, manufactured by Beckman Coulter) and purified by use of a multiscreen 96-hole filter plate (manufactured by Millipore) was sequenced to find homology of gene sequences. A DNA sequence was determined by a dye terminator cycle sequencing method using a DNA sequencer (ABI PRISM 3700. PE Applied Biosystems, CA, USA). Based on the GenBank/EMBL database, the homology of sequences was found by using the BLAST program.

(5) Amplification of cDNA

λZAP and λ FLC-1 were used as a vector for constructing a cDNA library. The cDNA inserted in a vector for the library was amplified by PCR using complementary primers to the sequences of both sides of the cDNA.

The sequences of primers are as follows:

| | |
|---|---|
| FL forward 1224: | |
| 5′-CGCCAGGGTTTTCCCAGTCACGA | (SEQ ID NO: 165) |
| FL reverse 1233: | |
| 5′-AGCGGATAACAATTTCACACAGGA | (SEQ ID NO: 166) |

To 100 μl of a PCR solution mixture (0.25 mM dNTP, 0.2 μM PCR primer, 1×Ex Taq Buffer, and 1.25 U of Ex Taq polymerase (manufactured by Takara Shuzo)), a plasmid (1 to 2 ng) was added as a template. PCR was performed under the following conditions: initial reaction at 94° C. for 3 minutes, 35 cycles each consisting of 95° C. for one minute, 60° C. for 30 seconds, and 72° C. for 3 minutes, and a final reaction at 72° C. for 3 minutes. After a PCR product was precipitated with ethanol, the precipitate was dissolved in 25 μl of 3×SSC and subjected to electrophoresis using 0.7% agarose gel. The quality of the DNA obtained and amplification efficiency of PCR were conformed.

(6) Construction of cDNA Microarray

Using a gene tip microarray stamp machine GTMASS SYSTEM (manufactured by Nippon Laser & Electronics Lab.), 0.5 μl of a PCR product (500-1,000 ng/ml) was loaded from a 384-well microtiter plate and form spots of the PCR product (5 nl for each) at intervals of 300 μm on 48 micro slide glasses (model Super Aldehyde substrate, manufactured by Telechem International). After spotting, the slide was dried in an atmosphere having a relative humidity of 30% or less and irradiated with ultraviolet rays for mediating a cross-linking reaction.

Thereafter, the slide was treated in 0.2% SDS with shaking for 2 minutes three times and soaked in distilled water twice. Subsequently, the slides were placed on a slide rack, which was the transferred into a chamber containing hot water and allowed to stand for 2 minutes. Subsequently, to the chamber, a blocking solution (containing 1 g borohydride, 300 ml of PBS, and 90 ml of 100% ethanol) was poured. After the glass chamber housing the slide rack was moderately shaken, the slide rack was transferred to a chamber containing 0.2% SDS and gently shaken for one minute 3 times. Thereafter, the slide rack was transferred to a glass chamber containing distilled water, moderately shaken for one minute, and centrifuged for 20 minutes to dry.

(7) Plant Material and Isolation of RNA

As a plant material, use was made of a wild type Arabidopsis thaliana plant body which was seeded on an agar medium and grown for 3 weeks (Yamaguchi-Shinozaki and Shinozaki, 1994) and an Arabidopsis thaliana (Colombian species) plant body into which DREB1A cDNA (Kasuga et al., 1999) connected to a 35S promoter of a cauliflower mosaic virus was introduced. Dehydration- and cold-stress treatments were performed in accordance with the method of Yamaguchi-Shinozaki and Shinozaki (1994). More specifically, dehydration treatment was performed by pulling a plant body out of the agar medium, placing it on a filter, and dried at a temperature of 22° C. and a relative humidity of 60%. The cold treatment was performed by transferring a plant body grown at 22° C. to 4° C. High salt stress treatment was perfonned by growing a plant body at an aqueous solution containing 250 mM NaCl.

After wild type plant bodies were exposed to stress-treatment for 2 or 10 hours, a sample was taken from each of plant bodies and stored in cryogenic conditions with liquid nitrogen. Furthermore, wild type and DREB1A overexpresstion-type transformants cultured in an agar medium without kanamycin were subjected to an experiment for identifying a DREB1A target gene. The DREB1A overexpresstion-type transformant was not treated with stresses. The total RNA was isolated from the plant body by using ISOGEN (Nippon gene, Tokyo, Japan) and mRNA was isolated and purified by Oligotex-dT30 mRNA purification kit (Takara, Tokyo, Japan).

(8) Fluorescent Labeling of Probe

Each of the mRNA samples was subjected to a reverse transcription reaction in the presence of Cy3 dUTP or Cy5 dUTP (Amersham Pharmacia). More specifically, the reverse transcription reaction was performed in a total amount of 20 μl of 1× Superscript first-stand buffer (containing 50 mM Tris-HCl, pH 8.3, 75 mM KCl, 3 mM MgCl₂, and 20 mM DTT, manufactured by Life Technology), which contained:
1 μg of denatured poly (A)⁺ which contains 1 ng of λ poly A⁺RNA-A (TX802, manufactured by Takara Shuzo) serving as an internal standard;
50 ng/μl 12 to 18 mer oligo dT primer (manufactured by Life Technology);
0.5 mM dATP, 0.5 mM dGDP, 0.5 mM dCTP, and 0.2 mM dTTP;
0.1 mM Cy3 dUTP or Cy5 dUTP;
100 U of Rnase inhibitor;
10 mM DTT; and
200 U of Superscript II reverse transcriptase.

After the reaction solution of the aforementioned composition was incubated at 42° C. for 35 minutes, 200 U of Superscript II reverse transcriptase was added and further incubated at 42° C. for 35 minutes. To this reaction mixture, subsequently, 5 μl of 0.5 M EDTA, 10 μl of 1N NaOH, and 20 μl of distilled water were added, thereby terminating the enzyme reaction taking place in the reaction solution and simultaneously decomposing a template. The reaction solution was then incubated at 65° C. for 1 hour and thereafter neutralized with 1M Tris-HCL (pH 7.5).

The reaction solution was transferred to a Microcon 30 micro concentrator (manufactured by Amicon). 250 μl of TE buffer was added and centrifuged until the amount of the buffer reached 10 μl. The effulent was discarded. This step was repeated 4 times. Probes contained in the reaction solution were centrifugally collected and several μl of distilled water was added. To the obtained probes, 5.1 μl of 20×SSC, 2 μg/μl of Yeast tRNA, and 4.8 μl of 2% SDS were added. Further, the samples were denatured at 100° C. for 2 minutes, placed at room temperature for 5 minutes, and thereafter used in hybridization.

(9) Microarray Hybridization and Scanning

A probe was centrifuged for one minute by a benchtop micro centrifuge. To avoid generation of bubbles, the probe was placed at the center of an array and a cover slip was placed thereon. Four drops of 5 μl of 3×SSC were dropped on a slide glass and a chamber was kept at a suitable humidity to prevent the probe from being dried during hybridization. After the slide glass was placed in a cassette for hybridization (THC-1, BM machine) and the cassette was sealed, hybridization treatment was performed at 65° C. for 12 to 16 hours. The slide glass was taken out from the cassette and placed on the slide rack. After the cover slip was carefully removed in solution 1 (2×SSC, 0.03% SDS), the rack was washed while shaking and transferred into solution 2 (1×SSC) to wash for 2 minutes. The rack was further transferred to solution 3 (0.05×SSC), allowed to stand for 2 minutes, and centrifuged at 2500 g for 1 min to dry.

The microarray was scanned at a resolution of 10 μm per pixel by use of a scanning laser microscope (ScanArray 4000; GSI Lumonics, Watertown, Mass.). As a program for analyzing microarray data, QuantArray, Ver 2.0 (GSI Lumonics) was used. The background fluorescence was obtained through calculation based on fluorescent signals obtained from negative control genes (mouse nicotinic acetylcholine receptor epsilon subunit (nAChRE) gene and mouse glucocorticoid receptor homologous gene). Samples giving a fluorescent signal value of less than 1,000, which is equal to less than twice the background signal value, were not subjected to analysis. The cluster analysis of genes was performed by Genespring (manufactured by Silicon Genetic).

(10) Northern Analysis

Northern analysis was performed using total RNA, (Yamaguchi-Shinozaki and Shinozaki, 1994). DNA fragments were isolated from an Arabidopsis thaliana full-length cDNA library by a PCR method and used as probes for Northern hybridization.

(11) Determination of Gene Encoding Transcriptional Factor

Based on the genomic information of Arabidopsis thaliana in a data base (GenBank/EMBL, ABRC), a gene encoding transcriptional factor was analyzed by using the BLAST program for gene analysis.

2. Results

(1) Identification of Stress-Inducible Gene

Fluorescence-labeled cDNA was prepared by subjecting mRNA isolated from unstressed Arabidopsis thaliana to a reverse transcription reaction in the presence of Cy5-dUTP. A second probe labeled with Cy3-dUTP was prepared from a plant stress with dehydration, cold or high-salt. Both probes were simultaneously hybridized with a cDNA microarray containing about 7,000 Arabidopsis thaliana cDNA clones and pseudo color image was created.

Genes induced and suppressed by a stress are represented by a red signal and a green signal, respectively. Genes expressed at virtually the same level in both treatments are represented by a yellow signal. The intensity of each spot corresponds to the absolute value of the expression level of each gene. It is shown that a cold-inducible gene (rd29A) is represented by a red signal, whereas a control fragment (an internal control) is represented by a yellow signal.

As a result of scanning the microarray, 277 genes induced by dehydration treatment, 53 genes induced by cold treatment, and 194 genes induced by high salt treatment were identified. Note that genes whose expression ratio are not less than 5 times as large as that of a control fragment were determined as ones induced by a variety of stresses.

As a result of analysis using a database, 35 transcriptional factors, which were classified into the following families were identified. Note that RAFL05-21-L12 was not classified into the following families. However, when the nucleic acid base sequence, which was searched by the BLAST X based on amino acid sequence data registered in the GenBank Database, it exhibited E-value of e⁻¹⁰⁰, which means that RAFL05-21-L12 is homologous to a gene encoding a known transcriptional factor, that is, heat shock transcriptional factor-like protein. As a result, RAFL05-21-L12 was identified as a transcriptional factor. In conclusion, 36 types of transcriptional factors were identified in this example.
(1) DREB family: RAFL05-11-M11, RAFL06-11-K21, RAFL05-16-H23, RAFL08-16-D06;
(2) ERF family: RAFL08-16-G17, RAFL06-08-H20;
(3) Zinc finger family: RAFL07-10-G04, RAFL04-17-D16, RAFL05-19-M20, RAFL08-11-M13, RAFL04-15-K19, RAFL05-11-L01, RAFL05-14-C11, RAFL05-19-G24, RAFL05-20-N02;
(4) WRKY family: RAFL05-18-H12, RAFL05-19-E19, RAFL06-10-D22, RAFL06-12-M01;
(5) MYB family: RAFL05-14-D24, RAFL05-20-N17, RAFL04-17-F21;
(6) bHLH family: RAFL09-12-N16;
(7) NAC family: RAFL05-19-I05, RAFL05-21-I22, RAFL08-11-H20, RAFL05-21-C17, RAFL05-08-D06;
(8) Homeo domain family: RAFL05-20-M16, RAFL11-01-J18; RAFL11-09-C20; and
(9) bZIP family: RAFL05-18-N16, RAFL11-10-D10, RAFL04-17-N22, RAFL05-09-G15.
   (3) The Relationship between Treatment Time with Each Stress and Expression Ratio

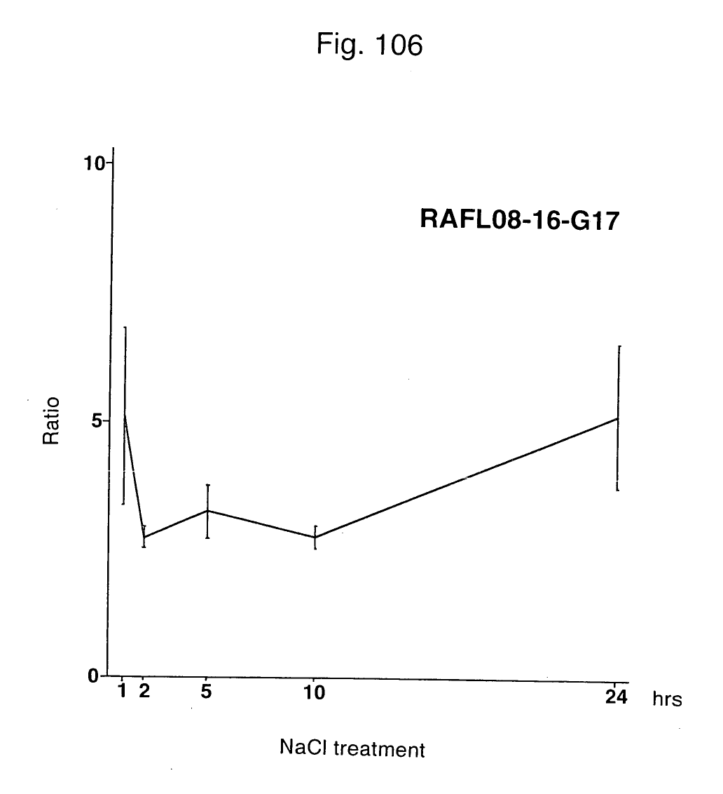

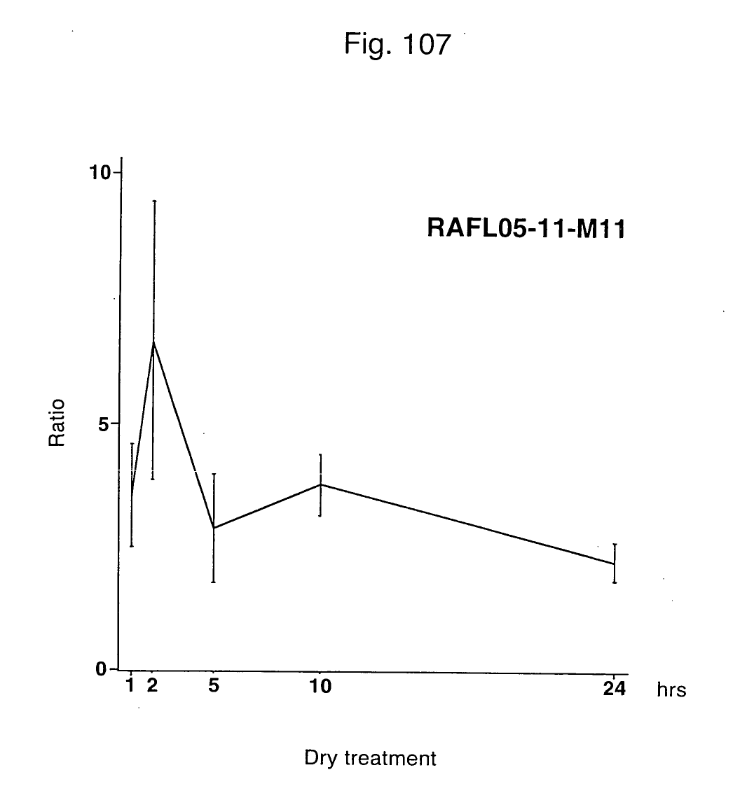

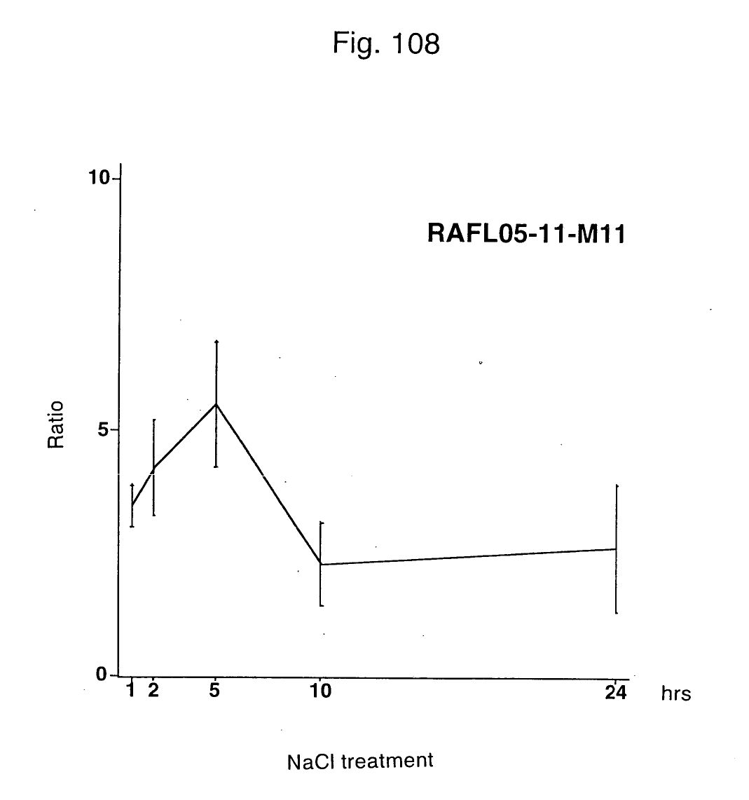

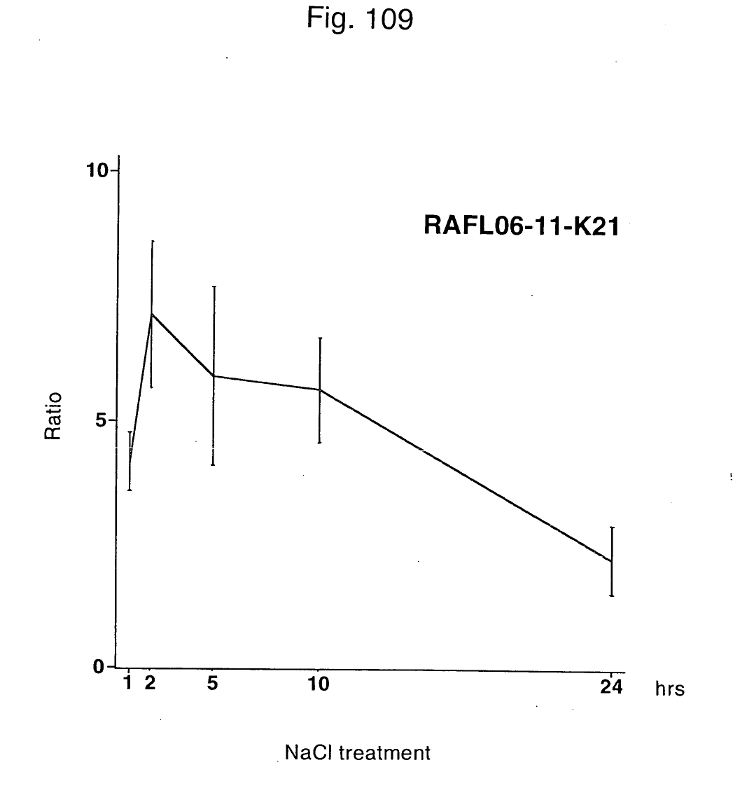

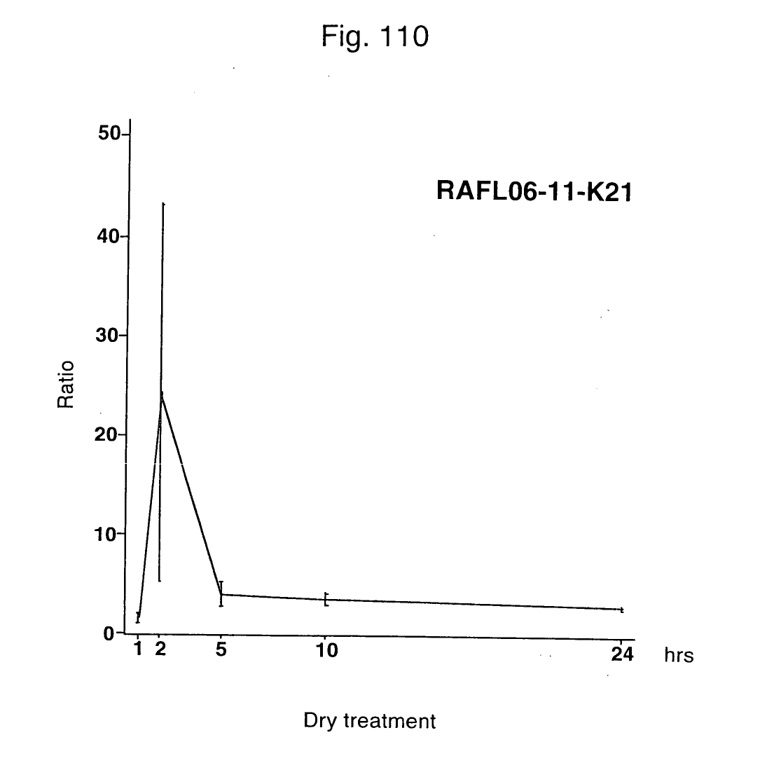

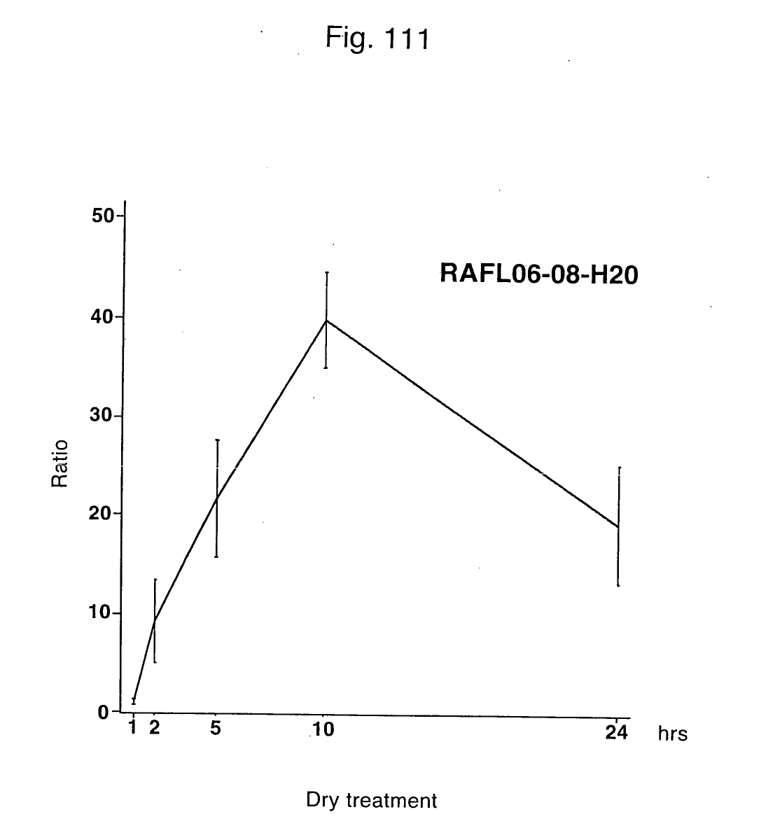

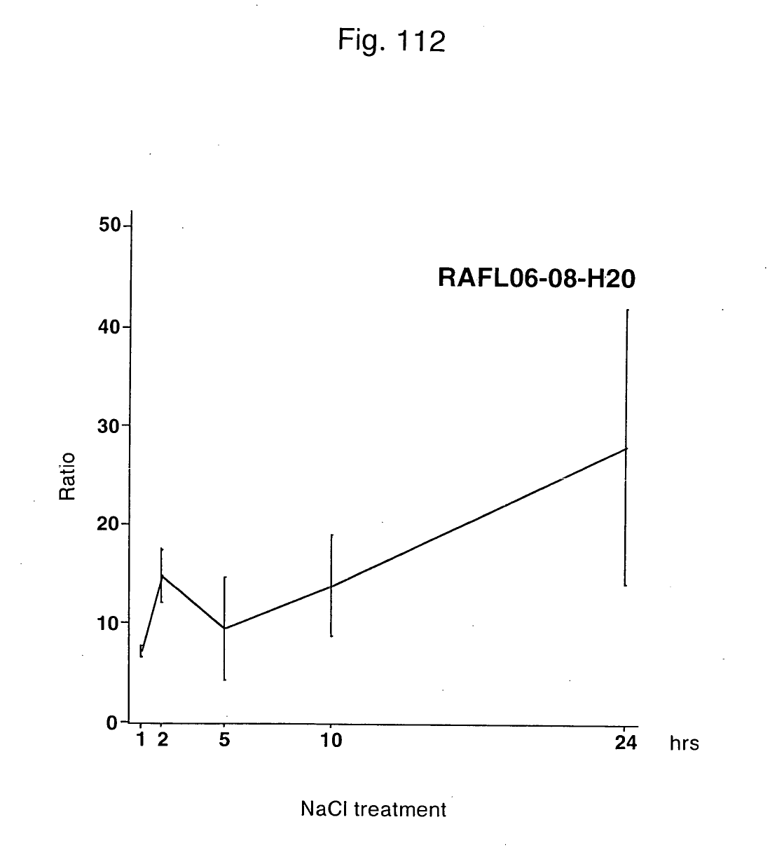

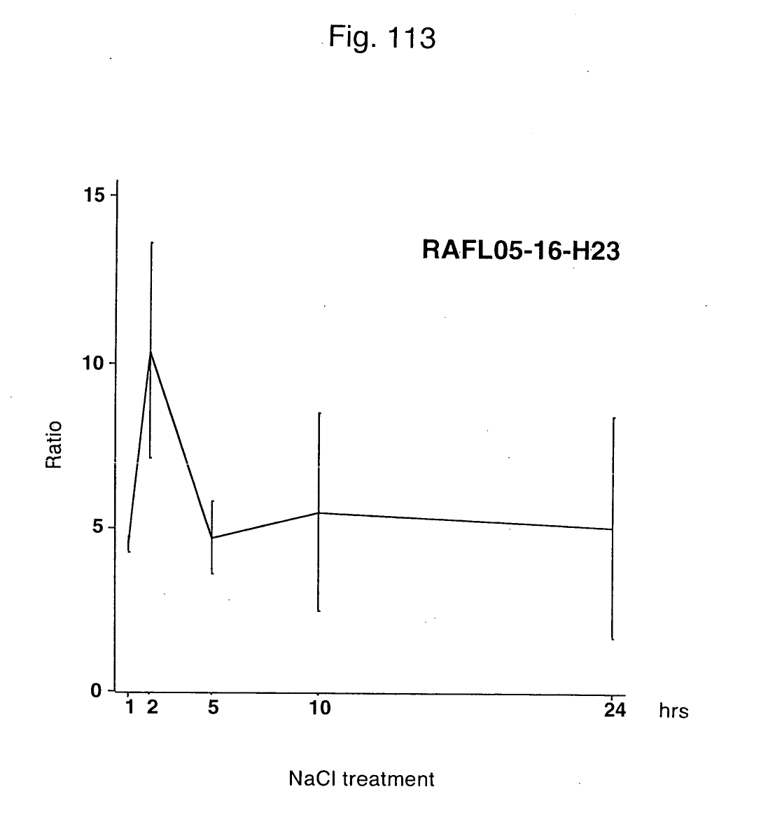

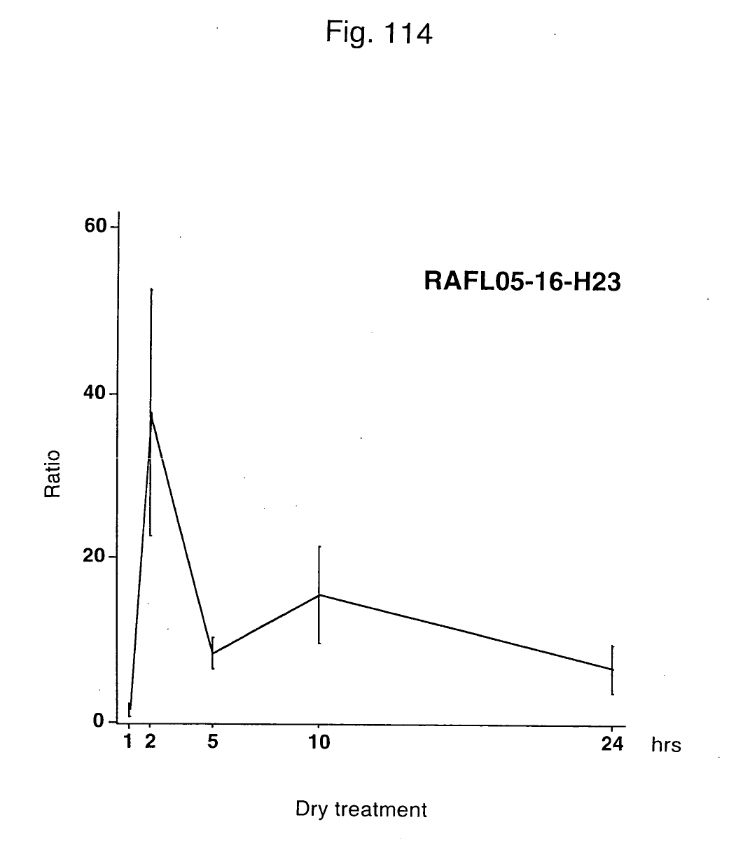

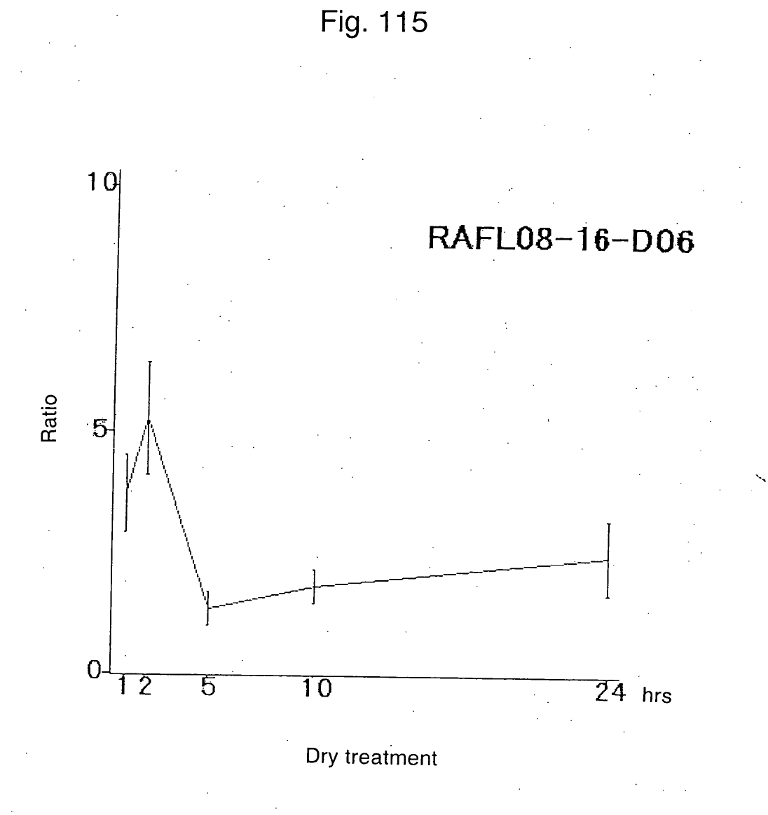

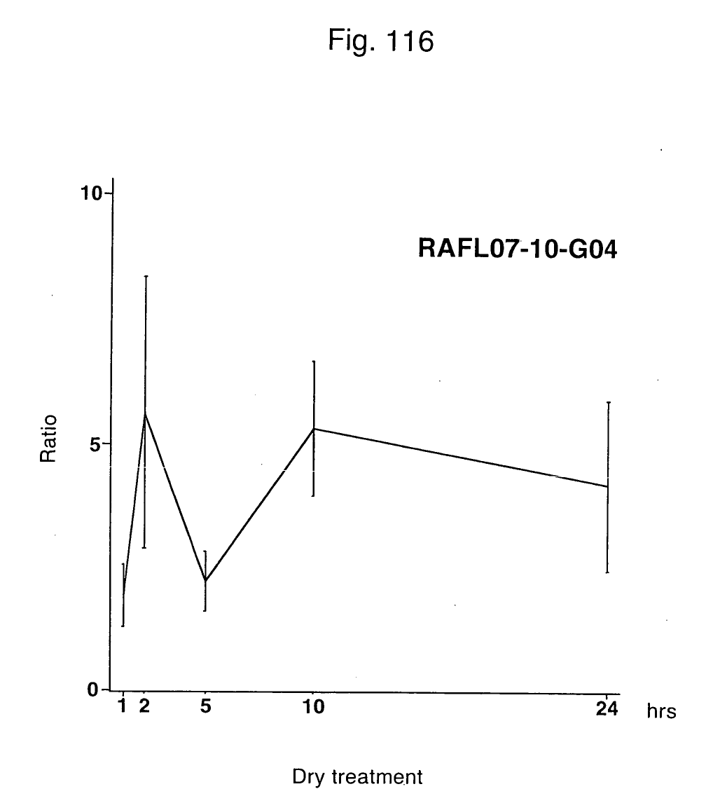

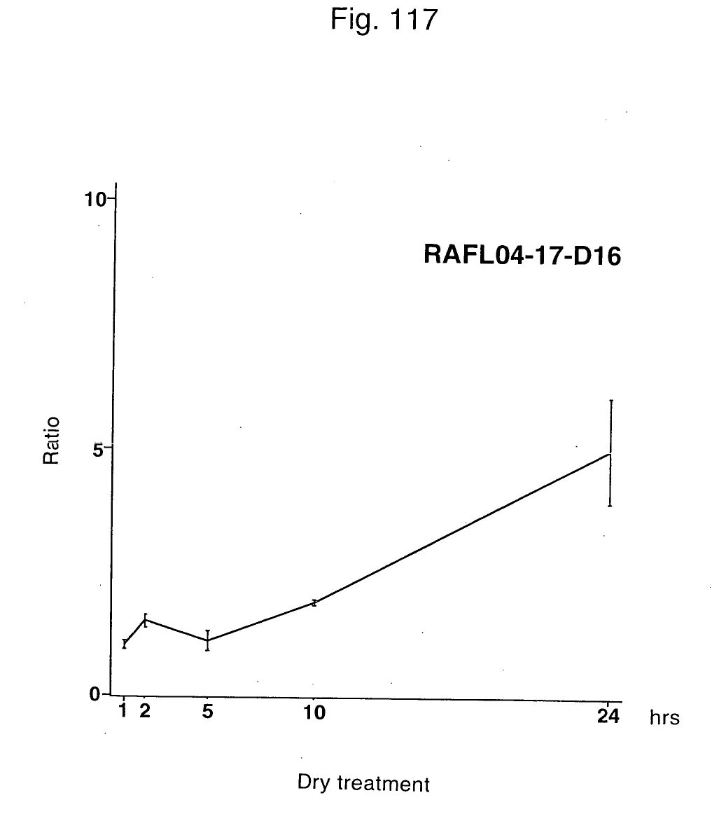

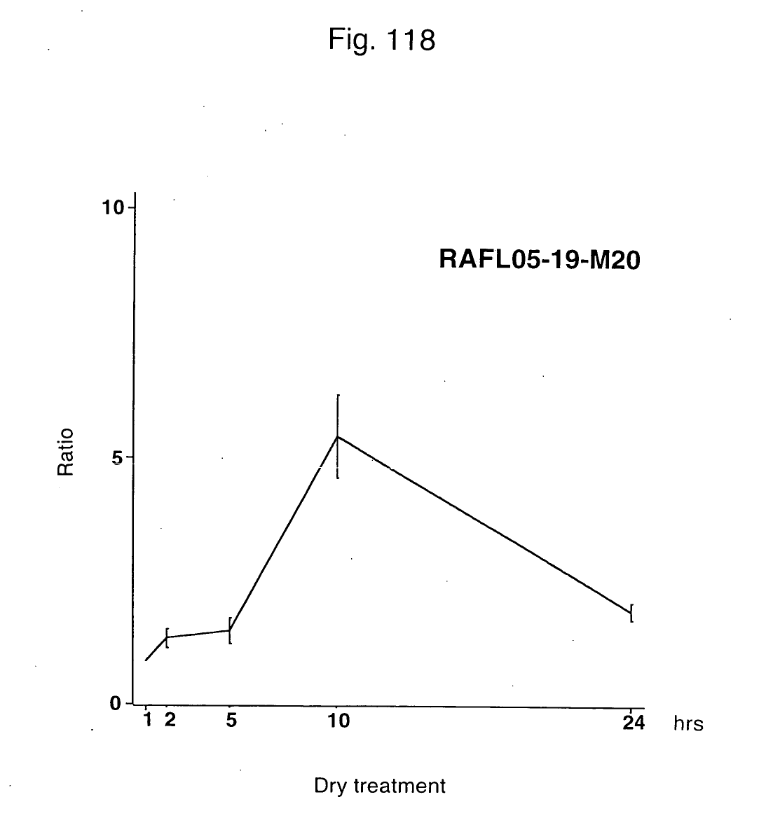

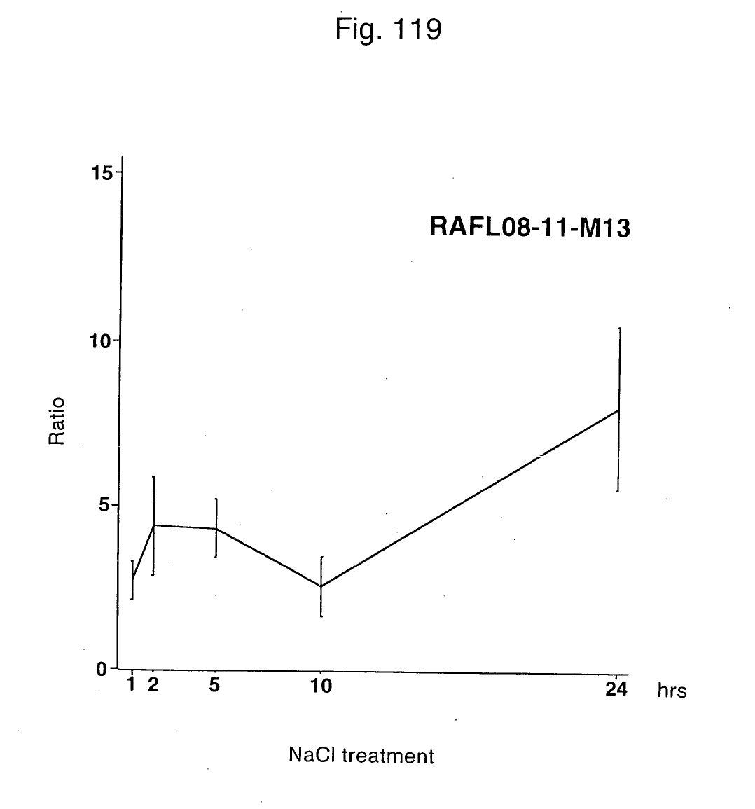

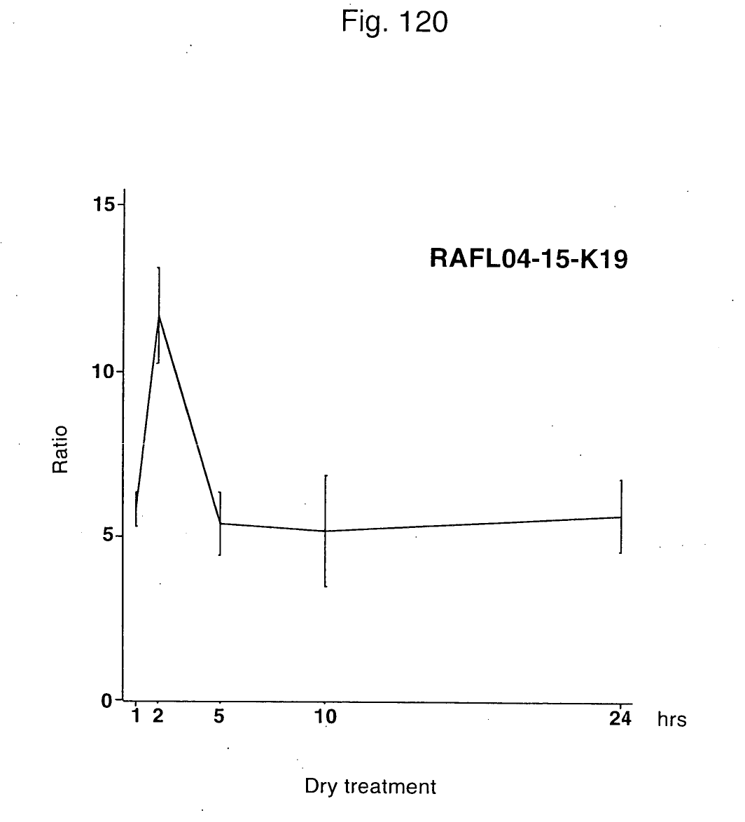

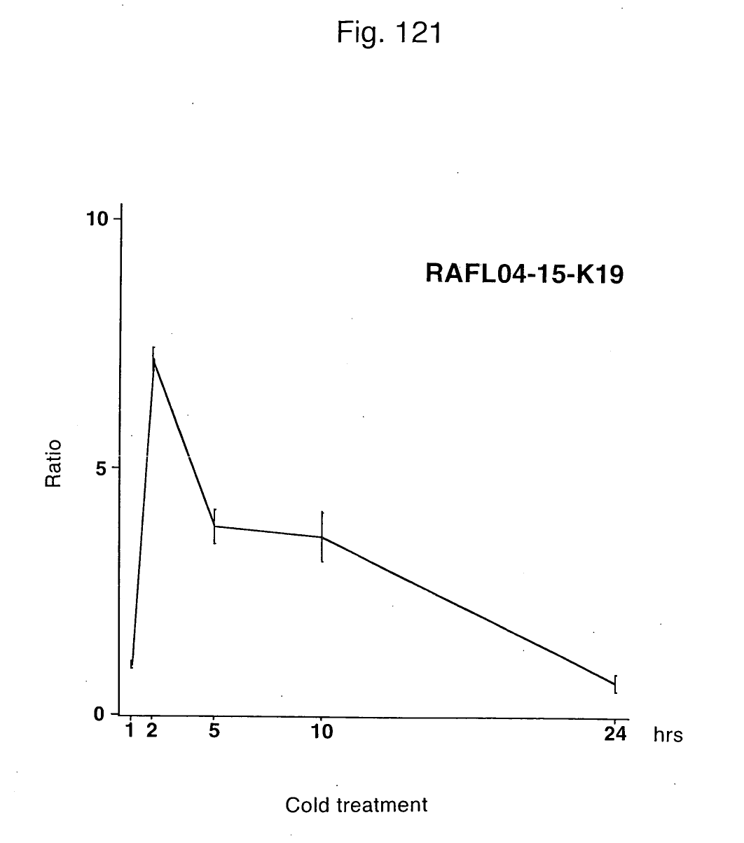

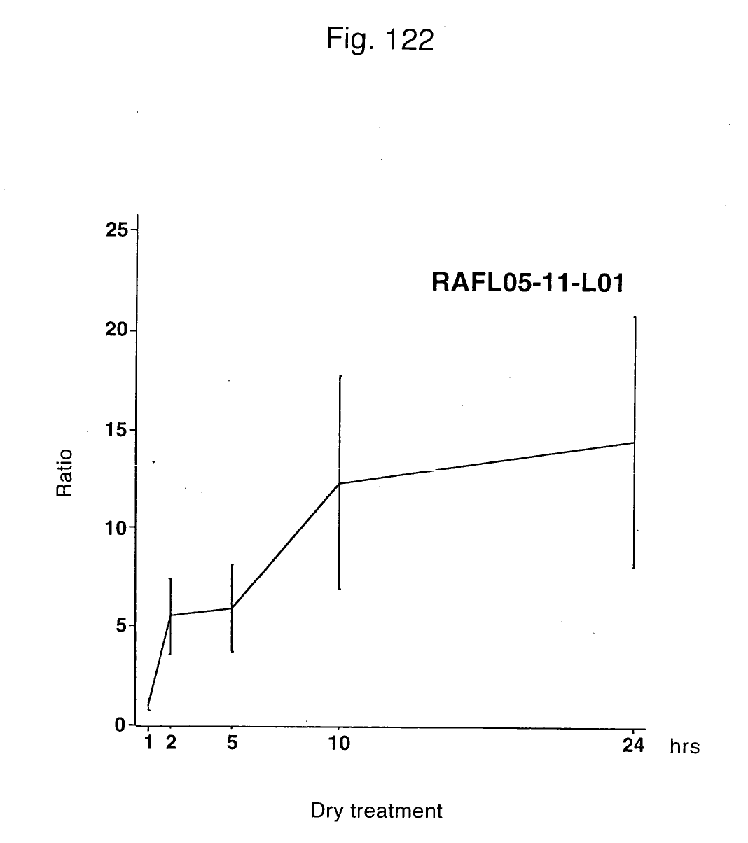

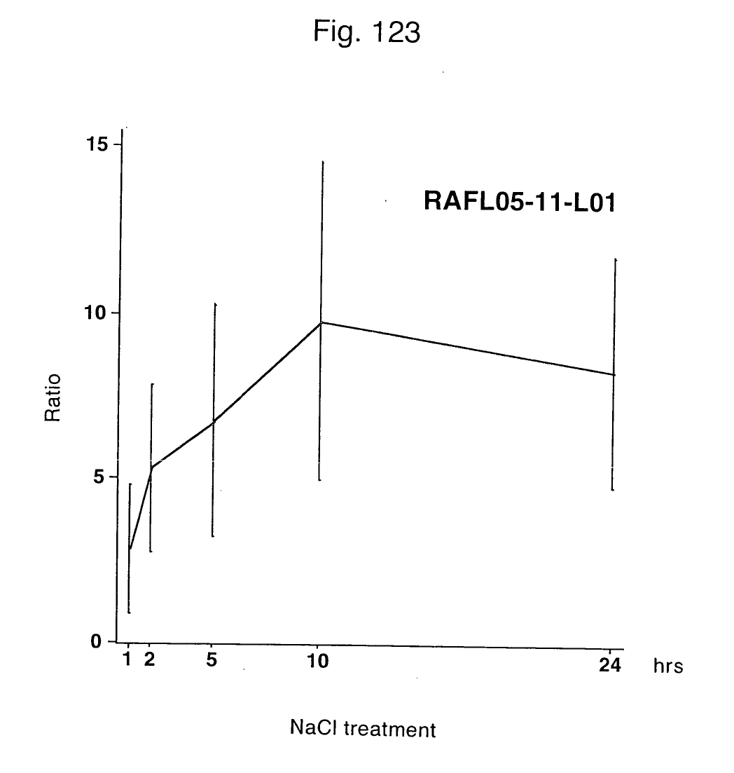

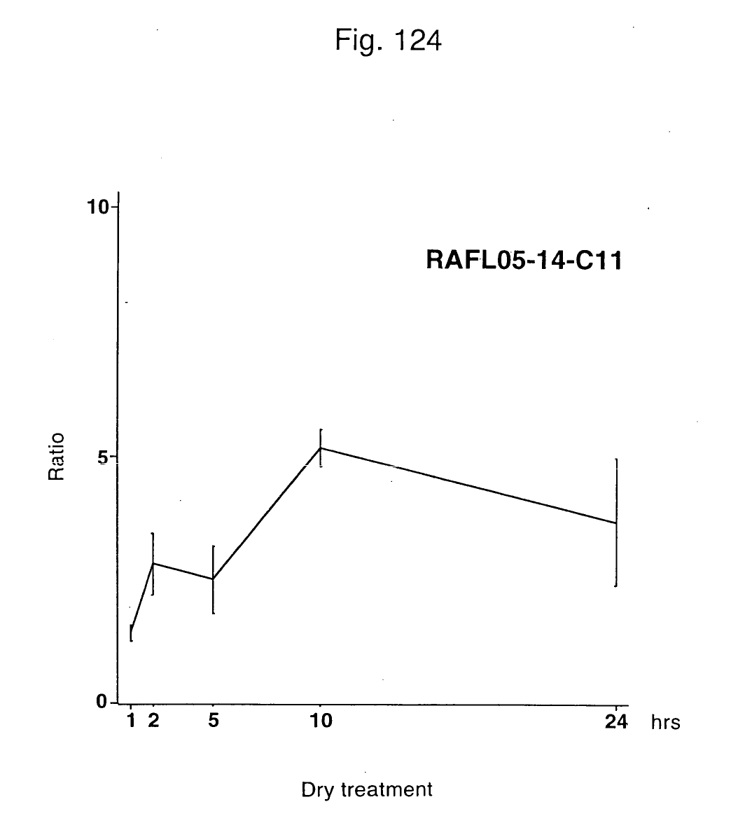

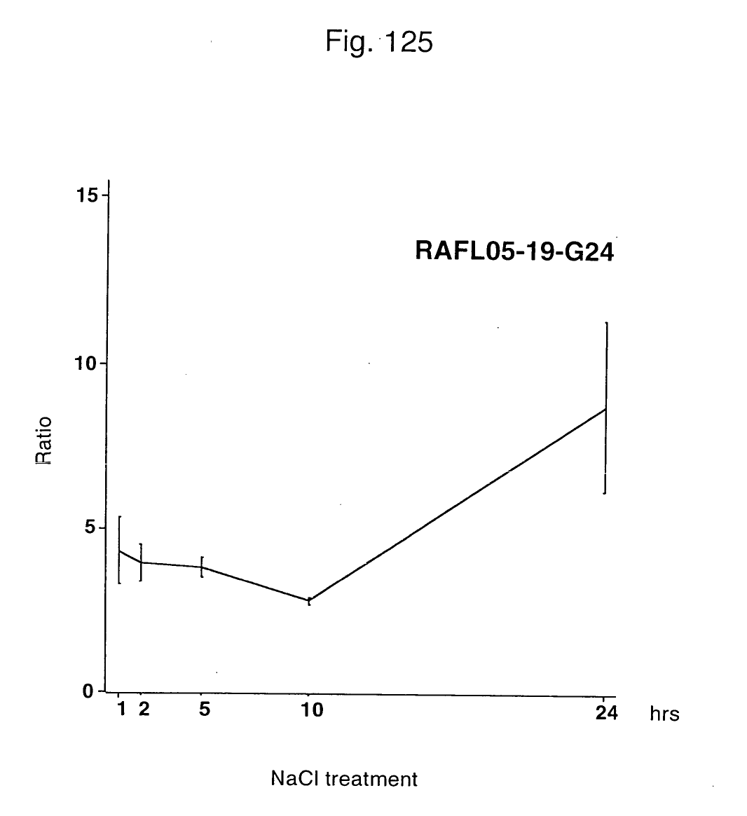

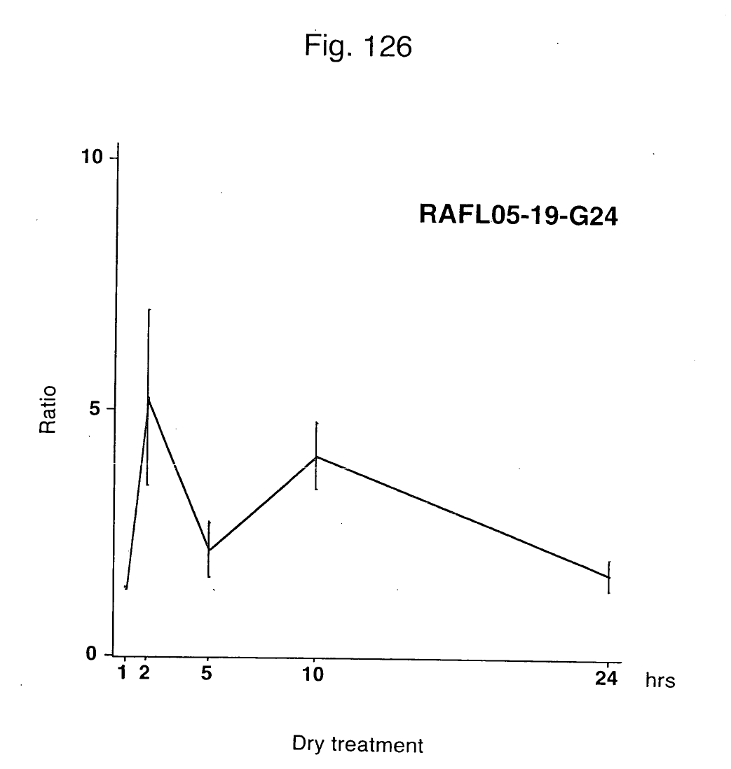

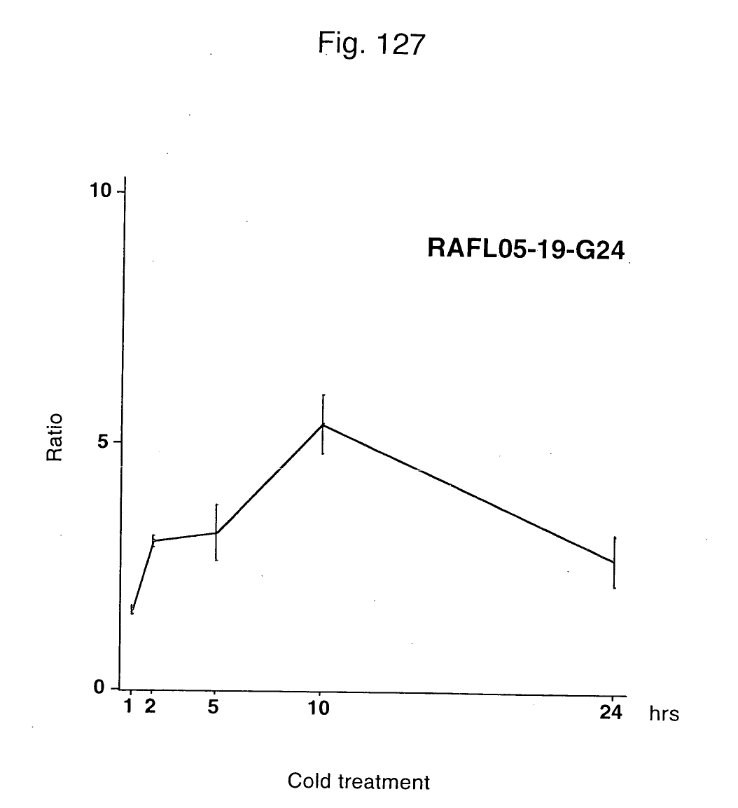

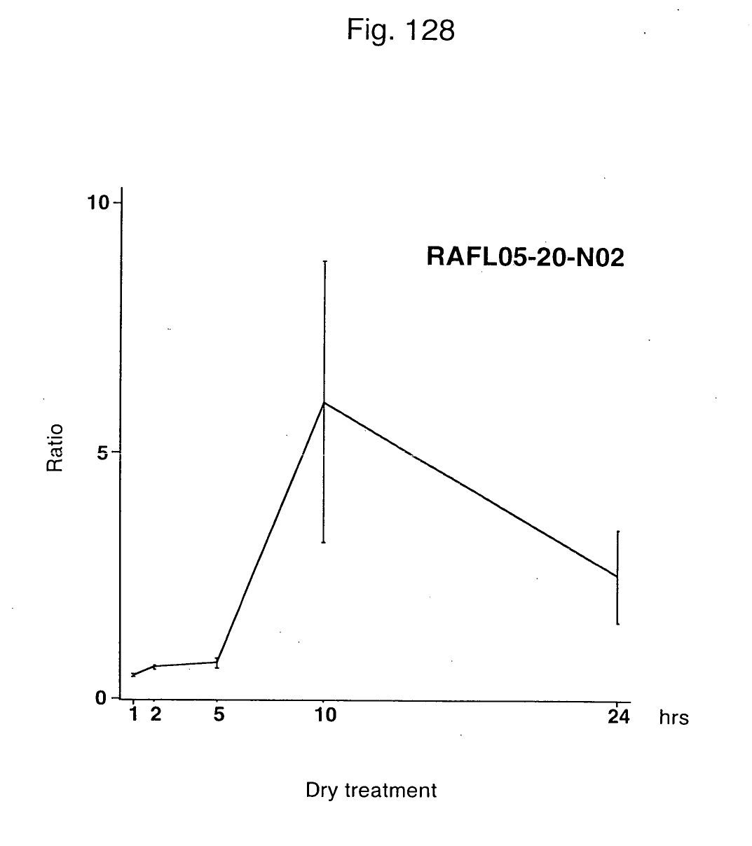

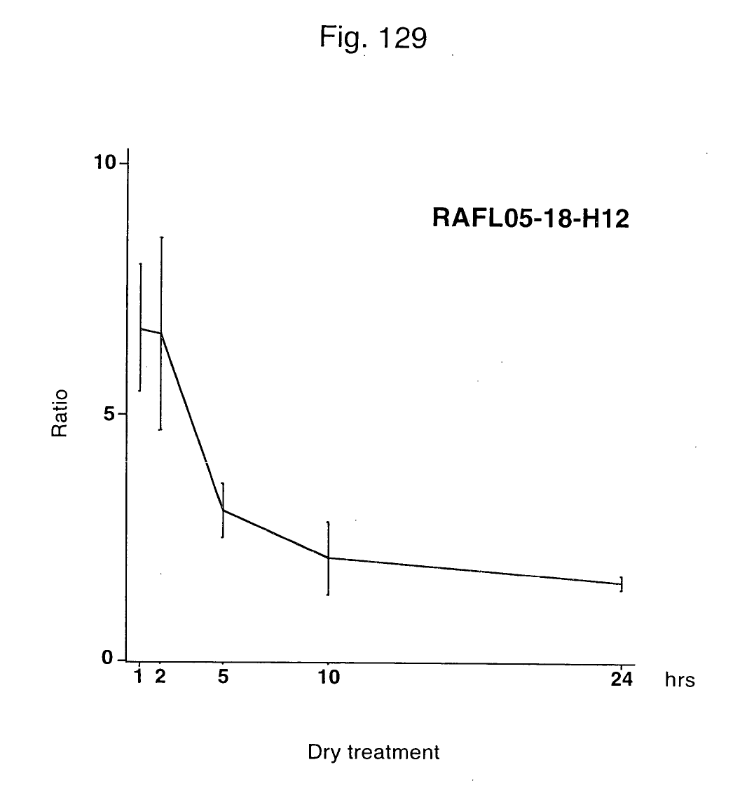

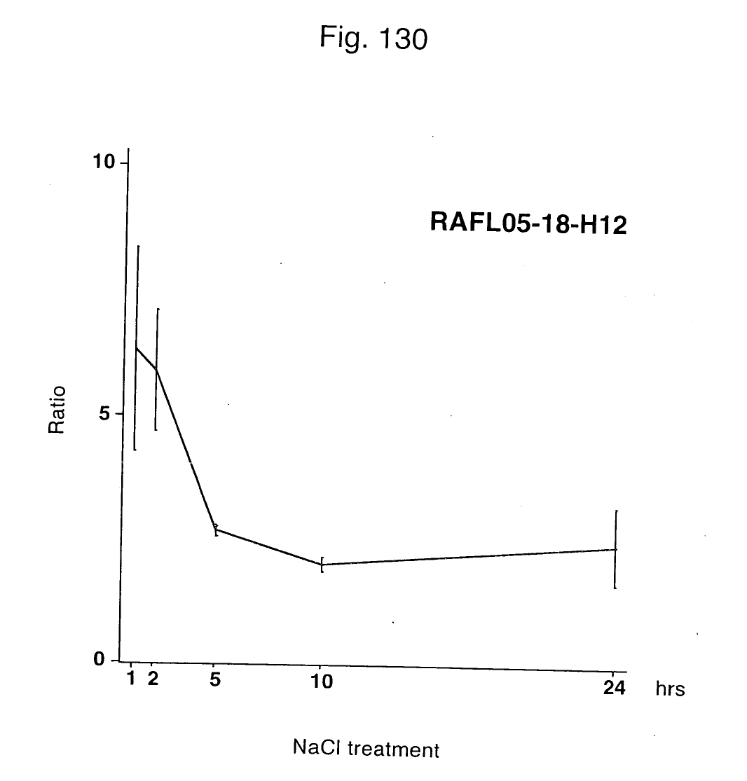

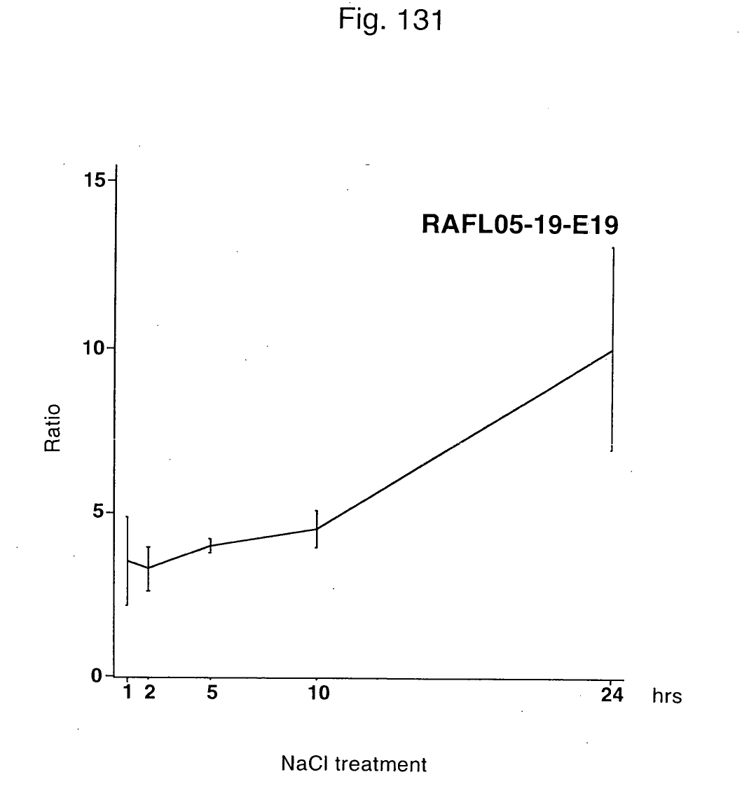

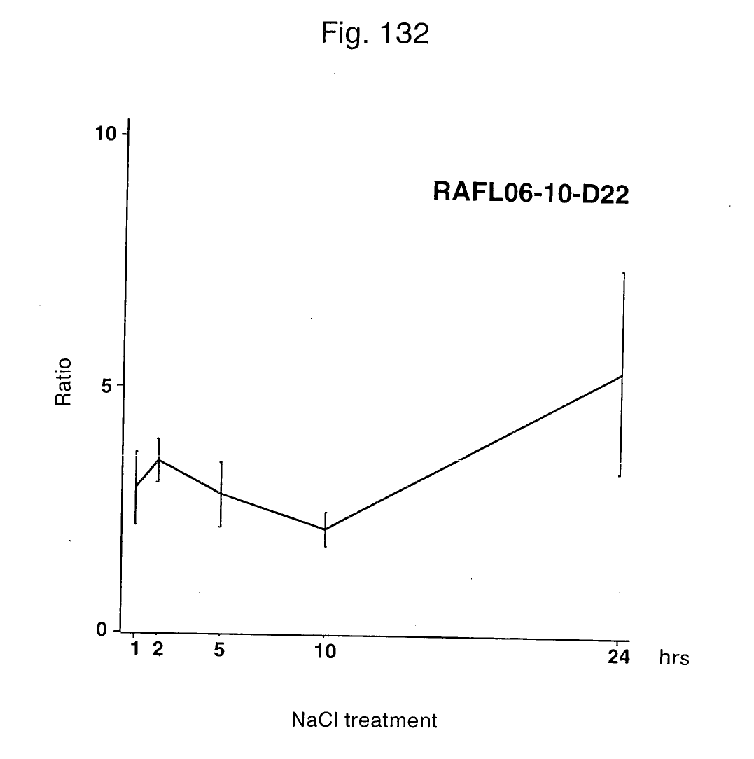

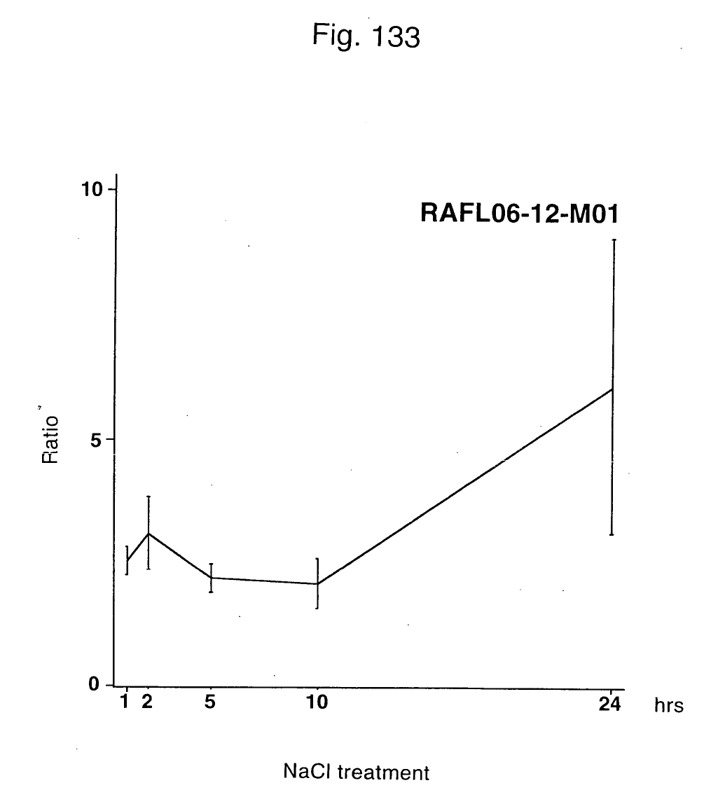

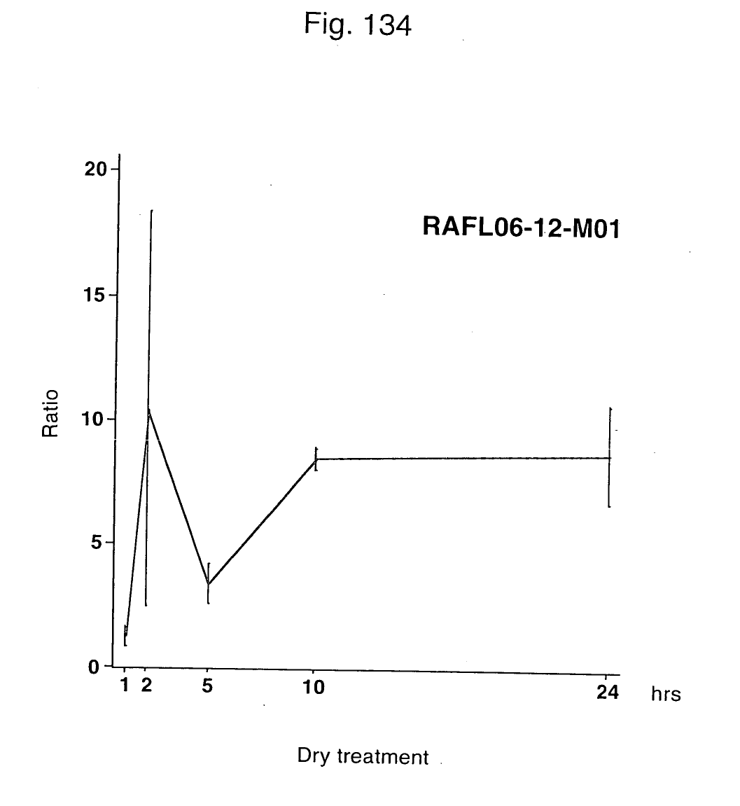

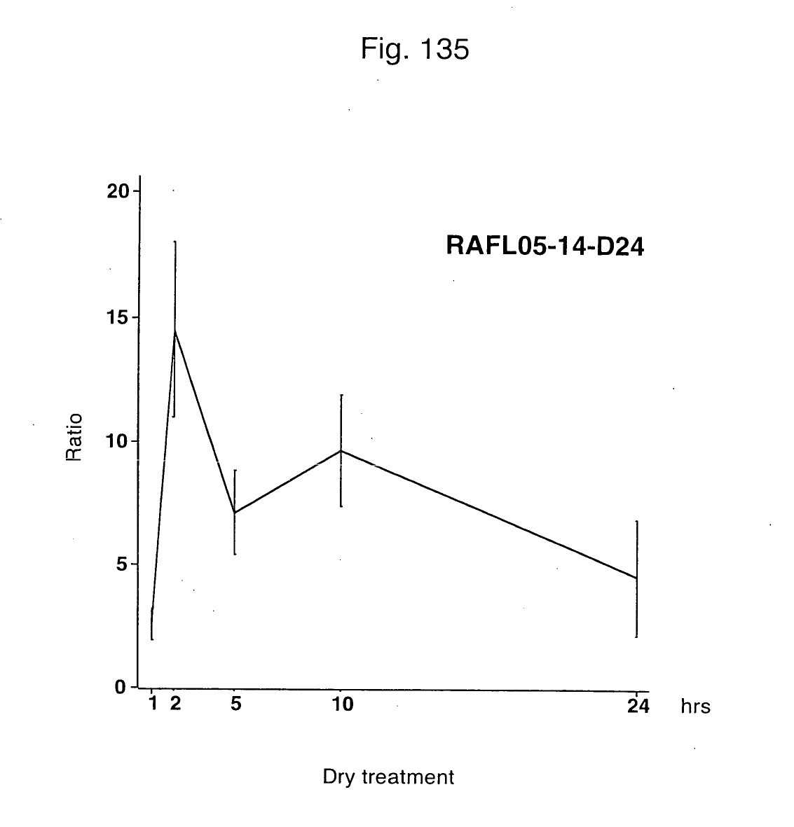

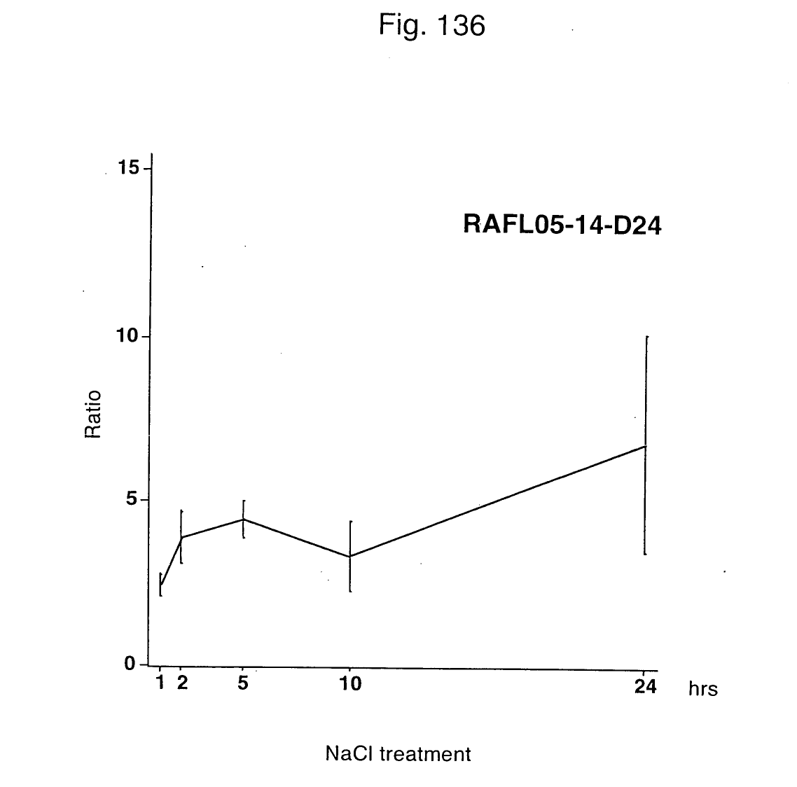

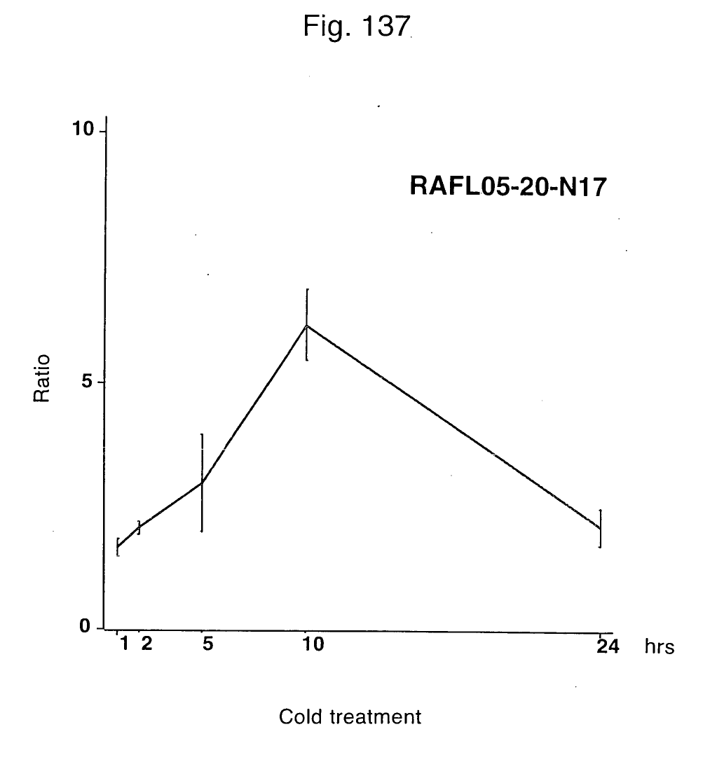

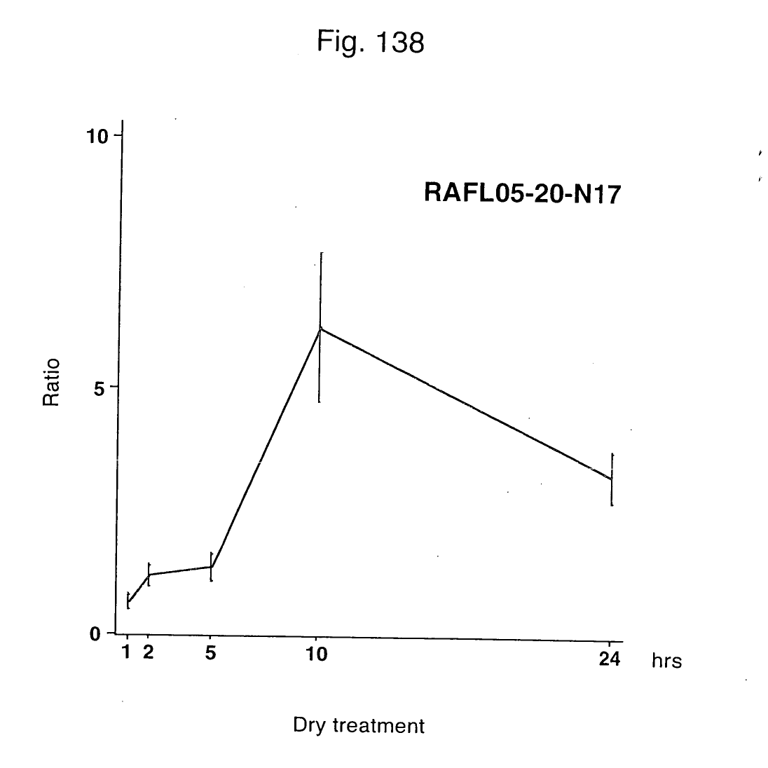

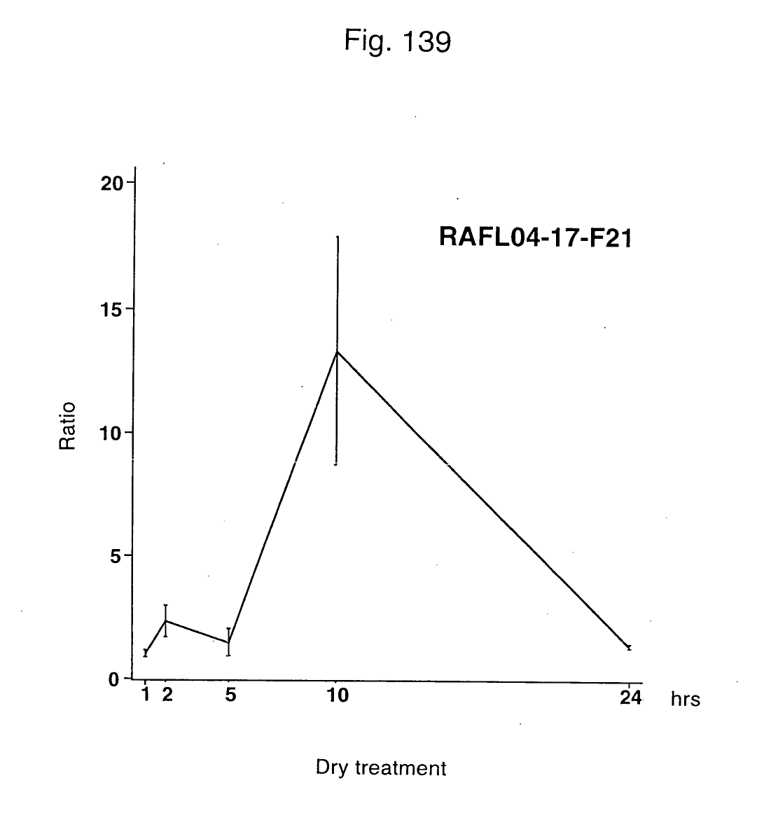

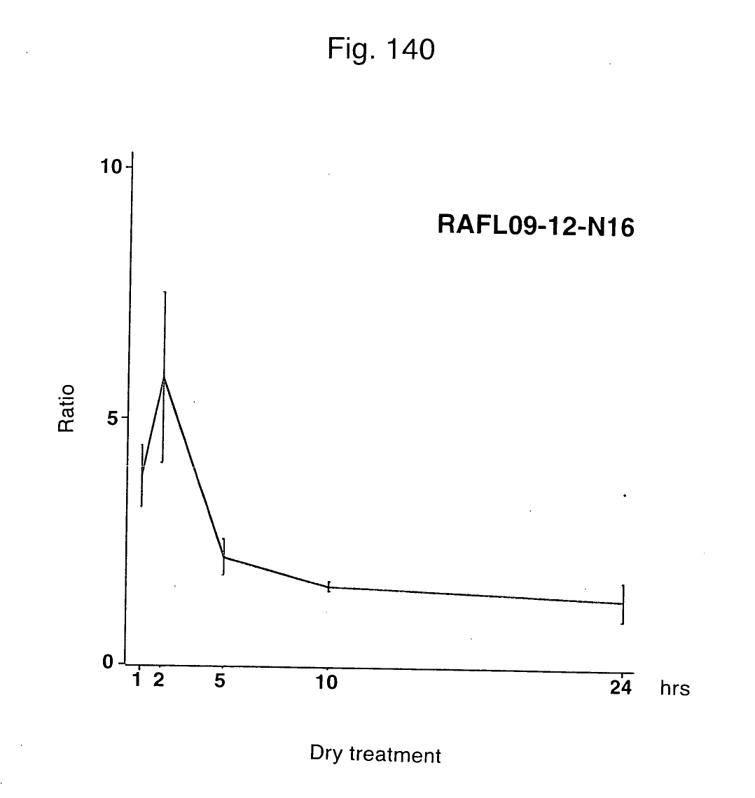

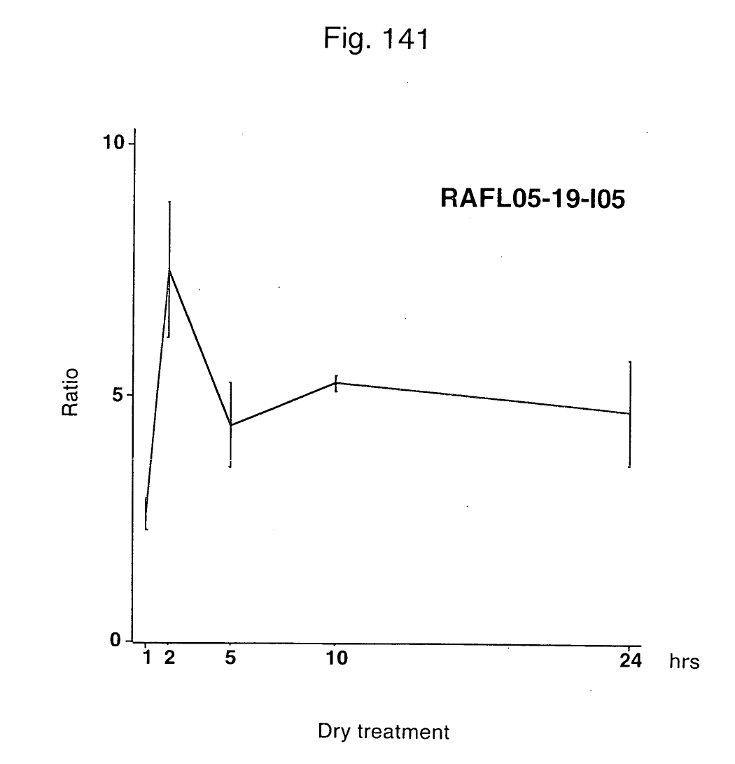

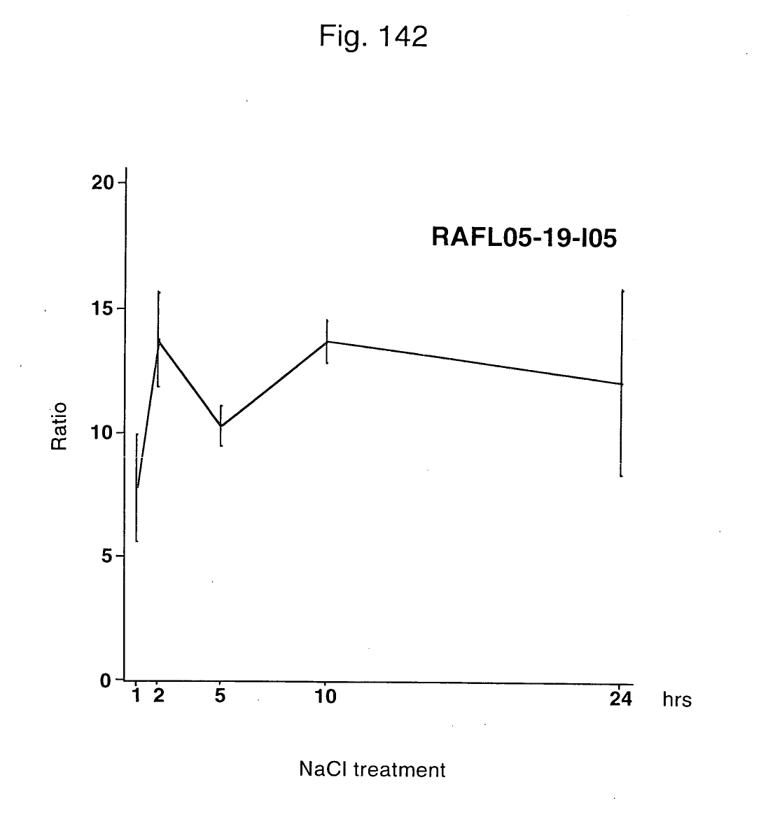

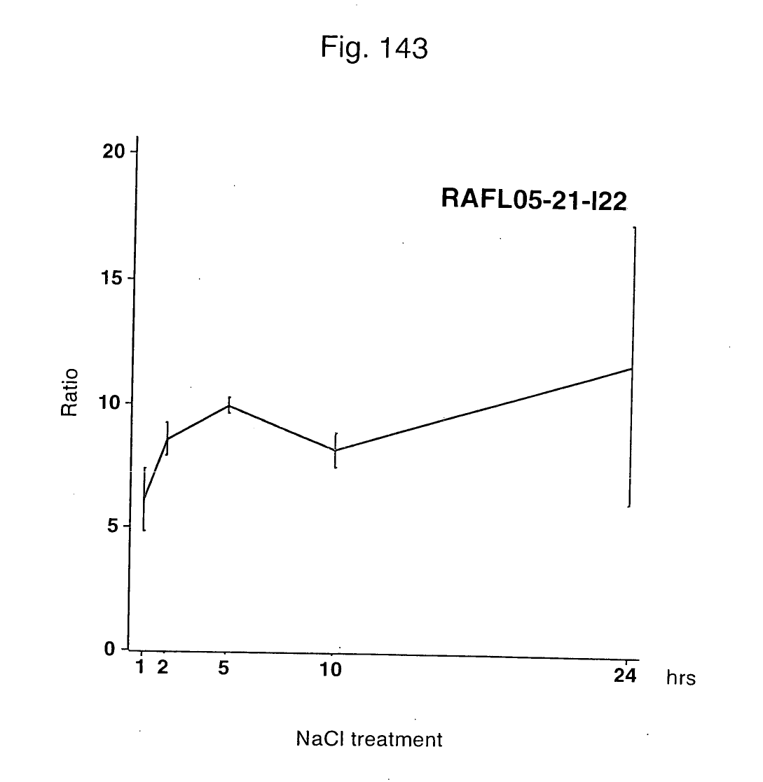

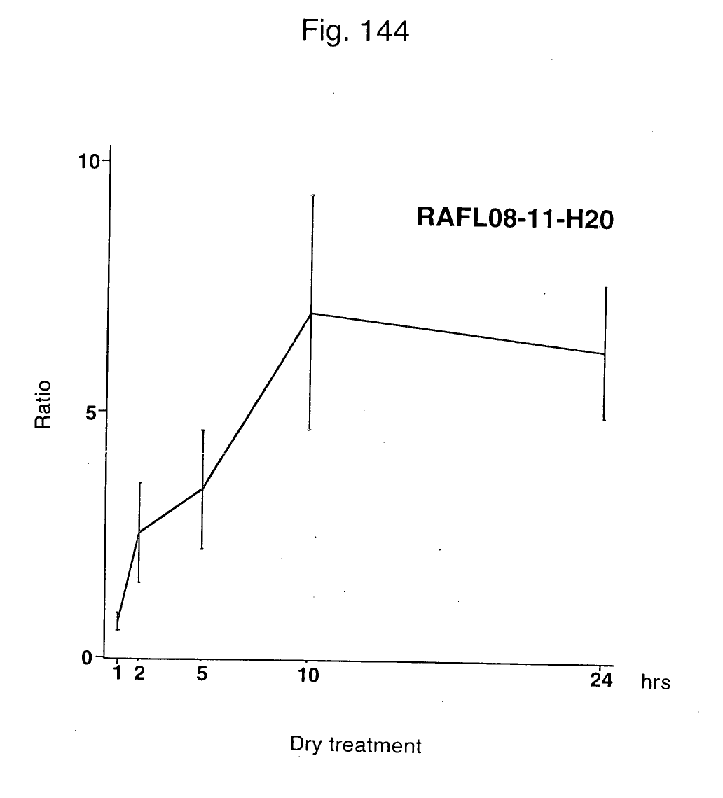

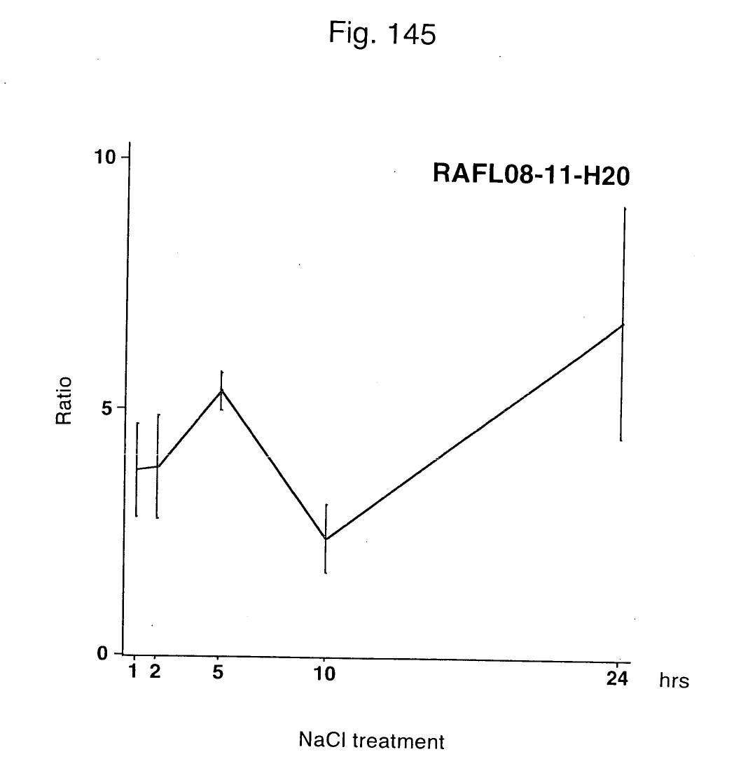

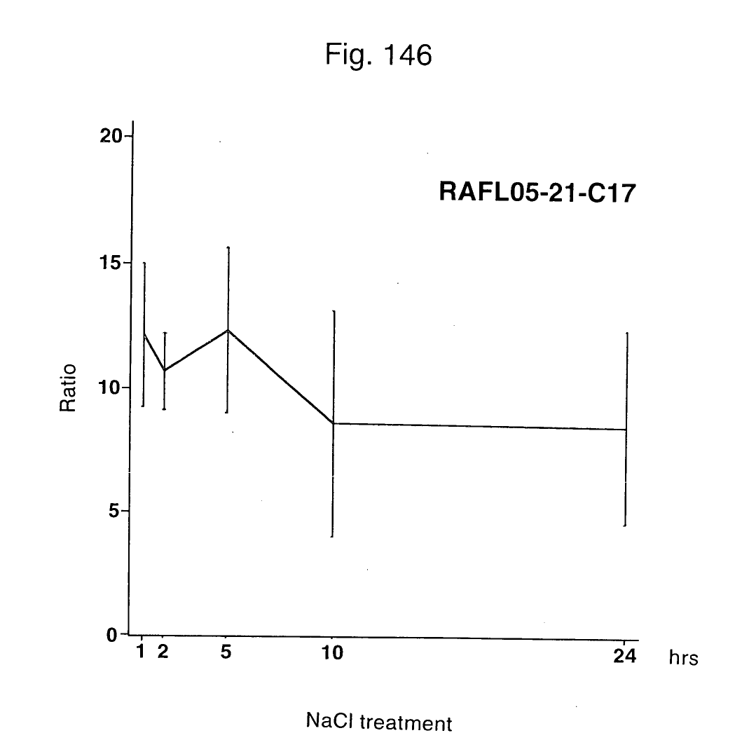

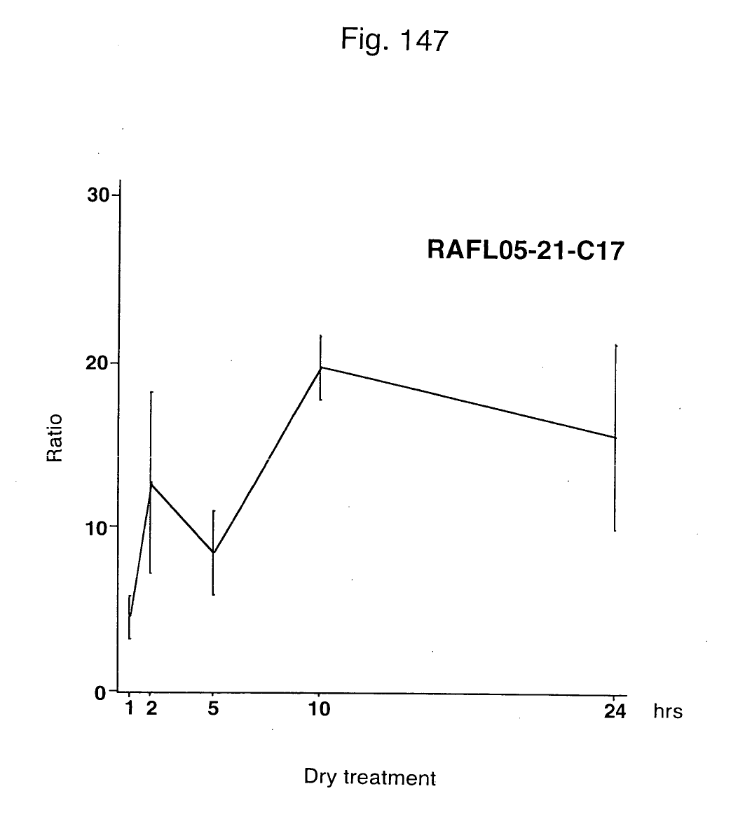

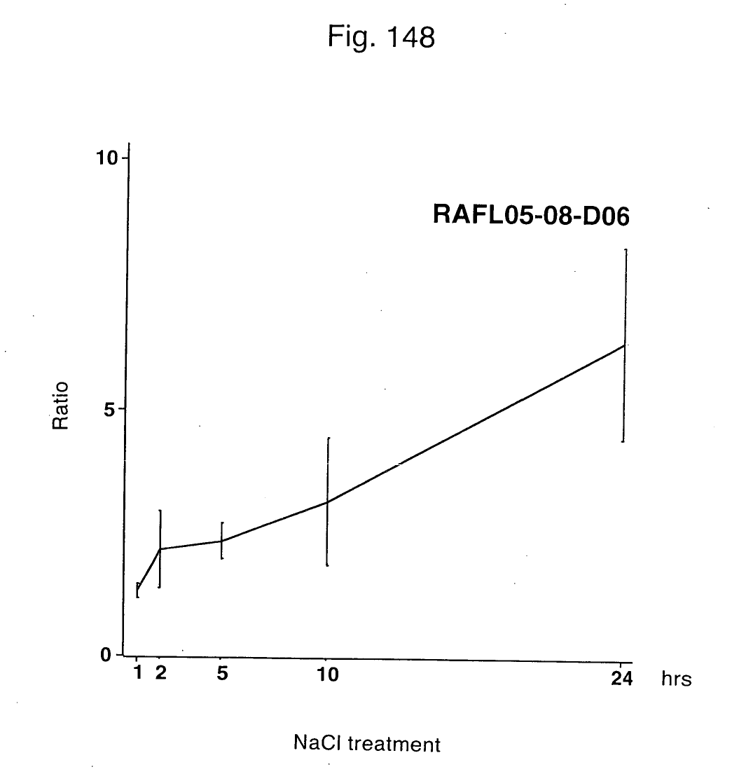

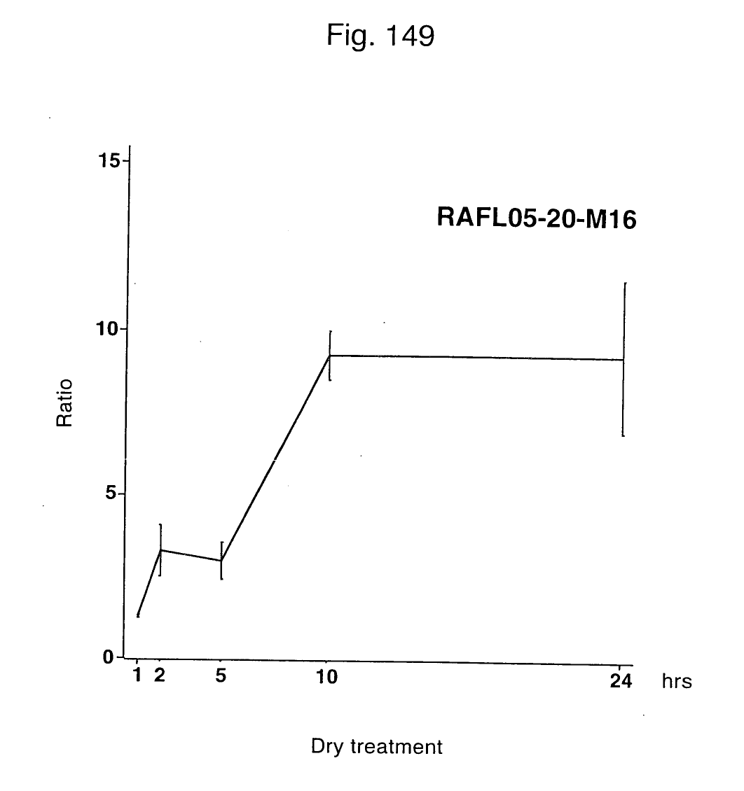

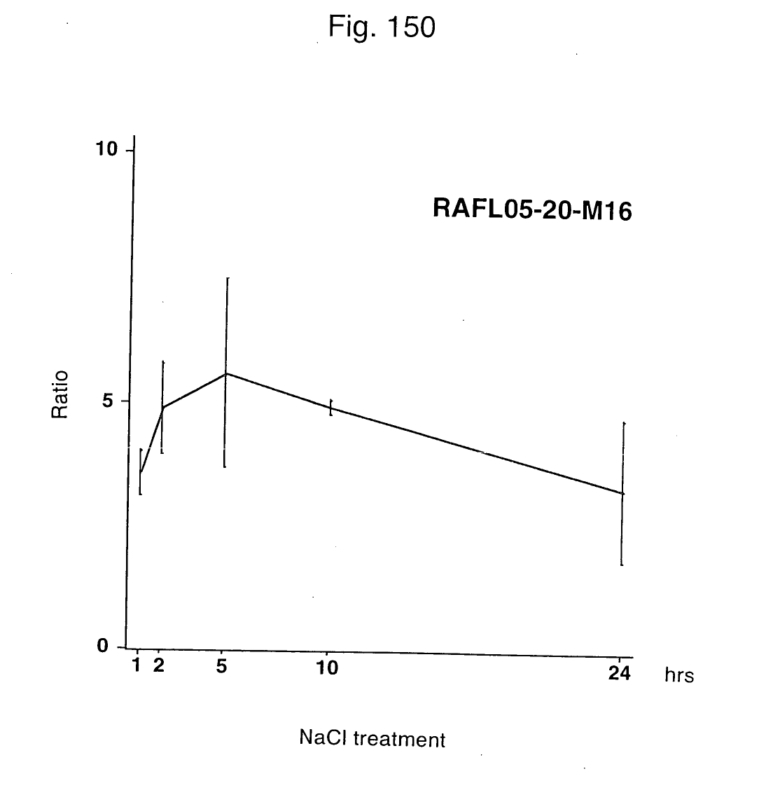

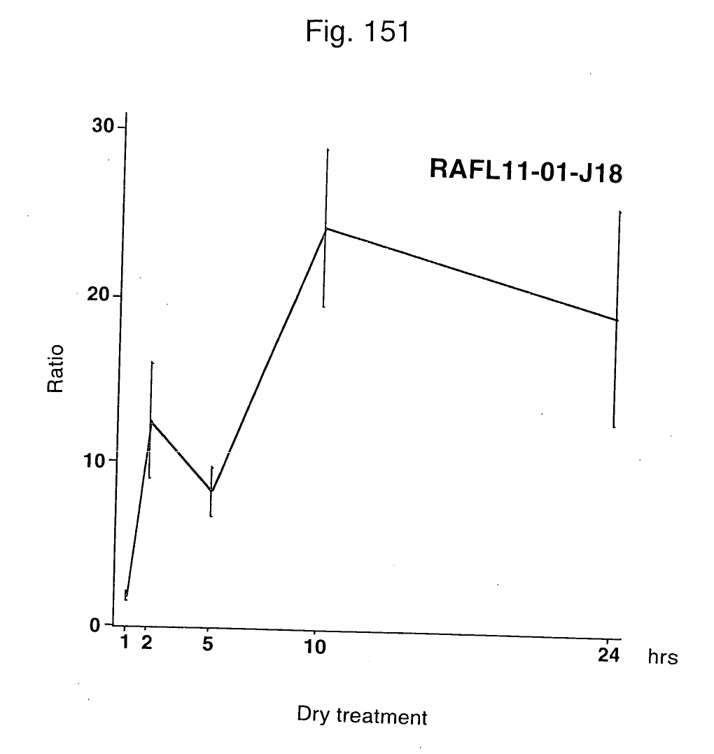

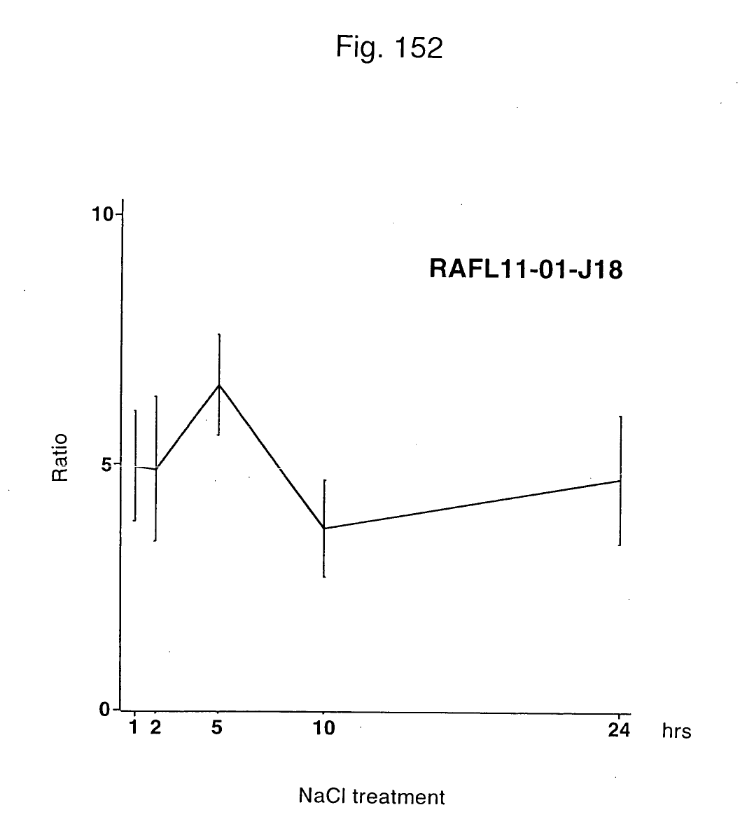

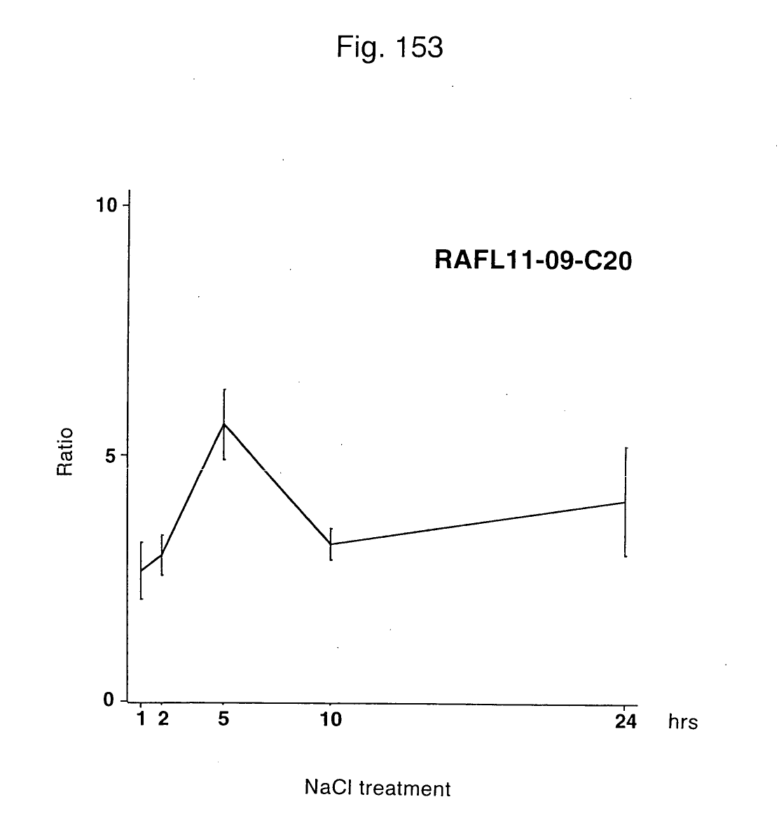

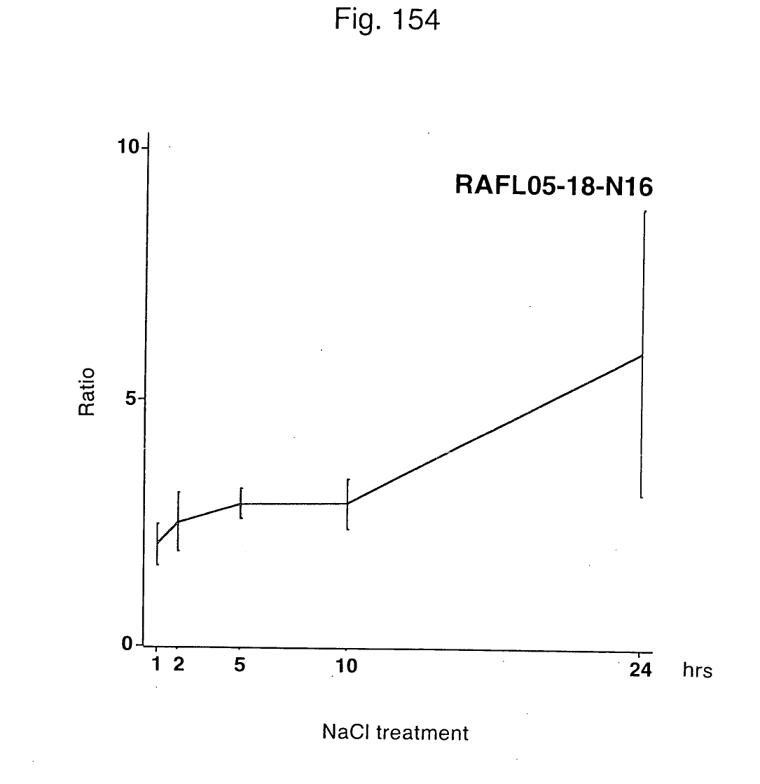

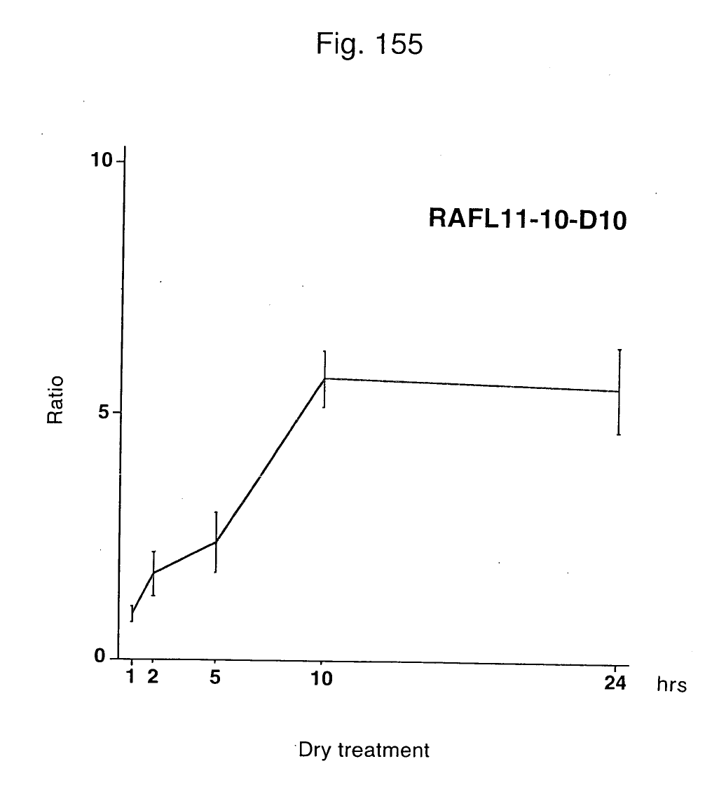

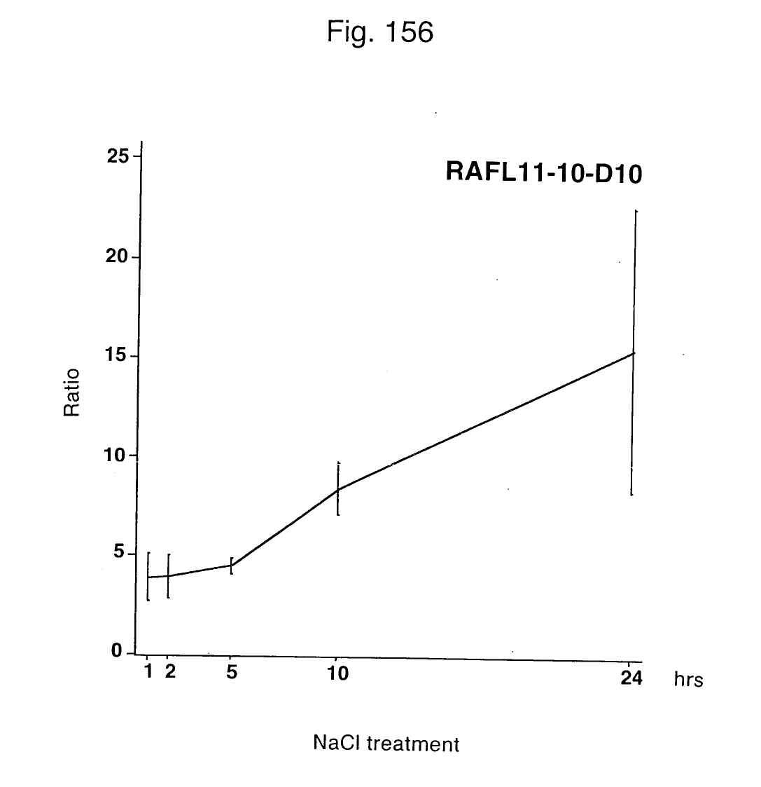

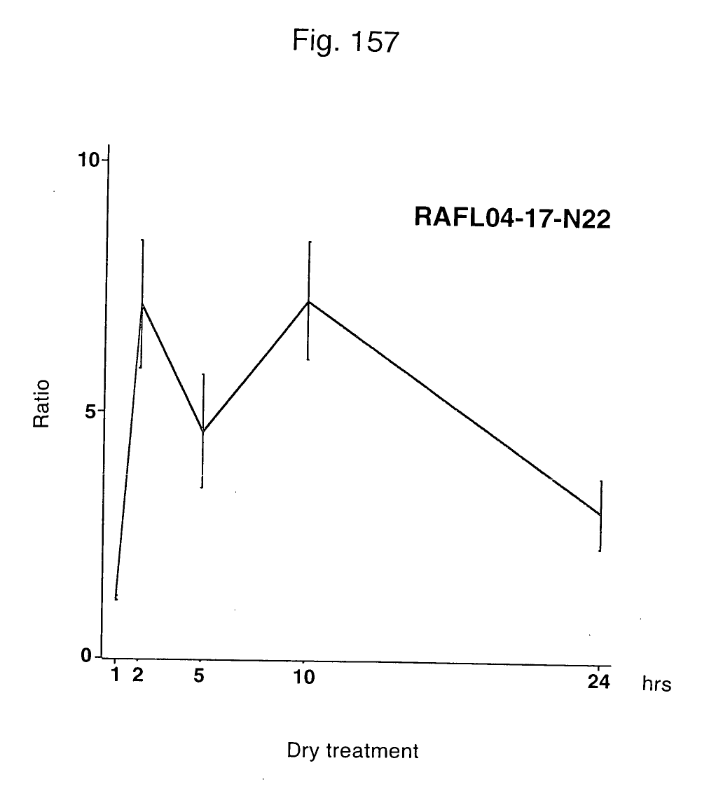

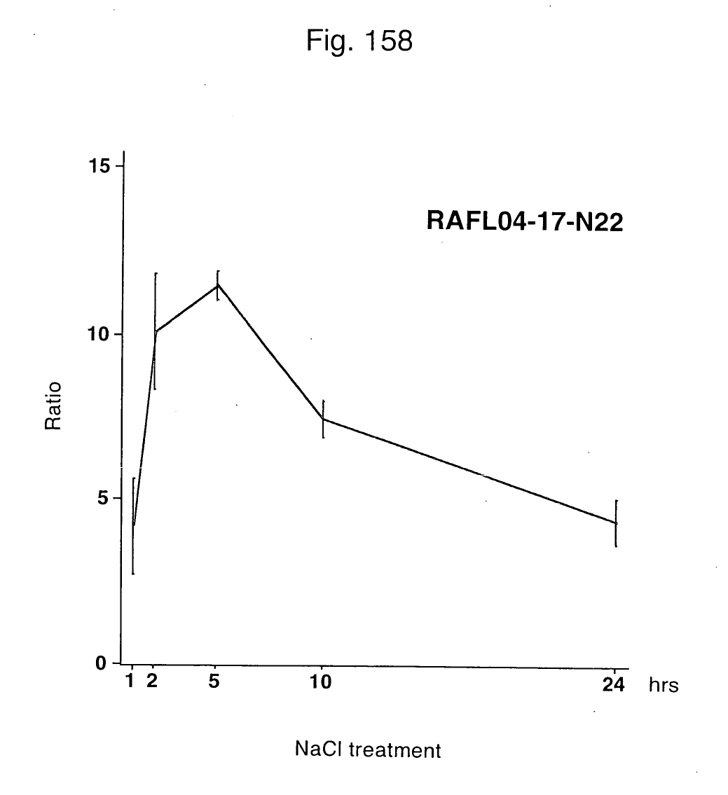

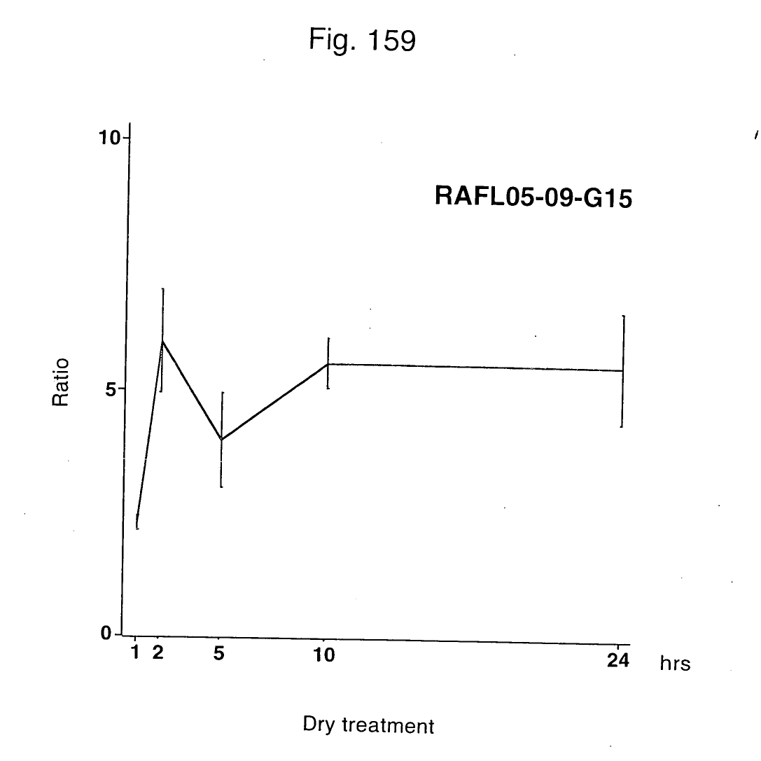

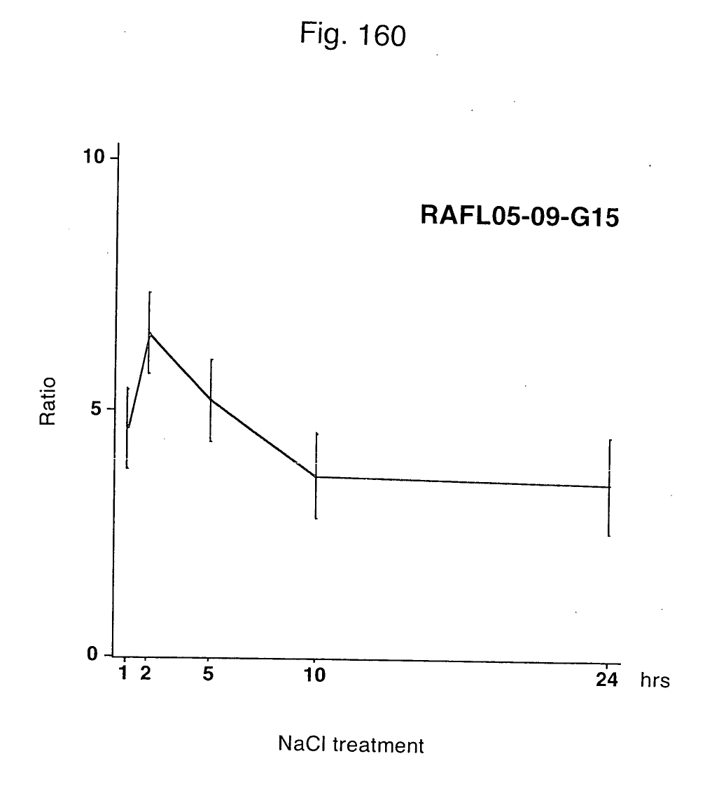

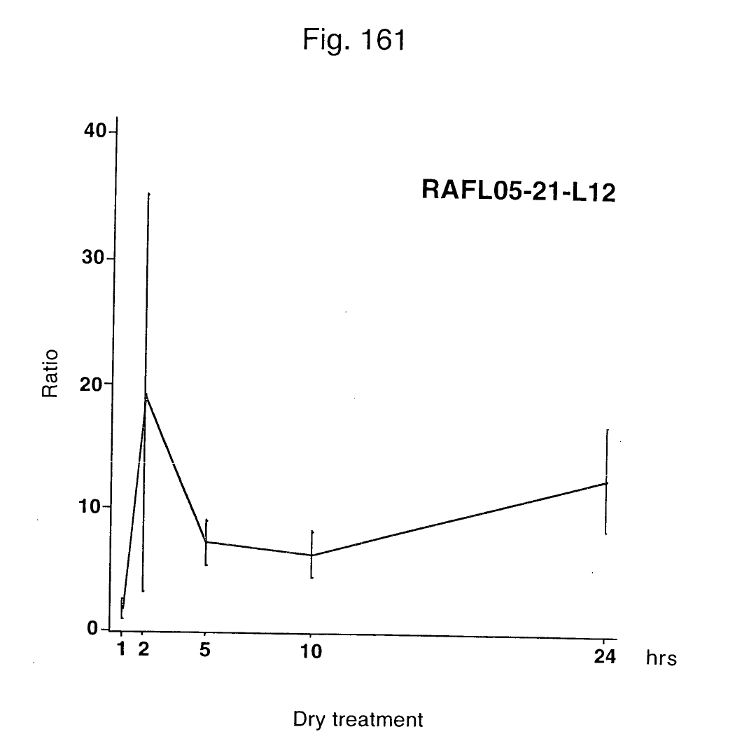

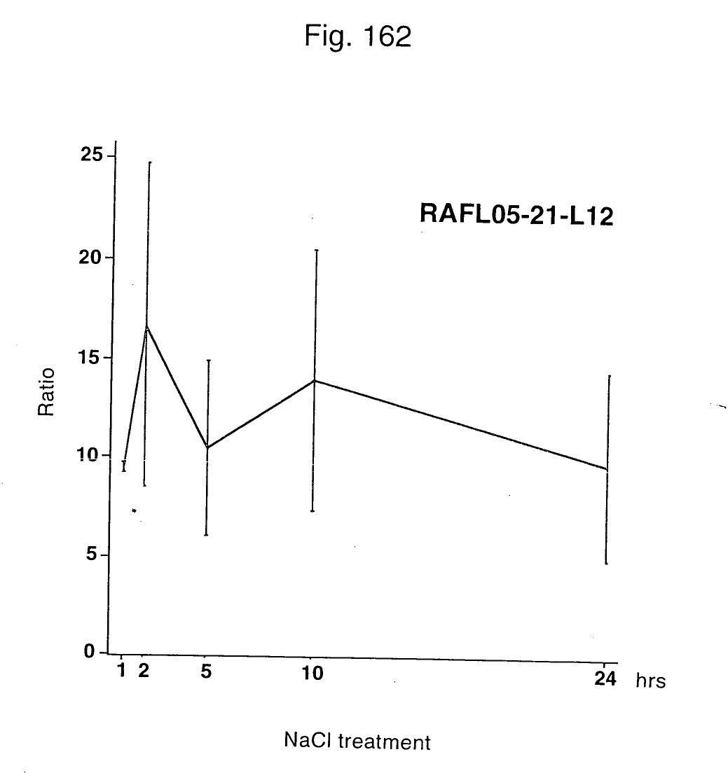

Genes encoding 36 types of stress responsive transcriptional factors isolated as described above were analyzed for the relationship between treatment time with each stress and expression ratio. The results are shown in FIGS. 106 to 162. The correspondence between the names of genes and stress treatment shown in FIGS. 106 to 162 is listed in Table 5.

TABLE 5

| Number of figure | Name of gene | Type of stress |
|---|---|---|
| | RAFL08-16-G17 | High level salt solution |
| | RAFL05-11-M11 | Dehydration |
| | RAFL05-11-M11 | High level salt solution |
| | RAFL06-11-K21 | High level salt solution |
| | RAFL06-11-K21 | Dehydration |
| | RAFL06-08-H20 | Dehydration |
| | RAFL06-08-H20 | High level salt solution |
| | RAFL05-16-H23 | High level salt solution |
| | RAFL05-16-H23 | Dehydration |
| | RAFL08-16-D06 | Dehydration |
| | RAFL07-10-G04 | Dehydration |
| | RAFL04-17-D16 | Dehydration |
| | RAFL05-19-M20 | Dehydration |
| | RAFL08-11-M13 | High level salt solution |
| | RAFL04-15-K19 | Dehydration |
| | RAFL04-15-K19 | Cold |
| | RAFL05-11-L01 | Dehydration |
| | RAFL05-11-L01 | High level salt solution |
| | RAFL05-14-C11 | Dehydration |
| | RAFL05-19-G24 | High level salt solution |
| | RAFL05-19-G24 | Dehydration |
| | RAFL05-19-G24 | Cold |
| | RAFL05-20-N02 | Dehydration |
| | RAFL05-18-H12 | Dehydration |
| | RAFL05-18-H12 | High level salt solution |
| | RAFL05-19-E19 | High level salt solution |
| | RAFL06-10-D22 | High level salt solution |
| | RAFL06-12-M01 | High level salt solution |
| | RAFL06-12-M01 | Dehydration |
| | RAFL05-14-D24 | Dehydration |
| | RAFL05-14-D24 | High level salt solution |
| | RAFL05-20-N17 | Cold |
| | RAFL05-20-N17 | Dehydration |
| | RAFL04-17-F21 | Dehydration |
| | RAFL09-12-N16 | Dehydration |
| | RAFL05-19-I05 | Dehydration |
| | RAFL05-19-I05 | High level salt solution |
| | RAFL05-21-I22 | High level salt solution |
| | RAFL08-11-H20 | Dehydration |
| | RAFL08-11-H20 | High level salt solution |
| | RAFL05-21-C17 | High level salt solution |
| | RAFL05-21-C17 | Dehydration |
| | RAFL05-08-D06 | High level salt solution |
| | RAFL05-20-M16 | Dehydration |
| | RAFL05-20-M16 | High level salt solution |
| | RAFL11-01-J18 | Dehydration |
| | RAFL11-01-J18 | High level salt solution |
| | RAFL11-09-C20 | High level salt solution |
| | RAFL05-18-N16 | High level salt solution |
| | RAFL11-10-D10 | Dehydration |
| | RAFL11-10-D10 | High level salt solution |
| | RAFL04-17-N22 | Dehydration |
| | RAFL04-17-N22 | High level salt solution |
| | RAFL05-09-G15 | Dehydration |
| | RAFL05-09-G15 | High level salt solution |
| | RAFL05-21-L12 | Dehydration |
| | RAFL05-21-L12 | High level salt solution |

In FIGS. 106 to 162, the vertical axis shows the expression ratio of a gene, which is calculated as follows:
Expression ratio=[(FI of cDNA molecule under stress)/(FI of cDNA molecule under no stress)]÷[(FI of control fragment under stress)/(FI of control fragment under no stress)]
where FI is the intensity of fluorescence.

As shown in FIGS. 106 to 162, the genes encoding stress responsive transcriptional factors isolated by a method according to the present invention exhibit different profiles; however, it is found that expression is induced by adding each stress.

### INDUSTRIAL APPLICABILITY

A stress responsive promoter and an environmental stress responsive transcriptional factor are provided by the present invention. The promoter of the present invention is useful in that it can be used for breeding of environmental stress resistant plants in a molecular level.

Sequencing Free Text

SEQ ID NOS: 91, 92, 165 and 166 are synthetic primers.

1. An environmental stress-responsive promoter, comprising:
(a) DNA consisting of any nucleotide sequence selected from SEQ ID NOS: 1 to 90;
(b) DNA consisting of a nucleotide sequence comprising a deletion, substitution or addition of one or more nucleotides relative to any nucleotide sequence selected from SEQ ID NOS: 1 to 90, and functioning as an environmental stress responsive promoter; and
(c) DNA hybridizing under stringent conditions to DNA consisting of any nucleotide sequence selected from SEQ ID NOS: 1 to 90, and functioning as an environmental stress responsive promoter.

2. The promoter according to claim 1, wherein the environmental stress is at least one selected from the group consisting of cold stress, drought stress, and salt stress.

3. An expression vector comprising a promoter according either one of claims 1 and 2.

4. An expression vector according to claim 3, further comprising an arbitrary gene incorporated therein.

5. A transformant comprising the expression vector according to claim 3.

6. A transgenic plant comprising the expression vector according to claim 3.

7. The transgenic plant according to claim 6, wherein the plant is a plant body, plant organ, plant tissue or plant culture cell.

8. A method for producing a stress-resistant plant, comprising culturing or cultivating the transgenic plant according to claim 6.

9. A gene encoding an environmental stress-responsive transcriptional factor comprising:
(a) any amino acid sequence selected from SEQ ID NOS: 2n (n is an integer from 47 to 82);
(b) an amino acid sequence comprising a deletion, substitution or addition of one or more amino acids relative to any amino acid sequence selected from SEQ ID NOS: 2n (n is an integer from 47 to 82), functioning as an environmental stress responsive promoter, and having a transcriptional-factor activating activity.

10. The gene according to claim 1, comprising:
(a) DNA consisting of any nucleotide sequence selected from SEQ ID NOS: 2n−1 (n is an integer from 47 to 82);
(b) DNA consisting of a nucleotide sequence comprising a deletion, substitution or addition of one or more nucleotides relative to any nucleotide sequence selected from SEQ ID NOS: 2n−1 (n is an integer from 47 to 82), and encoding an environmental stress responsive promoter; and
(c) DNA hybridizing under stringent conditions to DNA consisting of any nucleotide sequence selected from SEQ ID NOS: 2n−1 (n is an integer from 47 to 82), and encoding an environmental stress responsive promoter.

11. The gene according to claim 9 or 10, wherein the environmental stress is at least one selected from the group consisting of cold stress, drought stress, and salt stress.

12. An expression vector comprising a gene according to claim 9 or 10.

13. A transformant comprising the expression vector according to claim 12.

14. A transgenic plant comprising the expression vector according to claim 12.

15. The transgenic plant according to claim 14, wherein the plant is a plant body, plant organ, plant tissue or plant culture cell.

16. A method for producing a stress-resistant plant, comprising culturing or cultivating the transgenic plant according to claim 14.
